(19) United States
(12) Patent Application Publication
(10) Pub. No.: US 2024/0343681 A1
(43) Pub. Date: Oct. 17, 2024

(54) MOLECULES HAVING PESTICIDAL UTILITY AND INTERMEDIATES AND PROCESSES RELATED THERETO

(71) Applicant: CORTEVA AGRISCIENCE LLC, INDIANAPOLIS, IN (US)

(72) Inventors: JOSEPH D. ECKELBARGER, CARMEL, IN (US); RONALD J. HEEMSTRA, FISHERS, IN (US); RICKY HUNTER, WESTFIELD, IN (US); DANIEL KNUEPPEL, ZIONSVILLE, IN (US); TIMOTHY P. MARTIN, NOBLESVILLE, IN (US); TONY K. TRULLINGER, WESTFIELD, IN (US); MARTIN J. WALSH, CARMEL, IN (US)

(73) Assignee: CORTEVA AGRISCIENCE LLC, INDIANAPOLIS, IN (US)

(21) Appl. No.: 18/551,238

(22) PCT Filed: Mar. 22, 2022
(86) PCT No.: PCT/US2022/021254

§ 371 Date: Sep. 19, 2023

Related U.S. Application Data
(60) Provisional application No. 63/164,589, filed on Mar. 23, 2021.

(51) Int. Cl. C07C 233/62; A01N 53/00; C07D 207/16; C07D 239/28; C07D 261/12; C07D 277/56; C07D 307/68

(52) U.S. Cl. CPC C07C 233/62; A01N 53/00; C07D 207/16; C07D 239/28; C07D 261/12; C07D 277/56; C07D 307/68

(57) ABSTRACT

This disclosure relates to the field of molecules having pesticidal utility against pests in Phyla Arthropoda, Mollusca, and Nematoda, processes to produce such molecules, intermediates used in such processes, pesticidal compositions containing such molecules, and processes of using such pesticidal compositions against such pests. These pesticidal compositions may be used, for example, as acaricides, insecticides, miticides, molluscicides, and nematicides. This document discloses molecules having the following formula (Formula One).

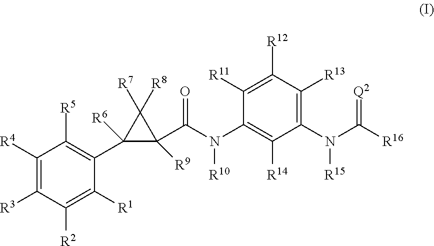

### CROSS REFERENCE TO RELATED TO APPLICATIONS

This application claims priority to International (PCT) Patent Application Serial No. PCT/US2022/021254, filed Mar. 22, 2022, and entitled “MOLECULES HAVING PESTICIDAL UTILITY AND INTERMEDIATES AND PROCESSES RELATED THERETO”, which claims priority to and the benefit of U.S. Provisional Patent Application Ser. No. 63/164,589 filed Mar. 23, 2021, the complete disclosures of which are expressly incorporated by reference herein.

### BACKGROUND FOR THIS DISCLOSURE

This disclosure relates to the field of molecules having pesticidal utility against pests in Phyla Arthropoda, Mollusca, and Nematoda, processes to produce such molecules, intermediates used in such processes, and processes of using such pesticidal compositions against such pests. These pesticidal compositions may be used, for example, as acaricides, insecticides, miticides, molluscicides, and nematicides.

“Many of the most dangerous human diseases are transmitted by insect vectors” (Rivero et al., Insect Control of Vector-Borne Diseases: When is Insect Resistance a Problem?Public Library of Science Pathogens, Vol. 6, No. 8, p. 1-9, 2010). “Historically, malaria, dengue, yellow fever, plague, filariasis, louse-borne typhus, trypanomiasis, leishmaniasis, and other vector borne diseases were responsible for more human disease and death in the 17^{th} through the early 20^{th} centuries than all other causes combined” (Gubler, D., Resurgent Vector-Borne Diseases as a Global Health Problem, Emerging Infectious Diseases, Vol. 4, No. 3, p. 442-450, 1998). Vector-borne diseases are responsible for about 17% of the global parasitic and infectious diseases. Malaria alone causes over 800,000 deaths a year, 85% of which occur in children under five years of age. Each year there are about 50 to about 100 million cases of dengue fever. A further 250,000 to 500,000 cases of dengue hemorrhagic fever occur each year (Matthews., Integrated Vector Management: Controlling Vectors of Malaria and Other Insect Vector Borne Diseases, Ch. 1, p. 1, 2011). Vector control plays a critical role in the prevention and control of infectious diseases. However, insecticide resistance, including resistance to multiple insecticides, has arisen in all insect species that are major vectors of human diseases (Rivero et al.). Recently, more than 550 arthropod species have developed resistance to at least one pesticide (Whalon et al., Analysis of Global Pesticide Resistance in Arthropods, Global Pesticide Resistance in Arthropods, Ch. 1, p. 5-33, 2008). Furthermore, the cases of insect resistance continue to exceed by far the number of cases of herbicide and fungicide resistance (Sparks et al., IRAC: Mode of action classification and insecticide resistance management, Pesticide Biochemistry and Physiology (2014) available online 4 Dec. 2014).

Each year, insects, plant pathogens, and weeds destroy more than 40% of all food production. This loss occurs despite the application of pesticides and the use of a wide array of non-chemical controls, such as crop rotations and biological controls. If just some of this food could be saved, it could be used to feed the more than three billion people in the world who are malnourished (Pimental, D., Pest Control in World Agriculture, Agricultural Sciences—Vol. II, 2009).

Plant parasitic nematodes are among the most widespread pests and are frequently one of the most insidious and costly. It has been estimated that losses attributable to nematodes are from about 9% in developed countries to about 15% in undeveloped countries. However, in the United States of America a survey of 35 States on various crops indicated nematode-derived losses of up to 25% (Nicol et al., Current Nematode Threats to World Agriculture, Genomic and Molecular Genetics of Plant-Nematode Interactions, p. 21-43, 2011).

It is noted that gastropods (slugs and snails) are pests of less economic importance than other arthropods or nematodes, but in certain places, they may reduce yields substantially, severely affecting the quality of harvested products, as well as, transmitting human, animal, and plant diseases. While only a few dozen species of gastropods are serious regional pests, a handful of species are important pests on a worldwide scale. In particular, gastropods affect a wide variety of agricultural and horticultural crops, such as arable, pastoral, and fiber crops; vegetables; bush and tree fruits; herbs; and ornamentals (Speiser, B., Molluscicides, Encyclopedia of Pest Management, Ch. 219, p. 506-508, 2002).

Termites cause damage to all types of private and public structures, as well as to agricultural and forestry resources. In 2005, it was estimated that termites cause over US$50 billion in damage worldwide each year (Korb, J., Termites, Current Biology, Vol. 17, No. 23, 2007).

Consequently, for many reasons, including those mentioned above, there is an on-going need for the costly (estimated to be about US$286 million per pesticide in 2014), time-consuming (on average about 11.3 years per pesticide), and difficult, development of new pesticides (Phillips McDougall, The Cost of New Agrochemical Product Discovery, Development and Registration in 1995, 2000, 20005-8 and 2010-2014. R&D expenditure in 2014 and expectations for 2019, 2016).

### DEFINITIONS FOR THIS DISCLOSURE

Examples provided herein are not exhaustive and should not be construed as limiting. It is understood that a substituent should comply with chemical bonding rules and steric compatibility constraints in relation to the particular molecule to which it is attached. These definitions are only to be used for the purposes of this disclosure.

The term “alkenyl” means an acyclic, unsaturated (at least one carbon-carbon double bond), branched or unbranched, substituent consisting of carbon and hydrogen, for example, vinyl, allyl, butenyl, pentenyl, and hexenyl.

The term “alkoxy” means an alkyl further consisting of a carbon-oxygen single bond, for example, methoxy, ethoxy, propoxy, isopropoxy, butoxy, isobutoxy, and tert-butoxy.

The term “alkyl” means an acyclic, saturated, branched or unbranched, substituent consisting of carbon and hydrogen, for example, methyl, ethyl, propyl, isopropyl, butyl, and tert-butyl.

The term “alkynyl” means an acyclic, unsaturated (at least one carbon-carbon triple bond), branched or unbranched, substituent consisting of carbon and hydrogen, for example, ethynyl, propargyl, butynyl, and pentynyl.

The term “aryl” means a cyclic, aromatic substituent consisting of hydrogen and carbon, for example, phenyl, naphthyl, and biphenyl.

The term “cycloalkyl” means a monocyclic or polycyclic, saturated substituent consisting of carbon and hydrogen, for example, cyclopropyl, cyclobutyl, cyclopentyl, norbornyl, bicyclo[2.2.2]octyl, and decahydronaphthyl.

The term “halo” means fluoro, chloro, bromo, and iodo.

The term “haloalkyl” means an alkyl further consisting of, from one to the maximum possible number of, identical or different, halos, for example, fluoromethyl, trifluoromethyl, 2,2-difluoropropyl, chloromethyl, trichloromethyl, and 1,1,2,2-tetrafluoroethyl.

The term “heterocyclyl” means a cyclic substituent that may be aromatic, fully saturated, or partially or fully unsaturated, where the cyclic structure contains at least one carbon and at least one heteroatom, where said heteroatom is nitrogen, sulfur, or oxygen. Examples are:
(1) aromatic heterocyclyl substituents include, but are not limited to, benzofuranyl, benzoisothiazolyl, benzoisoxazolyl, benzothienyl, benzothiazolyl, benzoxazolyl, cinnolinyl, furanyl, imidazolyl, indazolyl, indolyl, isoindolyl, isoquinolinyl, isothiazolyl, isoxazolyl, oxadiazolyl, oxazolinyl, oxazolyl, phthalazinyl, pyrazinyl, pyrazolinyl, pyrazolyl, pyridazinyl, pyridyl, pyrimidinyl, pyrrolyl, quinazolinyl, quinolinyl, quinoxalinyl, tetrazolyl, thiazolinyl, thiazolyl, thienyl, triazinyl, and triazolyl;
(2) fully saturated heterocyclyl substituents include, but are not limited to, piperazinyl, piperidinyl, morpholinyl, pyrrolidinyl, tetrahydrofuranyl, and tetrahydropyranyl;
(3) partially or fully unsaturated heterocyclyl substituents include, but are not limited to, 4,5-dihydro-isoxazolyl, 4,5-dihydro-oxazolyl, 4,5-dihydro-1H-pyrazolyl, 2,3-dihydro-[1,3,4]-oxadiazolyl, and 1,2,3,4-tetrahydro-quinolinyl; and
(4) Additional examples of heterocyclyls include the following:

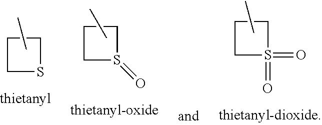

The term “ambient pressure” refers to pressures from about 80 kilopascals (kPa) to about 105 kPa.

The term “ambient temperature” or “room temperature” refers to temperatures ranging from about 20° C. to about 24° C.

The term “locus” means a habitat, breeding ground, plant, seed, soil, material, or environment, in which a pest is growing, may grow, or may traverse. For example, a locus may be: where crops, trees, fruits, cereals, fodder species, vines, turf, and/or ornamental plants, are growing; where domesticated animals are residing; the interior or exterior surfaces of buildings (such as places where grains are stored); the materials of construction used in buildings (such as impregnated wood); and the soil around buildings.

In this disclosure the terms “molecule” and “compound” may be used interchangeably.

The term “pest” means an organism that is detrimental to humans, or human concerns (such as, crops, food, livestock, etc.), where said organism is from Phyla Arthropoda, Mollusca, or Nematoda. Particular examples are ants, aphids, bed bugs, beetles, bristletails, caterpillars, cockroaches, crickets, earwigs, fleas, flies, grasshoppers, grubs, hornets, jassids, leafhoppers, lice, locusts, maggots, mealybugs, mites, moths, nematodes, plantbugs, planthoppers, psyllids, sawflies, scales, silverfish, slugs, snails, spiders, springtails, stink bugs, symphylans, termites, thrips, ticks, wasps, whiteflies, and wireworms.

Additional examples are pests in
(1) Subphyla Chelicerata, Myriapoda, and Hexapoda.
(2) Classes of Arachnida, Symphyla, and Insecta.
(3) Order Anoplura. A non-exhaustive list of particular genera includes, but is not limited to, Haematopinus spp., Hoplopleura spp., Linognathus spp., Pediculus spp., Polyplax spp., Solenopotes spp., and Neohaematopinis spp. A non-exhaustive list of particular species includes, but is not limited to, Haematopinus asini, Haematopinus suis, Linognathus setosus, Linognathus ovillus, Pediculus humanus capitis, Pediculus humanus humanus, and Pthirus pubis.
(4) Order Coleoptera. A non-exhaustive list of particular genera includes, but is not limited to, Acanthoscelides spp., Agriotes spp., Anthonomus spp., Apion spp., Apogonia spp., Araecerus spp., Aulacophora spp., Bruchus spp., Cerosterna spp., Cerotoma spp., Ceutorhynchus spp., Chaetocnema spp., Colaspis spp., Ctenicera spp., Curculio spp., Cyclocephala spp., Diabrotica spp., Dinoderus spp., Gnathocerus spp., Hemicoelus spp., Heterobostruchus spp., Hypera spp., Ips spp., Lyctus spp., Megascelis spp., Meligethes spp., Mezium spp., Niptus spp., Otiorhynchus spp., Pantomorus spp., Phyllophaga spp., Phyllotreta spp., Ptinus spp., Rhizotrogus spp., Rhynchites spp., Rhynchophorus spp., Scolytus spp., Sphenophorus spp., Sitophilus spp., Tenebrio spp., and Tribolium spp. A non-exhaustive list of particular species includes, but is not limited to, Acanthoscelides obtectus, Agrilus planipennis, Ahasverus advena, Alphitobius diaperinus, Anoplophora glabripennis, Anthonomus grandis, Anthrenus verbasci, Anthrenus falvipes, Ataenius spretulus, Atomaria linearis, Attagenus unicolor, Bothynoderes punctiventris, Bruchus pisorum, Callosobruchus maculatus, Carpophilus hemipterus, Cassida vittata, Cathartus quadricollis, Cerotoma trifurcata, Ceutorhynchus assimilis, Ceutorhynchus napi, Conoderus scalaris, Conoderus stigmosus, Conotrachelus nenuphar, Cotinis nitida, Crioceris asparagi, Cryptolestes ferrugineus, Cryptolestes pusillus, Cryptolestes turcicus, Cylindrocopturus adspersus, Deporaus marginatus, Dermestes lardarius, Dermestes maculatus, Epilachna varivestis, Euvrilletta peltata, Faustinus cubae, Hylobius pales, Hylotrupes bajulus, Hypera postica, Hypothenemus hampei, Lasioderma serricorne, Leptinotarsa decemlineata, Limonius canus, Liogenys fuscus, Liogenys suturalis, Lissorhoptrus oryzophilus, Lophocateres pusillus, Lyctus planicollis, Maecolaspis joliveti, Melanotus communis, Meligethes aeneus, Melolontha melolontha, Necrobia rufipes, Oberea brevis, Oberea linearis, Oryctes rhinoceros, Oryzaephilus mercator, Oryzaephilus surinamensis, Oulema melanopus, Oulema oryzae, Phyllophaga cuyabana, Polycaon stoutti, Popillia japonica, Prostephanus truncatus, Rhyzopertha dominica, Sitona lineatus, Sitophilus granarius, Sitophilus oryzae, Sitophilus zeamais, Stegobium paniceum, Tenebroides mauritanicus, Tribolium castaneum, Tribolium confusum, Trogoderma granarium, Trogoderma variabile, Xestobium rufovillosum, and Zabrus tenebrioides.
(5) Order Dermaptera. A non-exhaustive list of particular species includes, but is not limited to, Forficula auricularia.
(6) Order Blattaria. A non-exhaustive list of particular species includes, but is not limited to, Blattella germanica, Blattella asahinai, Blatta orientalis, Blatta lateralis, Parcoblatta pennsylvanica, Periplaneta americana, Periplaneta australasiae, Periplaneta brunnea, Periplaneta fuliginosa, Pycnoscelus surinamensis, and Supella longipalpa.
(7) Order Diptera. A non-exhaustive list of particular genera includes, but is not limited to, Aedes spp., Agromyza spp., Anastrepha spp., Anopheles spp., Bactrocera spp., Ceratitis spp., Chrysops spp., Cochlomyia spp., Contarinia spp., Culex spp., Culicoides spp., Dasineura spp., Delia spp., Drosophila spp., Fannia spp., Hylemya spp., Liriomyza spp., Musca spp., Phorbia spp., Pollenia spp., Psychoda spp., Simulium spp., Tabanus spp., and Tipula spp. A non-exhaustive list of particular species includes, but is not limited to, Agromyza frontella, Anastrepha suspensa, Anastrepha ludens, Anastrepha obliqua, Bactrocera cucurbitae, Bactrocera dorsalis, Bactrocera invadens, Bactrocera zonata, Ceratitis capitata, Dasineura brassicae, Delia platura, Fannia canicularis, Fannia scalaris, Gasterophilus intestinalis, Gracillia perseae, Haematobia irritans, Hypoderma lineatum, Liriomyza brassicae, Liriomyza sativa, Melophagus ovinus, Musca autumnalis, Musca domestica, Oestrus ovis, Oscinella frit, Pegomya betae, Piophila casei, Psila rosae, Rhagoletis cerasi, Rhagoletis pomonella, Rhagoletis mendax, Sitodiplosis mosellana, and Stomoxys calcitrans.
(8) Order Hemiptera. A non-exhaustive list of particular genera includes, but is not limited to, Adelges spp., Aulacaspis spp., Aphrophora spp., Aphis spp., Bemisia spp., Ceroplastes spp., Chionaspis spp., Chrysomphalus spp., Coccus spp., Empoasca spp., Euschistus spp., Lepidosaphes spp., Lagynotomus spp., Lygus spp., Macrosiphum spp., Nephotettix spp., Nezara spp., Nilaparvata spp., Philaenus spp., Phytocoris spp., Piezodorus spp., Planococcus spp., Pseudococcus spp., Rhopalosiphum spp., Saissetia spp., Therioaphis spp., Toumeyella spp., Toxoptera spp., Trialeurodes spp., Triatoma spp., and Unaspis spp. A non-exhaustive list of particular species includes, but is not limited to, Acrosternum hilare, Acyrthosiphon pisum, Aleyrodes proletella, Aleurodicus dispersus, Aleurothrixus floccosus, Amrasca biguttula biguttula, Aonidiella aurantii, Aphis fabae, Aphis gossypii, Aphis glycines, Aphis pomi, Aulacorthum solani, Bactericera cockerelli, Bagrada hilaris, Bemisia argentifolii, Bemisia tabaci, Blissus leucopterus, Boisea trivittata, Brachycorynella asparagi, Brevennia rehi, Brevicoryne brassicae, Cacopsylla pyri, Cacopsylla pyricola, Calocoris norvegicus, Ceroplastes rubens, Cimex hemipterus, Cimex lectularius, Coccus pseudomagnoliarum, Dagbertus fasciatus, Dichelops furcatus, Diuraphis noxia, Diaphorina citri, Dysaphis plantaginea, Dysdercus suturellus, Edessa meditabunda, Empoasca vitis, Eriosoma lanigerum, Erythroneura elegantula, Eurygaster maura, Euschistus conspersus, Euschistus heros, Euschistus servus, Halyomorpha halys, Helopeltis antonii, Hyalopterus pruni, Helopeltis antonii, Helopeltis theivora, Icerya purchasi, Idioscopus nitidulus, Jacobiasca formosana, Laodelphax striatellus, Lecanium corni, Leptocorisa oratorius, Leptocorisa varicornis, Lygus hesperus, Maconellicoccus hirsutus, Macrosiphum euphorbiae, Macrosiphum granarium, Macrosiphum rosae, Macrosteles quadrilineatus, Mahanarva frimbiolata, Megacopta cribraria, Metopolophium dirhodum, Mictis longicornis, Myzus persicae, Nasonovia ribisnigri, Nephotettix cincticeps, Neurocolpus longirostris, Nezara viridula, Nilaparvata lugens, Paracoccus marginatus, Paratrioza cockerelli, Parlatoria pergandii, Parlatoria ziziphi, Peregrinus maidis, Phylloxera vitifoliae, Physokermes piceae, Phytocoris ca/fornicus, Phytocoris relativus, Piezodorus guildinii, Planococcus citri, Planococcus fcus, Poecilocapsus lineatus, Psallus vaccinicola, Pseudacysta perseae, Pseudococcus brevipes, Quadraspidiotus perniciosus, Rhopalosiphum maidis, Rhopalosiphum padi, Saissetia oleae, Scaptocoris castanea, Schizaphis graminum, Sitobion avenae, Sogatella furcifera, Trialeurodes vaporariorum, Trialeurodes abutiloneus, Unaspis yanonensis, and Zulia entrerriana.
(9) Order Hymenoptera. A non-exhaustive list of particular genera includes, but is not limited to, Acromyrmex spp., Atta spp., Camponotus spp., Diprion spp., Dolichovespula spp., Formica spp., Monomorium spp., Neodiprion spp., Paratrechina spp., Pheidole spp., Pogonomyrmex spp., Polistes spp., Solenopsis spp., Technomyrmex, spp., Tetramorium spp., Vespula spp., Vespa spp., and Xylocopa spp. A non-exhaustive list of particular species includes, but is not limited to, Athalia rosae, Atta texana, Caliroa cerasi, Cimbex americana, Iridomyrmex humilis, Linepithema humile, Mellifera Scutellata, Monomorium minimum, Monomorium pharaonis, Neodiprion sertifer, Solenopsis invicta, Solenopsis geminata, Solenopsis molesta, Solenopsis richtery, Solenopsis xyloni, Tapinoma sessile, and Wasmannia auropunctata.
(10) Order Isoptera. A non-exhaustive list of particular genera includes, but is not limited to, Coptotermes spp., Cornitermes spp., Cryptotermes spp., Heterotermes spp., Kalotermes spp., Incisitermes spp., Macrotermes spp., Marginitermes spp., Microcerotermes spp., Procornitermes spp., Reticulitermes spp., Schedorhinotermes spp., and Zootermopsis spp. A non-exhaustive list of particular species includes, but is not limited to, Coptotermes acinaciformis, Coptotermes curvignathus, Coptotermes frenchi, Coptotermes formosanus, Coptotermes gestroi, Cryptotermes brevis, Heterotermes aureus, Heterotermes tenuis, Incisitermes minor, Incisitermes snyderi, Microtermes obesi, Nasutitermes corniger, Odontotermes formosanus, Odontotermes obesus, Reticulitermes banyulensis, Reticulitermes grassei, Reticulitermes flavipes, Reticulitermes hageni, Reticulitermes hesperus, Reticulitermes santonensis, Reticulitermes speratus, Reticulitermes tibialis, and Reticulitermes virginicus.
(11) Order Lepidoptera. A non-exhaustive list of particular genera includes, but is not limited to, Adoxophyes spp., Agrotis spp., Argyrotaenia spp., Cacoecia spp., Caloptilia spp., Chilo spp., Chrysodeixis spp., Colias spp., Crambus spp., Diaphania spp., Diatraea spp., Earias spp., Ephestia spp., Epimecis spp., Feltia spp., Gortyna spp., Helicoverpa spp., Heliothis spp., Indarbela spp., Lithocolletis spp., Loxagrotis spp., Malacosoma spp., Nemapogon spp., Peridroma spp., Phyllonorycter spp., Pseudaletia spp., Plutella spp., Sesamia spp., Spodoptera spp., Synanthedon spp., and Yponomeuta spp. A non-exhaustive list of particular species includes, but is not limited to, Achaea janata, Adoxophyes orana, Agrotis ipsilon, Alabama argillacea, Amorbia cuneana, Amyelois transitella, Anacamptodes defectaria, Anarsia lineatella, Anomis sabulfera, Anticarsia gemmatalis, Archips argyrospila, Archips rosana, Argyrotaenia citrana, Autographa gamma, Bonagota cranaodes, Borbo cinnara, Bucculatrix thurberiella, Capua reticulana, Carposina niponensis, Chlumetia transversa, Choristoneura rosaceana, Cnaphalocrocis medinalis, Conopomorpha cramerella, Corcyra cephalonica, Cossus cossus, Cydia caryana, Cydia funebrana, Cydia molesta, Cydia nigricana, Cydia pomonella, Darna diducta, Diaphania nitidalis, Diatraea saccharalis, Diatraea grandiosella, Earias insulana, Earias vittella, Ecdytolopha aurantianum, Elasmopalpus lignosellus, Ephestia cautella, Ephestia elutella, Ephestia kuehniella, Epinotia aporema, Epiphyas postvittana, Erionota thrax, Estigmene acrea, Eupoecilia ambiguella, Euxoa auxiliaris, Galleria mellonella, Grapholita molesta, Hedylepta indicata, Helicoverpa armigera, Helicoverpa zea, Heliothis virescens, Hellula undalis, Keiferia lycopersicella, Leucinodes orbonalis, Leucoptera coffeella, Leucoptera mahfoliella, Lobesia botrana, Loxagrotis albicosta, Lymantria dispar, Lyonetia clerkella, Mahasena corbetti, Mamestra brassicae, Manduca sexta, Maruca testulalis, Metisa plana, Mythimna unipuncta, Neoleucinodes elegantalis, Nymphula depunctalis, Operophtera brumata, Ostrinia nubilalis, Oxydia vesulia, Pandemis cerasana, Pandemis heparana, Papilio demodocus, Pectinophora gossypiella, Peridroma saucia, Perileucoptera coffeella, Phthorimaea operculella, Phyllocnistis citrella, Phyllonorycter blancardella, Pieris rapae, Plathypena scabra, Platynota idaeusalis, Plodia interpunctella, Plutella xylostella, Polychrosis viteana, Prays endocarpa, Prays oleae, Pseudaletia unipuncta, Pseudoplusia includens, Rachiplusia nu, Scirpophaga incertulas, Sesamia inferens, Sesamia nonagrioides, Setora nitens, Sitotroga cerealella, Sparganothis pilleriana, Spodoptera exigua, Spodoptera frugiperda, Spodoptera eridania, Thecla basilides, Tinea pellionella, Tineola bisselliella, Trichoplusia ni, Tuta absoluta, Zeuzera coffeae, and Zeuzea pyrina.
(12) Order Mallophaga. A non-exhaustive list of particular genera includes, but is not limited to, Anaticola spp., Bovicola spp., Chelopistes spp., Goniodes spp., Menacanthus spp., and Trichodectes spp. A non-exhaustive list of particular species includes, but is not limited to, Bovicola bovis, Bovicola caprae, Bovicola ovis, Chelopistes meleagridis, Goniodes dissimilis, Goniodes gigas, Menacanthus stramineus, Menopon gallinae, and Trichodectes canis.
(13) Order Orthoptera. A non-exhaustive list of particular genera includes, but is not limited to, Melanoplus spp. and Pterophylla spp. A non-exhaustive list of particular species includes, but is not limited to, Acheta domesticus, Anabrus simplex, Gryllotalpa africana, Gryllotalpa australis, Gryllotalpa brachyptera, Gryllotalpa hexadactyla, Locusta migratoria, Microcentrum retinerve, Schistocerca gregaria, and Scudderia furcata.
(14) Order Psocoptera. A non-exhaustive list of particular species includes, but is not limited to, Liposcelis decolor, Liposcelis entomophila, Lachesilla quercus, and Trogium pulsatorium.
(15) Order Siphonaptera. A non-exhaustive list of particular species includes, but is not limited to, Ceratophyllus gallinae, Ceratophyllus niger, Ctenocephalides canis, Ctenocephalides felis, and Pulex irritans.
(16) Order Thysanoptera. A non-exhaustive list of particular genera includes, but is not limited to, Caliothrips spp., Frankliniella spp., Scirtothrips spp., and Thrips spp. A non-exhaustive list of particular species includes, but is not limited to, Caliothrips phaseoli, Frankliniella bispinosa, Frankliniella fusca, Frankliniella occidentalis, Frankliniella schultzei, Frankliniella tritici, Frankliniella williamsi, Heliothrips haemorrhoidalis, Rhipiphorothrips cruentatus, Scirtothrips citri, Scirtothrips dorsalis, Taeniothrips rhopalantennalis, Thrips hawaiiensis, Thrips nigropilosus, Thrips orientalis, Thrips palmi, and Thrips tabaci.
(17) Order Thysanura. A non-exhaustive list of particular genera includes, but is not limited to, Lepisma spp. and Thermobia spp.
(18) Order Acarina. A non-exhaustive list of particular genera includes, but is not limited to, Acarus spp., Aculops spp., Argus spp., Boophilus spp., Demodex spp., Dermacentor spp., Epitrimerus spp., Eriophyes spp., Ixodes spp., Oligonychus spp., Panonychus spp., Rhizoglyphus spp., and Tetranychus spp. A non-exhaustive list of particular species includes, but is not limited to, Acarapis woodi, Acarus siro, Aceria mangiferae, Aculops lycopersici, Aculus pelekassi, Aculus schlechtendali, Amblyomma americanum, Brevipalpus obovatus, Brevipalpus phoenicis, Dermacentor variabilis, Dermatophagoides pteronyssinus, Eotetranychus carpini, Liponyssoides sanguineus, Notoedres cati, Oligonychus coffeae, Oligonychus ilicis, Ornithonyssus bacoti, Panonychus citri, Panonychus ulmi, Phyllocoptruta oleivora, Polyphagotarsonemus latus, Rhipicephalus sanguineus, Sarcoptes scabiei, Tegolophus perseaflorae, Tetranychus urticae, Tyrophagus longior, and Varroa destructor.
(19) Order Araneae. A non-exhaustive list of particular genera includes, but is not limited to, Loxosceles spp., Latrodectus spp., and Atrax spp. A non-exhaustive list of particular species includes, but is not limited to, Loxosceles reclusa, Latrodectus mactans, and Atrax robustus.
(20) Class Symphyla. A non-exhaustive list of particular species includes, but is not limited to, Scutigerella immaculata.
(21) Subclass Collembola. A non-exhaustive list of particular species includes, but is not limited to, Bourletiella hortensis, Onychiurus armatus, Onychiurus fimetarius, and Sminthurus viridis.
(22) Phylum Nematoda. A non-exhaustive list of particular genera includes, but is not limited to, Aphelenchoides spp., Belonolaimus spp., Criconemella spp., Ditylenchus spp., Globodera spp., Heterodera spp., Hirschmanniella spp., Hoplolaimus spp., Meloidogyne spp., Pratylenchus spp., and Radopholus spp. A non-exhaustive list of particular species includes, but is not limited to, Dirofilaria immitis, Globodera pallida, Heterodera glycines, Heterodera zeae, Meloidogyne incognita, Meloidogyne javanica, Onchocerca volvulus, Pratylenchus penetrans, Radopholus similis, and Rotylenchulus reniformis.
(23) Phylum Mollusca. A non-exhaustive list of particular species includes, but is not limited to, Arion vulgaris, Cornu aspersum, Deroceras reticulatum, Limax flavus, Milax gagates, and Pomacea canaliculata.

A particularly preferred pest group to control is sap-feeding pests. Sap-feeding pests, in general, have piercing and/or sucking mouthparts and feed on the sap and inner plant tissues of plants. Examples of sap-feeding pests of particular concern to agriculture include, but are not limited to, aphids, leafhoppers, moths, scales, thrips, psyllids, mealybugs, stinkbugs, and whiteflies. Specific examples of Orders that have sap-feeding pests of concern in agriculture include but are not limited to, Anoplura and Hemiptera. Specific examples of Hemiptera that are of concern in agriculture include, but are not limited to, Aulacaspis spp., Aphrophora spp., Aphis spp., Bemisia spp., Coccus spp., Euschistus spp., Lygus spp., Macrosiphum spp., Nezara spp., and Rhopalosiphum spp.

Another particularly preferred pest group to control is chewing pests. Chewing pests, in general, have mouthparts that allow them to chew on the plant tissue including roots, stems, leaves, buds, and reproductive tissues (including, but not limited to flowers, fruit, and seeds). Examples of chewing pests of particular concern to agriculture include, but are not limited to, caterpillars, beetles, grasshoppers, and locusts. Specific examples of Orders that have chewing pests of concern in agriculture include but are not limited to, Coleoptera and Lepidoptera. Specific examples of Coleoptera that are of concern in agriculture include, but are not limited to, Anthonomus spp., Cerotoma spp., Chaetocnema spp., Colaspis spp., Cyclocephala spp., Diabrotica spp., Hypera spp., Phyllophaga spp., Phyllotreta spp., Sphenophorus spp., Sitophilus spp.

The phrase “pesticidally effective amount” means the amount of a pesticide needed to achieve an observable effect on a pest, for example, the effects of necrosis, death, retardation, prevention, removal, destruction, or otherwise diminishing the occurrence and/or activity of a pest in a locus. This effect may come about when pest populations are repulsed from a locus, pests are incapacitated in, or around, a locus, and/or pests are exterminated in, or around, a locus. Of course, a combination of these effects can occur. Generally, pest populations, activity, or both are desirably reduced more than fifty percent, preferably more than 90 percent, and most preferably more than 99 percent. In general, a pesticidally effective amount, for agricultural purposes, is from about 0.0001 grams per hectare to about 5000 grams per hectare, preferably from about 0.0001 grams per hectare to about 500 grams per hectare, and it is even more preferably from about 0.0001 grams per hectare to about 50 grams per hectare.

All references, including publications, patent applications, and patents, referred to herein are incorporated by reference herein to the same extent as if each reference were individually and specifically indicated to be incorporated by reference and were set forth in its entirety.

The use of the terms “a” and “an” and “the” and “at least one” and similar referents in the context of this disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the disclosure and does not pose a limitation on the scope of the disclosure unless otherwise claimed. No language in the specification should be construed as indicating any non-claimed element as essential to the practice of any embodiment herein.

### DETAILED DESCRIPTION FOR THIS DISCLOSURE

This document discloses molecules of Formula One

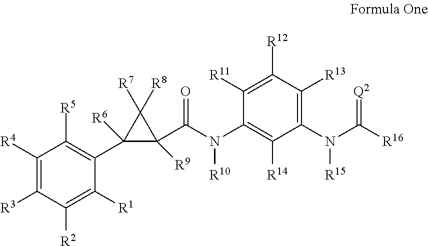

wherein:
(A) R¹ is selected from the group consisting of H, F, Cl, Br, I, and (C₁-C₃)haloalkyl;
(B) R² is selected from the group consisting of H, F, Cl, Br, I, and (C₁-C₃)haloalkyl;
(C) R³ is selected from the group consisting of H, F, Cl, Br, I, and (C₁-C₃)haloalkyl;
(D) R⁴ is selected from the group consisting of H, F, Cl, Br, I, and (C₁-C₃)haloalkyl;
(E) R⁵ is selected from the group consisting of H, F, Cl, Br, I, and (C₁-C₃)haloalkyl;
(F) R⁶ is H;
(G) R⁷ is selected from the group consisting of F, Cl, and Br;
(H) R⁸ is selected from the group consisting of F, Cl, and Br;
(I) R⁹ is H;
(J) Q¹ is selected from the group consisting of O and S;
(K) Q² is selected from the group consisting of O and S;
(L) R¹⁰ is selected from the group consisting of H, (C₁-C₃)alkyl, (C₂-C₃)alkenyl, and (C₂-C₃)alkynyl;
(M) R¹¹ is selected from the group consisting of H, F, Cl, Br, I, (C₁-C₃)alkyl, (C₁-C₃)haloalkyl, and (C₁-C₃)alkoxy;
(N) R¹² is selected from the group consisting of H, F, Cl, Br, I, (C₁-C₃)alkyl, (C₁-C₃)haloalkyl, and (C₁-C₃)alkoxy;
(O) R¹³ is selected from the group consisting of H, F, Cl, Br, I, (C₁-C₃)alkyl, (C₁-C₃)haloalkyl, and (C₁-C₃)alkoxy;
(P) R¹⁴ is selected from the group consisting of H, F, Cl, Br, I, (C₁-C₃)alkyl, (C₁-C₃)haloalkyl, and (C₁-C₃)alkoxy;
(Q) R¹⁵ is selected from the group consisting of H, (C₁-C₃)alkyl, (C₂-C₃)alkenyl, and (C₂-C₃)alkynyl;
(R) R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl, has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, NHC(═O)(C₁-C₆)alkyl-S—(C₁-C₆)haloalkyl; and
resolved stereoisomers of the molecules of Formula One.

The molecules of Formula One may exist in different geometric or optical isomeric or different tautomeric forms. One or more centers of chirality may be present in which case molecules of Formula One may be present as pure enantiomers, mixtures of enantiomers, pure diastereomers or mixtures of diastereomers. It will be appreciated by those skilled in the art that one stereoisomer may be more active than the other stereoisomers. Individual stereoisomers may be obtained by known selective synthetic procedures, by conventional synthetic procedures using resolved starting materials, or by conventional resolution procedures. There may be double bonds present in the molecule, in which case compounds of Formula One may exist as single geometric isomers (cis or trans, E or Z) or mixtures of geometric isomers (cis and trans, E and Z). Centers of tautomerisation may be present. This disclosure covers all such isomers, tautomers, and mixtures thereof, in all proportions. The structures disclosed in the present disclosure may be drawn in only one geometric form for clarity, but are intended to represent all geometric forms of the molecule.

In one embodiment the molecules of Formula One, the carboxamido, and the phenyl, which are bonded to the cyclopropane, are in the R,R configuration, as shown below, in Formula Two which is a sub-set of Formula One.

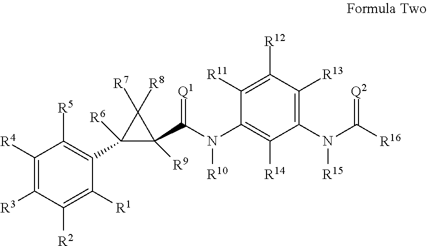

In another embodiment a molecule according to Formula One and Formula Two wherein R¹ is selected from the group consisting of H, F, Cl, Br, I, and CF₃.

In another embodiment a molecule according to Formula One and Formula Two wherein R² is selected from the group consisting of H, F, Cl, Br, I, and CF₃.

In another embodiment a molecule according to Formula One and Formula Two wherein R³ is selected from the group consisting of H, F, Cl, Br, I, and CF₃.

In another embodiment a molecule according to Formula One and Formula Two wherein R⁴ is selected from the group consisting of H, F, Cl, Br, I, and CF₃.

In another embodiment a molecule according to Formula One and Formula Two wherein R⁵ is selected from the group consisting of H, F, Cl, Br, I, and CF₃.

In another embodiment a molecule according to Formula One and Formula Two wherein at least one of R², R³, and R⁴ is CF₃.

In another embodiment a molecule according to Formula One and Formula Two wherein R⁷ is Cl.

In another embodiment a molecule according to Formula One and Formula Two wherein R⁸ is Cl.

In another embodiment a molecule according to Formula One and Formula Two wherein Q¹ is O.

In another embodiment a molecule according to Formula One and Formula Two wherein Q² is O.

In another embodiment a molecule according to Formula One and Formula Two wherein R¹⁰ is H.

In another embodiment a molecule according to Formula One and Formula Two wherein R¹¹ is H.

In another embodiment a molecule according to Formula One and Formula Two wherein R¹² is H.

In another embodiment a molecule according to Formula One and Formula Two wherein R¹³ is selected from the group consisting of H, F, and Cl.

In another embodiment a molecule according to Formula One and Formula Two wherein R¹⁴ is selected from the group consisting of H and F.

In another embodiment a molecule according to Formula One and Formula Two wherein R¹¹ is H and CH₃.

In another embodiment a molecule according to Formula One and Formula Two wherein R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl, wherein each said alkyl, alkenyl, alkynyl, cycloalkyl, haloalkyl, phenyl, substituted phenyl, heterocyclyl, and substituted heterocyclyl has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, NHC(═O)(C₁-C₆)alkyl-S—(C₁-C₆)haloalkyl.

In another embodiment a molecule according to Formula One and Formula Two wherein R¹⁶ is a heterocyclyl selected from the group consisting of furanyl, isoxazolonyl, isoxazolyl, morpholinyl, substituted pyridinyl, pyrimidinyl, substituted pyrrolidinyl, tetrahydrofuranyl, and substituted thiazolyl, wherein each substituted pyridinyl, substituted pyrrolidinyl, and substituted thiazolyl has one or more substituents selected from the group consisting of H, F, Cl, Br, CN, (C₁-C₆)alkyl, and (C₁-C₆)haloalkyl.

In another embodiment a molecule according to Formula One and Formula Two wherein R¹⁶ is selected from the group consisting of substituted pyridinyl, substituted pyrrolidinyl, and substituted thiazolyl, wherein each substituted pyridinyl, substituted pyrrolidinyl, and substituted thiazolyl has one or more substituents selected from the group consisting of H, F, CF₃, and CH₂CF₃.

In another embodiment a molecule according to Formula One and Formula Two wherein R¹⁶ is a substituted phenyl, wherein each substituted phenyl has one or more substituents selected from the group consisting of H, F, Cl, CN, NH₂, NO₂, (C₁-C₆)alkyl, (C₂-C₆)alkynyl, (C₁-C₆)haloalkyl, (C₁-C₆)alkoxy, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, and NHC(═O)(C₁-C₆)alkyl-S—(C₁-C₆)haloalkyl.

In another embodiment a molecule according to Formula One and Formula Two wherein R¹⁶ is a substituted phenyl, wherein each substituted phenyl has one or more substituents selected from the group consisting of C≡CH, CF₃, CH₃, Cl, CN, F, H, NH₂, NHC(═O)CF₃, NHC(═O)CH₃, NHC(═O)CHF₂, NHC(═O)CH₂CF₃, NHC(═O)CH₂CH₂CF₃, NHC(═O)CH₂OCH₃, NHC(═O)CH₂SCF₃, NHC(═O)CH₂OCH₂CF₃, NO₂, and OCH₃.

In another embodiment a molecule according to Formula One and Formula Two wherein:
R¹ is H;
R² is selected from the group consisting of H, Cl, Br, and CF₃;
R³ is selected from the group consisting of H, F, and Cl;
R⁴ is selected from the group consisting of H, Cl, Br, and CF₃;
R⁵ is H;
R⁶ is H;
R⁷ is Cl;
R⁸ is Cl;
R⁹ is H;
Q¹ is O;
Q² is O;
R¹⁰ is H;
R¹¹ is H;
R¹² is H;
R¹³ is selected from the group consisting of H, F, and Cl;
R¹⁴ is selected from the group consisting of H and F;
R¹⁵ is selected from the group consisting of H and CH₃;
R¹⁶ is selected from the group consisting of CF₂CF₂CF₃, CF₂CH₂CH₃, CF₂CHF₂, CF₂Cl, CF₂phenyl, CF₃, CH(CH₃)Ophenyl, CH(CH₃)CF₃, CH(CH₃)CN, CH(CH₃)OCH₂CH₃, CH₂morpholinyl, CH₂CN, CH₂CF₃, CH₂CH(CF₃)CH₃, CH₂CH═CH₂, CH₂CH₂CF₃, CH₂CH₂CH₂CF₃, CH₂CH₂CH₃, CH₂NHC(═O)CH₃, CH₂OCH₂CF₃, CH₂OCH₃, CH₂phenyl, substituted CH₂phenyl, CH₃, cyclopropyl, substituted cyclopropyl, furanyl, isoxazolonyl, isoxazolyl, phenyl, substituted phenyl, substituted pyridinyl, pyrimidinyl, substituted pyrrolidinyl, tetrahydrofuranyl, and substituted thiazolyl, wherein each said substituted CH₂phenyl, substituted cyclopropyl, substituted phenyl, substituted pyridinyl, substituted pyrrolidinyl, and substituted thiazolyl has one or more substituents selected from the group consisting of C≡CH, CF₃, CH₂CF₃, CH₃, Cl, CN, F, H, NH₂, NHC(═O)CF₃, NHC(═O)CH₃, NHC(═O)CHF₂, NHC(═O)CH₂CF₃, NHC(═O)CH₂CH₂CF₃, NHC(═O)CH₂OCH₃, NHC(═O)CH₂SCF₃, NHC(═O)CH₂OCH₂CF₃, NO₂, and OCH₃.

In another embodiment a molecule selected from Table 1, wherein said molecule is selected from the group consisting of F1, F2, F3, F4, F5, F6, F7, F8, F9, F10, F11, F12, F13, F14, F15, F18, F20, F24, F25, F26, F27, F30, F32, F34, F36, F37, F38, F39, F40, F41, F42, F43, F44, F47, F48, F49, F50, F51, F52, F53, F54, F55, F56, F57, F58, F60, F61, F62, F63, F64, F65, F66, F67, F68, F69, F70, F71, F72, F73, F74, F75, F77, F78, F79, F81, F82, F83, F84, F85, F87, F88, F89, F90, F91, F92, F93, F94, F95, F96, F97, F98, F99, F100, F102, F103, F104, F105, F106, F109, F110, F111, F112, F113, F114, F115, F116, F117, F118, F119, F120, and F121.

In another embodiment a molecule selected from Table 1, wherein said molecule is selected from the group consisting of F11, F27, F30, F32, F34, F36, F37, F38, F52, F60, F93, F96, F97, F98, F99, F106, F109, and F110.

In another embodiment a molecules selected from Table 1, wherein said molecule is selected from the group consisting of F27, F60, F93, F96, F97, F98, and F99.

### Preparation of Molecules of Formula One

### Preparation of Cyclopropyl Amides

Cyclopropyl carboxylic acids have been prepared in the art, for example in WO 2016/168056 A1, WO 2016/168058 A1, WO 2016/168059 A1, WO 2018/071320 A1, and WO 2018/071327 A1.

Cyclopropyl amides 1-3, wherein R¹, R², R³, R⁴, R⁵, R⁶, R⁷, R⁸, R⁹, Q¹, R¹⁰, R¹¹, R¹², R¹³, and R¹⁴ are as previously disclosed, may be prepared by treatment with amines or amine salts 1-2, wherein R¹⁰, R¹¹, R¹², R¹³, and R¹⁴ are as previously disclosed, and activated carboxylic acids 1-1, wherein Q¹ is O, A is an activating group, and R¹, R², R³, R⁴, R⁵, R⁶, R⁷, R⁸, and R⁹ are as previously disclosed, with a base, such as triethylamine, diisopropylethylamine, 4-methylmorpholine, or 4-dimethylaminopyridine in an anhydrous aprotic solvent such as dichloromethane, tetrahydrofuran, 1,2-dichloroethane, dimethylformamide, or any combination thereof, at temperatures between about 0° C. and about 120° C. (Scheme 1, step a).

Cyclopropyl amides 1-5, wherein R¹, R², R³, R⁴, R⁵, R⁶, R⁷, R⁸, R⁹, Q¹, R¹⁰, R¹¹, R¹², R¹³, and R¹⁴ are as previously disclosed, may be prepared by treatment with amines or amine salts 1-4, wherein R¹⁰, R¹¹, R¹², R¹³, and R¹⁴ are as previously disclosed, and activated carboxylic acids 1-1, wherein Q¹ is O, A is an activating group, and R¹, R², R³, R⁴, R⁵, R⁶, R⁷, R⁸, and R⁹ are as previously disclosed, with a base, such as triethylamine, diisopropylethylamine, 4-methylmorpholine, or 4-dimethylaminopyridine in an anhydrous aprotic solvent such as dichloromethane, tetrahydrofuran, 1,2-dichloroethane, dimethylformamide, or any combination thereof, at temperatures between about 0° C. and about 60° C. (Scheme 1, step b).

Activated carboxylic acids 1-1 may be an acid halide, such as an acid chloride, an acid bromide, or an acid fluoride; a carboxylic ester, such as a para-nitrophenyl ester, a pentafluorophenyl ester, an ethyl (hydroxyiminio)cyanoacetate ester, a methyl ester, an ethyl ester, a benzyl ester, an N-hydroxysuccinimidyl ester, a hydroxybenzotriazol-1-yl ester, or a hydroxypyridyltriazol-1-yl ester; an O-acylisourea; an acid anhydride; or a thioester. Acid chlorides may be prepared from the corresponding carboxylic acids by treatment with a dehydrating chlorinating reagent, such as oxalyl chloride or thionyl chloride. Activated carboxylic esters 1-1 may be prepared from carboxylic acids in situ with a uronium salt, such as 1-[bis(dimethylamino)methylene]-1H-1,2,3-triazolo[4,5-b]pyridinium 3-oxid hexafluorophosphate (HATU), 0-(benzotriazol-1-yl)-N,N,N′,N′-tetramethyluronium hexafluorophosphate (HBTU), 2-(1H-benzotriazole-1-yl)-1,1,3,3-tetramethylaminium tetrafluoroborate (TBTU), or (1-cyano-2-ethoxy-2-oxoethylidenaminooxy)dimethylamino-morpholino-carbenium hexafluorophosphate (COMU). Activated carboxylic esters 1-1 may also be prepared from carboxylic acids in situ with a phosphonium salt such as benzotriazol-1-yl-oxytripyrrolidinophosphonium hexafluorophosphate (PyBop). Activated carboxylic esters 1-1 may also be prepared from carboxylic acids in situ with a coupling reagent such as 1-(3-dimethylaminopropyl)-3-ethylcarbodiimide hydrochloride, or dicyclohexylcarbodiimide in the presence of a triazole such as hydroxybenzotriazole monohydrate (HOBt) or 1-hydroxy-7-azabenzotriazole (HOAt). O-Acylisoureas may be prepared with a dehydrating carbodimide such as 1-(3-dimethylamino propyl)-3-ethylcarbodiimide hydrochloride or dicyclohexylcarbodiimide. Activated carboxylic esters 1-1 may also be prepared from carboxylic acids in situ with a coupling reagent such as 2-chloro-1,3-dimethylimidazolidinium hexafluorophosphate (CIP) in the presence of a triazole such as 1-hydroxy-7-azabenzotriazole (HOAt). Activated carboxylic esters 1-1 may also be prepared from carboxylic acids in situ with a coupling reagent such as 2,4,6-tripropyl-1,3,5,2,4,6-trioxatriphosphinane 2,4,6-trioxide (T3P®) in the presence of a base such as pyridine.

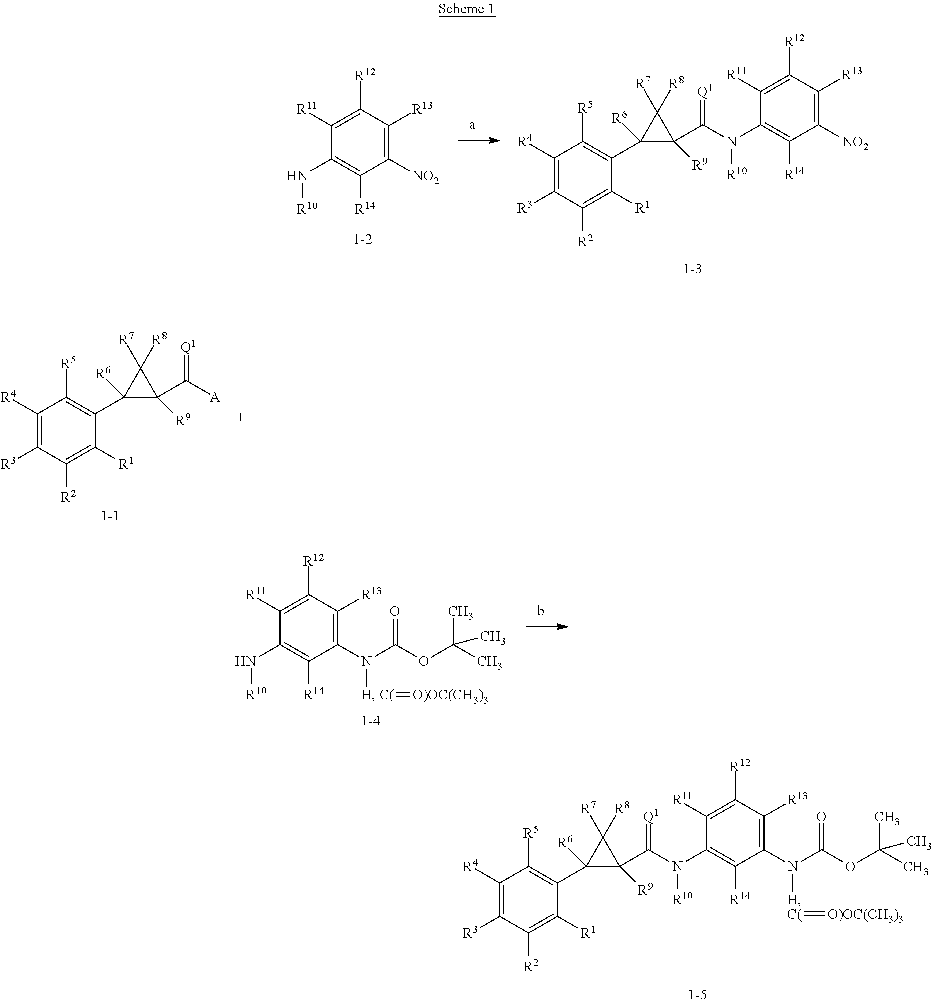

Cyclopropyl amides 2-1, wherein R¹, R², R³, R⁴, R⁵, R⁶, R⁷, R⁸, R⁹, Q¹, R¹⁰, R¹¹, R¹², R¹³, and R¹⁴ are as previously disclosed, may be prepared by treatment of 1-3, wherein R¹, R², R³, R⁴, R⁵, R⁶, R⁷, R⁸, R⁹, Q¹, R¹⁰, R¹¹, R¹², R¹³, and R¹⁴ are as previously disclosed, with a metal such as palladium on carbon in the presence of a reducing agent such as hydrogen gas in a solvent such as ethyl acetate or with a metal such as iron in the presence of a reducing agent such as ammonium chloride in a solvent mixture such as methanol and water or tetrahydrofuran, ethanol, and water at a temperature of about 25° C. to about 90° C. (Scheme 2, step a). Alternatively, cyclopropyl amides 2-1, wherein R¹, R², R³, R⁴, R⁵, R⁶, R⁷, R⁸, R⁹, Q¹, R¹⁰, R¹¹, R¹², R¹³, and R¹⁴ are as previously disclosed, may be prepared by treatment of 1-5 wherein R¹, R², R³, R⁴, R⁵, R⁶, R⁷, R⁸, R⁹, Q¹, R¹⁰, R¹¹, R¹², R¹³, and R¹⁴ are as previously disclosed, with an anhydrous acid solution such as hydrochloric acid in 1,4-dioxane at a temperature of about 25° C. (Scheme 2, step b).

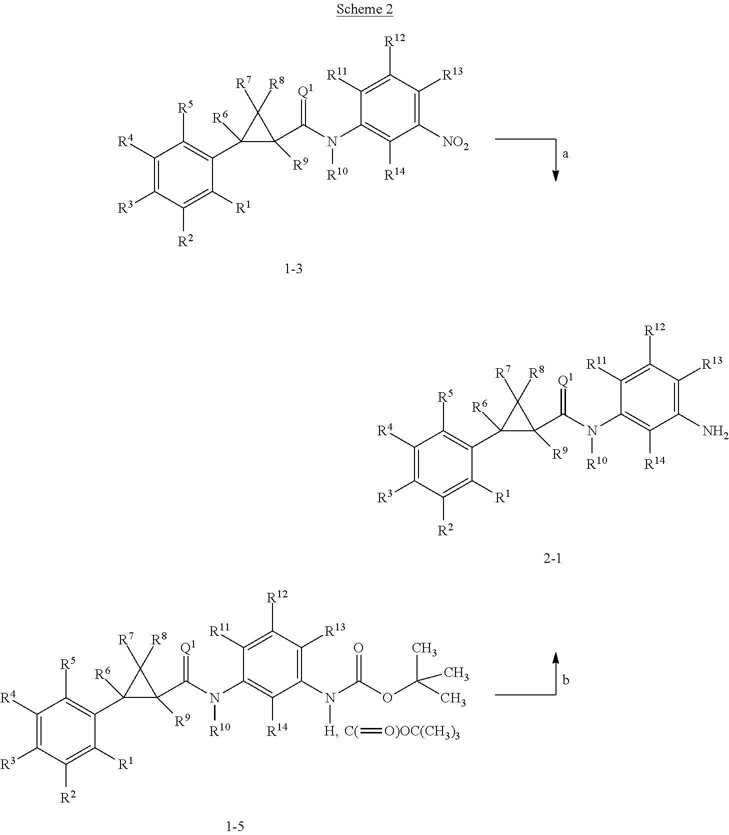

Cyclopropyl amides 3-1, or molecules of Formula One, wherein R¹, R², R³, R⁴, R⁵, R⁶, R⁷, R⁸, R⁹, Q¹, R¹⁰, R¹¹, R¹², R¹³, R¹⁴, R¹⁵, Q², and R¹⁶ are as previously disclosed, may be prepared by treatment of amines or amine salts 2-1 and carboxylic acids or activated carboxylic acids 3-2, wherein Q² is O, A is an activating group and R¹⁶ is as previously disclosed, with a base, such as triethylamine, diisopropylethylamine, 4-methylmorpholine, or 4-dimethylaminopyridine in an anhydrous aprotic solvent such as dichloromethane, tetrahydrofuran, 1,2-dichloroethane, dimethylformamide, or any combination thereof, at temperatures between about 0° C. and about 120° C. (Scheme 3, step a).

Activated carboxylic acids 3-2 may be an acid halide, such as an acid chloride, an acid bromide, or an acid fluoride; a carboxylic ester, such as a para-nitrophenyl ester, a pentafluorophenyl ester, an ethyl (hydroxyimino)cyanoacetate ester, a methyl ester, an ethyl ester, a benzyl ester, an N-hydroxysuccinimidyl ester, a hydroxybenzotriazol-1-yl ester, or a hydroxypyridyltriazol-1-yl ester; an O-acylisourea; an acid anhydride; or a thioester. Acid chlorides may be prepared from the corresponding carboxylic acids by treatment with a dehydrating chlorinating reagent, such as oxalyl chloride or thionyl chloride. Activated carboxylic esters 3-2 may be prepared from carboxylic acids in situ with a uronium salt, such as 1-[bis(dimethylamino)methylene]-1H-1,2,3-triazolo[4,5-b]pyridinium 3-oxid hexafluorophosphate (HATU), 0-(benzotriazol-1-yl)-N,N,N′,N′-tetramethyluronium hexafluorophosphate (HBTU), 2-(1H-benzotriazole-1-yl)-1,1,3,3-tetramethylaminium tetrafluoroborate (TBTU), or (1-cyano-2-ethoxy-2-oxoethylidenaminooxy)dimethylamino-morpholino-carbenium hexafluorophosphate (COMU). Activated carboxylic esters 3-2 may also be prepared from carboxylic acids in situ with a phosphonium salt such as benzotriazol-1-yl-oxytripyrrolidinophosphonium hexafluorophosphate (PyBop). Activated carboxylic esters 3-2 may also be prepared from carboxylic acids in situ with a coupling reagent such as 1-(3-dimethylaminopropyl)-3-ethylcarbodiimide hydrochloride, or dicyclohexylcarbodiimide in the presence of a triazole such as hydroxybenzotriazole monohydrate (HOBt) or 1-hydroxy-7-azabenzotriazole (HOAt). O-Acylisoureas may be prepared with a dehydrating carbodimide such as 1-(3-dimethylaminopropyl)-3-ethylcarbodiimide hydrochloride or dicyclohexylcarbodiimide. Activated carboxylic esters 3-2 may also be prepared from carboxylic acids in situ with a coupling reagent such as 2-chloro-1,3-dimethylimidazolidinium hexafluorophosphate (CIP) in the presence of a triazole such as 1-hydroxy-7-azabenzotriazole (HOAt). Activated carboxylic esters 3-2 may also be prepared from carboxylic acids in situ with a coupling reagent such as 2,4,6-tripropyl-1,3,5,2,4,6-trioxatriphosphinane 2,4,6-trioxide (T3P®) in the presence of a base such as pyridine. Alternatively carboxylic esters 3-2, for example methyl and ethyl esters, wherein Q² is O, A is OMe or OEt, respectively, and R¹⁶ is as previously disclosed, may be reacted with amines or amine salts 2-1 in the presence of trimethylaluminum in an aprotic solvent such as toluene at room temperature to provide cyclopropyl amides 3-1, wherein R¹, R², R³, R⁴, R⁵, R⁶, R⁷, R⁸, R⁹, Q¹, R¹⁰, R¹¹, R¹², R¹³, R¹⁴, R¹⁵, Q², and R¹⁶ are as previously disclosed. Additionally, amines or amine salts 2-1 may be treated with acid anhydrides 3-2, for example trifluoroacetic anhydride or acetic anhydride, in the presence of an aprotic solvent such as dichloromethane at room temperature to provide cyclopropyl amides 3-1, wherein R¹, R², R³, R⁴, R⁵, R⁶, R⁷, R⁸, R⁹, Q¹, R¹⁰, R¹¹, R¹², R¹³, R¹⁴, R¹⁵, Q², and R¹⁶ are as previously disclosed.

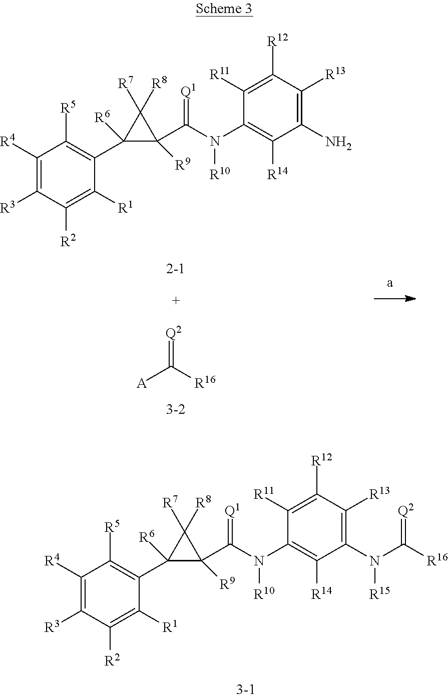

Cyclopropyl amides 3-1, or molecules of Formula One, wherein R¹, R², R³, R⁴, R⁵, R⁶, R⁷, R⁸, R⁹, Q¹, R¹⁰, R¹¹, R¹², R¹³, R¹⁴, R¹⁵, Q², and R¹⁶ are as previously disclosed, may be prepared by treatment of amines or amine salts 4-2, wherein R¹⁰, R¹¹, R¹², R¹³, R¹⁴, R¹⁵, Q², and R¹⁶ are as previously disclosed, and activated carboxylic acids 1-1, wherein Q¹ is O, A is an activating group, and R¹, R², R³, R⁴, R⁵, R⁶, R⁷, R⁸, and R⁹ are as previously disclosed, with a base, such as triethylamine, diisopropylethylamine, 4-methylmorpholine, or 4-dimethylaminopyridine in an anhydrous aprotic solvent such as dichloromethane, tetrahydrofuran, 1,2-dichloroethane, dimethylformamide, or any combination thereof, at temperatures between about 0° C. and about 120° C. (Scheme 1, step a).

Activated carboxylic acids 1-1 may be an acid halide, such as an acid chloride, an acid bromide, or an acid fluoride; a carboxylic ester, such as a para-nitrophenyl ester, a pentafluorophenyl ester, an ethyl (hydroxyiminio)cyanoacetate ester, a methyl ester, an ethyl ester, a benzyl ester, an N-hydroxysuccinimidyl ester, a hydroxybenzotriazol-1-yl ester, or a hydroxypyridyltriazol-1-yl ester; an O-acylisourea; an acid anhydride; or a thioester. Acid chlorides may be prepared from the corresponding carboxylic acids by treatment with a dehydrating chlorinating reagent, such as oxalyl chloride or thionyl chloride. Activated carboxylic esters 1-1 may be prepared from carboxylic acids in situ with a uronium salt, such as 1-[bis(dimethylamino)methylene]-1H-1,2,3-triazolo[4,5-b]pyridinium 3-oxid hexafluorophosphate (HATU), 0-(benzotriazol-1-yl)-N,N,N′,N′-tetramethyluronium hexafluorophosphate (HBTU), 2-(1H-benzotriazole-1-yl)-1,1,3,3-tetramethylaminium tetrafluoroborate (TBTU), or (1-cyano-2-ethoxy-2-oxoethylidenaminooxy)dimethylamino-morpholino-carbenium hexafluorophosphate (COMU). Activated carboxylic esters 1-1 may also be prepared from carboxylic acids in situ with a phosphonium salt such as benzotriazol-1-yl-oxytripyrrolidinophosphonium hexafluorophosphate (PyBop). Activated carboxylic esters 1-1 may also be prepared from carboxylic acids in situ with a coupling reagent such as 1-(3-dimethylaminopropyl)-3-ethylcarbodiimide hydrochloride, or dicyclohexylcarbodiimide in the presence of a triazole such as hydroxybenzotriazole monohydrate (HOBt) or 1-hydroxy-7-azabenzotriazole (HOAt). O-Acylisoureas may be prepared with a dehydrating carbodimide such as 1-(3-dimethylamino propyl)-3-ethylcarbodiimide hydrochloride or dicyclohexylcarbodiimide. Activated carboxylic esters 1-1 may also be prepared from carboxylic acids in situ with a coupling reagent such as 2-chloro-1,3-dimethylimidazolidinium hexafluorophosphate (CIP) in the presence of a triazole such as 1-hydroxy-7-azabenzotriazole (HOAt). Activated carboxylic esters 1-1 may also be prepared from carboxylic acids in situ with a coupling reagent such as 2,4,6-tripropyl-1,3,5,2,4,6-trioxatriphosphinane 2,4,6-trioxide (T3P®) in the presence of a base such as pyridine.

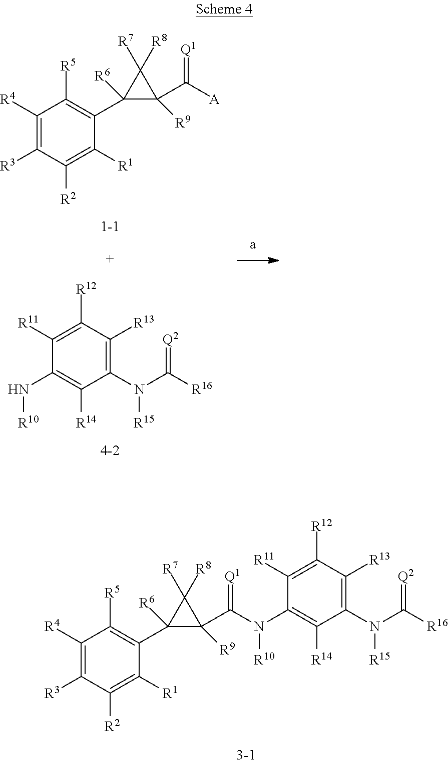

Cyclopropyl amides 3-1, wherein R¹, R², R³, R⁴, R⁵, R⁶, R⁷, R⁸, R⁹, Q¹, R¹⁰, R¹¹, R¹², R¹³, R¹⁴, R¹⁵, and Q² are as previously disclosed, R¹⁶ is a substituted phenyl further substituted with one or more amido (—NHC(═O)L) groups, and L is (C₁-C₆)alkyl, (C₁-C₆)haloalkyl, (C₁-C₆)alkyl-O(C₁-C₆)alkyl, (C₁-C₆)alkyl-O(C₁-C₆)haloalkyl, (C₁-C₆)alkyl-S(C₁-C₆)alkyl, and (C₁-C₆)alkyl-S(C₁-C₆)haloalkyl, wherein each alkyl and haloalkyl may be optionally substituted with one or more substituents selected from F, Cl, Br, I, CN, OH, NH₂, NO₂, oxo, (C₁-C₆)alkyl, (C₁-C₆)alkoxy, and C(═O)O(C₁-C₆)alkyl, may be prepared by treatment of cyclopropyl amide 3-1, wherein R¹, R², R³, R⁴, R⁵, R⁶, R⁷, R⁸, R⁹, Q¹, R¹⁰, R¹¹, R¹², R¹³, R¹⁴, R¹⁵, and Q² are as previously disclosed and R¹⁶ is a substituted phenyl further substituted with one or more amine (—NH₂) groups, and activated carboxylic acids 5-1, wherein A is an activating group and L is as previously disclosed, with a base, such as triethylamine, diisopropylethylamine, 4-methylmorpholine, 4-dimethylaminopyridine, or pyridine, in an anhydrous aprotic solvent such as dichloromethane, tetrahydrofuran, 1,2-dichloroethane, N,N-dimethylformamide, or any combination thereof, at temperatures between about 0° C. and about 120° C. (Scheme 5, step a).

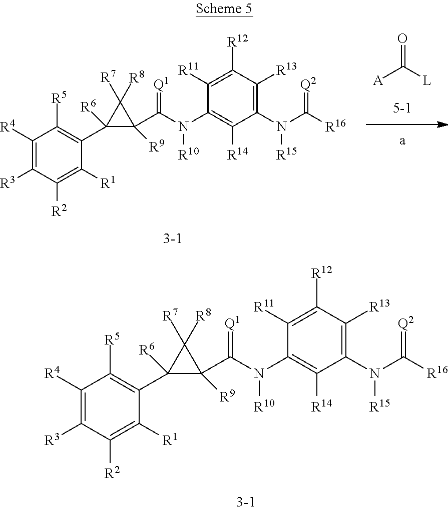

The following examples further illustrate the variations of the molecules of Formula One or Formula Two within the disclosure but, of course, should not be construed as in any way limiting its scope.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Molecules are given their known names, named according to the naming program within ChemDraw (version 17.1.0.105 (19)). If such a program is unable to name a molecule, such molecule is named using conventional naming rules. ¹H NMR spectral data are in ppm (δ) and were recorded at 300, 400, and 500 MHz; ¹³C NMR spectral data are in ppm (δ) and were recorded at 101 and 126 MHz; and ¹⁹F NMR spectral data are in ppm (δ) and were recorded at 376 and 471 MHz, unless otherwise stated.

### Example 1

### Preparation of N-(3-((1R,3R)-2,2-dichloro-3-(3,4,5-trichlorophenyl)cyclopropane-1-carboxamido)-2,6-difluorophenyl)-2,4-difluorobenzamide (F10)

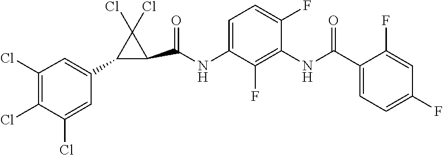 [SMILES: O=C(Nc1c(F)ccc(NC(=O)[C@H]2[C@H](c3cc(Cl)c(Cl)c(Cl)c3)C2(Cl)Cl)c1F)c1ccc(F)cc1F]

To a solution of (1R,3R)—N-(3-amino-2,4-difluorophenyl)-2,2-dichloro-3-(3,4,5-trichlorophenyl)cyclopropane-1-carboxamide (C1, 0.075 grams (g), 0.163 millimoles (mmol)) in ethyl acetate (2 milliliters (mL)) were added sequentially 2,4-difluorobenzoic acid (0.026 g, 0.163 mmol) and pyridine (0.026 mL, 0.326 mmol). A 50% solution of 2,4,6-tripropyl-1,3,5,2,4,6-trioxatriphosphinane 2,4,6-trioxide (T3P, 0.155 g, 0.244 mmol) in ethyl acetate was added and the reaction mixture was warmed to 50° C. for 18 hours. The reaction mixture was cooled to room temperature and was concentrated under a stream of nitrogen. The residue was purified by column chromatography eluting with 0-35% ethyl acetate in hexanes to afford the title compound as a light yellow solid (0.073 g, 75%).

The following compounds were prepared in accordance with the procedure in Example 1:

### N-(3-((1R,3R)-2,2-Dichloro-3-(3,4,5-trichlorophenyl)cyclopropane-1-carboxamido)-2,6-difluorophenyl)tetrahydrofuran-2-carboxamide (F11)

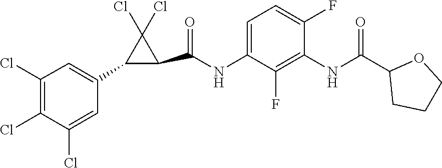 [SMILES: O=C(Nc1c(F)ccc(NC(=O)[C@H]2[C@H](c3cc(Cl)c(Cl)c(Cl)c3)C2(Cl)Cl)c1F)C1CCCO1]

The title compound was prepared and was isolated as a white foam (0.076 g, 83%).

### (1R,3R)-2,2-Dichloro-N-(3-(2-ethoxypropanamido)-2,4-difluorophenyl)-3-(3,4,5-trichlorophenyl)cyclopropane-1-carboxamide (F12)

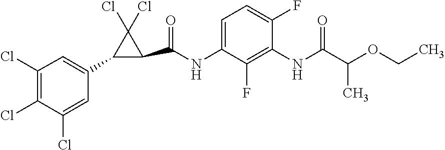 [SMILES: CCOC(C)C(=O)Nc1c(F)ccc(NC(=O)[C@H]2[C@H](c3cc(Cl)c(Cl)c(Cl)c3)C2(Cl)Cl)c1F]

The title compound was prepared and was isolated as a white foam (0.073 g, 80%).

### (1R,3R)-2,2-Dichloro-N-(2,4-difluoro-3-(2,2,3,3,4,4,4-heptafluorobutanamido)phenyl)-3-(3,4,5-trichlorophenyl)cyclopropane-1-carboxamide (F13)

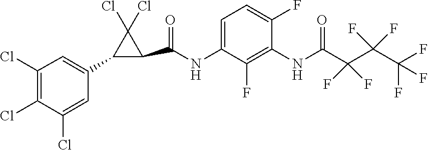 [SMILES: O=C(Nc1ccc(F)c(NC(=O)C(F)(F)C(F)(F)C(F)(F)F)c1F)[C@H]1[C@H](c2cc(Cl)c(Cl)c(Cl)c2)C1(Cl)Cl]

The title compound was prepared and was isolated as a white foam (0.070 g, 66%).

### (1R,3R)—N-(3-Butyramido-2,4-difluorophenyl)-2,2-dichloro-3-(3,4,5-trichlorophenyl)cyclopropane-1-carboxamide (F14)

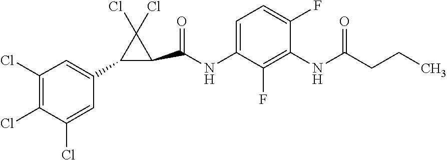 [SMILES: CCCC(=O)Nc1c(F)ccc(NC(=O)[C@H]2[C@H](c3cc(Cl)c(Cl)c(Cl)c3)C2(Cl)Cl)c1F]

The title compound was prepared and was isolated as a white foam (0.082 g, 94%).

### (1R,3R)-2,2-Dichloro-3-(3,5-dichlorophenyl)-N-(2-fluoro-3-(4,4,4-trifluoro-3-methylbutanamido)phenyl)cyclopropane-1-carboxamide (F15)

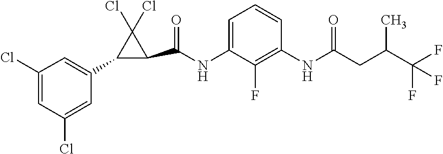 [SMILES: CC(CC(=O)Nc1cccc(NC(=O)[C@H]2[C@H](c3cc(Cl)cc(Cl)c3)C2(Cl)Cl)c1F)C(F)(F)F]

The title compound was prepared and was isolated as a white foam (0.105 g, 78%).

### N-(3-((1R,3R)-2,2-Dichloro-3-(3,5-dichlorophenyl)cyclopropane-1-carboxamido)-2-fluorophenyl)-2,4-difluorobenzamide (F16)

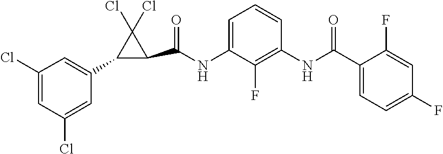 [SMILES: O=C(Nc1cccc(NC(=O)[C@H]2[C@H](c3cc(Cl)cc(Cl)c3)C2(Cl)Cl)c1F)c1ccc(F)cc1F]

The title compound was prepared and was isolated as a light yellow solid (0.92 g, 68%).

### N-(3-((1R,3R)-2,2-Dichloro-3-(3,5-dichlorophenyl)cyclopropane-1-carboxamido)-2-fluorophenyl)tetrahydrofuran-2-carboxamide (F17)

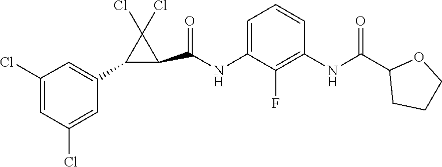 [SMILES: O=C(Nc1cccc(NC(=O)[C@H]2[C@H](c3cc(Cl)cc(Cl)c3)C2(Cl)Cl)c1F)C1CCCO1]

The title compound was prepared and was isolated as a white foam (0.110 g, 88%).

### (1R,3R)-2,2-Dichloro-3-(3,5-dichlorophenyl)-N-(3-(2-ethoxypropanamido)-2-fluorophenyl)cyclopropane-1-carboxamide (F18)

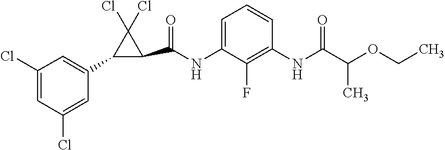 [SMILES: CCOC(C)C(=O)Nc1cccc(NC(=O)[C@H]2[C@H](c3cc(Cl)cc(Cl)c3)C2(Cl)Cl)c1F]

The title compound was prepared and was isolated as a white foam (0.100 g, 80%).

### (1R,3R)-2,2-Dichloro-3-(3,5-dichlorophenyl)-N-(2-fluoro-3-(2,2,3,3,4,4,4-heptafluorobutanamido)phenyl)cyclopropane-1-carboxamide (F19)

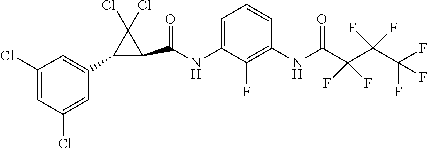 [SMILES: O=C(Nc1cccc(NC(=O)C(F)(F)C(F)(F)C(F)(F)F)c1F)[C@H]1[C@H](c2cc(Cl)cc(Cl)c2)C1(Cl)Cl]

The title compound was prepared and was isolated as a white foam (0.082 g, 56%).

### (1R,3R)—N-(3-Butyramido-2-fluorophenyl)-2,2-dichloro-3-(3,5-dichlorophenyl)cyclopropane-1-carboxamide (F20)

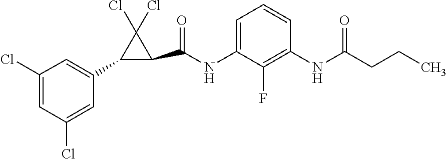 [SMILES: CCCC(=O)Nc1cccc(NC(=O)[C@H]2[C@H](c3cc(Cl)cc(Cl)c3)C2(Cl)Cl)c1F]

The title compound was prepared and was isolated as a white foam (0.099 g, 85%).

### (1R,3R)-2,2-Dichloro-N-(3-(2-cyclopropylacetamido)-2-fluorophenyl)-3-(3,5-dichlorophenyl)cyclopropane-1-carboxamide (F21)

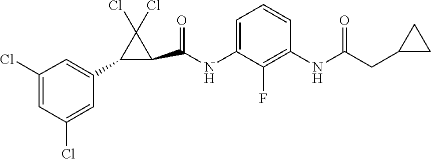 [SMILES: O=C(CC1CC1)Nc1cccc(NC(=O)[C@H]2[C@H](c3cc(Cl)cc(Cl)c3)C2(Cl)Cl)c1F]

The title compound was prepared and was isolated as a white foam (0.083 g, 69%).

### (1R,3R)-2,2-Dichloro-N-(3-(2-cyanopropanamido)-2-fluorophenyl)-3-(3,5-dichlorophenyl)cyclopropane-1-carboxamide (F22)

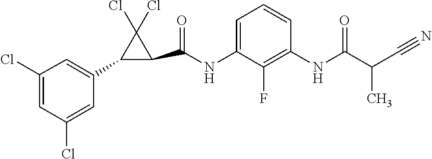 [SMILES: CC(C#N)C(=O)Nc1cccc(NC(=O)[C@H]2[C@H](c3cc(Cl)cc(Cl)c3)C2(Cl)Cl)c1F]

The title compound was prepared and was isolated as a light yellow solid (0.097 g, 81%).

### (1R,3R)—N-(3-(But-3-enamido)-2-fluorophenyl)-2,2-dichloro-3-(3,5-dichlorophenyl)cyclopropane-1-carboxamide (F23)

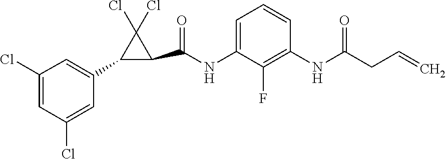 [SMILES: C=CCC(=O)Nc1cccc(NC(=O)[C@H]2[C@H](c3cc(Cl)cc(Cl)c3)C2(Cl)Cl)c1F]

The title compound was prepared and was isolated as a white foam (0.095 g, 82%).

### trans-rac-2,2-Dichloro-N-(4-chloro-3-(2,2-difluorocyclopropane-1-carboxamido)phenyl)-3-(3,5-dichlorophenyl)cyclopropane-1-carboxamide (F44)

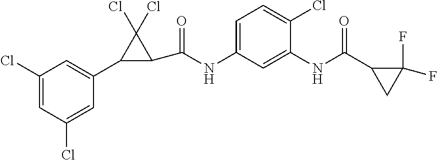 [SMILES: O=C(Nc1cc(NC(=O)C2C(c3cc(Cl)cc(Cl)c3)C2(Cl)Cl)ccc1Cl)C1CC1(F)F]

The title compound was prepared and was isolated as a white foam (0.06 g, 46%).

### trans-rac-4-Cyano-N-(5-(2,2-dichloro-3-(3,5-dichlorophenyl)cyclopropane-1-carboxamido)-2-fluorophenyl)-2-methylbenzamide (F60)

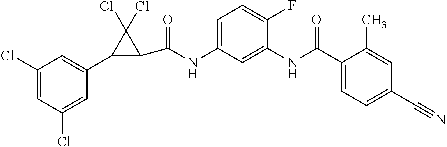 [SMILES: Cc1cc(C#N)ccc1C(=O)Nc1cc(NC(=O)C2C(c3cc(Cl)cc(Cl)c3)C2(Cl)Cl)ccc1F]

The title compound was prepared and was isolated as a light brown foam (0.081 g, 60%).

### trans-rac-N-(5-(2,2-Dichloro-3-(3,5-dichlorophenyl)cyclopropane-1-carboxamido)-2-fluorophenyl)-2,6-difluorobenzamide (F61)

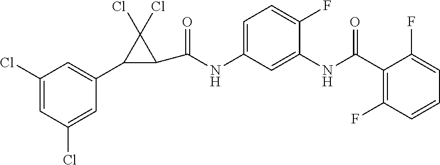 [SMILES: O=C(Nc1cc(NC(=O)C2C(c3cc(Cl)cc(Cl)c3)C2(Cl)Cl)ccc1F)c1c(F)cccc1F]

The title compound was prepared and was isolated as a white foam (0.086 g, 64%).

### trans-rac-N-(5-(2,2-Dichloro-3-(3,5-dichlorophenyl)cyclopropane-1-carboxamido)-2-fluorophenyl)-3,5-difluorobenzamide (F62)

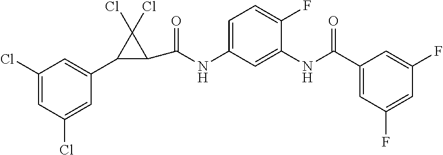 [SMILES: O=C(Nc1cc(NC(=O)C2C(c3cc(Cl)cc(Cl)c3)C2(Cl)Cl)ccc1F)c1cc(F)cc(F)c1]

The title compound was prepared and was isolated as a white solid (0.088 g, 66%).

### trans-rac-2,2-Dichloro-3-(3,5-dichlorophenyl)-N-(4-fluoro-3-(2-morpholinoacetamido)phenyl)cyclopropane-1-carboxamide (F63)

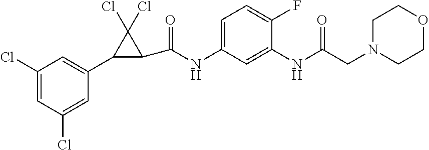 [SMILES: O=C(CN1CCOCC1)Nc1cc(NC(=O)C2C(c3cc(Cl)cc(Cl)c3)C2(Cl)Cl)ccc1F]

The title compound was prepared and was isolated as a white foam (0.042 g, 32%).

### trans-rac-2,2-Dichloro-3-(3,5-dichlorophenyl)-N-(4-fluoro-3-(4,4,4-trifluoro-3-methylbutanamido)phenyl)cyclopropane-1-carboxamide (F64)

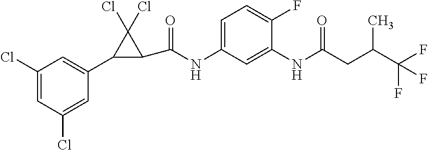 [SMILES: CC(CC(=O)Nc1cc(NC(=O)C2C(c3cc(Cl)cc(Cl)c3)C2(Cl)Cl)ccc1F)C(F)(F)F]

The title compound was prepared and was isolated as a white solid (0.102 g, 76%).

### trans-rac-N-(2-Chloro-5-(2,2-dichloro-3-(3,5-dichlorophenyl)cyclopropane-1-carboxamido)phenyl)-4-(trifluoromethyl)benzamide (F82)

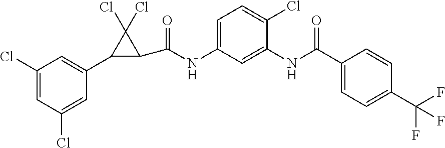 [SMILES: O=C(Nc1cc(NC(=O)C2C(c3cc(Cl)cc(Cl)c3)C2(Cl)Cl)ccc1Cl)c1ccc(C(F)(F)F)cc1]

The title compound was prepared and was isolated as an off-white solid (0.09 g, 20%).

### trans-rac-4-Acetamido-N-(2-chloro-5-(2,2-dichloro-3-(4-fluoro-3-(trifluoromethyl)phenyl)cyclopropane-1-carboxamido)phenyl)-3-fluoro-2-methylbenzamide (F113)

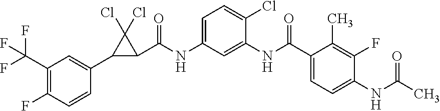 [SMILES: CC(=O)Nc1ccc(C(=O)Nc2cc(NC(=O)C3C(c4ccc(F)c(C(F)(F)F)c4)C3(Cl)Cl)ccc2Cl)c(C)c1F]

The title compound was prepared and was isolated as an off-white solid (0.07 g, 33%).

### trans-rac-N-(2-Chloro-5-(2,2-dichloro-3-(4-fluoro-3-(trifluoromethyl)phenyl)cyclopropane-1-carboxamido)phenyl)-3-fluoro-2-methyl-4-(3,3,3-trifluoropropanamido)benzamide (F114)

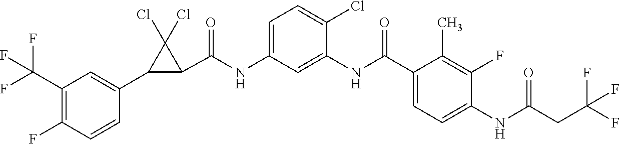 [SMILES: Cc1c(C(=O)Nc2cc(NC(=O)C3C(c4ccc(F)c(C(F)(F)F)c4)C3(Cl)Cl)ccc2Cl)ccc(NC(=O)CC(F)(F)F)c1F]

The title compound was prepared and was isolated as an off-white solid (0.13 g, 46%).

### trans-rac-N-(2-Chloro-5-(2,2-dichloro-3-(4-fluoro-3-(trifluoromethyl)phenyl)cyclopropane-1-carboxamido)phenyl)-3-fluoro-4-(2-methoxyacetamido)-2-methylbenzamide (F115)

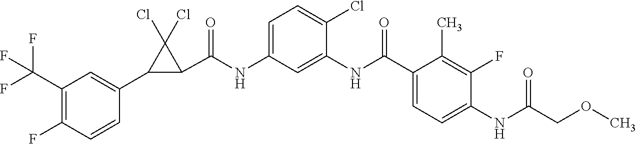 [SMILES: COCC(=O)Nc1ccc(C(=O)Nc2cc(NC(=O)C3C(c4ccc(F)c(C(F)(F)F)c4)C3(Cl)Cl)ccc2Cl)c(C)c1F]

The title compound was prepared and was isolated as an off-white solid (0.07 g, 31%).

### trans-rac-N-(2-Chloro-5-(2,2-dichloro-3-(4-fluoro-3-(trifluoromethyl)phenyl)cyclopropane-1-carboxamido)phenyl)-3-fluoro-2-methyl-4-(2-(2,2,2-trifluoroethoxy)acetamido)benzamide (F116)

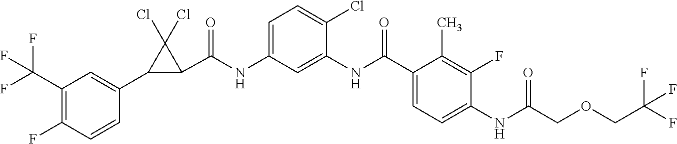 [SMILES: Cc1c(C(=O)Nc2cc(NC(=O)C3C(c4ccc(F)c(C(F)(F)F)c4)C3(Cl)Cl)ccc2Cl)ccc(NC(=O)COCC(F)(F)F)c1F]

The title compound was prepared and was isolated as an off-white solid (0.14 g, 58%).

### trans-rac-4-Acetamido-N-(2-chloro-5-(2,2-dichloro-3-(4-fluoro-3-(trifluoromethyl)phenyl)cyclopropane-1-carboxamido)phenyl)-2-fluorobenzamide (F117)

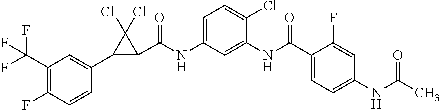 [SMILES: CC(=O)Nc1ccc(C(=O)Nc2cc(NC(=O)C3C(c4ccc(F)c(C(F)(F)F)c4)C3(Cl)Cl)ccc2Cl)c(F)c1]

The title compound was prepared and was isolated as an off-white solid (0.1 g, 38%).

### trans-rac-N-(2-Chloro-5-(2,2-dichloro-3-(4-fluoro-3-(trifluoromethyl)phenyl)cyclopropane-1-carboxamido)phenyl)-2-fluoro-4-(2-methoxyacetamido)benzamide (F118)

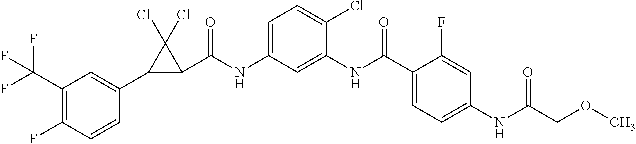 [SMILES: COCC(=O)Nc1ccc(C(=O)Nc2cc(NC(=O)C3C(c4ccc(F)c(C(F)(F)F)c4)C3(Cl)Cl)ccc2Cl)c(F)c1]

The title compound was prepared and was isolated as an off-white solid (0.2 g, 60%).

### trans-rac-N-(2-Chloro-5-(2,2-dichloro-3-(4-fluoro-3-(trifluoromethyl)phenyl)cyclopropane-1-carboxamido)phenyl)-2-fluoro-4-(2-(2,2,2-trifluoroethoxy)acetamido)benzamide (F119)

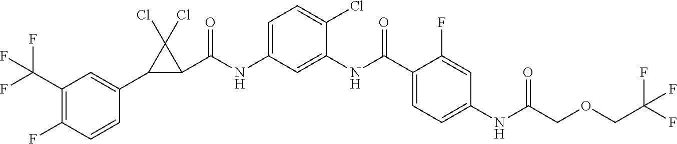 [SMILES: O=C(COCC(F)(F)F)Nc1ccc(C(=O)Nc2cc(NC(=O)C3C(c4ccc(F)c(C(F)(F)F)c4)C3(Cl)Cl)ccc2Cl)c(F)c1]

The title compound was prepared and was isolated as an off-white solid (0.16 g, 42%).

### trans-rac-N-(2-Chloro-5-(2,2-dichloro-3-(4-fluoro-3-(trifluoromethyl)phenyl)cyclopropane-1-carboxamido)phenyl)-2-fluoro-4-(4,4,4-trifluorobutanamido)benzamide (F120)

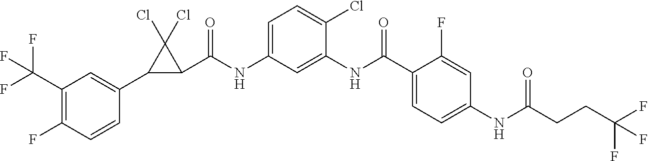 [SMILES: O=C(CCC(F)(F)F)Nc1ccc(C(=O)Nc2cc(NC(=O)C3C(c4ccc(F)c(C(F)(F)F)c4)C3(Cl)Cl)ccc2Cl)c(F)c1]

The title compound was prepared and was isolated as an off-white solid (0.28 g, 46%).

### trans-rac-N-(2-Chloro-5-(2,2-dichloro-3-(4-fluoro-3-(trifluoromethyl)phenyl)cyclopropane-1-carboxamido)phenyl)-2-fluoro-4-(2-((trifluoromethyl)thio)acetamido)benzamide (F121)

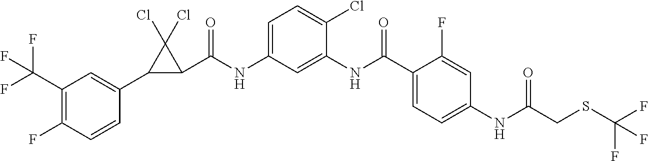 [SMILES: O=C(CSC(F)(F)F)Nc1ccc(C(=O)Nc2cc(NC(=O)C3C(c4ccc(F)c(C(F)(F)F)c4)C3(Cl)Cl)ccc2Cl)c(F)c1]

The title compound was prepared and was isolated as an off-white solid (0.14 g, 37%).

### trans-rac-N-(2-Chloro-5-(2,2-dichloro-3-(4-fluoro-3-(trifluoromethyl)phenyl)cyclopropane-1-carboxamido)phenyl)-2-fluoro-4-nitrobenzamide (C2)

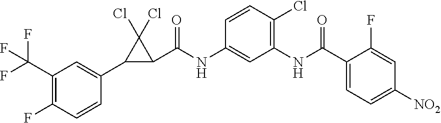 [SMILES: O=C(Nc1cc(NC(=O)C2C(c3ccc(F)c(C(F)(F)F)c3)C2(Cl)Cl)ccc1Cl)c1ccc([N+](=O)[O-])cc1F]

The title compound was prepared and was isolated as a pale-yellow solid (1.2 g, 85%); ESIMS m/z 608 ([M−H]⁻).

### Example 2

### Preparation of trans-rac-2,2-dichloro-N-(4-chloro-3-(4,4,4-trifluorobutanamido)phenyl)-3-(3,5-dichlorophenyl)cyclopropane-1-carboxamide (F7)

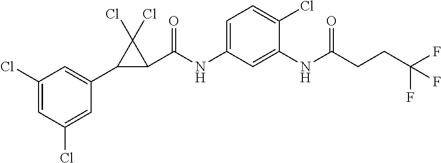 [SMILES: O=C(CCC(F)(F)F)Nc1cc(NC(=O)C2C(c3cc(Cl)cc(Cl)c3)C2(Cl)Cl)ccc1Cl]

Step 1. To a mixture of 4,4,4-trifluorobutanoic acid (28 mg, 0.20 mmol) in dichloroethane (1 mL) were added N,N-dimethylformamide (1 drop) followed by oxalyl chloride (1.49 g, 1.0 mL, 11.7 mmol), and the resulting solution was stirred at room temperature for 1.5 hours. The solvent was concentrated under reduced pressure. The residue was dissolved in dichloroethane and concentrated under reduced pressure at 15-20° C. (2×) to give 4,4,4-trifluorobutanoyl chloride as a white solid.

To a suspension of trans-rac-N-(3-amino-4-chlorophenyl)-2,2-dichloro-3-(3,5-dichlorophenyl)cyclopropane-1-carboxamide (C3, 90 mg, 0.20 mmol) in dichloromethane (1.5 mL) was added diisopropylethylamine (76 mg, 102 μL, 0.59 mmol). The resulting solution was treated with a solution of the freshly prepared trifluorobutanoyl chloride from above and was stirred at room temperature for 16 hours. To the mixture was added catalytic N,N-dimethylaminopyridine, and the reaction mixture was stirred at room temperature (˜64 hours). The reaction mixture was warmed to 38° C. and stirred for 16 hours, which resulted in little change. The mixture was cooled to room temperature, was treated with additional trifluorobutanoyl chloride (0.032 g, 1 molar equivalent), and was stirred for 16 hours. The reaction mixture was washed with water and the organic phase was isolated and dried by passing through a phase separator cartridge. The solvent was evaporated under a positive stream of nitrogen to give a dark oil (317 mg) which was purified by reverse phase flash chromatography (C18; 40→100% acetonitrile in water). The product fractions were combined and concentrated under reduced pressure. The aqueous residue was dissolved in acetonitrile (˜150 mL) and the solvent was evaporated under reduced pressure (2×) to give the title compound as a white solid (0.048 g, 45%).

The following compounds were prepared in accordance with the procedure in Example 2:

### trans-rac-2,2-Dichloro-N-(4-chloro-3-(3,3,3-trifluoropropanamido)phenyl)-3-(3,5-dichlorophenyl)cyclopropane-1-carboxamide (F5)

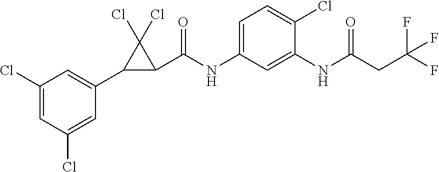 [SMILES: O=C(CC(F)(F)F)Nc1cc(NC(=O)C2C(c3cc(Cl)cc(Cl)c3)C2(Cl)Cl)ccc1Cl]

The title compound was prepared and was isolated as a white solid (0.046 g, 47%).

### trans-rac-2,2-Dichloro-N-(4-chloro-3-(3,3,3-trifluoro-2-methylpropanamido)phenyl)-3-(3,5-dichlorophenyl)cyclopropane-1-carboxamide (F6)

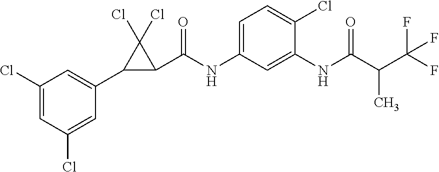 [SMILES: CC(C(=O)Nc1cc(NC(=O)C2C(c3cc(Cl)cc(Cl)c3)C2(Cl)Cl)ccc1Cl)C(F)(F)F]

The title compound was prepared and was isolated as a white solid (0.022 g, 20%).

### trans-rac-2,2-Dichloro-N-(4-chloro-3-(4,4,4-trifluoro-2-methylbutanamido)phenyl)-3-(3,5-dichlorophenyl)cyclopropane-1-carboxamide (F8)

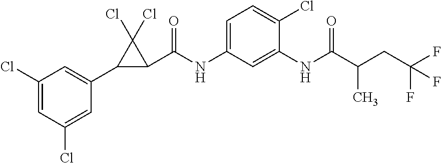 [SMILES: CC(CC(F)(F)F)C(=O)Nc1cc(NC(=O)C2C(c3cc(Cl)cc(Cl)c3)C2(Cl)Cl)ccc1Cl]

The title compound was prepared and was isolated as a white solid (0.031 g, 28%).

### trans-rac-2,2-Dichloro-N-(4-chloro-3-(4,4,4-trifluoro-3-methylbutanamido)phenyl)-3-(3,5-dichlorophenyl)cyclopropane-1-carboxamide (F9)

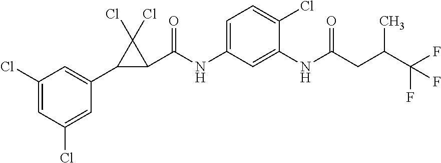 [SMILES: CC(CC(=O)Nc1cc(NC(=O)C2C(c3cc(Cl)cc(Cl)c3)C2(Cl)Cl)ccc1Cl)C(F)(F)F]

The title compound was prepared and was isolated as a white solid (0.040 g, 36%).

### trans-rac-N-(2-Chloro-5-(2,2-dichloro-3-(3,5-dichlorophenyl)cyclopropane-1-carboxamido)phenyl)-2,4-difluorobenzamide (F24)

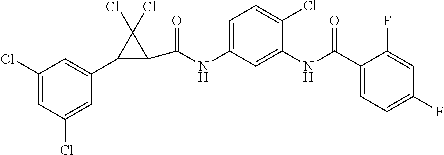 [SMILES: O=C(Nc1cc(NC(=O)C2C(c3cc(Cl)cc(Cl)c3)C2(Cl)Cl)ccc1Cl)c1ccc(F)cc1F]

The title compound was prepared and was isolated as a white solid (0.032 g, 38%).

### trans-rac-N-(2-Chloro-5-(2,2-dichloro-3-(3,5-dichlorophenyl)cyclopropane-1-carboxamido)phenyl)-2,6-difluorobenzamide (F25)

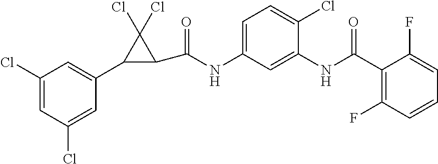 [SMILES: O=C(Nc1cc(NC(=O)C2C(c3cc(Cl)cc(Cl)c3)C2(Cl)Cl)ccc1Cl)c1c(F)cccc1F]

The title compound was prepared and was isolated as a white solid (0.1 g, 47%).

### trans-rac-N-(2-Chloro-5-(2,2-dichloro-3-(3,5-dichlorophenyl)cyclopropane-1-carboxamido)phenyl)-4-cyano-2-methylbenzamide (F27)

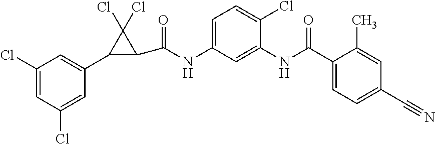 [SMILES: Cc1cc(C#N)ccc1C(=O)Nc1cc(NC(=O)C2C(c3cc(Cl)cc(Cl)c3)C2(Cl)Cl)ccc1Cl]

The title compound was prepared and was isolated as a white solid (0.035 g, 48%).

### trans-rac-N-(2-Chloro-5-(2,2-dichloro-3-(3,5-dichlorophenyl)cyclopropane-1-carboxamido)phenyl)furan-2-carboxamide (F34)

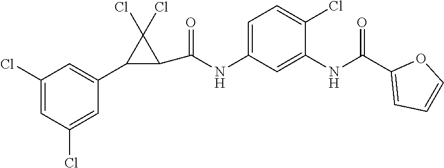 [SMILES: O=C(Nc1cc(NC(=O)C2C(c3cc(Cl)cc(Cl)c3)C2(Cl)Cl)ccc1Cl)c1ccco1]

The title compound was prepared and was isolated as an off-white solid (0.06 g, 58%).

### trans-rac-N-(2-Chloro-5-(2,2-dichloro-3-(3,5-dichlorophenyl)cyclopropane-1-carboxamido)phenyl)tetrahydrofuran-2-carboxamide (F38)

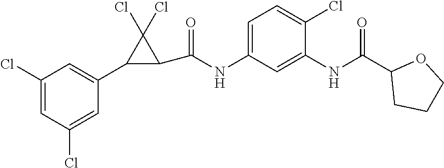 [SMILES: O=C(Nc1cc(NC(=O)C2C(c3cc(Cl)cc(Cl)c3)C2(Cl)Cl)ccc1Cl)C1CCCO1]

The title compound was prepared and was isolated as an off-white solid (0.05 g, 55%).

### trans-rac-2,2-Dichloro-N-(4-chloro-3-(2-phenoxypropanamido)phenyl)-3-(3,5-dichlorophenyl)cyclopropane-1-carboxamide (F41)

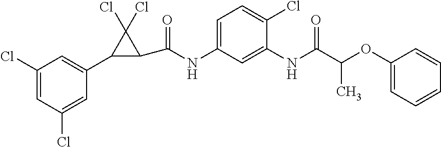 [SMILES: CC(Oc1ccccc1)C(=O)Nc1cc(NC(=O)C2C(c3cc(Cl)cc(Cl)c3)C2(Cl)Cl)ccc1Cl]

The title compound was prepared and was isolated as an off-white solid (0.05 g, 55%).

### trans-rac-N-(3-Butyramido-4-chlorophenyl)-2,2-dichloro-3-(3,5-dichlorophenyl)cyclopropane-1-carboxamide (F45)

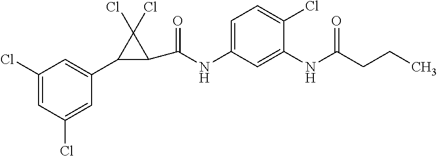 [SMILES: CCCC(=O)Nc1cc(NC(=O)C2C(c3cc(Cl)cc(Cl)c3)C2(Cl)Cl)ccc1Cl]

The title compound was prepared and was isolated as an off-white solid (0.069 g, 55%).

### trans-rac-2,2-Dichloro-N-(4-chloro-3-pentanamidophenyl)-3-(3,5-dichlorophenyl)cyclopropane-1-carboxamide (F46)

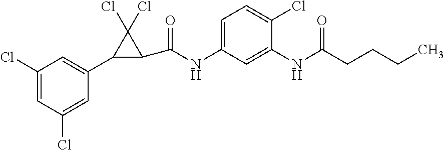 [SMILES: CCCCC(=O)Nc1cc(NC(=O)C2C(c3cc(Cl)cc(Cl)c3)C2(Cl)Cl)ccc1Cl]

The title compound was prepared and was isolated as an off-white solid (0.055 g, 55%).

### trans-rac-2,2-Dichloro-N-(4-chloro-3-(2-morpholinoacetamido)phenyl)-3-(3,5-dichlorophenyl)cyclopropane-1-carboxamide (F50)

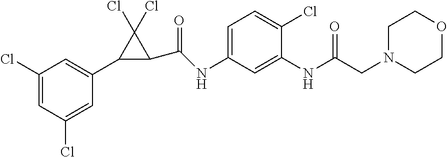 [SMILES: O=C(CN1CCOCC1)Nc1cc(NC(=O)C2C(c3cc(Cl)cc(Cl)c3)C2(Cl)Cl)ccc1Cl]

The title compound was prepared and was isolated as an off-white solid (0.059 g, 55%).

### trans-rac-N-(2-Chloro-5-(2,2-dichloro-3-(3,5-dichlorophenyl)cyclopropane-1-carboxamido)phenyl)-2-fluorobenzamide (F65)

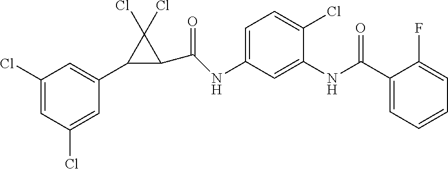 [SMILES: O=C(Nc1cc(NC(=O)C2C(c3cc(Cl)cc(Cl)c3)C2(Cl)Cl)ccc1Cl)c1ccccc1F]

The title compound was prepared and was isolated as an off-white solid (0.09 g, 23%).

### trans-rac-N-(2-Chloro-5-(2,2-dichloro-3-(3,5-dichlorophenyl)cyclopropane-1-carboxamido)phenyl)-3-fluorobenzamide (F66)

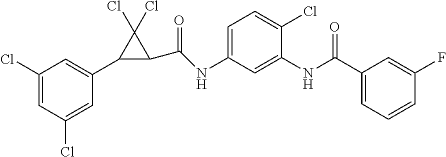 [SMILES: O=C(Nc1cc(NC(=O)C2C(c3cc(Cl)cc(Cl)c3)C2(Cl)Cl)ccc1Cl)c1cccc(F)c1]

The title compound was prepared and was isolated as an off-white solid (0.05 g, 20%).

### trans-rac-N-(2-Chloro-5-(2,2-dichloro-3-(3,5-dichlorophenyl)cyclopropane-1-carboxamido)phenyl)-3,4-difluorobenzamide (F67)

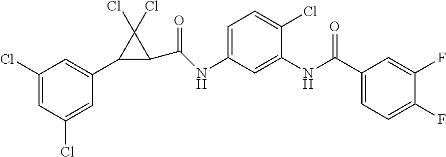 [SMILES: O=C(Nc1cc(NC(=O)C2C(c3cc(Cl)cc(Cl)c3)C2(Cl)Cl)ccc1Cl)c1ccc(F)c(F)c1]

The title compound was prepared and was isolated as a pale yellow solid (0.08 g, 9%).

### trans-rac-N-(2-Chloro-5-(2,2-dichloro-3-(3,5-dichlorophenyl)cyclopropane-1-carboxamido)phenyl)-2,3-difluorobenzamide (F68)

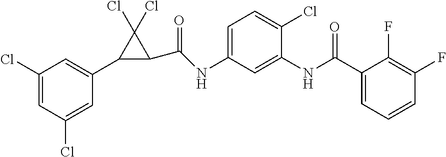 [SMILES: O=C(Nc1cc(NC(=O)C2C(c3cc(Cl)cc(Cl)c3)C2(Cl)Cl)ccc1Cl)c1cccc(F)c1F]

The title compound was prepared and was isolated as an off-white solid (0.09 g, 10%).

### trans-rac-N-(2-Chloro-5-(2,2-dichloro-3-(3,5-dichlorophenyl)cyclopropane-1-carboxamido)phenyl)-2,5-difluorobenzamide (F69)

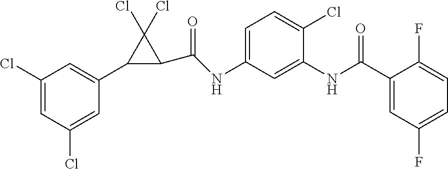 [SMILES: O=C(Nc1cc(NC(=O)C2C(c3cc(Cl)cc(Cl)c3)C2(Cl)Cl)ccc1Cl)c1cc(F)ccc1F]

The title compound was prepared and was isolated as an off-white solid (0.1 g, 25%).

### trans-rac-N-(2-Chloro-5-(2,2-dichloro-3-(3,5-dichlorophenyl)cyclopropane-1-carboxamido)phenyl)-4-methylbenzamide (F75)

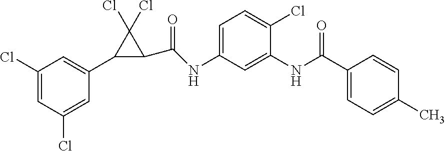 [SMILES: Cc1ccc(C(=O)Nc2cc(NC(=O)C3C(c4cc(Cl)cc(Cl)c4)C3(Cl)Cl)ccc2Cl)cc1]

The title compound was prepared and was isolated as an off-white solid (0.17 g, 47%).

### trans-rac-N-(2-Chloro-5-(2,2-dichloro-3-(3,5-dichlorophenyl)cyclopropane-1-carboxamido)phenyl)-3,5-dimethylbenzamide (F76)

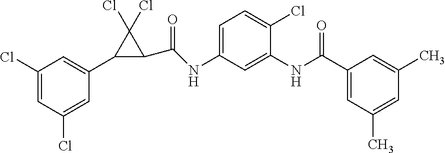 [SMILES: Cc1cc(C)cc(C(=O)Nc2cc(NC(=O)C3C(c4cc(Cl)cc(Cl)c4)C3(Cl)Cl)ccc2Cl)c1]

The title compound was prepared and was isolated as an off-white solid (0.17 g, 53%).

### trans-rac-N-(2-Chloro-5-(2,2-dichloro-3-(3,5-dichlorophenyl)cyclopropane-1-carboxamido)phenyl)-4-fluoro-2-methylbenzamide (F77)

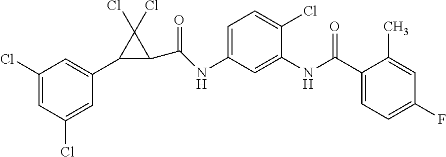 [SMILES: Cc1cc(F)ccc1C(=O)Nc1cc(NC(=O)C2C(c3cc(Cl)cc(Cl)c3)C2(Cl)Cl)ccc1Cl]

The title compound was prepared and was isolated as a white solid (0.1 g, 30%).

### trans-rac-2-Chloro-N-(2-chloro-5-(2,2-dichloro-3-(3,5-dichlorophenyl)cyclopropane-1-carboxamido)phenyl)benzamide (F78)

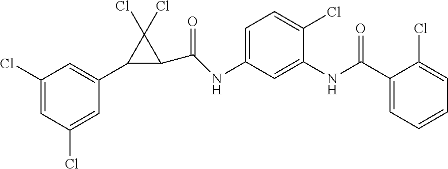 [SMILES: O=C(Nc1cc(NC(=O)C2C(c3cc(Cl)cc(Cl)c3)C2(Cl)Cl)ccc1Cl)c1ccccc1Cl]

The title compound was prepared and was isolated as a white solid (0.14 g, 35%).

### trans-rac-4-Chloro-N-(2-chloro-5-(2,2-dichloro-3-(3,5-dichlorophenyl)cyclopropane-1-carboxamido)phenyl)benzamide (F79)

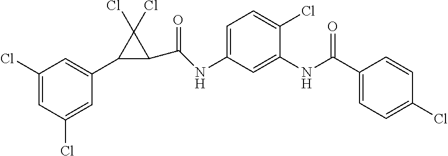 [SMILES: O=C(Nc1cc(NC(=O)C2C(c3cc(Cl)cc(Cl)c3)C2(Cl)Cl)ccc1Cl)c1ccc(Cl)cc1]

The title compound was prepared and was isolated as a white solid (0.18 g, 48%).

### trans-rac-N-(2-Chloro-5-(2,2-dichloro-3-(3,5-dichlorophenyl)cyclopropane-1-carboxamido)phenyl)-4-fluoro-3-(trifluoromethyl)benzamide (F83)

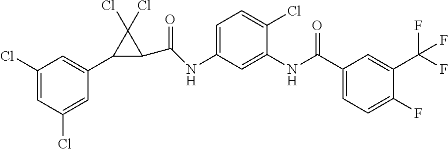 [SMILES: O=C(Nc1cc(NC(=O)C2C(c3cc(Cl)cc(Cl)c3)C2(Cl)Cl)ccc1Cl)c1ccc(F)c(C(F)(F)F)c1]

The title compound was prepared and was isolated as a white solid (0.08 g, 43%).

### trans-rac-N-(2-Chloro-5-(2,2-dichloro-3-(3,5-dichlorophenyl)cyclopropane-1-carboxamido)phenyl)-3-methoxybenzamide (F84)

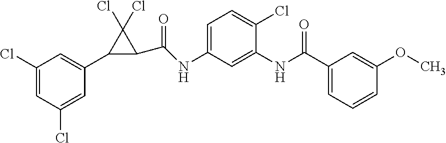 [SMILES: COc1cccc(C(=O)Nc2cc(NC(=O)C3C(c4cc(Cl)cc(Cl)c4)C3(Cl)Cl)ccc2Cl)c1]

The title compound was prepared and was isolated as a white solid (0.22 g, 55%).

### trans-rac-N-(2-Chloro-5-(2,2-dichloro-3-(3,5-dichlorophenyl)cyclopropane-1-carboxamido)phenyl)-4-methoxybenzamide (F85)

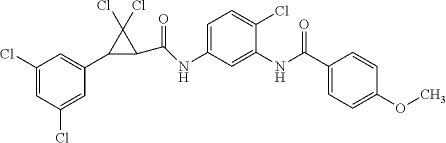 [SMILES: COc1ccc(C(=O)Nc2cc(NC(=O)C3C(c4cc(Cl)cc(Cl)c4)C3(Cl)Cl)ccc2Cl)cc1]

The title compound was prepared and was isolated as a white solid (0.17 g, 51%).

### trans-rac-N-(2-Chloro-5-(2,2-dichloro-3-(3,5-dichlorophenyl)cyclopropane-1-carboxamido)phenyl)benzamide (F87)

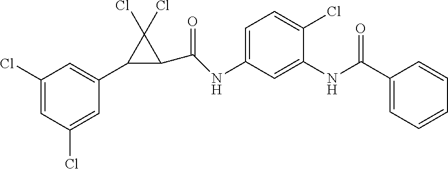 [SMILES: O=C(Nc1cc(NC(=O)C2C(c3cc(Cl)cc(Cl)c3)C2(Cl)Cl)ccc1Cl)c1ccccc1]

The title compound was prepared and was isolated as an off-white solid (0.05 g, 25%).

### N-(2-Chloro-5-nitrophenyl)acetamide (C4)

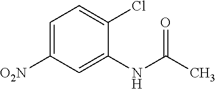

The title compound was prepared from 2-chloro-5-nitroaniline and was isolated as a brown solid (0.395 g, 63%): ¹H NMR (400 MHz, CDCl₃) δ 9.33 (d, J=2.5 Hz, 1H), 7.92 (dd, J=8.8, 2.7 Hz, 1H), 7.70 (s, 1H), 7.54 (d, J=8.8 Hz, 1H), 2.30 (s, 3H); EIMS m/z 214.

### N-(2-Chloro-5-nitrophenyl)-4-fluorobenzamide (C5)

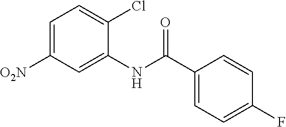 [SMILES: O=C(Nc1cc([N+](=O)[O-])ccc1Cl)c1ccc(F)cc1]

The title compound was prepared from 2-chloro-5-nitroaniline and was isolated as a yellow solid (0.127 g, 13%): ¹H NMR (400 MHz, CDCl₃) δ 9.51 (d, J=2.7 Hz, 1H), 8.46 (s, 1H), 8.01-7.92 (m, 3H), 7.61 (d, J=8.8 Hz, 1H), 7.26-7.20 (m, 2H); EIMS m/z 294.

### N-(2-Chloro-5-nitrophenyl)cyclopropanecarboxamide (C6)

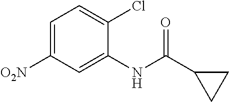 [SMILES: O=C(Nc1cc([N+](=O)[O-])ccc1Cl)C1CC1]

The title compound was prepared from 2-chloro-5-nitroaniline and was isolated as a brown solid (0.354 g, 46%): ¹H NMR (400 MHz, CDCl₃) δ 9.36 (d, J=2.7 Hz, 1H), 7.95 (s, 1H), 7.90 (dd, J=8.8, 2.7 Hz, 1H), 7.54 (d, J=8.8 Hz, 1H), 1.66-1.58 (m, 1H), 1.20-1.16 (m, 2H), 1.00-0.94 (m, 2H); EIMS m/z 239.

### Example 3

### Preparation of trans-rac-N-(2-chloro-5-(2,2-dichloro-3-(3,5-dichlorophenyl)cyclopropane-1-carboxamido)phenyl)picolinamide (F28)

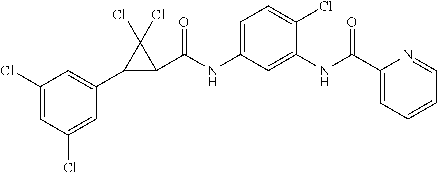 [SMILES: O=C(Nc1cc(NC(=O)C2C(c3cc(Cl)cc(Cl)c3)C2(Cl)Cl)ccc1Cl)c1ccccn1]

To a solution of trans-rac-N-(3-amino-4-chlorophenyl)-2,2-dichloro-3-(3,5-dichlorophenyl)cyclopropane-1-carboxamide (C3, 0.15 g, 0.35 mmol) in N,N-dimethylformamide (5 mL) stirred at 0° C. was added picolinic acid (0.6 g, 4.87 mmol). 1-Ethyl-3-(3-dimethylaminopropyl)carbodiimide hydrochloride (EDC·HCl, 0.14 g, 0.71 mmol) and 1-hydroxybenzotriazole (HOBt, 0.95 g, 0.71 mmol) were added. The reaction mixture was warmed to 60° C. for 16 hours. The reaction mixture was cooled to room temperature, diluted with water and extracted with ethyl acetate (3×30 mL). The combined organic layers were washed with saturated aqueous sodium bicarbonate, washed with brine, dried over anhydrous sodium sulfate, filtered, and concentrated under reduced pressure. Purification of the resulting product by column chromatography eluting with 20-30% ethyl acetate in petroleum ether afforded the title compound as an off-white solid (0.1 g, 58%).

The following compounds were prepared in accordance with the procedure in Example 3:

### trans-rac-N-(2-Chloro-5-(2,2-dichloro-3-(3,5-dichlorophenyl)cyclopropane-1-carboxamido)phenyl)pyrimidine-2-carboxamide (F29)

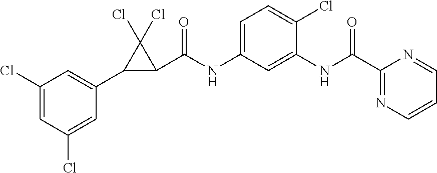 [SMILES: O=C(Nc1cc(NC(=O)C2C(c3cc(Cl)cc(Cl)c3)C2(Cl)Cl)ccc1Cl)c1ncccn1]

The title compound was prepared and was isolated as an off-white solid (0.07 g, 40%).

### trans-rac-N-(2-Chloro-5-(2,2-dichloro-3-(3,5-dichlorophenyl)cyclopropane-1-carboxamido)phenyl)-5-fluoropicolinamide (F30)

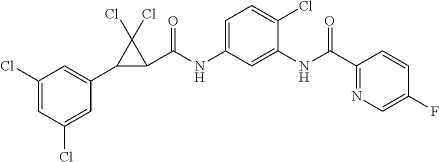 [SMILES: O=C(Nc1cc(NC(=O)C2C(c3cc(Cl)cc(Cl)c3)C2(Cl)Cl)ccc1Cl)c1ccc(F)cn1]

The title compound was prepared and was isolated as an off-white solid (0.07 g, 40%).

### trans-rac-N-(2-Chloro-5-(2,2-dichloro-3-(3,5-dichlorophenyl)cyclopropane-1-carboxamido)phenyl)-3,5-difluoropicolinamide (F31)

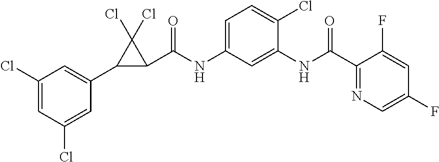 [SMILES: O=C(Nc1cc(NC(=O)C2C(c3cc(Cl)cc(Cl)c3)C2(Cl)Cl)ccc1Cl)c1ncc(F)cc1F]

The title compound was prepared and was isolated as an off-white solid (0.06 g, 34%).

### trans-rac-N-(2-Chloro-5-(2,2-dichloro-3-(3,5-dichlorophenyl)cyclopropane-1-carboxamido)phenyl)pyrimidine-4-carboxamide (F32)

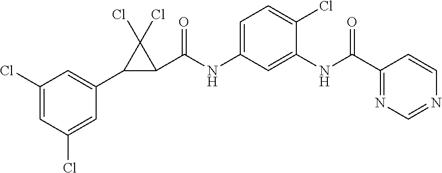 [SMILES: O=C(Nc1cc(NC(=O)C2C(c3cc(Cl)cc(Cl)c3)C2(Cl)Cl)ccc1Cl)c1ccncn1]

The title compound was prepared and was isolated as an off-white solid (0.075 g, 43%).

### Example 4

### Preparation of trans-rac-2,2-dichloro-N-(4-chloro-3-(2-ethoxypropanamido)phenyl)-3-(3,5-dichlorophenyl)cyclopropane-1-carboxamide (F40)

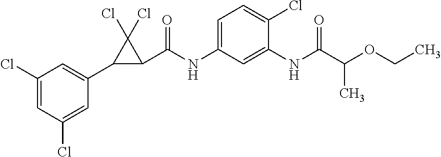 [SMILES: CCOC(C)C(=O)Nc1cc(NC(=O)C2C(c3cc(Cl)cc(Cl)c3)C2(Cl)Cl)ccc1Cl]

To a solution of trans-rac-N-(3-amino-4-chlorophenyl)-2,2-dichloro-3-(3,5-dichlorophenyl)cyclopropane-1-carboxamide (C3, 0.15 g, 0.35 mmol) in dichloromethane (5 mL) were added 2-ethoxypropanoic acid (0.55 g, 4.65 mmol), 2-(1H-benzotriazole-1-yl)-1,1,3,3-tetramethylaminium tetrafluoroborate (TBTU, 0.14 g, 0.46 mmol) and triethylamine (0.1 mL, 0.72 mmol). The reaction mixture was stirred at room temperature for 16 hours. The reaction mixture was diluted with water and was extracted with ethyl acetate (2×20 mL). The combined organic layers were dried over anhydrous sodium sulfate, filtered, and concentrated under reduced pressure. Purification of the resulting product by column chromatography eluting with 20-30% ethyl acetate in petroleum ether afforded the title compound as an off-white solid (0.09 g, 52%).

The following compounds were prepared in accordance with the procedure in Example 4:

### trans-rac-2,2-Dichloro-3-(3,5-dichlorophenyl)-N-(3-(2-methoxyacetamido)phenyl)cyclopropane-1-carboxamide (F4)

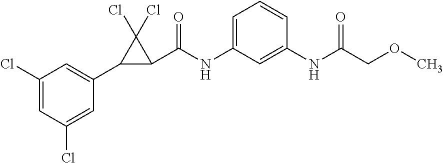 [SMILES: COCC(=O)Nc1cccc(NC(=O)C2C(c3cc(Cl)cc(Cl)c3)C2(Cl)Cl)c1]

The title compound was prepared from trans-2,2-dichloro-3-(3,5-dichlorophenyl)cyclopropane-1-carboxylic acid and was isolated as a white solid (0.200 g, 87%).

### trans-rac-2,2-Dichloro-N-(4-chloro-3-(2-phenylacetamido)phenyl)-3-(3,5-dichlorophenyl)cyclopropane-1-carboxamide (F48)

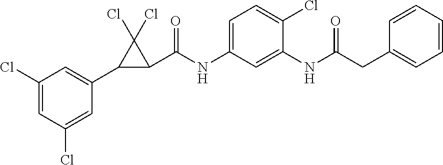 [SMILES: O=C(Cc1ccccc1)Nc1cc(NC(=O)C2C(c3cc(Cl)cc(Cl)c3)C2(Cl)Cl)ccc1Cl]

The title compound was prepared and was isolated as a pale yellow solid (0.075 g, 62%).

### trans-rac-2,2-Dichloro-N-(4-chloro-3-(2-(2,4,6-trifluorophenyl)acetamido)phenyl)-3-(3,5-dichlorophenyl)cyclopropane-1-carboxamide (F49)

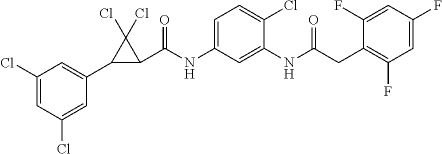 [SMILES: O=C(Cc1c(F)cc(F)cc1F)Nc1cc(NC(=O)C2C(c3cc(Cl)cc(Cl)c3)C2(Cl)Cl)ccc1Cl]

The title compound was prepared and was isolated as an off-white solid (0.06 g, 42%).

### trans-rac-2,2-Dichloro-N-(4-chloro-3-(2,2-difluoro-2-phenylacetamido)phenyl)-3-(3,5-dichlorophenyl)cyclopropane-1-carboxamide (F57)

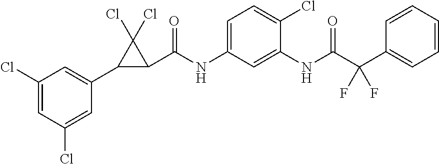 [SMILES: O=C(Nc1ccc(Cl)c(NC(=O)C(F)(F)c2ccccc2)c1)C1C(c2cc(Cl)cc(Cl)c2)C1(Cl)Cl]

The title compound was prepared and was isolated as a pale yellow solid (0.06 g, 46%).

### Example 5

### Preparation of trans-rac-N-(2-chloro-5-(2,2-dichloro-3-(3,5-dichlorophenyl)cyclopropane-1-carboxamido)phenyl)-3,5-difluorobenzamide (F26)

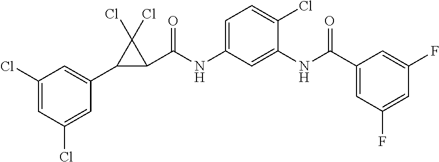 [SMILES: O=C(Nc1cc(NC(=O)C2C(c3cc(Cl)cc(Cl)c3)C2(Cl)Cl)ccc1Cl)c1cc(F)cc(F)c1]

To a solution of trans-rac-N-(3-amino-4-chlorophenyl)-2,2-dichloro-3-(3,5-dichlorophenyl)cyclopropane-1-carboxamide (C3, 0.15 g, 0.35 mmol) in dry toluene (10 mL) was added a 2 molar (M) solution of trimethylaluminum (0.5 mL, 1.06 mmol) in toluene. The reaction mixture was stirred at room temperature for 1 hour. Methyl 3,5-difluorobenzoate (0.07 g, 0.39 mmol) was added, and the reaction mixture was stirred at room temperature for 16 hours. The reaction mixture was poured into water and was extracted with ethyl acetate (3×20 mL). The organic layer was washed with brine, dried over sodium sulfate, filtered, and concentrated under reduced pressure. The resulting product was triturated with n-pentane and the solids were collected by filtration to afford the title compound as an off-white solid (0.08 g, 40%).

The following compounds were prepared in accordance with the procedure in Example 5:

### trans-rac-N-(2-Chloro-5-(2,2-dichloro-3-(3,5-dichlorophenyl)cyclopropane-1-carboxamido)phenyl)isoxazole-5-carboxamide (F35)

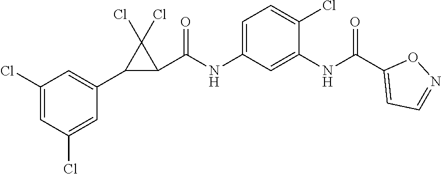 [SMILES: O=C(Nc1cc(NC(=O)C2C(c3cc(Cl)cc(Cl)c3)C2(Cl)Cl)ccc1Cl)c1ccno1]

The title compound was prepared and was isolated as an off-white solid (0.1 g, 55%).

### trans-rac-N-(3-(2-Acetamidoacetamido)-4-chlorophenyl)-2,2-dichloro-3-(3,5-dichlorophenyl)cyclopropane-1-carboxamide (F51)

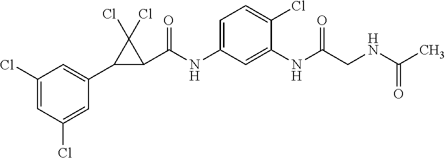 [SMILES: CC(=O)NCC(=O)Nc1cc(NC(=O)C2C(c3cc(Cl)cc(Cl)c3)C2(Cl)Cl)ccc1Cl]

The title compound was prepared and was isolated as an off-white solid (0.05 g, 41%).

### trans-rac-N-(2-Chloro-5-(2,2-dichloro-3-(3,5-dichlorophenyl)cyclopropane-1-carboxamido)phenyl)-1-(2,2,2-trifluoroethyl)pyrrolidine-2-carboxamide (F52)

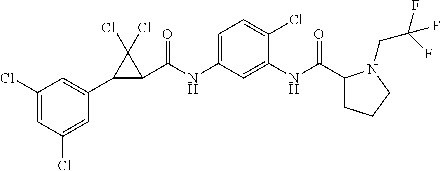 [SMILES: O=C(Nc1cc(NC(=O)C2C(c3cc(Cl)cc(Cl)c3)C2(Cl)Cl)ccc1Cl)C1CCCN1CC(F)(F)F]

The title compound was prepared and was isolated as an off-white solid (0.045 g, 26%).

### Example 6

### Preparation of trans-rac-N-(2-chloro-5-(2,2-dichloro-3-(3,5-dichlorophenyl)cyclopropane-1-carboxamido)phenyl)-6-oxo-1,6-dihydropyridine-2-carboxamide (F33)

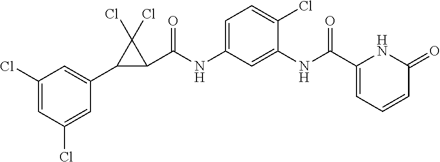 [SMILES: O=C(Nc1cc(NC(=O)C2C(c3cc(Cl)cc(Cl)c3)C2(Cl)Cl)ccc1Cl)c1cccc(=O)[nH]1]

To a solution of trans-rac-N-(3-amino-4-chlorophenyl)-2,2-dichloro-3-(3,5-dichlorophenyl)cyclopropane-1-carboxamide (C3, 0.15 g, 0.35 mmol) in dichloromethane (10 mL) was added 6-oxo-1,6-dihydropyridine-2-carboxylic acid (0.03 g, 0.39 mmol). EDC·HCl (0.1 g, 0.53 mmol) and 4-dimethylaminopyridine (DMAP, 0.04 g, 0.39 mmol) were then added. The reaction mixture was stirred at room temperature for 16 hours and the reaction was quenched with water. The mixture was extracted with ethyl acetate (3×30 mL). The organic layer was washed with 1 normal (N) aqueous hydrogen chloride, with saturated aqueous sodium bicarbonate, and with brine, dried over anhydrous sodium sulfate, filtered, and concentrated under reduced pressure. Purification of the resulting product by column chromatography eluting with 50-80% ethyl acetate in petroleum ether afforded the title compound as a pale yellow solid (0.06 g, 34%).

The following compounds were prepared in accordance with the procedure in Example 6:

### trans-rac-N-(2-Chloro-5-(2,2-dichloro-3-(3,5-dichlorophenyl)cyclopropane-1-carboxamido)phenyl)-4-(trifluoromethyl)thiazole-2-carboxamide (F36)

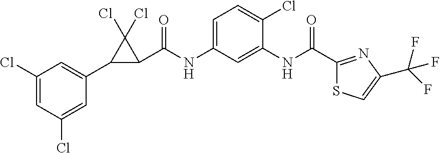 [SMILES: O=C(Nc1cc(NC(=O)C2C(c3cc(Cl)cc(Cl)c3)C2(Cl)Cl)ccc1Cl)c1nc(C(F)(F)F)cs1]

The title compound was prepared and was isolated as an off-white solid (0.65 g, 37%).

### trans-rac-N-(2-Chloro-5-(2,2-dichloro-3-(3,5-dichlorophenyl)cyclopropane-1-carboxamido)phenyl)-3-oxo-2,3-dihydroisoxazole-5-carboxamide (F37)

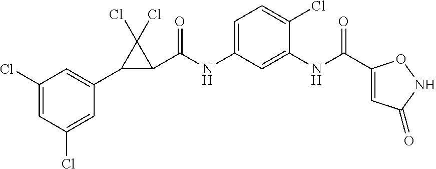 [SMILES: O=C(Nc1cc(NC(=O)C2C(c3cc(Cl)cc(Cl)c3)C2(Cl)Cl)ccc1Cl)c1cc(=O)[nH]o1]

The title compound was prepared and was isolated as an off-white solid (0.06 g, 29%).

### trans-rac-2,2-Dichloro-N-(4-chloro-3-(2-methoxyacetamido)phenyl)-3-(3,5-dichlorophenyl)cyclopropane-1-carboxamide (F39)

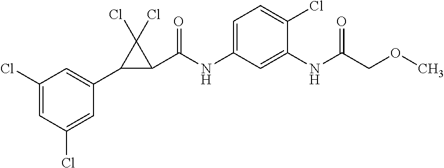 [SMILES: COCC(=O)Nc1cc(NC(=O)C2C(c3cc(Cl)cc(Cl)c3)C2(Cl)Cl)ccc1Cl]

The title compound was prepared and was isolated as an off-white solid (0.09 g, 51%).

### trans-rac-2,2-Dichloro-N-(4-chloro-3-(2-cyanoacetamido)phenyl)-3-(3,5-dichlorophenyl)cyclopropanecarboxamide (F42)

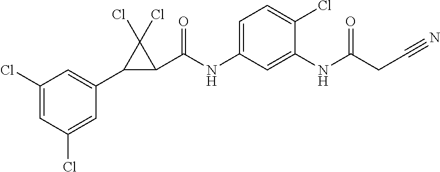 [SMILES: N#CCC(=O)Nc1cc(NC(=O)C2C(c3cc(Cl)cc(Cl)c3)C2(Cl)Cl)ccc1Cl]

The title compound was prepared and was isolated as an off-white solid (0.1 g, 63%).

### trans-rac-2,2-Dichloro-N-(4-chloro-3-(1-cyanocyclopropane-1-carboxamido)phenyl)-3-(3,5-dichlorophenyl)cyclopropane-1-carboxamide (F43)

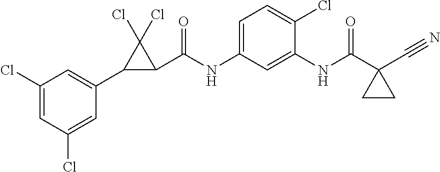 [SMILES: N#CC1(C(=O)Nc2cc(NC(=O)C3C(c4cc(Cl)cc(Cl)c4)C3(Cl)Cl)ccc2Cl)CC1]

The title compound was prepared and was isolated as an off-white solid (0.04 g, 15%).

### trans-rac-N-(3-But-3-enamido-4-chlorophenyl)-2,2-dichloro-3-(3,5-dichlorophenyl)cyclopropane-1-carboxamide (F47)

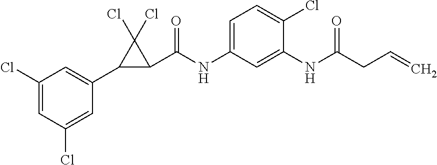 [SMILES: C=CCC(=O)Nc1cc(NC(=O)C2C(c3cc(Cl)cc(Cl)c3)C2(Cl)Cl)ccc1Cl]

The title compound was prepared and was isolated as an off-white solid (0.07 g, 40%).

### trans-rac-2,2-Dichloro-N-(4-chloro-3-(2-chloro-2,2-difluoroacetamido)phenyl)-3-(3,5-dichlorophenyl)cyclopropane-1-carboxamide (F54)

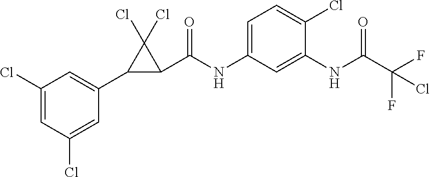 [SMILES: O=C(Nc1ccc(Cl)c(NC(=O)C(F)(F)Cl)c1)C1C(c2cc(Cl)cc(Cl)c2)C1(Cl)Cl]

The title compound was prepared and was isolated as an off-white solid (0.13 g, 56%).

### trans-rac-2,2-Dichloro-N-(4-chloro-3-(2,2,3,3-tetrafluoropropanamido)phenyl)-3-(3,5-dichlorophenyl)cyclopropane-1-carboxamide (F55)

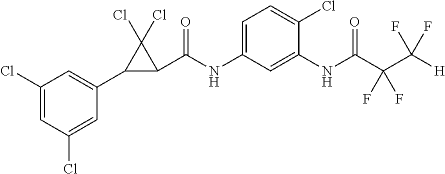 [SMILES: O=C(Nc1ccc(Cl)c(NC(=O)C(F)(F)C(F)F)c1)C1C(c2cc(Cl)cc(Cl)c2)C1(Cl)Cl]

The title compound was prepared and was isolated as an off-white solid (0.08 g, 42%).

### trans-rac-2,2-Dichloro-N-(4-chloro-3-(2,2,3,3,4,4,4-heptafluorobutanamido)phenyl)-3-(3,5-dichlorophenyl)cyclopropane-1-carboxamide (F56)

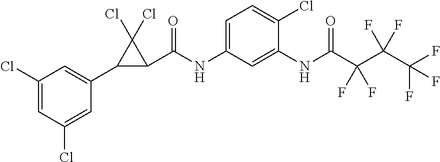 [SMILES: O=C(Nc1ccc(Cl)c(NC(=O)C(F)(F)C(F)(F)C(F)(F)F)c1)C1C(c2cc(Cl)cc(Cl)c2)C1(Cl)Cl]

The title compound was prepared and was isolated as an off-white solid (0.08 g, 40%).

### trans-rac-2,2-Dichloro-N-(4-chloro-3-(2,2-difluorobutanamido)phenyl)-3-(3,5-dichlorophenyl)cyclopropane-1-carboxamide (F58)

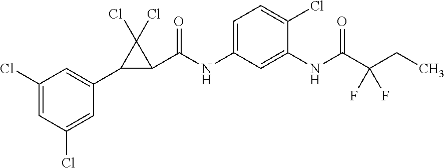 [SMILES: CCC(F)(F)C(=O)Nc1cc(NC(=O)C2C(c3cc(Cl)cc(Cl)c3)C2(Cl)Cl)ccc1Cl]

The title compound was prepared and was isolated as an off-white solid (0.08 g, 46%).

### trans-rac-N-(2-Chloro-5-(2,2-dichloro-3-(3,5-dichlorophenyl)cyclopropane-1-carboxamido)phenyl)-4-cyanobenzamide (F70)

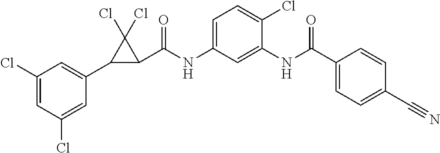 [SMILES: N#Cc1ccc(C(=O)Nc2cc(NC(=O)C3C(c4cc(Cl)cc(Cl)c4)C3(Cl)Cl)ccc2Cl)cc1]

The title compound was prepared and was isolated as an off-white solid (0.09 g, 28%).

### trans-rac-N-(2-Chloro-5-(2,2-dichloro-3-(3,5-dichlorophenyl)cyclopropane-1-carboxamido)phenyl)-4-cyano-2-fluorobenzamide (F71)

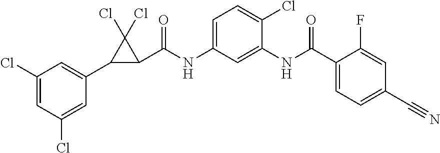 [SMILES: N#Cc1ccc(C(=O)Nc2cc(NC(=O)C3C(c4cc(Cl)cc(Cl)c4)C3(Cl)Cl)ccc2Cl)c(F)c1]

The title compound was prepared and was isolated as a white solid (0.1 g, 25%).

### trans-rac-N-(2-Chloro-5-(2,2-dichloro-3-(3,5-dichlorophenyl)cyclopropane-1-carboxamido)phenyl)-4-cyano-3-fluorobenzamide (F72)

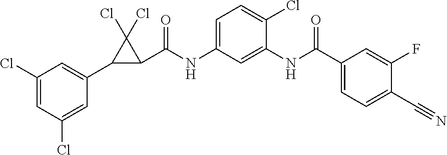 [SMILES: N#Cc1ccc(C(=O)Nc2cc(NC(=O)C3C(c4cc(Cl)cc(Cl)c4)C3(Cl)Cl)ccc2Cl)cc1F]

The title compound was prepared and was isolated as an off-white solid (0.1 g, 25%).

### trans-rac-N-(2-Chloro-5-(2,2-dichloro-3-(3,5-dichlorophenyl)cyclopropane-1-carboxamido)phenyl)-2-methylbenzamide (F73)

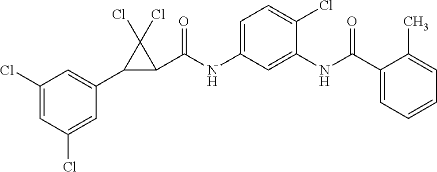 [SMILES: Cc1ccccc1C(=O)Nc1cc(NC(=O)C2C(c3cc(Cl)cc(Cl)c3)C2(Cl)Cl)ccc1Cl]

The title compound was prepared and was isolated as an off-white solid (0.05 g, 13%).

### trans-rac-N-(2-Chloro-5-(2,2-dichloro-3-(3,5-dichlorophenyl)cyclopropane-1-carboxamido)phenyl)-3-methylbenzamide (F74)

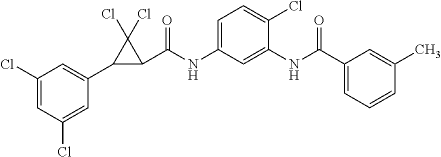 [SMILES: Cc1cccc(C(=O)Nc2cc(NC(=O)C3C(c4cc(Cl)cc(Cl)c4)C3(Cl)Cl)ccc2Cl)c1]

The title compound was prepared and was isolated as an off-white solid (0.08 g, 21%).

### trans-rac-3,5-Dichloro-N-(2-chloro-5-(2,2-dichloro-3-(3,5-dichlorophenyl)cyclopropane-1-carboxamido)phenyl)benzamide (F80)

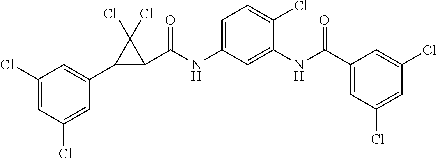 [SMILES: O=C(Nc1cc(NC(=O)C2C(c3cc(Cl)cc(Cl)c3)C2(Cl)Cl)ccc1Cl)c1cc(Cl)cc(Cl)c1]

The title compound was prepared and was isolated as an off-white solid (0.1 g, 34%).

### trans-rac-N-(2-Chloro-5-(2,2-dichloro-3-(3,5-dichlorophenyl)cyclopropane-1-carboxamido)phenyl)-3-(trifluoromethyl)benzamide (F81)

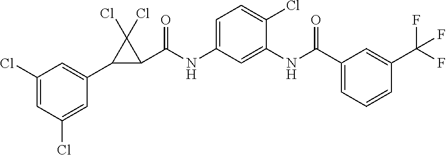 [SMILES: O=C(Nc1cc(NC(=O)C2C(c3cc(Cl)cc(Cl)c3)C2(Cl)Cl)ccc1Cl)c1cccc(C(F)(F)F)c1]

The title compound was prepared and was isolated as an off-white solid (0.06 g, 22%).

### trans-rac-N-(2-Chloro-5-(2,2-dichloro-3-(3,5-dichlorophenyl)cyclopropane-1-carboxamido)phenyl)-4-ethynylbenzamide (F91)

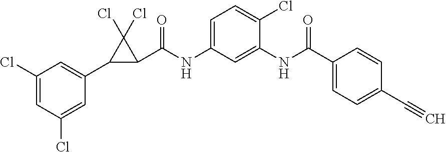 [SMILES: C#Cc1ccc(C(=O)Nc2cc(NC(=O)C3C(c4cc(Cl)cc(Cl)c4)C3(Cl)Cl)ccc2Cl)cc1]

The title compound was prepared and was isolated as an off-white solid (0.03 g, 5%).

### trans-rac-N-(2-Chloro-5-(2,2-dichloro-3-(3,5-dichlorophenyl)cyclopropane-1-carboxamido)phenyl)-3,5-bis(trifluoromethyl)benzamide (F86)

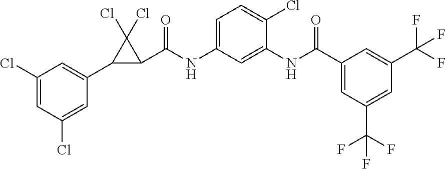 [SMILES: O=C(Nc1cc(NC(=O)C2C(c3cc(Cl)cc(Cl)c3)C2(Cl)Cl)ccc1Cl)c1cc(C(F)(F)F)cc(C(F)(F)F)c1]

The title compound was prepared and was isolated as an off-white solid (0.07 g, 21%).

### trans-rac-N-(3-Acetamido-4-chlorophenyl)-2,2-dichloro-3-(3,5-dichlorophenyl)cyclopropane-1-carboxamide (F1)

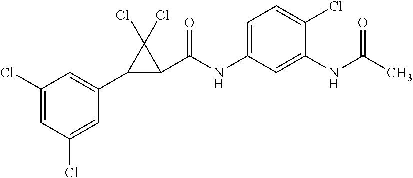 [SMILES: CC(=O)Nc1cc(NC(=O)C2C(c3cc(Cl)cc(Cl)c3)C2(Cl)Cl)ccc1Cl]

The title compound was prepared from trans-2,2-dichloro-3-(3,5-dichlorophenyl)cyclopropane-1-carboxylic acid and N-(5-amino-2-chlorophenyl)acetamide (C7) and was isolated as a brown solid (0.142 g, 64%).

### trans-rac-N-(2-Chloro-5-(2,2-dichloro-3-(3,5-dichlorophenyl)cyclopropane-1-carboxamido)phenyl)-4-fluorobenzamide (F2)

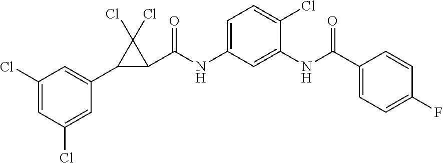 [SMILES: O=C(Nc1cc(NC(=O)C2C(c3cc(Cl)cc(Cl)c3)C2(Cl)Cl)ccc1Cl)c1ccc(F)cc1]

The title compound was prepared from trans-2,2-dichloro-3-(3,5-dichlorophenyl)cyclopropane-1-carboxylic acid and N-(5-amino-2-chlorophenyl)-4-fluorobenzamide (C8) and was isolated as a light brown solid (0.081 g, 55%).

### trans-rac-2,2-Dichloro-N-(4-chloro-3-(cyclopropanecarboxamido)phenyl)-3-(3,5-dichlorophenyl)cyclopropane-1-carboxamide (F3)

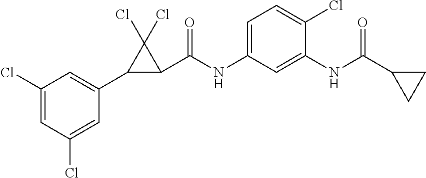 [SMILES: O=C(Nc1cc(NC(=O)C2C(c3cc(Cl)cc(Cl)c3)C2(Cl)Cl)ccc1Cl)C1CC1]

The title compound was prepared from trans-2,2-dichloro-3-(3,5-dichlorophenyl)cyclopropane-1-carboxylic acid and N-(5-amino-2-chlorophenyl)cyclopropanecarboxamide (C9) and was isolated as a brown solid (0.120 g, 57%).

### Example 7

### Preparation of trans-rac-2,2-dichloro-N-(4-chloro-3-(2,2,2-trifluoroacetamido)phenyl)-3-(3,5-dichlorophenyl)cyclopropane-1-carboxamide (F53)

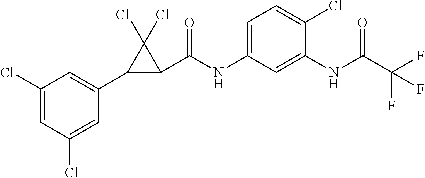 [SMILES: O=C(Nc1ccc(Cl)c(NC(=O)C(F)(F)F)c1)C1C(c2cc(Cl)cc(Cl)c2)C1(Cl)Cl]

To a stirred solution of trans-rac-N-(3-amino-4-chlorophenyl)-2,2-dichloro-3-(3,5-dichlorophenyl)cyclopropane-1-carboxamide (C3, 0.2 g, 0.47 mmol) in dichloromethane (5 mL) at 0° C. was added trifluoroacetic anhydride (0.2 g, 0.94 mmol). The reaction mixture was stirred at room temperature for 3 hours, was diluted with dichloromethane (15 mL), and was washed with water and brine (2×10 mL). The organic layer was dried over anhydrous sodium sulfate, filtered, and concentrated under reduced pressure. Purification of the resulting product by column chromatography eluting with 10-15% ethyl acetate in petroleum ether afforded the title compound as an off-white solid (0.1 g, 55%).

### Example 8

### Preparation of N-(5-((1R,3R)-3-(3,5-bis(trifluoromethyl)phenyl)-2,2-dichlorocyclopropane-1-carboxamido)-2-chlorophenyl)-4-cyano-2-methylbenzamide (F96)

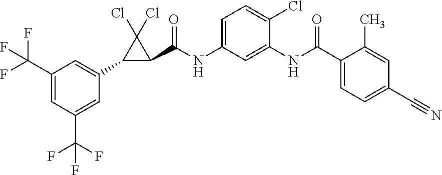 [SMILES: Cc1cc(C#N)ccc1C(=O)Nc1cc(NC(=O)[C@H]2[C@H](c3cc(C(F)(F)F)cc(C(F)(F)F)c3)C2(Cl)Cl)ccc1Cl]

To a solution of (1R,3R)-3-(3,5-bis(trifluoromethyl)phenyl)-2,2-dichlorocyclopropane-1-carboxylic acid (0.065 g, 0.177 mmol) in ethyl acetate (2 ml) were added N-(5-amino-2-chlorophenyl)-4-cyano-2-methylbenzamide (C10, 0.051 g, 0.177 mmol) and pyridine (0.029 ml, 0.354 mmol). A 50% solution of 2,4,6-tripropyl-1,3,5,2,4,6-trioxatriphosphinane 2,4,6-trioxide (0.169 g, 0.266 mmol) in ethyl acetate was added and the reaction mixture was warmed to 65° C. for 48 hours. The reaction mixture was cooled to room temperature and concentrated under a stream of nitrogen. Purification of the residue by column chromatography eluting with 0-50% ethyl acetate in hexanes afforded the title compound as a white foam (0.108 g, 96%).

The following compounds were prepared in accordance with the procedure in Example 8:

### trans-rac-2,2-Dichloro-N-(2,4-difluoro-3-(2-(2,2,2-trifluoroethoxy)acetamido)phenyl)-3-(4-fluoro-3-(trifluoromethyl)phenyl)cyclopropane-1-carboxamide (F95)

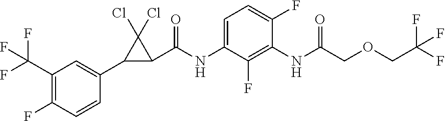 [SMILES: O=C(COCC(F)(F)F)Nc1c(F)ccc(NC(=O)C2C(c3ccc(F)c(C(F)(F)F)c3)C2(Cl)Cl)c1F]

The title compound was prepared from trans-2,2-dichloro-3-(4-fluoro-3-(trifluoromethyl)phenyl)cyclopropane-1-carboxylic acid and was isolated as a white solid (0.088 g, 41%).

### N-(2-Chloro-5-((1R,3R)-2,2-dichloro-3-(4-fluoro-3-(trifluoromethyl)phenyl)cyclopropane-1-carboxamido)phenyl)-4-cyano-2-methylbenzamide (F97)

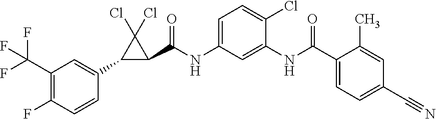 [SMILES: Cc1cc(C#N)ccc1C(=O)Nc1cc(NC(=O)[C@H]2[C@H](c3ccc(F)c(C(F)(F)F)c3)C2(Cl)Cl)ccc1Cl]

The title compound was prepared from (1R,3R)-2,2-dichloro-3-(4-fluoro-3-(trifluoromethyl)phenyl)cyclopropane-1-carboxylic acid and was isolated as a white foam (0.100 g, 98%).

### N-(5-((1R,3R)-3-(3-Bromo-4-fluorophenyl)-2,2-dichlorocyclopropane-1-carboxamido)-2-chlorophenyl)-4-cyano-2-methylbenzamide (F98)

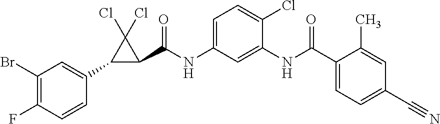 [SMILES: Cc1cc(C#N)ccc1C(=O)Nc1cc(NC(=O)[C@H]2[C@H](c3ccc(F)c(Br)c3)C2(Cl)Cl)ccc1Cl]

The title compound was prepared from (1R,3R)-2,2-dichloro-3-(3-bromo-4-fluorophenyl)cyclopropane-1-carboxylic acid and was isolated as a white foam (0.089 g, 84%).

### N-(2-Chloro-5-((1R,3R)-2,2-dichloro-3-(3,4-dichlorophenyl)cyclopropane-1-carboxamido)phenyl)-4-cyano-2-methylbenzamide (F99)

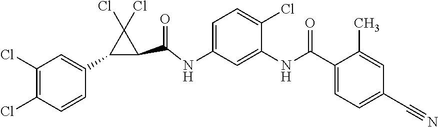 [SMILES: Cc1cc(C#N)ccc1C(=O)Nc1cc(NC(=O)[C@H]2[C@H](c3ccc(Cl)c(Cl)c3)C2(Cl)Cl)ccc1Cl]

The title compound was prepared from (1R,3R)-2,2-dichloro-3-(3,4-dichlorophenyl)cyclopropane-1-carboxylic acid and was isolated as a white foam (0.081 g, 81%).

### N-(2-Chloro-5-((1R,3R)-2,2-dichloro-3-(4-fluoro-3-(trifluoromethyl)phenyl)cyclopropane-1-carboxamido)phenyl)-3,5-difluorobenzamide (F100)

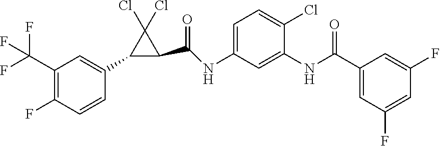 [SMILES: O=C(Nc1cc(NC(=O)[C@H]2[C@H](c3ccc(F)c(C(F)(F)F)c3)C2(Cl)Cl)ccc1Cl)c1cc(F)cc(F)c1]

The title compound was prepared from (1R,3R)-2,2-dichloro-3-(4-fluoro-3-(trifluoromethyl)phenyl)cyclopropane-1-carboxylic acid and was isolated as a white foam (0.147 g, 89%).

### tert-Butyl (3-((2-chloro-5-((1R,3R)-2,2-dichloro-3-(4-fluoro-3-(trifluoromethyl)phenyl)cyclopropane-1-carboxamido)phenyl)carbamoyl)-2,6-difluorophenyl)carbamate (F101)

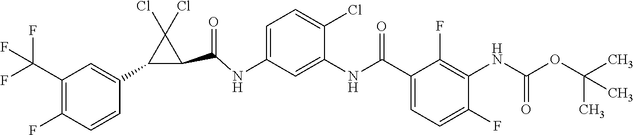 [SMILES: CC(C)(C)OC(=O)Nc1c(F)ccc(C(=O)Nc2cc(NC(=O)[C@H]3[C@H](c4ccc(F)c(C(F)(F)F)c4)C3(Cl)Cl)ccc2Cl)c1F]

The title compound was prepared from (1R,3R)-2,2-dichloro-3-(4-fluoro-3-(trifluoromethyl)phenyl)cyclopropane-1-carboxylic acid and was isolated as a white solid (0.354 g, 49%).

### (1R,3R)-2,2-Dichloro-N-(4-chloro-3-(4,4,4-trifluoro-3-methylbutanamido)phenyl)-3-(4-fluoro-3-(trifluoromethyl)phenyl)cyclopropane-1-carboxamide (F102)

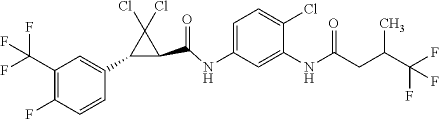 [SMILES: CC(CC(=O)Nc1cc(NC(=O)[C@H]2[C@H](c3ccc(F)c(C(F)(F)F)c3)C2(Cl)Cl)ccc1Cl)C(F)(F)F]

The title compound was prepared from (1R,3R)-2,2-dichloro-3-(4-fluoro-3-(trifluoromethyl)phenyl)cyclopropane-1-carboxylic acid and was isolated as a white foam (0.090 g, 73%).

### N-(2-Chloro-5-((1R,3R)-2,2-dichloro-3-(3,5-dichlorophenyl)cyclopropane-1-carboxamido)phenyl)-3,5-difluoropicolinamide (F106)

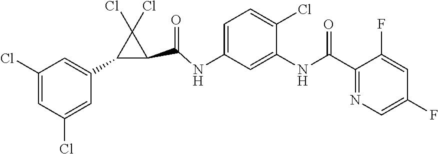 [SMILES: O=C(Nc1cc(NC(=O)[C@H]2[C@H](c3cc(Cl)cc(Cl)c3)C2(Cl)Cl)ccc1Cl)c1ncc(F)cc1F]

The title compound was prepared from (1R,3R)-2,2-dichloro-3-(3,5-dichlorophenyl)cyclopropane-1-carboxylic acid and was isolated as a pale pink solid (0.155 g, 23%).

### trans-rac-2,2-Dichloro-N-(4-chloro-3-nitrophenyl)-3-(4-fluoro-3-(trifluoromethyl)phenyl)cyclopropane-1-carboxamide (C11)

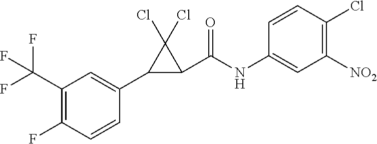 [SMILES: O=C(Nc1ccc(Cl)c([N+](=O)[O-])c1)C1C(c2ccc(F)c(C(F)(F)F)c2)C1(Cl)Cl]

The title compound was prepared from trans-2,2-dichloro-3-(4-fluoro-3-(trifluoromethyl)phenyl)cyclopropane-1-carboxylic acid and was isolated as a pale yellow solid (11.0 g, 73%): ¹H NMR (400 MHz, CDCl₃) δ 8.26 (d, J=2.4 Hz, 1H), 8.10 (br, 1H), 7.84 (dd, J=2.7, 8.7 Hz, 1H), 7.55-7.49 (m, 3H), 7.28-7.21 (m, 1H), 3.65 (d, J=8.1 Hz, 1H), 2.85 (d, J=8.1 Hz, 1H); ESIMS m/z 471 ([M−H]⁻).

### tert-Butyl N-tert-butoxycarbonyl-(3-((1R,3R)-2,2-dichloro-3-(3,4,5-trichlorophenyl)cyclopropane-1-carboxamido)-2,6-difluorophenyl)carbamate (C12)

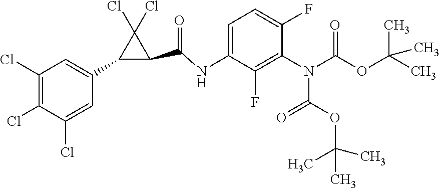 [SMILES: CC(C)(C)OC(=O)N(C(=O)OC(C)(C)C)c1c(F)ccc(NC(=O)[C@H]2[C@H](c3cc(Cl)c(Cl)c(Cl)c3)C2(Cl)Cl)c1F]

The title compound was prepared from (1R,3R)-2,2-dichloro-3-(3,4,5-trichlorophenyl)cyclopropane-1-carboxylic acid and tert-butyl N-(3-amino-2,6-difluorophenyl)-N-tert-butoxycarbonylcarbamate and was isolated as a pale yellow solid (0.62 g, 91%): mp 165-168° C.; ¹H NMR (400 MHz, DMSO-d₆) δ 10.56 (s, 1H), 7.90 (td, J=8.9, 5.8 Hz, 1H), 7.78 (s, 2H), 7.26 (td, J=9.3, 1.8 Hz, 1H), 3.68 (d, J=8.6 Hz, 1H), 3.63 (d, J=8.5 Hz, 1H), 1.40 (d, J=1.6 Hz, 18H); ¹⁹F NMR (376 MHz, DMSO-d₆) δ −124.68, −128.85; HRMS-ESI (m/z) [M]⁺ calcd for C₂₆H₂₅Cl₅F2N₂O₅, 658.0184; found, 658.0184.

### trans-rac-tert-Butyl (tert-butoxycarbonyl)(2-chloro-5-(2,2-dichloro-3-(3,5-dichlorophenyl)cyclopropane-1-carboxamido)phenyl)carbamate (C13)

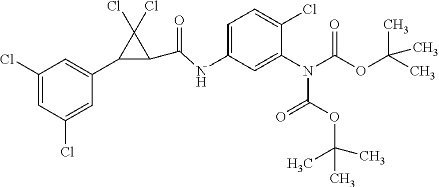 [SMILES: CC(C)(C)OC(=O)N(C(=O)OC(C)(C)C)c1cc(NC(=O)C2C(c3cc(Cl)cc(Cl)c3)C2(Cl)Cl)ccc1Cl]

The title compound was prepared from trans-2,2-dichloro-3-(3,5-dichlorophenyl)cyclopropane-1-carboxylic acid and tert-butyl (5-amino-2-chlorophenyl)(tert-butoxycarbonyl)carbamate (C14) and was isolated as a brown solid (9.2 g, 85%): ¹H NMR (500 MHz, DMSO-d₆) δ 10.84 (s, 1H), 7.70 (d, J=2.4 Hz, 1H), 7.64-7.60 (m, 2H), 7.57-7.52 (m, 3H), 3.63 (d, J=8.5 Hz, 1H), 3.49 (d, J=8.5 Hz, 1H), 1.38 (s, 18H); ¹³C NMR (101 MHz, DMSO-d₆) δ 163.06, 150.62, 138.58, 137.79, 137.12, 134.53, 130.09, 128.40, 128.16, 126.38, 120.87, 120.39, 83.10, 62.64, 38.88, 37.25, 27.88; ESIMS m/z 623 ([M+H]⁺).

### (1R,3R)-2,2-Dichloro-3-(3,5-dichlorophenyl)-N-(2-fluoro-3-nitrophenyl)cyclopropane-1-carboxamide (C15)

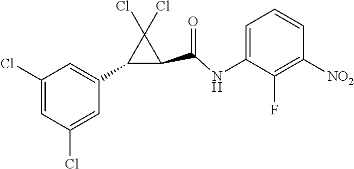 [SMILES: O=C(Nc1cccc([N+](=O)[O-])c1F)[C@H]1[C@H](c2cc(Cl)cc(Cl)c2)C1(Cl)Cl]

The title compound was prepared from (1R,3R)-2,2-dichloro-3-(3,5-dichlorophenyl)cyclopropane-1-carboxylic acid and 2-fluoro-3-nitroaniline and was isolated as an off-white solid (1.9 g, 87%): ¹H NMR (400 MHz, DMSO-d₆) δ 10.83 (s, 1H), 8.33 (ddd, J=8.4, 6.8, 1.7 Hz, 1H), 7.94 (ddd, J=8.4, 6.9, 1.7 Hz, 1H), 7.64 (t, J=1.9 Hz, 1H), 7.58-7.51 (m, 2H), 7.45 (td, J=8.3, 1.5 Hz, 1H), 3.75 (d, J=8.6 Hz, 1H), 3.64 (d, J=8.5 Hz, 1H); ¹⁹F NMR (376 MHz, DMSO-d₆) δ −130.71; ESIMS m/z 439 ([M+H]⁺).

### trans-rac-tert-Butyl (tert-butoxycarbonyl)(5-(2,2-dichloro-3-(3,5-dichlorophenyl)cyclopropane-1-carboxamido)-2-fluorophenyl)carbamate (C16)

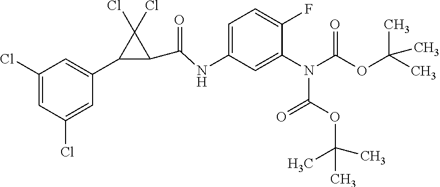 [SMILES: CC(C)(C)OC(=O)N(C(=O)OC(C)(C)C)c1cc(NC(=O)C2C(c3cc(Cl)cc(Cl)c3)C2(Cl)Cl)ccc1F]

The title compound was prepared from trans-2,2-dichloro-3-(3,5-dichlorophenyl)cyclopropane-1-carboxylic acid and tert-butyl (5-amino-2-fluorophenyl)(tert-butoxycarbonyl)carbamate and was isolated as a white solid (0.97 g, 96%): ¹H NMR (400 MHz, DMSO-d₆) δ 10.75 (s, 1H), 7.66 (dd, J=7.1, 2.6 Hz, 1H), 7.62 (t, J=1.9 Hz, 1H), 7.61-7.52 (m, 3H), 7.31 (t, J=9.4 Hz, 1H), 3.62 (d, J=8.5 Hz, 1H), 3.48 (d, J=8.6 Hz, 1H), 1.39 (s, 18H); ¹⁹F NMR (376 MHz, DMSO-d₆) δ −128.18; ESIMS m/z 607 ([M−H]⁻).

### N-(2-Chloro-5-nitrophenyl)-4-cyano-2-methylbenzamide (C17)

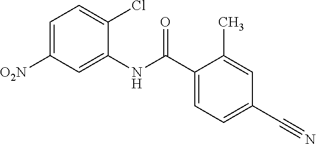 [SMILES: Cc1cc(C#N)ccc1C(=O)Nc1cc([N+](=O)[O-])ccc1Cl]

The title compound was prepared from 4-cyano-2-methylbenzoic acid and 2-chloro-5-nitroaniline and was isolated as a light yellow solid (0.97 g, 96%): ¹H NMR (400 MHz, DMSO-d₆) δ 10.61 (s, 1H), 8.66 (d, J=2.8 Hz, 1H), 8.14 (dd, J=8.8, 2.8 Hz, 1H), 7.91-7.77 (m, 3H), 7.77 (d, J=7.9 Hz, 1H), 2.48 (s, 3H); ESIMS m/z 316 ([M+H]⁺).

### N-(2-Chloro-5-nitrophenyl)-3,5-difluoropicolinamide (C18)

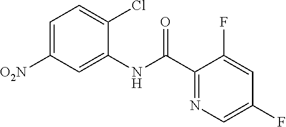 [SMILES: O=C(Nc1cc([N+](=O)[O-])ccc1Cl)c1ncc(F)cc1F]

The title compound was prepared from 3,5-difluoropicolinic acid and 2-chloro-5-nitroaniline and was isolated as a light brown solid (2.0 g, 55%): ¹H NMR (300 MHz, DMSO-d₆) δ 10.56 (s, 1H), 9.09 (d, J=2.6 Hz, 1H), 8.73 (d, J=2.2 Hz, 1H), 8.25 (ddd, J=11.1, 9.1, 2.2 Hz, 1H), 8.08 (dd, J=8.8, 2.6 Hz, 1H), 7.91 (d, J=8.8 Hz, 1H); ESIMS m/z 314 ([M+H]⁺).

### N-(2,6-Difluoro-3-nitrophenyl)-2-(2,2,2-trifluoroethoxy)acetamide (C19)

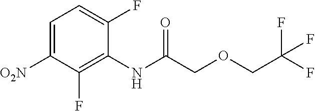 [SMILES: O=C(COCC(F)(F)F)Nc1c(F)ccc([N+](=O)[O-])c1F]

The title compound was prepared from 2,6-difluoro-3-nitroaniline and 2-(2,2,2-trifluoroethoxy)acetic acid) and was isolated as an off-white solid (1.41 g, 77%): ¹H NMR (500 MHz, CDCl₃) δ 8.11 (ddd, J=9.5, 8.0, 5.4 Hz, 1H), 7.89 (s, 1H), 7.15 (ddd, J=9.4, 8.1, 2.0 Hz, 1H), 4.40 (s, 2H), 4.06 (q, J=8.3 Hz, 2H); ESIMS m/z 313 ([M−H]⁻).

### Example 9

### Preparation of trans-rac-N-(2-chloro-5-(2,2-dichloro-3-(3,5-dichlorophenyl)cyclopropane-1-carboxamido)phenyl)-3-nitrobenzamide (F88)

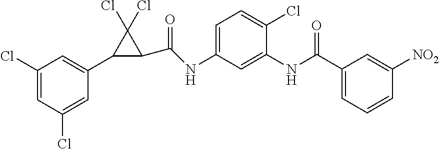 [SMILES: O=C(Nc1cc(NC(=O)C2C(c3cc(Cl)cc(Cl)c3)C2(Cl)Cl)ccc1Cl)c1cccc([N+](=O)[O-])c1]

Step 1. To a solution of 4-chloro-3-nitroaniline (1.60 g, 9.27 mmol) in dichloromethane (40 mL) at 0° C. were added triethylamine (3.23 mL, 23.2 mmol) and DMAP (0.057 g, 0.46 mmol), followed by di-tert-butyl dicarbonate (4.25 g, 19.5 mmol). The reaction mixture was stirred at room temperature for 16 hours. The reaction mixture was diluted with dichloromethane (50 mL) and was washed with water (2×50 mL) and brine (25 mL). The organic layer was dried over anhydrous sodium sulfate, filtered, and concentrated under reduced pressure to afford tert-butyl (tert-butoxycarbonyl)(4-chloro-3-nitrophenyl)carbamate as an off-white solid (3.5 g), which was used in step 2 without analysis or purification.

Step 2. To a solution of the resultant intermediate from step 1 (3.5 g) in ethyl acetate (100 mL) was added 10% palladium on carbon (0.35 g). The reaction mixture was placed under hydrogen atmosphere (45 pounds per square inch (psi)) on a Parr shaker for 6 hours. The reaction mixture was filtered through a pad of Celite®, and the pad was washed with ethyl acetate. The filtrate concentrated under reduced pressure to afford tert-butyl (3-amino-4-chlorophenyl)(tert-butoxycarbonyl)carbamate as a brown solid (2.5 g), which was used in step 3 without analysis or purification.

Step 3. To a solution of 3-nitrobenzoic acid (0.275 g, 1.65 mmol) in dichloromethane (20 mL) at 0° C. was added the resultant intermediate from step 2 (0.565 g, 1.65 mmol). EDC·HCl (0.412 g, 2.15 mmol) and DMAP (0.222 g, 1.82 mmol) were added. The reaction mixture was stirred at room temperature for 16 hours. The reaction was quenched with water, and the mixture was extracted with ethyl acetate (3×30 mL). The organic layer was washed with 1 N aqueous hydrogen chloride (50 mL), saturated aqueous sodium bicarbonate (50 mL), and brine (20 mL). The organic layer was dried over anhydrous sodium sulfate, filtered, and concentrated under reduced pressure to afford tert-butyl (tert-butoxycarbonyl)(4-chloro-3-(3-nitrobenzamido)phenyl)carbamate as a brown solid (0.8 g), which was used in step 4 without analysis or purification.

Step 4. To a solution of the resultant intermediate from step 3 (0.421 g, 0.857 mmol) in dichloromethane (10 mL) was added a 4 M solution of hydrogen chloride (1.07 mL, 4.28 mmol) in dioxane. The reaction mixture was stirred at room temperature for 16 hours and then concentrated under reduced pressure. The resulting residue was partitioned between ethyl acetate (15 mL) and saturated aqueous sodium bicarbonate (15 mL). The phases were separated. The organic phase was washed with brine (2×5 mL), dried over sodium sulfate, filtered, and concentrated under reduced pressure to afford N-(5-amino-2-chlorophenyl)-3-nitrobenzamide as a pale yellow solid (0.25 g), which was used in step 5 without purification: ESIMS m/z 292 ([M+H]⁺).

Step 5. To a solution of the resulting aniline from step 4 (0.203 g, 0.697 mmol) and trans-2,2-dichloro-3-(3,5-dichlorophenyl)cyclopropane-1-carboxylic acid (0.252 g, 0.841 mmol) in dichloromethane (20 mL) were added EDC·HCl (0.203 g, 1.06 mmol) and DMAP (0.094 g, 0.769 mmol). The reaction mixture was stirred at room temperature for 16 hours, was diluted with water, and was extracted with ethyl acetate (3×30 mL). The combined organic layers were washed with saturated aqueous sodium bicarbonate and with brine, dried over anhydrous sodium sulfate, filtered, and concentrated under reduced pressure. Purification of the resulting product by column chromatography eluting with 20-30% ethyl acetate in petroleum ether afforded the title compound as an off-white solid (0.08 g, 20%).

The following compounds were prepared in accordance with the procedure in Example 9:

### trans-rac-N-(2-Chloro-5-(2,2-dichloro-3-(3,5-dichlorophenyl)cyclopropane-1-carboxamido)phenyl)-4-nitrobenzamide (F89)

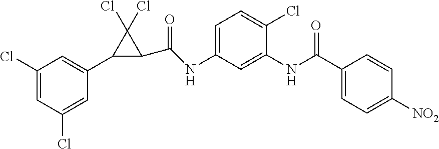 [SMILES: O=C(Nc1cc(NC(=O)C2C(c3cc(Cl)cc(Cl)c3)C2(Cl)Cl)ccc1Cl)c1ccc([N+](=O)[O-])cc1]

The title compound was prepared and was isolated as an off-white solid (0.06 g, 14%).

### trans-rac-N-(2-Chloro-5-(2,2-dichloro-3-(3,5-dichlorophenyl)cyclopropane-1-carboxamido)phenyl)-2-methyl-4-nitrobenzamide (F90)

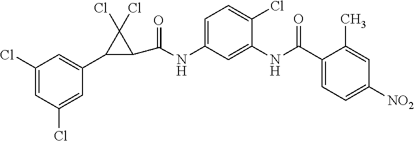 [SMILES: Cc1cc([N+](=O)[O-])ccc1C(=O)Nc1cc(NC(=O)C2C(c3cc(Cl)cc(Cl)c3)C2(Cl)Cl)ccc1Cl]

The title compound was prepared and was isolated as an off-white solid (0.031 g, 8%).

### Example 10

### Preparation of 3-amino-N-(2-chloro-5-((1R,3R)-2,2-dichloro-3-(4-fluoro-3-(trifluoromethyl)phenyl)cyclopropane-1-carboxamido)phenyl)-2,4-difluorobenzamide (F103)

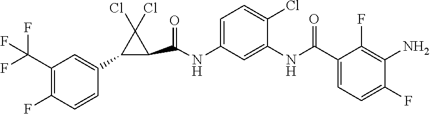 [SMILES: Nc1c(F)ccc(C(=O)Nc2cc(NC(=O)[C@H]3[C@H](c4ccc(F)c(C(F)(F)F)c4)C3(Cl)Cl)ccc2Cl)c1F]

To a suspension of tert-butyl (3-((2-chloro-5-((1R,3R)-2,2-dichloro-3-(4-fluoro-3-(trifluoromethyl)phenyl)cyclopropane-1-carboxamido)phenyl)carbamoyl)-2,6-difluorophenyl)carbamate (F101, 0.320 g, 0.459 mmol) in dichloromethane (5 mL) was added a 4 M solution of hydrogen chloride in dioxane (0.574 mL, 2.296 mmol). The reaction mixture was stirred at room temperature and monitored by liquid chromatography-mass spectrometry (LC-MS) for progress. After 18 hours, starting material remained. To the reaction mixture was added additional 4 M solution of hydrogen chloride in dioxane (0.5 mL), and the reaction mixture was stirred for 18 hours at room temperature. Analysis by LC-MS indicated unconsumed starting material. Methanol was added to the mixture until a homogenous solution was achieved. The solution was stirred at room temperature for 18 hours, at which point analysis by LC-MS showed the reaction to be complete. The reaction mixture was concentrated under a stream of nitrogen and the residue was diluted with ethyl acetate (20 mL). The solution was washed with saturated aqueous sodium bicarbonate (3×10 mL) and brine. The organic layer was passed through a phase separator to dry and the filtrate was concentrated under reduced pressure to afford the title compound as a light brown foam (0.270 g, 99%).

### Example 11

### Preparation of N-(2-chloro-5-((1R,3R)-2,2-dichloro-3-(4-fluoro-3-(trifluoromethyl)phenyl)cyclopropane-1-carboxamido)phenyl)-3-(2,2-difluoroacetamido)-2,4-difluorobenzamide (F104)

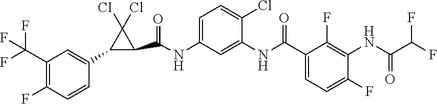 [SMILES: O=C(Nc1cc(NC(=O)[C@H]2[C@H](c3ccc(F)c(C(F)(F)F)c3)C2(Cl)Cl)ccc1Cl)c1ccc(F)c(NC(=O)C(F)F)c1F]

To a solution of 3-amino-N-(2-chloro-5-((1R,3R)-2,2-dichloro-3-(4-fluoro-3-(trifluoromethyl)phenyl)cyclopropane-1-carboxamido)phenyl)-2,4-difluorobenzamide (F103, 0.100 g, 0.168 mmol) in dichloromethane (2 mL) was added 2,2-difluoroacetic anhydride (0.044 g, 0.251 mmol) at room temperature. After 1 hour, the reaction mixture was directly loaded onto a Celite® loading cartridge. Purification by column chromatography eluting with 0-60% acetone in hexanes afforded the title compound as a white solid (0.080 g, 71%).

The following compounds were prepared in accordance with the procedure in Example 11:

### trans-rac-N-(2-Chloro-5-(2,2-dichloro-3-(4-fluoro-3-(trifluoromethyl)phenyl)cyclopropane-1-carboxamido)phenyl)-4-(2,2-difluoroacetamido)-2-fluorobenzamide (F111)

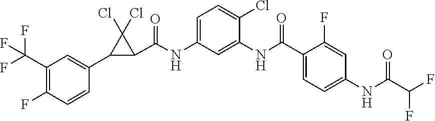 [SMILES: O=C(Nc1cc(NC(=O)C2C(c3ccc(F)c(C(F)(F)F)c3)C2(Cl)Cl)ccc1Cl)c1ccc(NC(=O)C(F)F)cc1F]

The title compound was prepared and was isolated as a cream-colored solid (0.18 g, 63%).

### trans-rac-N-(2-Chloro-5-(2,2-dichloro-3-(4-fluoro-3-(trifluoromethyl)phenyl)cyclopropane-1-carboxamido)phenyl)-2-fluoro-4-(2,2,2-trifluoroacetamido)benzamide (F112)

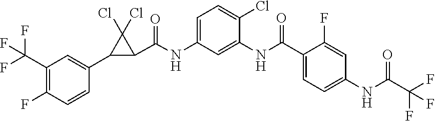 [SMILES: O=C(Nc1cc(NC(=O)C2C(c3ccc(F)c(C(F)(F)F)c3)C2(Cl)Cl)ccc1Cl)c1ccc(NC(=O)C(F)(F)F)cc1F]

The title compound was prepared and was isolated as an off-white solid (0.17 g, 58%).

### Example 12

### Preparation of N-(2-chloro-5-((1R,3R)-2,2-dichloro-3-(4-fluoro-3-(trifluoromethyl)phenyl)cyclopropane-1-carboxamido)phenyl)-2,4-difluoro-3-(2-methoxyacetamido)benzamide (F105)

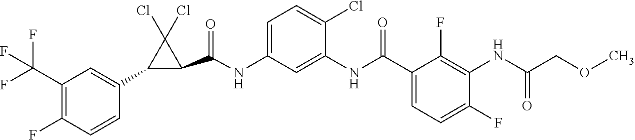 [SMILES: COCC(=O)Nc1c(F)ccc(C(=O)Nc2cc(NC(=O)[C@H]3[C@H](c4ccc(F)c(C(F)(F)F)c4)C3(Cl)Cl)ccc2Cl)c1F]

To a solution of 3-amino-N-(2-chloro-5-((1R,3R)-2,2-dichloro-3-(4-fluoro-3-(trifluoromethyl)phenyl)cyclopropane-1-carboxamido)phenyl)-2,4-difluorobenzamide (F103, 0.100 g, 0.168 mmol) in ethyl acetate (2 mL) was added 2-methoxyacetic acid (0.015 g, 0.168 mmol). Pyridine (0.027 mL, 0.335 mmol), followed by a 50% solution of 2,4,6-tripropyl-1,3,5,2,4,6-trioxatriphosphinane 2,4,6-trioxide (0.160 g, 0.251 mmol) in ethyl acetate, were added, and the reaction mixture was warmed to 45° C. for 18 hours. The reaction mixture was concentrated under a stream of nitrogen. Purification of the residue by column chromatography eluting with 0-60% acetone in hexanes afforded the title compound as a white solid (0.086 g, 77%).

### Example 13

### Preparation of N-(2-chloro-5-((1R,3R)-2,2-dichloro-3-(4-fluoro-3-(trifluoromethyl)phenyl)cyclopropane-1-carboxamido)phenyl)-2,6-difluoronicotinamide and N-(2-chloro-5-((1S,3S)-2,2-dichloro-3-(4-fluoro-3-(trifluoromethyl)phenyl)cyclopropane-1-carboxamido)phenyl)-2,6-difluoronicotinamide (F107 and F108)

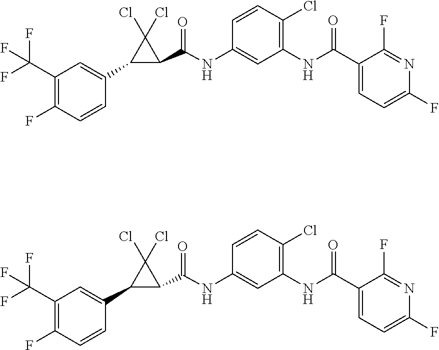 [SMILES: O=C(Nc1cc(NC(=O)[C@H]2[C@H](c3ccc(F)c(C(F)(F)F)c3)C2(Cl)Cl)ccc1Cl)c1ccc(F)nc1F ; O=C(Nc1cc(NC(=O)[C@@H]2[C@@H](c3ccc(F)c(C(F)(F)F)c3)C2(Cl)Cl)ccc1Cl)c1ccc(F)nc1F]

trans-rac-N-(3-Amino-4-chlorophenyl)-2,2-dichloro-3-(4-fluoro-3-(trifluoromethyl)phenyl)cyclopropane-1-carboxamide (C20, 1.0 g, 2.27 mmol) was added to a solution of 2,6-difluoronicotinic acid (0.36 g, 2.27 mmol) in toluene (25 mL). Pyridine (0.72 mL, 9.05 mmol) was added dropwise, and 2,4,6-tripropyl-1,3,5,2,4,6-trioxatriphosphinane 2,4,6-trioxide as a 50% solution in ethyl acetate (5.8 mL, 9.05 mmol) was added at room temperature. The reaction mixture was warmed to 110° C. and stirred for 16 hours, cooled to room temperature, and concentrated under reduced pressure. The residue was diluted with water (80 mL) and was extracted with ethyl acetate (2×80 mL). The combined organic phases were washed with saturated aqueous sodium chloride, dried over anhydrous sodium sulfate, and filtered, and the filtrate was concentrated under reduced pressure. The resulting product was purified by automated flash chromatography (SiO₂, 40→60% ethyl acetate in petroleum ether), and the diastereomers were separated by supercritical fluid chromatography ((5μ CHIRALCEL® OJ-H (30×250 mm), 20% isopropanol in CO₂, 70 g/min, 30° C., 100 bar back pressure) to give the R,R and S,S pairs of trans-diastereomers as cream-colored solids. The absolute configuration was determined by subsequent screening against target pests, the results of which indicated Peak 1 is the S,S configuration and Peak 2 is the R,R configuration, as the efficacy of Peak-2 was significantly greater than Peak-1.

Compound 107 was isolated as a cream-colored solid (0.32 g, 48%).

Compound 108 was isolated as a cream-colored solid (0.32 g, 48%).

The following compounds were prepared in accordance with the procedure in Example 13:

### N-(2-Chloro-5-((1R,3R)-2,2-dichloro-3-(4-fluoro-3-(trifluoromethyl)phenyl)cyclopropane-1-carboxamido)phenyl)-5,6-difluoronicotinamide (F109)

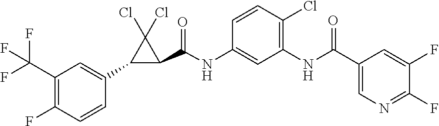 [SMILES: O=C(Nc1cc(NC(=O)[C@H]2[C@H](c3ccc(F)c(C(F)(F)F)c3)C2(Cl)Cl)ccc1Cl)c1cnc(F)c(F)c1]

The title compound was prepared and was isolated as a cream-colored solid (0.23 g, 40%).

### N-(2-Chloro-5-((1S,3S)-2,2-dichloro-3-(4-fluoro-3-(trifluoromethyl)phenyl)cyclopropane-1-carboxamido)phenyl)-5,6-difluoronicotinamide (F110)

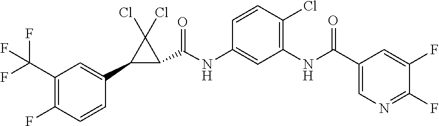 [SMILES: O=C(Nc1cc(NC(=O)[C@@H]2[C@@H](c3ccc(F)c(C(F)(F)F)c3)C2(Cl)Cl)ccc1Cl)c1cnc(F)c(F)c1]

The title compound was prepared and was isolated as a cream-colored solid (0.16 g, 32%).

### N-(2-Chloro-5-nitrophenyl)-3,5-difluorobenzamide (C21)

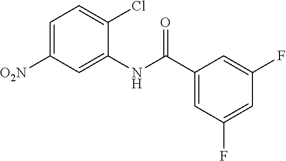 [SMILES: O=C(Nc1cc([N+](=O)[O-])ccc1Cl)c1cc(F)cc(F)c1]

The title compound was prepared from 3,5-difluorobenzoic acid and 2-chloro-5-nitroaniline and was isolated as a yellow solid (0.34 g, 94%): ¹H NMR (400 MHz, DMSO-d₆) δ 10.58 (s, 1H), 8.53 (d, J=2.7 Hz, 1H), 8.16 (dd, J=8.9, 2.8 Hz, 1H), 7.90 (d, J=8.9 Hz, 1H), 7.76-7.67 (m, 2H), 7.66-7.55 (m, 1H); ¹⁹F NMR (376 MHz, DMSO-d₆) δ −108.50; ESIMS m/z 313 ([M+H]⁺).

### tert-Butyl (3-((2-chloro-5-nitrophenyl)carbamoyl)-2,6-difluorophenyl)carbamate (C22)

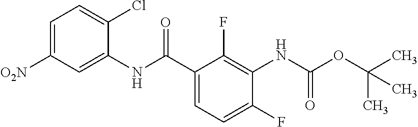 [SMILES: CC(C)(C)OC(=O)Nc1c(F)ccc(C(=O)Nc2cc([N+](=O)[O-])ccc2Cl)c1F]

The title compound was prepared from 3-((tert-butoxycarbonyl)amino)-2,4-difluorobenzoic acid and 2-chloro-5-nitroaniline and was isolated as a yellow solid (0.44 g, 89%): ¹H NMR (400 MHz, DMSO-d₆) δ 10.41 (d, J=2.5 Hz, 1H), 9.04 (s, 1H), 8.73 (d, J=2.8 Hz, 1H), 8.12 (dd, J=8.9, 2.8 Hz, 1H), 7.92-7.85 (m, 1H), 7.80-7.69 (m, 1H), 7.32 (td, J=9.2, 1.7 Hz, 1H), 1.45 (s, 9H); ¹⁹F NMR (376 MHz, DMSO-d₆) δ −112.67, −118.21; ESIMS m/z 426 ([M−H]⁻).

### N-(2-Chloro-5-nitrophenyl)-4,4,4-trifluoro-3-methylbutanamide (C23)

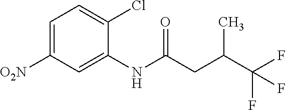 [SMILES: CC(CC(=O)Nc1cc([N+](=O)[O-])ccc1Cl)C(F)(F)F]

The title compound was prepared from 4,4,4-trifluoro-3-methylbutanoic acid and 2-chloro-5-nitroaniline and was isolated as a yellow solid (0.33 g, 92%): ¹H NMR (400 MHz, DMSO-d₆) δ 10.09 (s, 1H), 8.71 (d, J=2.8 Hz, 1H), 8.03 (dd, J=8.8, 2.8 Hz, 1H), 7.82 (d, J=8.8 Hz, 1H), 2.91 (tdd, J=9.0, 6.9, 4.9 Hz, 1H), 2.84 (dd, J=15.3, 4.8 Hz, 1H), 2.62 (dd, J=15.3, 8.9 Hz, 1H), 1.16 (d, J=6.8 Hz, 3H); ¹⁹F NMR (376 MHz, DMSO-d₆) δ −72.07; ESIMS m/z 311 ([M+H]⁺).

### Example 14

### Preparation of (1R,3R)—N-(3-amino-2,4-difluorophenyl)-2,2-dichloro-3-(3,4,5-trichlorophenyl)cyclopropane-1-carboxamide (C1)

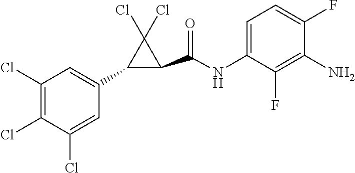 [SMILES: Nc1c(F)ccc(NC(=O)[C@H]2[C@H](c3cc(Cl)c(Cl)c(Cl)c3)C2(Cl)Cl)c1F]

To a solution of tert-butyl N-tert-butoxycarbonyl-(3-((1R,3R)-2,2-dichloro-3-(3,4,5-trichlorophenyl)cyclopropane-1-carboxamido)-2,6-difluorophenyl)carbamate (C12, 0.610 g, 0.923 mmol) in dioxane (4.62 mL) was added a 4 M solution of hydrogen chloride in dioxane (2.31 mL, 9.23 mmol). The resulting solution was stirred at room temperature for 18 hours and was concentrated under a stream of nitrogen. The residue was partitioned between ethyl acetate (15 mL) and saturated aqueous sodium bicarbonate. The phases were separated. The organic phase was washed with brine (2×5 mL), dried over sodium sulfate, filtered, and concentrated under a stream of nitrogen to give an amber oil. The resulting oil was dissolved in diethyl ether (3 mL) and treated with hexanes to affect precipitation. The resulting solids were collected via vacuum filtration to afford the title compound (0.412 g, 90%) as a light orange solid: mp 81-86° C.; ¹H NMR (400 MHz, DMSO-d₆) δ 10.22 (s, 1H), 7.75 (s, 2H), 7.01 (ddd, J=9.0, 8.1, 5.6 Hz, 1H), 6.88 (ddd, J=10.8, 9.0, 1.9 Hz, 1H), 5.32 (s, 2H), 3.68 (d, J=8.6 Hz, 1H), 3.60 (d, J=8.5 Hz, 1H); ¹⁹F NMR (376 MHz, DMSO-d₆) δ −134.85, −134.89, −140.66, −140.69; HRMS-ESI (m/z) [M]⁺ calcd for C₁₆H₉C₁₅F₂N₂O, 457.9126; found, 457.9131.

The following compounds were prepared in accordance with the procedure in Example 14:

### trans-rac-N-(3-Amino-4-chlorophenyl)-2,2-dichloro-3-(3,5-dichlorophenyl)cyclopropane-1-carboxamide (C3)

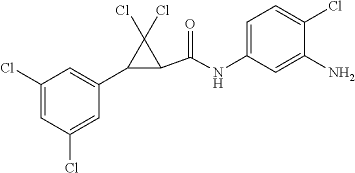 [SMILES: Nc1cc(NC(=O)C2C(c3cc(Cl)cc(Cl)c3)C2(Cl)Cl)ccc1Cl]

The title compound was prepared from trans-rac-tert-butyl (tert-butoxycarbonyl)(2-chloro-5-(2,2-dichloro-3-(3,5-dichlorophenyl)cyclopropane-1-carboxamido)phenyl)carbamate (C13) and was isolated as an off-white solid (5.7 g, 65%): ¹H NMR (300 MHz, DMSO-d₆) δ 10.42 (s, 1H), 7.62-7.61 (m, 1H), 7.54-7.52 (m, 2H), 7.22 (d, J=2.0 Hz, 1H), 7.14-7.11 (m, 1H), 6.80-6.77 (m, 1H), 5.42 (s, 2H), 3.56 (d, J=8.8 Hz, 1H), 3.44 (d, J=8.4 Hz, 1H); ESIMS m/z 423 ([M+H]⁺).

### trans-rac-N-(3-Amino-4-fluorophenyl)-2,2-dichloro-3-(3,5-dichlorophenyl)cyclopropane-1-carboxamide (C24)

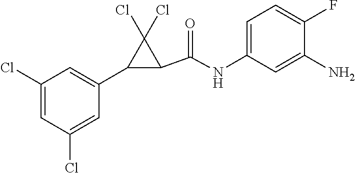 [SMILES: Nc1cc(NC(=O)C2C(c3cc(Cl)cc(Cl)c3)C2(Cl)Cl)ccc1F]

The title compound was prepared from trans-rac-tert-butyl (tert-butoxycarbonyl)(5-(2,2-dichloro-3-(3,5-dichlorophenyl)cyclopropane-1-carboxamido)-2-fluorophenyl)carbamate (C16) and was isolated as a white foam (0.61 g, 95%): ¹H NMR (400 MHz, DMSO-d₆) δ 10.34 (s, 1H), 7.62 (t, J=1.9 Hz, 1H), 7.52 (dd, J=1.9, 0.6 Hz, 2H), 7.16 (dd, J=8.3, 2.6 Hz, 1H), 6.93 (dd, J=11.3, 8.7 Hz, 1H), 6.73 (ddd, J=8.7, 4.0, 2.6 Hz, 1H), 5.22 (s, 2H), 3.56 (d, J=8.5 Hz, 1H), 3.43 (d, J=8.5 Hz, 1H); ¹⁹F NMR (376 MHz, DMSO-d₆) δ −139.93; ESIMS m/z 409 ([M+H]+).

### Example 15

### Preparation of trans-rac-N-(3-amino-4-chlorophenyl)-2,2-dichloro-3-(4-fluoro-3-(trifluoromethyl)phenyl)cyclopropane-1-carboxamide (C20)

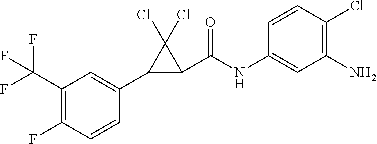 [SMILES: Nc1cc(NC(=O)C2C(c3ccc(F)c(C(F)(F)F)c3)C2(Cl)Cl)ccc1Cl]

To a room temperature solution of trans-rac-2,2-dichloro-N-(4-chloro-3-nitrophenyl)-3-(4-fluoro-3-(trifluoromethyl)phenyl)cyclopropane-1-carboxamide (C11, 13 g, 27.6 mmol) in a mixture of tetrahydrofuran, ethanol, and water (2:2:1; 30 mL total) were added sequentially iron powder (15 g, 276.5 mmol) and ammonium chloride (114.8 g, 220.8 mmol). The reaction mixture was warmed to 80° C. and stirred for 6 hours. The reaction mixture was cooled to room temperature and filtered through a pad of Celite®. The filter cake was washed with methanol and the filtrate was concentrated under reduced pressure. The residue was diluted with water and was extracted with ethyl acetate (3×180 mL). The combined extracts were washed with saturated aqueous sodium chloride, dried over anhydrous sodium sulfate, filtered, and concentrated under reduced pressure. The residue was purified by flash chromatography (SiO₂; 40-60% ethyl acetate in petroleum ether) to afford the title compound as a cream-colored solid (10 g, 83%): ¹H NMR (400 MHz, CDCl₃) δ 7.57-7.49 (m, 3H), 7.35 (d, J=2.4 Hz, 1H), 7.23-7.49 (m, 2H), 6.68 (dd, J=2.4, 8.4 Hz, 1H), 3.61 (d, J=8.0 Hz, 1H), 2.77 (d, J=8.0 Hz, 1H); ESIMS m/z 441 ([M+H]⁺).

The following compounds were prepared in accordance with the procedure in Example 15:

### trans-rac-4-Amino-N-(2-chloro-5-(2,2-dichloro-3-(4-fluoro-3-(trifluoromethyl)phenyl)cyclopropane-1-carboxamido)phenyl)-2-fluorobenzamide (C25)

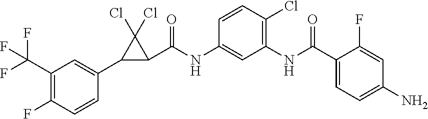 [SMILES: Nc1ccc(C(=O)Nc2cc(NC(=O)C3C(c4ccc(F)c(C(F)(F)F)c4)C3(Cl)Cl)ccc2Cl)c(F)c1]

The title compound was prepared and was isolated as a cream-colored solid (1.2 g, 66%): 1H NMR (400 MHz, CDCl₃) δ 9.11 (d, J=17.6 Hz, 1H), 8.61 (d, J=2.8 Hz, 1H), 8.40 (br s, 1H), 8.00-7.96 (m, 1H), 7.80 (dd, J=2.8, 8.8 Hz, 1H), 7.49 (d, J=6.8 Hz, 1H), 7.41-7.37 (m, 2H), 7.26-7.17 (m, 1H), 6.55 (dd, J=2.4, 8.8 Hz, 1H), 6.41 (dd, J=2.0, 14.4 Hz, 1H), 4.23 (br s, 2H), 3.57 (d, J=8.0 Hz, 1H), 2.75 (d, J=8.0 Hz, 1H); ESIMS m/z 578 ([M+H]⁺).

### (1R,3R)—N-(3-Amino-2-fluorophenyl)-2,2-dichloro-3-(3,5-dichlorophenyl)cyclopropane-1-carboxamide (C26)

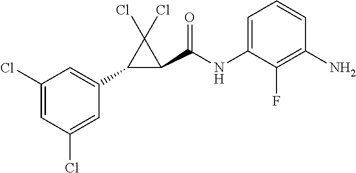 [SMILES: Nc1cccc(NC(=O)[C@H]2[C@H](c3cc(Cl)cc(Cl)c3)C2(Cl)Cl)c1F]

The title compound was prepared from (1R,3R)-2,2-dichloro-3-(3,5-dichlorophenyl)-N-(2-fluoro-3-nitrophenyl)cyclopropane-1-carboxamide (C15) and was isolated as an orange foam (1.3 g, 57%): ¹H NMR (400 MHz, DMSO-d₆) δ 10.18 (s, 1H), 7.63 (q, J=2.0 Hz, 1H), 7.51 (d, J=1.9 Hz, 2H), 7.10 (ddd, J=8.3, 6.7, 1.6 Hz, 1H), 6.82 (td, J=8.0, 1.3 Hz, 1H), 6.57 (td, J=8.2, 1.6 Hz, 1H), 5.22 (s, 2H), 3.71 (d, J=8.6 Hz, 1H), 3.59 (d, J=8.6 Hz, 1H); ¹⁹F NMR (376 MHz, DMSO-d₆) δ −147.11; ESIMS m/z 409 ([M+H]⁺).

### N-(5-Amino-2-chlorophenyl)-3,5-difluoropicolinamide (C27)

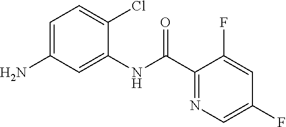 [SMILES: Nc1ccc(Cl)c(NC(=O)c2ncc(F)cc2F)c1]

The title compound was prepared from N-(2-chloro-5-nitrophenyl)-3,5-difluoropicolinamide (C18) and was isolated as a white solid (1.2 g, 44%): ESIMS m/z 284 ([M+H]⁺).

### N-(5-Amino-2-chlorophenyl)acetamide (C7)

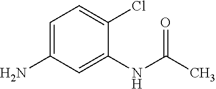

The title compound was prepared from N-(2-chloro-5-nitrophenyl)acetamide (C4) and was isolated as a yellow solid (0.204 g, 59%): ¹H NMR (400 MHz, CDCl₃) δ 7.83 (d, J=2.2 Hz, 1H), 7.56 (s, 1H), 7.09 (d, J=8.6 Hz, 1H), 6.36 (dd, J=8.6, 2.7 Hz, 1H), 3.74 (s, 2H), 2.22 (s, 3H); EIMS m/z 264.

### N-(5-Amino-2-chlorophenyl)-4-fluorobenzamide (C8)

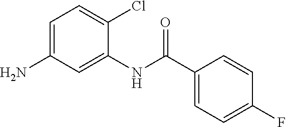 [SMILES: Nc1ccc(Cl)c(NC(=O)c2ccc(F)cc2)c1]

The title compound was prepared from N-(2-chloro-5-nitrophenyl)-4-fluorobenzamide (C5) and was isolated as an off-white solid (0.084 g, 67%): ¹H NMR (400 MHz, CDCl₃) δ 8.33 (s, 1H), 7.99 (d, J=2.7 Hz, 1H), 7.96-7.88 (m, 2H), 7.23-7.17 (m, 2H), 7.15 (d, J=8.6 Hz, 1H), 6.41 (dd, J=8.6, 2.7 Hz, 1H), 3.79 (s, 2H); EIMS m/z 264.

### N-(5-Amino-2-chlorophenyl)cyclopropanecarboxamide (C9)

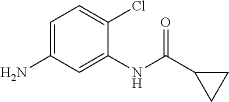 [SMILES: Nc1ccc(Cl)c(NC(=O)C2CC2)c1]

The title compound was prepared from N-(2-chloro-5-nitrophenyl)cyclopropanecarboxamide (C6) and was isolated as a brown solid (0.249 g, 73%): ¹H NMR (400 MHz, DMSO-d₆) δ 9.39 (s, 1H), 7.04 (d, J=8.6 Hz, 1H), 6.96 (d, J=1.7 Hz, 1H), 6.35 (dd, J=8.6, 2.7 Hz, 1H), 5.25 (s, 2H), 2.02-1.90 (m, 1H), 0.81-0.76 (m, 4H); EIMS m/z 210.

### Example 16

### Preparation of tert-butyl (5-amino-2-chlorophenyl)(tert-butoxycarbonyl)carbamate (C14)

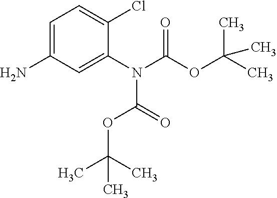 [SMILES: CC(C)(C)OC(=O)N(C(=O)OC(C)(C)C)c1cc(N)ccc1Cl]

A solution of tert-butyl (tert-butoxycarbonyl)(2-chloro-5-nitrophenyl)carbamate (C28, 2.065 g, 5.54 mmol) in ethyl acetate (22 mL) was sparged with nitrogen for 5 minutes and the solution was treated with 10% palladium on carbon (0.25 g), while sparging was continued. The flask was fitted with a three-way stopcock and gently evacuated under house vacuum. The flask was back-filled with nitrogen and the process was repeated (2×). The flask was gently evacuated and back-filled with hydrogen from a balloon (2×). The reaction mixture was stirred under an atmosphere of hydrogen (balloon) for 16 hours. The reaction flask was evacuated under gentle vacuum and backfilled with nitrogen (2×). The reaction mixture was sparged with nitrogen for 5 minutes. The mixture was filtered through a pad of Celite® and the pad was rinsed with ethyl acetate. The filtrate was concentrated to give the title compound as a white solid (1.87 g, 98%): mp 84-90° C.; ¹H NMR (400 MHz, CDCl₃) δ 7.15 (d, J=8.5 Hz, 1H), 6.56 (m, 2H), 3.70 (s, 2H), 1.42 (s, 18H); ¹³C NMR (101 MHz, CDCl₃) δ 150.87, 145.66, 137.45, 129.70, 121.67, 116.21, 115.68, 82.72, 27.87.

The following compounds were prepared in accordance with the procedure in Example 16:

### N-(5-Amino-2-chlorophenyl)-4-cyano-2-methylbenzamide (C10)

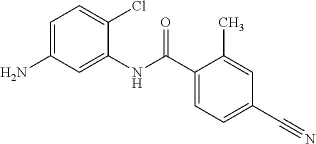 [SMILES: Cc1cc(C#N)ccc1C(=O)Nc1cc(N)ccc1Cl]

The title compound was prepared from N-(2-chloro-5-nitrophenyl)-4-cyano-2-methylbenzamide (C17) and was isolated as a white solid (0.211 g, 65%): ¹H NMR (400 MHz, DMSO-d₆) δ 9.93 (s, 1H), 7.80 (d, J=18.9 Hz, 2H), 7.61 (d, J=7.9 Hz, 1H), 7.11 (d, J=8.6 Hz, 1H), 6.88-6.83 (m, 1H), 6.47 (dd, J=8.6, 2.7 Hz, 1H), 5.36 (s, 2H), 2.45 (s, 3H); ESIMS m/z 286 ([M+H]⁺).

### N-(5-Amino-2-chlorophenyl)-3,5-difluorobenzamide (C29)

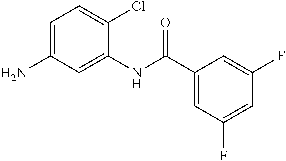 [SMILES: Nc1ccc(Cl)c(NC(=O)c2cc(F)cc(F)c2)c1]

The title compound was prepared from N-(2-chloro-5-nitrophenyl)-3,5-difluorobenzamide (C21) and was isolated as a white solid (0.285 g, 93%): ¹H NMR (400 MHz, DMSO-d₆) δ 9.99 (s, 1H), 7.71-7.61 (m, 2H), 7.55 (tt, J=9.1, 2.3 Hz, 1H), 7.13 (d, J=8.6 Hz, 1H), 6.74 (d, J=2.7 Hz, 1H), 6.50 (dd, J=8.6, 2.7 Hz, 1H), 5.38 (s, 2H); ¹⁹F NMR (376 MHz, DMSO-d₆) δ −108.64; ESIMS m/z 283 ([M+H]⁺).

### tert-Butyl (3-((5-amino-2-chlorophenyl)carbamoyl)-2,6-difluorophenyl)carbamate (C30)

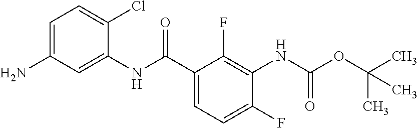 [SMILES: CC(C)(C)OC(=O)Nc1c(F)ccc(C(=O)Nc2cc(N)ccc2Cl)c1F]

The title compound was prepared from tert-butyl (3-((2-chloro-5-nitrophenyl)carbamoyl)-2,6-difluorophenyl)carbamate (C22) and was isolated as a white solid (0.420 g, 100%): ¹H NMR (400 MHz, DMSO-d₆) δ 9.71 (d, J=3.2 Hz, 1H), 8.99 (s, 1H), 7.65 (q, J=7.9 Hz, 1H), 7.27 (t, J=8.9 Hz, 1H), 7.11 (d, J=8.6 Hz, 1H), 7.07-6.98 (m, 1H), 6.45 (dd, J=8.6, 2.7 Hz, 1H), 5.37 (s, 2H), 1.44 (s, 9H); ¹⁹F NMR (376 MHz, DMSO-d₆) δ −113.61, −118.80 (d, J=8.6 Hz); ESIMS m/z 396 ([M−H]⁻).

### N-(5-Amino-2-chlorophenyl)-4,4,4-trifluoro-3-methylbutanamide (C31)

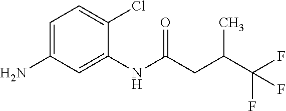 [SMILES: CC(CC(=O)Nc1cc(N)ccc1Cl)C(F)(F)F]

The title compound was prepared from N-(2-chloro-5-nitrophenyl)-4,4,4-trifluoro-3-methylbutanamide (C23) and was isolated as a white foam (0.305 g, 100%): ¹H NMR (400 MHz, DMSO-d₆) δ 9.43 (s, 1H), 7.06 (d, J=8.6 Hz, 1H), 6.90 (dd, J=7.0, 2.5 Hz, 1H), 6.39 (dd, J=8.6, 2.7 Hz, 1H), 5.30 (s, 2H), 2.85 (tdd, J=9.4, 7.1, 4.8 Hz, 1H), 2.68 (dd, J=15.0, 4.9 Hz, 1H), 2.50-2.41 (m, 1H), 1.13 (d, J=6.9 Hz, 3H); ¹⁹F NMR (376 MHz, DMSO-d₆) δ −72.07.

### N-(3-Amino-2,6-difluorophenyl)-2-(2,2,2-trifluoroethoxy)acetamide (C32)

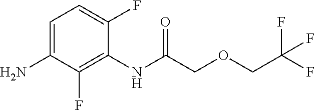 [SMILES: Nc1ccc(F)c(NC(=O)COCC(F)(F)F)c1F]

The title compound was prepared from N-(2,6-difluoro-3-nitrophenyl)-2-(2,2,2-trifluoroethoxy)acetamide (C19) and was isolated as a grey solid (1.16 g, 88%): mp 95-100° C.; ¹H NMR (500 MHz, CDCl₃) δ 7.75 (s, 1H), 6.78 (td, J=9.1, 2.0 Hz, 1H), 6.66 (td, J=9.1, 5.2 Hz, 1H), 4.35 (s, 2H), 4.03 (q, J=8.4 Hz, 2H), 3.89 (s, 2H); ESIMS m/z 285 ([M+H]⁺).

### Example 17

### Preparation of tert-butyl (tert-butoxycarbonyl)(2-chloro-5-nitrophenyl)carbamate

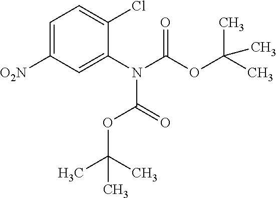 [SMILES: CC(C)(C)OC(=O)N(C(=O)OC(C)(C)C)c1cc([N+](=O)[O-])ccc1Cl]

To a solution of 2-chloro-5-nitroaniline (2.5 g, 14.5 mmol) in tetrahydrofuran (24 mL) were added DMAP (0.088 g, 0.72 mmol) followed by di-tert-butyl dicarbonate (3.48 g, 15.9 mmol). The dark reaction mixture was warmed to 50° C. and stirred for 16 hours. The dark mixture was treated with additional DMAP (1.77 g, 14.5 mmol) and di-tert-butyl dicarbonate (3.48 g, 15.9 mmol), was warmed to reflux, and was stirred for 16 hours. The reaction mixture was concentrated to give a viscous, dark oil which was partitioned between dichloromethane (50 mL) and saturated aqueous ammonium chloride (50 mL). The phases were separated and the aqueous phase was extracted with additional dichloromethane (2×50 mL). The combined organic phases were dried by passing through a phase separator, and the solution was treated with Celite® (10 g). The solvent was evaporated and the adsorbed material was purified by automated flash chromatography (SiO₂; 0→50% ethyl acetate in hexanes) to give the title compound as a peach-colored solid (4.44 g, 82%): mp 99-102° C.; ¹H NMR (400 MHz, CDCl₃) δ 8.15 (m, 2H), 7.63 (d, J=8.7 Hz, 1H), 1.42 (s, 18H); ¹³C NMR (101 MHz, CDCl₃) δ 149.85, 146.70, 140.25, 138.32, 130.27, 125.31, 123.70, 84.05, 27.81.

### Example 18

### Preparation of trans-rac-2,2-dichloro-N-(4-chloro-3-(4,4,4-trifluoro-N,3-dimethylbutanamido)phenyl)-3-(3,5-dichlorophenyl)cyclopropane-1-carboxamide (F92)

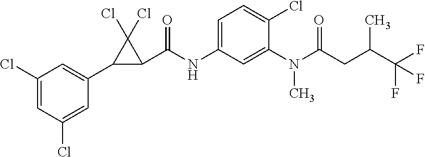 [SMILES: CC(CC(=O)N(C)c1cc(NC(=O)C2C(c3cc(Cl)cc(Cl)c3)C2(Cl)Cl)ccc1Cl)C(F)(F)F]

Step 1. To a suspension of N-(2-chloro-5-nitrophenyl)-4,4,4-trifluoro-N,3-dimethylbutanamide (C33, 0.129 g, 0.397 mmol) in 3:1 methanol/water (5.3 mL) were added ammonium chloride (0.064 g, 1.19 mmol) and iron powder (0.111 g, 1.99 mmol). The reaction mixture was stirred vigorously and warmed to 50° C. for 18 h. The mixture was cooled to room temperature and filtered over a bed of Celite®, which was washed with methanol. The filtrate was concentrated under reduced pressure, and the residue was partitioned between ethyl acetate (25 mL) and 1:1 brine/water (20 mL). The organic layer was passed through a phase separator to dry and the solvent was concentrated to yield N-(5-amino-2-chlorophenyl)-4,4,4-trifluoro-N,3-dimethylbutanamide, which was used in step 2 without purification (0.101 g, 87%): ESIMS m/z 295 ([M+H]⁺).

Step 2. To a solution of 2,2-dichloro-3-(3,5-dichlorophenyl)cyclopropane-1-carboxylic acid (0.102 g, 0.339 mmol) and the aniline from step 1 (0.100 g, 0.339 mmol) in ethyl acetate (1 mL) were added pyridine (0.055 mL, 0.679 mmol) followed by a 50% solution of 2,4,6-tripropyl-1,3,5,2,4,6-trioxatriphosphinane 2,4,6-trioxide (0.324 g, 0.509 mmol) in ethyl acetate. The reaction mixture was warmed to 45° C. and was stirred for 16 hours. The reaction mixture was cooled to room temperature and concentrated under a stream of nitrogen. The residue was purified by column chromatography eluting with 0-40% ethyl acetate in hexanes to afford the title compound as a yellow foam (0.088 g, 45%).

The following compounds were prepared in accordance with the procedure in Example 18:

### trans-rac-N-(2-Chloro-5-(2,2-dichloro-3-(3,5-dichlorophenyl)cyclopropane-1-carboxamido)phenyl)-4-cyano-N,2-dimethylbenzamide (F93)

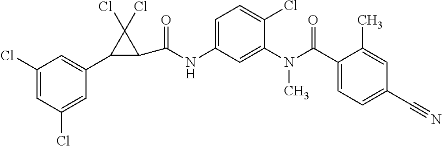 [SMILES: Cc1cc(C#N)ccc1C(=O)N(C)c1cc(NC(=O)C2C(c3cc(Cl)cc(Cl)c3)C2(Cl)Cl)ccc1Cl]

The title compound was prepared from trans-2,2-dichloro-3-(3,5-dichlorophenyl)cyclopropane-1-carboxylic acid and was isolated as a white foam (0.122 g, 55%).

### trans-rac-N-(2-Chloro-5-(2,2-dichloro-3-(3,5-dichlorophenyl)cyclopropane-1-carboxamido)phenyl)-3,5-difluoro-N-methylbenzamide (F94)

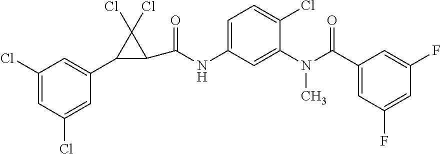 [SMILES: CN(C(=O)c1cc(F)cc(F)c1)c1cc(NC(=O)C2C(c3cc(Cl)cc(Cl)c3)C2(Cl)Cl)ccc1Cl]

The title compound was prepared from trans-2,2-dichloro-3-(3,5-dichlorophenyl)cyclopropane-1-carboxylic acid and was isolated as a white foam (0.106 g, 54%).

### Example 19

### Preparation of N-(2-chloro-5-nitrophenyl)-4,4,4-trifluoro-N,3-dimethylbutanamide (C33)

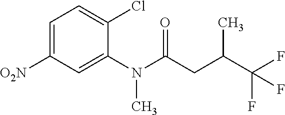 [SMILES: CC(CC(=O)N(C)c1cc([N+](=O)[O-])ccc1Cl)C(F)(F)F]

To a solution of N-(2-chloro-5-nitrophenyl)-4,4,4-trifluoro-3-methylbutanamide (C23, 0.128 g, 0.412 mmol) in dry N,N-dimethylformamide (5 mL) stirred in an ice bath was added sodium hydride (0.025 g, 0.618 mmol) in one portion. The ice bath was removed, and the reaction mixture was stirred for 45 minutes while warming to room temperature. Iodomethane (0.028 mL, 0.453 mmol) was added to the reaction mixture in one portion, and the reaction mixture was stirred at room temperature overnight. The reaction was quenched by careful addition of water (5 mL), and the mixture was extracted with ethyl acetate (30 mL). The organic layer was washed with 1:1 brine/water (3×20 mL) and then brine. The organic layer was passed through a phase separator to dry and was concentrated under reduced pressure to afford the title compound as an orange oil (0.129 g, 96%): ¹H NMR (400 MHz, DMSO-d₆) δ 8.52 (t, J=2.6 Hz, 1H), 8.36-8.27 (m, 1H), 7.99 (dd, J=8.9, 3.0 Hz, 1H), 3.14 (d, J=2.8 Hz, 3H), 2.89-2.82 (m, 2H), 2.12 (dd, J=8.1, 6.3 Hz, 1H), 1.04 (dd, J=13.5, 6.9 Hz, 3H), 1:1 mixture of rotational isomers; ¹⁹F NMR (376 MHz, DMSO-d₆) δ −71.98, −72.14, 1:1 mixture of rotational isomers; ESIMS m/z 325 ([M+H]⁺).

The following compounds were prepared in accordance with the procedure in Example 19:

### N-(2-Chloro-5-nitrophenyl)-4-cyano-N,2-dimethylbenzamide (C34)

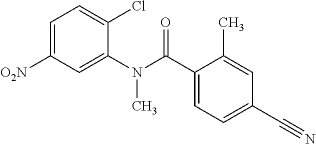 [SMILES: Cc1cc(C#N)ccc1C(=O)N(C)c1cc([N+](=O)[O-])ccc1Cl]

The title compound was prepared from N-(2-chloro-5-nitrophenyl)-4-cyano-2-methylbenzamide (C17) and was isolated as an off-white solid (0.145 g, 98%): ¹H NMR (400 MHz, DMSO-d₆) δ 8.69 (d, J=2.7 Hz, 1H), 8.50 (d, J=2.7 Hz, 1H), 8.29 (dd, J=8.9, 2.8 Hz, 1H), 8.09 (dd, J=8.9, 2.7 Hz, 1H), 7.96 (d, J=8.8 Hz, 1H), 7.91-7.82 (m, 2H), 7.76 (d, J=8.9 Hz, 2H), 7.69-7.61 (m, 1H), 7.47 (dd, J=7.9, 1.7 Hz, 1H), 7.32 (d, J=8.0 Hz, 1H), 3.36 (s, 3H), 3.06 (s, 3H), 2.44 (s, 3H), 2.39 (s, 3H), 1:1 mixture of rotational isomers; ESIMS m/z 330 ([M+H]⁺).

### N-(2-Chloro-5-nitrophenyl)-3,5-difluoro-N-methylbenzamide (C35)

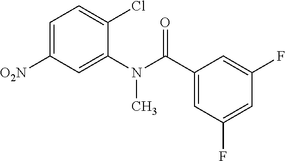 [SMILES: CN(C(=O)c1cc(F)cc(F)c1)c1cc([N+](=O)[O-])ccc1Cl]

The title compound was prepared from N-(2-chloro-5-nitrophenyl)-3,5-difluorobenzamide (C21) and was isolated as an off-white solid (0.110 g, 97%): ¹H NMR (400 MHz, DMSO-d₆) δ 8.63 (d, J=2.8 Hz, 1H), 8.14 (d, J=9.0 Hz, 1H), 7.88-7.74 (m, 1H), 7.53-7.32 (m, 1H), 7.30-7.15 (m, 1H), 7.06 (s, 1H), 3.31 (s, 3H); ¹⁹F NMR (376 MHz, DMSO-d₆) δ −108.73; ESIMS m/z 327 ([M+H]⁺).

### Example 20

### Preparation of 4-((tert-butoxycarbonyl)amino)-3-fluoro-2-methylbenzoic acid (C36)

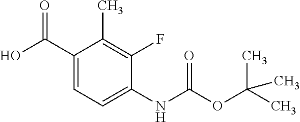 [SMILES: Cc1c(C(=O)O)ccc(NC(=O)OC(C)(C)C)c1F]

To a solution of methyl 4-((tert-butoxycarbonyl)amino)-3-fluoro-2-methylbenzoate (C38, 0.7 g, 2.47 mmol) in dichloroethane (25 mL) was added trimethyltin hydroxide (1.33 g, 7.42 mmol), and the reaction mixture was warmed to 100° C. and stirred for 72 hours. The reaction mixture was cooled to room temperature, concentrated under reduced pressure, and the residue suspended in ice water (50 mL). The mixture was extracted with ethyl acetate (3×40 mL). The combined organic extracts were washed successively with water (3×100 mL) and saturated aqueous sodium chloride (100 mL), dried over anhydrous sodium sulfate, filtered and concentrated under reduced pressure to give a solid. The solid was purified by flash chromatography (Silica gel, 40→50% ethyl acetate in petroleum ether) to afford the title compound as a cream-colored solid (0.16 g, 24%): mp 175-179° C.; ¹H NMR (400 MHz, DMSO-d₆) δ 9.09 (s, 1H), 7.61-7.53 (m, 2H), 2.41 (d, J=2.8, Hz, 3H), 1.47 (s, 9H); ¹⁹F NMR (376 MHz, DMSO-d₆) δ −128.43; ESIMS m/z 268 ([M−H]⁺).

The following compound was prepared in accordance with the procedure in Example 20:

### 4-((tert-Butoxycarbonyl)amino)-2-fluorobenzoic acid (C37)

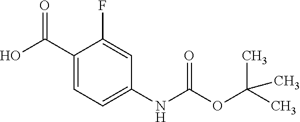 [SMILES: CC(C)(C)OC(=O)Nc1ccc(C(=O)O)c(F)c1]

The title compound was prepared and was isolated as an off-white solid (0.42 g, 45%); mp 196-200° C.; ¹H NMR (400 MHz, DMSO-d₆) δ 9.79 (s, 1H), 8.98 (br, 1H), 7.71-7.67 (m, 1H), 7.36 (dd, J=2.0, 13.6 Hz, 1H), 7.22 (dd, J=1.6, 8.4 Hz, 1H), 1.48 (s, 9H); ¹⁹F NMR (376 MHz, DMSO-d₆) δ −109.30; ESIMS m/z 256 ([M+H]⁺).

### Example 21

### Preparation of methyl 4-((tert-butoxycarbonyl)amino)-3-fluoro-2-methylbenzoate (C38)

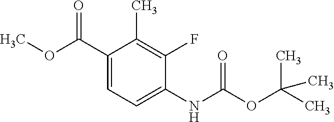 [SMILES: COC(=O)c1ccc(NC(=O)OC(C)(C)C)c(F)c1C]

To a solution of methyl 4-amino-3-fluoro-2-methylbenzoate (C40, 1.1 g, 6.01 mmol) in tetrahydrofuran (25 mL) were added triethylamine (2.1 mL, 15.0 mmol) and di-tert-butyl dicarbonate (1.58 g, 7.2 mmol), and the reaction mixture was stirred at 80° C. for 16 hours. The reaction mixture was cooled to room temperature and concentrated under reduced pressure. The residue was suspended in water (50 mL) and extracted with ethyl acetate (3×40 mL). The combined organic extracts were washed successively with water (3×100 mL) and saturated aqueous sodium chloride (100 mL), dried over anhydrous sodium sulfate, filtered, and concentrated under reduced pressure to give a solid. The solid was purified by flash chromatography (Silica gel, 10→20% ethyl acetate in petroleum ether) to afford the title compound as a cream-colored solid (0.8 g, 47%): ESIMS m/z 282 ([M−H]⁺).

The following compound was prepared in accordance with the procedure in Example 21:

### Methyl 4-((tert-butoxycarbonyl)amino)-2-fluorobenzoate (C39)

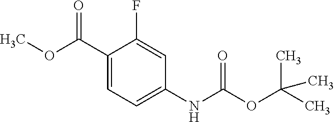 [SMILES: COC(=O)c1ccc(NC(=O)OC(C)(C)C)cc1F]

The title compound was prepared and was isolated as an off-white solid (0.38 g, 50%); mp 194-198° C.; ¹H NMR (400 MHz, DMSO-d₆) δ 9.98 (s, 1H), 7.82-7.78 (m, 1H), 7.47 (dd, J=2.0, 14.0 Hz, 1H), 7.73 (dd, J=2.0, 8.8 Hz, 1H), 3.80 (s, 3H), 1.48 (s, 9H); ESIMS m/z 270 ([M+H]⁺).

### Example 22

### Preparation of methyl 4-amino-3-fluoro-2-methylbenzoate (C40)

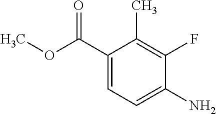

To a solution of 4-amino-3-fluoro-2-methylbenzoic acid (C41, 1.2 g, 7.1 mmol) in methanol (25 mL) was added sulfuric acid (0.3 mL), and the reaction mixture was stirred at 80° C. for 16 hours. The reaction mixture was concentrated to dryness and the residue was diluted with ice water (50 mL) and extracted with ethyl acetate (3×60 mL). The combined organic extracts were washed successively with cold water (3×100 mL) and saturated aqueous sodium chloride (100 mL), dried over anhydrous sodium sulfate, filtered, and concentrated under reduced pressure. The residue was purified by flash chromatography (Silica gel, 30→40% ethyl acetate in petroleum ether) to afford the title compound as a cream-colored solid (0.9 g, 69%): ¹H NMR (400 MHz, CDCl₃) δ 7.63-7.60 (m, 1H), 6.59-6.55 (m, 1H), 4.05 (d, J=13.6 Hz, 2H), 3.83 (s, 3H), 2.51-2.50 (m, 3H); ESIMS m/z 184 ([M+H]⁺).

### Example 23

### Preparation of 4-amino-3-fluoro-2-methylbenzoic acid (C41)

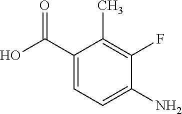

To a solution of 4-amino-3-fluoro-2-methylbenzonitrile (1.5 g, 10 mmol) in a mixture of aqueous methanol (1:1; 40 mL total) was added potassium hydroxide (6.72 g, 120.0 mmol) at room temperature. The reaction mixture was warmed to 100° C. and stirred for 48 hours, cooled to room temperature, and the pH adjusted to ˜3 with aqueous hydrogen chloride (˜3 mL). The solution was extracted with a 10% solution of methanol in dichloromethane (3×100 mL), and the combined organic phases were washed successively with cold water (3×100 mL) and saturated aqueous sodium chloride (100 mL), dried over anhydrous sodium sulfate, filtered, and concentrated under reduced pressure to afford a solid. The solid compound was suspended in toluene and concentrated (2×50 mL) to afford the title compound as a pale yellow solid (1.3 g, 77%): ESIMS m/z 170 ([M+H]⁺).

The following intermediates were prepared in accordance with methods previously disclosed.
(1R,3R)-3-(3,5-Bis(trifluoromethyl)phenyl)-2,2-dichlorocyclopropane-1-carboxylic acid was prepared from trans-3-(3,5-bis(trifluoromethyl)phenyl)-2,2-dichlorocyclopropane-1-carboxylic (C8) according to methods described in PCT International Application Publication WO 2016/168059 A1 and in the art.
(1R,3R)-2,2-Dichloro-3-(4-fluoro-3-(trifluoromethyl)phenyl)cyclopropane-1-carboxylic acid (C95) was prepared according to PCT International Application Publication WO 2018/071327 A1.
(1R,3R)-2,2-Dichloro-3-(3-bromo-4-fluorophenyl)cyclopropane-1-carboxylic acid (C243) was prepared according to PCT International Application Publication WO 2018/071327 A1.
(1R,3R)-2,2-Dichloro-3-(3,4-dichlorophenyl)cyclopropane-1-carboxylic acid (C92) was prepared according to PCT International Application Publication WO 2018/071327 A1.
(1R,3R)-2,2-Dichloro-3-(3,5-dichlorophenyl)cyclopropane-1-carboxylic acid (C91) was prepared according to PCT International Application Publication WO 2018/071327 A1.
trans-2,2-Dichloro-3-(3,5-dichlorophenyl)cyclopropane-1-carboxylic acid (C1) was prepared according to PCT International Application Publication WO 2016/168059 A1.
(1R,3R)-2,2-Dichloro-3-(3,4,5-trichlorophenyl)cyclopropane-1-carboxylic acid (C94) was prepared according to PCT International Application Publication WO 2018/071327 A1.
trans-2,2-Dichloro-3-(4-fluoro-3-(trifluoromethyl)phenyl)cyclopropane-1-carboxylic acid (C76) was prepared according to PCT International Application Publication WO 2018/071327 A1.
tert-Butyl N-(3-amino-2,6-difluorophenyl)-N-tert-butoxycarbonylcarbamate (C397) was prepared according to PCT International Application Publication WO 2016/168059 A1.
tert-Butyl (5-amino-2-fluorophenyl)(tert-butoxycarbonyl)carbamate (C410) was prepared according to PCT International Application Publication WO 2016/168059 A1.

It is recognized that some reagents and reaction conditions may not be compatible with certain functionalities that may be present in certain molecules of Formula One or certain molecules used in the preparation of certain molecules of Formula One. In such cases, it may be necessary to employ standard protection and deprotection protocols comprehensively reported in the literature and well known to a person skilled in the art. In addition, in some cases it may be necessary to perform further routine synthetic steps not described herein to complete the synthesis of desired molecules. A person skilled in the art will also recognize that it may be possible to achieve the synthesis of desired molecules by performing some of the steps of the synthetic routes in a different order to that described. A person skilled in the art will also recognize that it may be possible to perform standard functional group interconversions or substitution reactions on desired molecules to introduce or modify substituents.

### Biological Assays

The following bioassays against beet armyworm (Spodoptera exigua), cabbage looper (Trichoplusia ni), and yellow fever mosquito (Aedes aegypti), are included herein due to the damage they inflict. Furthermore, the beet armyworm and cabbage looper are two good indicator species for a broad range of chewing pests. The results with these two indicator species along with the yellow fever mosquito show the broad usefulness of the molecules of Formula One in controlling pests in Phyla Arthropoda, Mollusca, and Nematoda (Drewes et al., High-Throughput Screening in Agrochemical Research, Modern Methods in Crop Protection Research, Part I, Methods for the Design and Optimization of New Active Ingredients, Edited by Jeschke, P., Kramer, W., Schirmer, U., and Matthias W., p. 1-20, 2012)

### Example A: Bioassays on Beet Armyworm (Spodoptera exigua, LAPHEG) (“BAW”) and Cabbage Looper (Trichoplusia ni, TRIPNI) (“CL”)

Beet armyworm is a serious pest of economic concern for alfalfa, asparagus, beets, citrus, corn, cotton, onions, peas, peppers, potatoes, soybeans, sugar beets, sunflowers, tobacco, and tomatoes, among other crops. It is native to Southeast Asia but is now found in Africa, Australia, Japan, North America, and Southern Europe. The larvae may feed in large swarms causing devastating crop losses. It is known to be resistant to several pesticides.

Cabbage looper is a serious pest found throughout the world. It attacks alfalfa, beans, beets, broccoli, Brussel sprouts, cabbage, cantaloupe, cauliflower, celery, collards, cotton, cucumbers, eggplant, kale, lettuce, melons, mustard, parsley, peas, peppers, potatoes, soybeans, spinach, squash, tomatoes, turnips, and watermelons, among other crops. This species is very destructive to plants due to its voracious appetite. The larvae consume three times their weight in food daily. The feeding sites are marked by large accumulations of sticky, wet, fecal material, which may contribute to higher disease pressure thereby causing secondary problems on the plants in the site. It is known to be resistant to several pesticides.

Consequently, because of the above factors control of these pests is important. Furthermore, molecules that control these pests (BAW and CL), which are known as chewing pests, will be useful in controlling other pests that chew on plants.

Certain molecules disclosed in this document were tested against BAW and CL using procedures described in the following examples.

### Bioassays on BAW

Bioassays on BAW were conducted using a 128-well diet tray assay. One second instar BAW larva was placed in each well (8 wells total) of the diet tray that had been previously filled with 1 mL of artificial diet to which 50 μg/cm² of the test molecule (dissolved in 50 μL of 90:10 acetone-water mixture) had been applied (to each of eight wells) and then allowed to dry. Trays were covered with a clear self-adhesive cover, vented to allow gas exchange, and held at 25° C., 14:10 light-dark for five days. Percent mortality was recorded for the larvae in each well; activity in the eight wells was then averaged.

In this assay, F1, F2, F3, F4, F5, F6, F7, F8, F9, F10, F11, F12, F13, F14, F15, F16, F18, F19, F20, F22, F24, F25, F26, F27, F30, F32, F34, F35, F36, F37, F38, F39, F40, F41, F42, F43, F44, F47, F48, F49, F50, F51, F52, F53, F54, F55, F56, F57, F58, F60, F61, F62, F63, F64, F65, F66, F67, F68, F69, F70, F71, F72, F73, F74, F75, F76, F77, F78, F79, F80, F81, F82, F83, F84, F85, F87, F88, F89, F90, F91, F92, F93, F94, F95, F96, F97, F98, F99, F100, F101, F102, F103, F104, F105, F106, F109, F110, F111, F112, F113, F114, F115, F116, F117, F118, F119, F120, and F121 exhibited activity at 50 μg/cm².

### Bioassays on CL

Bioassays on CL were conducted using a 128-well diet tray assay. One second instar CL larva was placed in each well (8 wells total) of the diet tray that had been previously filled with 1 mL of artificial diet to which 50 μg/cm² of the test molecule (dissolved in 50 μL of 90:10 acetone-water mixture) had been applied (to each of eight wells) and then allowed to dry. Trays were covered with a clear self-adhesive cover, vented to allow gas exchange, and held at 25° C., 14:10 light-dark for five days. Percent mortality was recorded for the larvae in each well; activity in the eight wells was then averaged.

In this test, F1, F2, F3, F4, F5, F6, F7, F8, F9, F10, F11, F12, F13, F14, F15, F16, F17, F18, F19, F20, F21, F22, F23, F24, F25, F26, F27, F29, F30, F31, F32, F34, F35, F36, F37, F38, F39, F40, F41, F42, F43, F44, F45, F46, F47, F48, F49, F50, F51, F52, F53, F54, F55, F56, F57, F58, F60, F61, F62, F63, F64, F65, F66, F67, F68, F69, F70, F71, F72, F73, F74, F75, F77, F78, F79, F80, F81, F82, F83, F84, F85, F87, F88, F89, F90, F91, F92, F93, F94, F95, F96, F97, F98, F99, F100, F102, F103, F104, F105, F106, F107, F109, F110, F111, F112, F113, F114, F115, F116, F117, F118, F119, F120, and F121 exhibited activity at 50 μg/cm².

### Example B: Bioassays on Yellow Fever Mosquito (Aedes aegypti, AEDSAE) (“YFM”)

YFM prefers to feed on humans during the daytime and is most frequently found in or near human habitations. YFM is a vector for transmitting several diseases. It is a mosquito that can spread the dengue fever and yellow fever viruses. Yellow fever is the second most dangerous mosquito-borne disease after malaria. Yellow fever is an acute viral hemorrhagic disease and up to 50% of severely affected persons without treatment will die from yellow fever. There are an estimated 200,000 cases of yellow fever, causing 30,000 deaths worldwide each year. Dengue fever is a nasty, viral disease; it is sometimes called “breakbone fever” or “break-heart fever” because of the intense pain it can produce. Dengue fever kills about 20,000 people annually. Consequently, because of the above factors control of this pest is important. Furthermore, molecules that control this pest (YFM), which is known as a sucking pest, are useful in controlling other pests that cause human and animal suffering.

Certain molecules disclosed in this document were tested against YFM using procedures described in the following paragraph.

Master plates containing 400 μg of a molecule dissolved in 100 μL of dimethyl sulfoxide (DMSO) (equivalent to a 4000 ppm solution) are used. A master plate of assembled molecules contains 15 μL per well. To this plate, 135 μL of a 90:10 water/acetone mixture is added to each well. A robot (Biomek® NXP Laboratory Automation Workstation) is programmed to dispense 15 μL aspirations from the master plate into an empty 96-well shallow plate (“daughter” plate). There are 6 reps (“daughter” plates) created per master. The created “daughter” plates are then immediately infested with YFM larvae.

The day before plates are to be treated, mosquito eggs are placed in Millipore water containing liver powder to begin hatching (4 g into 400 mL). After the “daughter” plates are created using the robot, they are infested with 220 μL of the liver powder/larval mosquito mixture (about 1 day-old larvae). After plates are infested with mosquito larvae, a non-evaporative lid is used to cover the plate to reduce drying. Plates are held at room temperature for 3 days prior to grading. After 3 days, each well is observed and scored based on mortality.

In this test, F1, F2, F3, F4, F7, F10, F11, F14, F15, F16, F18, F19, F20, F21, F22, F25, F28, F35, F39, F40, F42, F45, F48, F50, F53, F54, F56, F58, F59, F60, F61, F62, F64, F67, F68, F69, F70, F71, F72, F74, F75, F77, F78, F79, F80, F82, F83, F84, F85, F95, F100, F106, F109, F111, F112, F113, F114, F115, F116, F117, and F118 exhibited activity at 4000 ppm.

Agriculturally acceptable acid addition salts, salt derivatives, solvates, ester derivatives, polymorphs, isotopes, and radionuclides

Molecules of Formula One may be formulated into agriculturally acceptable acid addition salts. By way of a non-limiting example, an amine function can form salts with hydrochloric, hydrobromic, sulfuric, phosphoric, acetic, benzoic, citric, malonic, salicylic, malic, fumaric, oxalic, succinic, tartaric, lactic, gluconic, ascorbic, maleic, aspartic, benzenesulfonic, methanesulfonic, ethanesulfonic, hydroxyl-methanesulfonic, and hydroxyethanesulfonic acids. Additionally, by way of a non-limiting example, an acid function can form salts including those derived from alkali or alkaline earth metals and those derived from ammonia and amines. Examples of preferred cations include sodium, potassium, and magnesium.

Molecules of Formula One may be formulated into salt derivatives. By way of a non-limiting example, a salt derivative may be prepared by contacting a free base with a sufficient amount of the desired acid to produce a salt. A free base may be regenerated by treating the salt with a suitable dilute aqueous base solution such as dilute aqueous sodium hydroxide, potassium carbonate, ammonia, and sodium bicarbonate. As an example, in many cases, a pesticide, such as 2,4-D, is made more water-soluble by converting it to its dimethylamine salt.

Molecules of Formula One may be formulated into stable complexes with a solvent, such that the complex remains intact after the non-complexed solvent is removed. These complexes are often referred to as “solvates.” However, it is particularly desirable to form stable hydrates with water as the solvent.

Molecules of Formula One containing an acid functionality may be made into ester derivatives. These ester derivatives can then be applied in the same manner as the molecules disclosed in this document are applied.

Molecules of Formula One may be made as various crystal polymorphs. Polymorphism is important in the development of agrochemicals since different crystal polymorphs or structures of the same molecule can have vastly different physical properties and biological performances.

Molecules of Formula One may be made with different isotopes. Of particular importance are molecules having ²H (also known as deuterium) or ³H (also known as tritium) in place of ¹H. Molecules of Formula One may be made with different radionuclides. Of particular importance are molecules having ¹⁴C (also known as radiocarbon). Molecules of Formula One having deuterium, tritium, or ¹⁴C may be used in biological studies allowing tracing in chemical and physiological processes and half-life studies, as well as, mode of action studies.

### Formulations

A pesticide is many times not suitable for application in its pure form. It is usually necessary to add other substances so that the pesticide may be used at the required concentration and in an appropriate form, permitting ease of application, handling, transportation, storage, and maximum pesticide activity. Thus, pesticides are formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra-low volume solutions.

Pesticides are applied most often as aqueous suspensions or emulsions prepared from concentrated formulations of such pesticides. Such water-soluble, water-suspendable, or emulsifiable formulations are either solids, usually known as wettable powders, water dispersible granules, liquids usually known as emulsifiable concentrates, or aqueous suspensions. Wettable powders, which may be compacted to form water dispersible granules, comprise an intimate mixture of the pesticide, a carrier, and surfactants. The concentration of the pesticide is usually from about 10% to about 90% by weight. The carrier is usually selected from among the attapulgite clays, the montmorillonite clays, the diatomaceous earths, or the purified silicates. Effective surfactants, comprising from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates of pesticides comprise a convenient concentration of a pesticide, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums may, also be added to increase the density and viscosity of the aqueous carrier. It is often most effective to grind and mix the pesticide at the same time by preparing the aqueous mixture and homogenizing it in an implement such as a sand mill, ball mill, or piston-type homogenizer. The pesticide in suspension might be microencapsulated in plastic polymer.

Oil dispersions (OD) comprise suspensions of organic solvent-insoluble pesticides finely dispersed in a mixture of organic solvent and emulsifiers at a concentration in the range from about 2% to about 50% by weight. One or more pesticide might be dissolved in the organic solvent. Useful organic solvents include aromatics, especially xylenes and petroleum fractions, especially the high-boiling naphthalenic and olefinic portions of petroleum such as heavy aromatic naphtha. Other solvents may include vegetable oils, seed oils, and esters of vegetable and seed oils. Suitable emulsifiers for oil dispersions are selected from conventional anionic and non-ionic surfactants. Thickeners or gelling agents are added in the formulation of oil dispersions to modify the rheology or flow properties of the liquid and to prevent separation and settling of the dispersed particles or droplets.

Pesticides may also be applied as granular compositions that are particularly useful for applications to the soil. Granular compositions usually contain from about 0.5% to about 10% by weight of the pesticide, dispersed in a carrier that comprises clay or a similar substance. Such compositions are usually prepared by dissolving the pesticide in a suitable solvent and applying it to a granular carrier, which has been pre-formed to the appropriate particle size, in the range of from about 0.5 mm to about 3 mm. Such compositions may also be formulated by making a dough or paste of the carrier and molecule, and then crushing and drying to obtain the desired granular particle size. Another form of granules is a water emulsifiable granule (EG). It is a formulation consisting of granules to be applied as a conventional oil-in-water emulsion of the active ingredient(s), either solubilized or diluted in an organic solvent, after disintegration and dissolution in water. Water emulsifiable granules comprise one or several active ingredient(s), either solubilized or diluted in a suitable organic solvent that is (are) absorbed in a water soluble polymeric shell or some other type of soluble or insoluble matrix.

Dusts containing a pesticide are prepared by intimately mixing the pesticide in powdered form with a suitable dusty agricultural carrier, such as kaolin clay, ground volcanic rock, and the like. Dusts can suitably contain from about 1% to about 10% of the pesticide. Dusts may be applied as a seed dressing or as a foliage application with a dust blower machine.

It is equally practical to apply a pesticide in the form of a solution in an appropriate organic solvent, usually petroleum oil, such as the spray oils, which are widely used in agricultural chemistry.

Pesticides can also be applied in the form of an aerosol composition. In such compositions, the pesticide is dissolved or dispersed in a carrier, which is a pressure-generating propellant mixture. The aerosol composition is packaged in a container from which the mixture is dispensed through an atomizing valve.

Pesticide baits are formed when the pesticide is mixed with food or an attractant or both. When the pests eat the bait, they also consume the pesticide. Baits may take the form of granules, gels, flowable powders, liquids, or solids. Baits may be used in pest harborages.

Fumigants are pesticides that have a relatively high vapor pressure and hence can exist as a gas in sufficient concentrations to kill pests in soil or enclosed spaces. The toxicity of the fumigant is proportional to its concentration and the exposure time. They are characterized by a good capacity for diffusion and act by penetrating the pest's respiratory system or being absorbed through the pest's cuticle. Fumigants are applied to control stored product pests under gas proof sheets, in gas sealed rooms or buildings, or in special chambers.

Pesticides may be microencapsulated by suspending the pesticide particles or droplets in plastic polymers of various types. By altering, the chemistry of the polymer or by changing factors in the processing, microcapsules may be formed of various sizes, solubility, wall thicknesses, and degrees of penetrability. These factors govern the speed with which the active ingredient within is released, which in turn, affects the residual performance, speed of action, and odor of the product. The microcapsules might be formulated as suspension concentrates or water dispersible granules.

Oil solution concentrates are made by dissolving pesticide in a solvent that will hold the pesticide in solution. Oil solutions of a pesticide usually provide faster knockdown and kill of pests than other formulations due to the solvents themselves having pesticidal action and the dissolution of the waxy covering of the integument increasing the speed of uptake of the pesticide. Other advantages of oil solutions include better storage stability, better penetration of crevices, and better adhesion to greasy surfaces.

Another embodiment is an oil-in-water emulsion, wherein the emulsion comprises oily globules which are each provided with a lamellar liquid crystal coating and are dispersed in an aqueous phase, wherein each oily globule comprises at least one molecule which is agriculturally active, and is individually coated with a monolamellar or oligolamellar layer comprising: (1) at least one non-ionic lipophilic surface-active agent, (2) at least one non-ionic hydrophilic surface-active agent, and (3) at least one ionic surface-active agent, wherein the globules having a mean particle diameter of less than 800 nanometers.

### Other Formulation Components

Generally, when the molecules disclosed in Formula One are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate, sodium dioctyl sulfosuccinate, alkyl phenol ethoxylates, and aliphatic alcohol ethoxylates.

A dispersing agent is a substance that adsorbs onto the surface of particles, helps to preserve the state of dispersion of the particles, and prevents them from reaggregating. Dispersing agents are added to agrochemical formulations to facilitate dispersion and suspension during manufacture, and to ensure the particles redisperse into water in a spray tank. They are widely used in wettable powders, suspension concentrates, and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium-naphthalene-sulfonate-formaldehyde-condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates, sodium naphthalene sulfonate formaldehyde condensates, tristyrylphenol-ethoxylate-phosphate-esters, aliphatic alcohol ethoxylates, alkyl ethoxylates, EO-PO block copolymers, and graft copolymers.

An emulsifying agent is a substance that stabilizes a suspension of droplets of one liquid phase in another liquid phase. Without the emulsifying agent, the two liquids would separate into two immiscible liquid phases. The most commonly used emulsifier blends contain an alkylphenol or an aliphatic alcohol with twelve or more ethylene oxide units and the oil-soluble calcium salt of dodecylbenzenesulfonic acid. A range of hydrophile-lipophile balance (“HLB”) values from about 8 to about 18 will normally provide good, stable emulsions. Emulsion stability can sometimes be improved by the addition of a small amount of an EO-PO block copolymer surfactant.

A solubilizing agent is a surfactant that will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alkyl ethoxylates, linear aliphatic alcohol ethoxylates, and aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules, and water-dispersible granules.

Organic solvents are used mainly in the formulation of emulsifiable concentrates, oil-in-water emulsions, suspoemulsions, oil dispersions, and ultra-low volume formulations, and to a lesser extent, granular formulations. Sometimes mixtures of solvents are used. The first main groups of solvents are aliphatic paraffinic oils such as kerosene or refined paraffins. The second main group (and the most common) comprises the aromatic solvents such as xylene and higher molecular weight fractions of C9 and C10 aromatic solvents. Chlorinated hydrocarbons are useful as cosolvents to prevent crystallization of pesticides when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, oil dispersions, emulsions and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate and oil dispersion formulations using clays and silicas. Examples of these types of materials, include, but are not limited to, montmorillonite, bentonite, magnesium aluminum silicate, and attapulgite. Water-soluble polysaccharides in water based suspension concentrates have been used as thickening-gelling agents for many years. The types of polysaccharides most commonly used are natural extracts of seeds and seaweeds or are synthetic derivatives of cellulose. Examples of these types of materials include, but are not limited to, guar gum, locust bean gum, carrageenam, alginates, methyl cellulose, sodium carboxymethyl cellulose (SCMC), and hydroxyethyl cellulose (HEC). Other types of anti-settling agents are based on modified starches, polyacrylates, polyvinyl alcohol, and polyethylene oxide. Another good anti-settling agent is xanthan gum.

Microorganisms can cause spoilage of formulated products. Therefore, preservation agents are used to eliminate or reduce their effect. Examples of such agents include, but are not limited to: propionic acid and its sodium salt, sorbic acid and its sodium or potassium salts, benzoic acid and its sodium salt, p-hydroxybenzoic acid sodium salt, methyl p-hydroxybenzoate, and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g. plant and animal sources. Specific examples are: vegetable oils, seed oils, and esters thereof, also alkoxylated alkyl polyglucosides.

### Applications

Molecules of Formula One may be applied to any locus. Particular loci to apply such molecules include loci where alfalfa, almonds, apples, barley, beans, canola, corn, cotton, crucifers, flowers, fodder species (Rye Grass, Sudan Grass, Tall Fescue, Kentucky Blue Grass, and Clover), fruits, lettuce, oats, oil seed crops, oranges, peanuts, pears, peppers, potatoes, rice, sorghum, soybeans, strawberries, sugarcane, sugarbeets, sunflowers, tobacco, tomatoes, wheat (for example, Hard Red Winter Wheat, Soft Red Winter Wheat, White Winter Wheat, Hard Red Spring Wheat, and Durum Spring Wheat), and other valuable crops are growing or the seeds thereof are going to be planted.

Molecules of Formula One may also be applied where plants, such as crops, are growing and where there are low levels (even no actual presence) of pests that can commercially damage such plants. Applying such molecules in such locus is to benefit the plants being grown in such locus. Such benefits, may include, but are not limited to: helping the plant grow a better root system; helping the plant better withstand stressful growing conditions; improving the health of a plant; improving the yield of a plant (e.g. increased biomass and/or increased content of valuable ingredients); improving the vigor of a plant (e.g. improved plant growth and/or greener leaves); improving the quality of a plant (e.g. improved content or composition of certain ingredients); and improving the tolerance to abiotic and/or biotic stress of the plant.

Molecules of Formula One may be applied with ammonium sulfate when growing various plants as this may provide additional benefits.

Molecules of Formula One may be applied on, in, or around plants genetically modified to express specialized traits, such as Bacillus thuringiensis (for example, Cry1Ab, Cry1Ac, Cry1Fa, Cry1A.105, Cry2Ab, Vip3A, mCry3A, Cry3Ab, Cry3Bb, Cry34Abl/Cry35Abl), other insecticidal toxins, or those expressing herbicide tolerance, or those with “stacked” foreign genes expressing insecticidal toxins, herbicide tolerance, nutrition-enhancement, or any other beneficial traits.

Molecules of Formula One may be applied to the foliar and/or fruiting portions of plants to control pests. Either such molecules will come in direct contact with the pest, or the pest will consume such molecules when eating the plant or while extracting sap or other nutrients from the plant.

Molecules of Formula One may also be applied to the soil, and when applied in this manner, root and stem feeding pests may be controlled. The roots may absorb such molecules thereby taking it up into the foliar portions of the plant to control above ground chewing and sap feeding pests.

Systemic movement of pesticides in plants may be utilized to control pests on one portion of the plant by applying (for example by spraying a locus) a molecule of Formula One to a different portion of the plant. For example, control of foliar-feeding insects may be achieved by drip irrigation or furrow application, by treating the soil with for example pre- or post-planting soil drench, or by treating the seeds of a plant before planting.

Molecules of Formula One may be used with baits. Generally, with baits, the baits are placed in the ground where, for example, termites can come into contact with, and/or be attracted to, the bait. Baits can also be applied to a surface of a building, (horizontal, vertical, or slant surface) where, for example, ants, termites, cockroaches, and flies, can come into contact with, and/or be attracted to, the bait.

Molecules of Formula One may be encapsulated inside, or placed on the surface of a capsule. The size of the capsules can range from nanometer size (about 100-900 nanometers in diameter) to micrometer size (about 10-900 microns in diameter).

Molecules of Formula One may be applied to eggs of pests. Because of the unique ability of the eggs of some pests to resist certain pesticides, repeated applications of such molecules may be desirable to control newly emerged larvae.

Molecules of Formula One may be applied as seed treatments. Seed treatments may be applied to all types of seeds, including those from which plants genetically modified to express specialized traits will germinate. Representative examples include those expressing proteins toxic to invertebrate pests, such as Bacillus thuringiensis or other insecticidal toxins, those expressing herbicide tolerance, such as “Roundup Ready” seed, or those with “stacked” foreign genes expressing insecticidal toxins, herbicide tolerance, nutrition-enhancement, drought tolerance, or any other beneficial traits. Furthermore, such seed treatments with molecules of Formula One may further enhance the ability of a plant to withstand stressful growing conditions better. This results in a healthier, more vigorous plant, which can lead to higher yields at harvest time. Generally, about 1 gram of such molecules to about 500 grams per 100,000 seeds is expected to provide good benefits, amounts from about 10 grams to about 100 grams per 100,000 seeds is expected to provide better benefits, and amounts from about 25 grams to about 75 grams per 100,000 seeds is expected to provide even better benefits.

Molecules of Formula One may be applied with one or more active ingredients in a soil amendment.

Molecules of Formula One may be used for controlling endoparasites and ectoparasites in the veterinary medicine sector or in the field of non-human-animal keeping. Such molecules may be applied by oral administration in the form of, for example, tablets, capsules, drinks, granules, by dermal application in the form of, for example, dipping, spraying, pouring on, spotting on, and dusting, and by parenteral administration in the form of, for example, an injection.

Molecules of Formula One may also be employed advantageously in livestock keeping, for example, cattle, chickens, geese, goats, pigs, sheep, and turkeys. They may also be employed advantageously in pets such as, horses, dogs, and cats. Particular pests to control would be flies, fleas, and ticks that are bothersome to such animals. Suitable formulations are administered orally to the animals with the drinking water or feed. The dosages and formulations that are suitable depend on the species.

Molecules of Formula One may also be used for controlling parasitic worms, especially of the intestine, in the animals listed above.

Molecules of Formula One may also be employed in therapeutic methods for human health care. Such methods include, but are limited to, oral administration in the form of, for example, tablets, capsules, drinks, granules, and by dermal application.

Molecules of Formula One may also be applied to invasive pests. Pests around the world have been migrating to new environments (for such pests) and thereafter becoming a new invasive species in such new environment. Such molecules may also be used on such new invasive species to control them in such new environments.

In light of the above and the Table section below, the following details are additionally provided.

1d. A molecule having the following formula

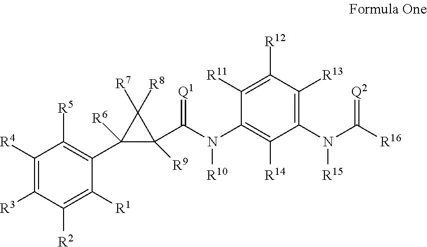

wherein:
(A) R¹ is selected from the group consisting of H, F, Cl, Br, I, and (C₁-C₃)haloalkyl;
(B) R² is selected from the group consisting of H, F, Cl, Br, I, and (C₁-C₃)haloalkyl;
(C) R³ is selected from the group consisting of H, F, Cl, Br, I, and (C₁-C₃)haloalkyl;
(D) R⁴ is selected from the group consisting of H, F, Cl, Br, I, and (C₁-C₃)haloalkyl;
(E) R⁵ is selected from the group consisting of H, F, Cl, Br, I, and (C₁-C₃)haloalkyl;
(F) R⁶ is H;
(G) R⁷ is selected from the group consisting of F, Cl, and Br;
(H) R⁸ is selected from the group consisting of F, Cl, and Br;
(I) R⁹ is H;
(J) Q¹ is selected from the group consisting of O and S;
(K) Q² is selected from the group consisting of O and S;
(L) R¹⁰ is selected from the group consisting of H, (C₁-C₃)alkyl, (C₂-C₃)alkenyl, and (C₂-C₃)alkynyl;
(M) R¹¹ is selected from the group consisting of H, F, Cl, Br, I, (C₁-C₃)alkyl, (C₁-C₃)haloalkyl, and (C₁-C₃)alkoxy;
(N) R¹² is selected from the group consisting of H, F, Cl, Br, I, (C₁-C₃)alkyl, (C₁-C₃)haloalkyl, and (C₁-C₃)alkoxy;
(O) R¹³ is selected from the group consisting of H, F, Cl, Br, I, (C₁-C₃)alkyl, (C₁-C₃)haloalkyl, and (C₁-C₃)alkoxy;
(P) R¹⁴ is selected from the group consisting of H, F, Cl, Br, I, (C₁-C₃)alkyl, (C₁-C₃)haloalkyl, and (C₁-C₃)alkoxy;
(Q) R¹⁵ is selected from the group consisting of H, (C₁-C₃)alkyl, (C₂-C₃)alkenyl, and (C₂-C₃)alkynyl;
(R) R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, NHC(═O)(C₁-C₆)alkyl-S—(C₁-C₆)haloalkyl; and resolved stereoisomers of the molecules of Formula One.

2d. A molecule according to 1d wherein:
(A) R¹ is selected from the group consisting of H, F, Cl, Br, I, and (C₁-C₃)haloalkyl;
(B) R² is selected from the group consisting of H, F, Cl, Br, I, and (C₁-C₃)haloalkyl;
(C) R³ is selected from the group consisting of H, F, Cl, Br, I, and (C₁-C₃)haloalkyl;
(D) R⁴ is selected from the group consisting of H, F, Cl, Br, I, and (C₁-C₃)haloalkyl;
(E) R⁵ is selected from the group consisting of H, F, Cl, Br, I, and (C₁-C₃)haloalkyl;
(F) R⁶ is H;
(G) R⁷ is selected from the group consisting of F, Cl, and Br;
(H) R⁸ is selected from the group consisting of F, Cl, and Br;
(I) R⁹ is H;
(J) Q¹ is selected from the group consisting of O and S;
(K) Q² is selected from the group consisting of O and S;
(L) R¹⁰ is selected from the group consisting of H, (C₁-C₃)alkyl, (C₂-C₃)alkenyl, and (C₂-C₃)alkynyl;
(M) R¹¹ is selected from the group consisting of H, F, Cl, Br, I, (C₁-C₃)alkyl, (C₁-C₃)haloalkyl, and (C₁-C₃)alkoxy;
(N) R¹² is selected from the group consisting of H, F, Cl, Br, I, (C₁-C₃)alkyl, (C₁-C₃)haloalkyl, and (C₁-C₃)alkoxy;
(O) R¹³ is selected from the group consisting of H, F, Cl, Br, I, (C₁-C₃)alkyl, (C₁-C₃)haloalkyl, and (C₁-C₃)alkoxy;
(P) R¹⁴ is selected from the group consisting of H, F, Cl, Br, I, (C₁-C₃)alkyl, (C₁-C₃)haloalkyl, and (C₁-C₃)alkoxy;
(Q) R¹⁵ is selected from the group consisting of H, (C₁-C₃)alkyl, (C₂-C₃)alkenyl, and (C₂-C₃)alkynyl;
(R) R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, NHC(═O)(C₁-C₆)alkyl-S—(C₁-C₆)haloalkyl.

3d. A molecule according to 1d and 2d wherein said molecule has the following formula

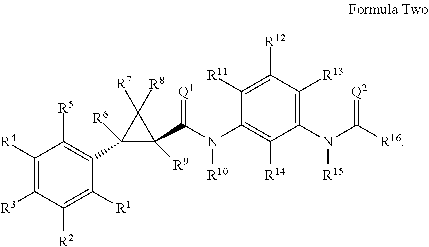

4d. A molecule according to any of the previous details wherein R¹ is selected from the group consisting of H, F, Cl, Br, I, and CF₃.

5d. A molecule according to any of the previous details wherein R² is selected from the group consisting of H, F, Cl, Br, I, and CF₃.

6d. A molecule according to any of the previous details wherein R³ is selected from the group consisting of H, F, Cl, Br, I, and CF₃.

7d. A molecule according to any of the previous details wherein R⁴ is selected from the group consisting of H, F, Cl, Br, I, and CF₃.

8d. A molecule according to any of the previous details wherein R⁵ is selected from the group consisting of H, F, Cl, Br, I, and CF₃.

9d. A molecule according to any of the previous details wherein at least one of R², R³, and R⁴ is CF₃.

10d. A molecule according to any of the previous details wherein R⁷ is Cl.

11d. A molecule according to any of the previous details wherein R⁸ is Cl.

12d. A molecule according to any of the previous details wherein Q¹ is O.

13d. A molecule according to any of the previous details wherein Q² is O.

14d. A molecule according to any of the previous details wherein R¹⁰ is H.

15d. A molecule according to any of the previous details wherein R¹¹ is H.

16d. A molecule according to any of the previous details wherein R¹² is H.

17d. A molecule according to any of the previous details wherein R¹³ is selected from the group consisting of H, F, and Cl.

18d. A molecule according to any of the previous details wherein R¹⁴ is selected from the group consisting of H and F.

19d. A molecule according to any of the previous details wherein R¹⁵ is selected from the group consisting of H and CH₃.

20d. A molecule according to any of the previous details wherein R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl, wherein each said alkyl, alkenyl, alkynyl, cycloalkyl, haloalkyl, phenyl, substituted phenyl, heterocyclyl, and substituted heterocyclyl has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, NHC(═O)(C₁-C₆)alkyl-S—(C₁-C₆)haloalkyl.

21d. A molecule according to any of the previous details wherein R¹⁶ is a heterocyclyl selected from the group consisting of furanyl, isoxazolonyl, isoxazolyl, morpholinyl, substituted pyridinyl, pyrimidinyl, substituted pyrrolidinyl, tetrahydrofuranyl, and substituted thiazolyl, wherein each substituted pyridinyl, substituted pyrrolidinyl, and substituted thiazolyl has one or more substituents selected from the group consisting of H, F, Cl, Br, CN, (C₁-C₆)alkyl, and (C₁-C₆)haloalkyl.

22d. A molecule according to any of the previous details wherein R¹⁶ is selected from the group consisting of substituted pyridinyl, substituted pyrrolidinyl, and substituted thiazolyl, wherein each said substituted pyridinyl, substituted pyrrolidinyl, and substituted thiazolyl has one or more substituents selected from the group consisting of H, F, CF₃, and CH₂CF₃.

23d. A molecule according to any of the previous details wherein R¹⁶ is a substituted phenyl wherein said substituted phenyl has one or more substituents selected from the group consisting of H, F, Cl, CN, NH₂, NO₂, (C₁-C₆)alkyl, (C₂-C₆)alkynyl, (C₁-C₆)haloalkyl, (C₁-C₆)alkoxy, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, and NHC(═O)(C₁-C₆)alkyl-S—(C₁-C₆)haloalkyl.

24d. A molecule according to any of the previous details, 1d through 23d inclusive, wherein
R¹ is selected from the group consisting of H, F, and Cl;
R² is selected from the group consisting of H, F, Cl, Br, and CF₃;
R³ is selected from the group consisting of H, F, Cl, Br, and CF₃;
R⁴ is selected from the group consisting of H, F, Cl, Br, and CF₃;
R⁵ is selected from the group consisting of H, F, and Cl;
R⁶ is H;
R⁷ is selected from the group consisting of F and Cl;
R⁸ is selected from the group consisting of F and Cl;
R⁹ is H;
Q¹ is selected from the group consisting of O and S;
Q² is selected from the group consisting of O and S;
R¹⁰ is selected from the group consisting of H and (C₁-C₃)alkyl;
R¹¹ is selected from the group consisting of H, F, and Cl;
R¹² is selected from the group consisting of H, F, and Cl;
R¹³ is selected from the group consisting of H, F, Cl, (C₁-C₃)alkyl, and (C₁-C₃)haloalkyl;
R¹⁴ is selected from the group consisting of H, F, and Cl;
R¹⁵ is selected from the group consisting of H and (C₁-C₃)alkyl;
R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl, has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, and NHC(═O)(C₁-C₆)alkyl-S—(C₁-C₆)haloalkyl.

25d. A molecule according to any of the previous details, 1d through 24d inclusive, wherein
R¹ is H;
R² is selected from the group consisting of H, Cl, Br, and CF₃;
R³ is selected from the group consisting of H, F, and Cl;
R⁴ is selected from the group consisting of H, Cl, Br, and CF₃;
R⁵ is H;
R⁶ is H;
R⁷ is Cl;
R⁸ is Cl;
R⁹ is H;
Q¹ is O;
Q² is O;
R¹⁰ is H;
R¹¹ is H;
R¹² is H,
R¹³ is selected from the group consisting of H, F, and Cl;
R¹⁴ is selected from the group consisting of H and F;
R¹⁵ is selected from the group consisting of H and CH₃;
R¹⁶ is selected from the group consisting of (C₁-C₄)alkyl, substituted (C₁-C₄)alkyl, ((C₁-C₄)alkyl)O((C₁-C₄)alkyl), ((C₁-C₄)alkyl)O substituted ((C₁-C₄)alkyl), substituted (C₁-C₄)alkylphenyl, (C₂-C₄)alkenyl, cyclopropyl, substituted cyclopropyl, furanyl, isoxazolonyl, phenyl, substituted phenyl, substituted pyridinyl, pyrimidinyl, substituted pyrrolidinyl, tetrahydrofuranyl, and substituted thiazolyl,
wherein said substituted (C₁-C₄)alkyl, substituted (C₁-C₄)alkylphenyl, substituted cyclopropyl, substituted phenyl, substituted pyridinyl, substituted pyrrolidinyl, and substituted thiazolyl has one or more substituents selected from the group consisting of C≡CH, CF₃, CH₂CF₃, CH₃, Cl, CN, F, H, morpholinyl, NH₂, NHC(═O)CF₃, NHC(═O)CH₃, NHC(═O)CHF₂, NHC(═O)CH₂CF₃, NHC(═O)CH₂CH₂CF₃, NHC(═O)CH₂OCH₃, NHC(═O)CH₂SCF₃, NHC(═O)CH₂OCH₂CF₃, NO₂, OCH₃, Ophenyl, and phenyl.

26d. A molecule selected from Table 1, wherein said molecule is selected from the group consisting of F1, F2, F3, F4, F5, F6, F7, F8, F9, F10, F11, F12, F13, F14, F15, F18, F20, F24, F25, F26, F27, F30, F32, F34, F36, F37, F38, F39, F40, F41, F42, F43, F44, F47, F48, F49, F50, F51, F52, F53, F54, F55, F56, F57, F58, F60, F61, F62, F63, F64, F65, F66, F67, F68, F69, F70, F71, F72, F73, F74, F75, F77, F78, F79, F81, F82, F83, F84, F85, F87, F88, F89, F90, F91, F92, F93, F94, F95, F96, F97, F98, F99, F100, F102, F103, F104, F105, F106, F109, F110, F111, F112, F113, F114, F115, F116, F117, F118, F119, F120, and F121.

27d. A molecule selected from Table 1, wherein said molecule is selected from the group consisting of F11, F27, F30, F32, F34, F36, F37, F38, F52, F60, F93, F96, F97, F98, F99, F106, F109, and F110.

28d. A seed treated with a molecule according to any of the previous details, 1d through 26d inclusive.

29d. A molecule according to any of the previous details, 1d through 28d inclusive, wherein said molecule is in the form of a resolved stereoisomer.

30d. A process to control a pest said process comprising applying to a locus, a pesticidally effective amount of a molecule according to any of the previous details, 1d through 29d inclusive.

31d. A process according to 30d wherein said pest is selected from the group consisting of ants, aphids, bed bugs, beetles, bristletails, caterpillars, cockroaches, crickets, earwigs, fleas, flies, grasshoppers, grubs, leafhoppers, lice, locusts, maggots, mites, nematodes, planthoppers, psyllids, sawflies, scales, silverfish, slugs, snails, spiders, springtails, stink bugs, symphylans, termites, thrips, ticks, wasps, whiteflies, and wireworms.

32d. A process according to 30d wherein said pest is selected from Subphyla Chelicerata, Myriapoda, or Hexapoda.

33d. A process according to 30d wherein said pest is selected from Class of Arachnida, Symphyla, or Insecta.

34d. A process according to 30d wherein said pest is selected from Order Anoplura.

35d. A process according to 30d wherein said pest is selected from Order Coleoptera.

36d. A process according to 30d wherein said pest is selected from Order Dermaptera.

37d. A process according to 30d wherein said pest is selected from Order Blattaria.

38d. A process according to 30d wherein said pest is selected from Order Diptera.

39d. A process according to 30d wherein said pest is selected from Order Hemiptera.

40d. A process according to 30d wherein said pest is selected from Order Hymenoptera.

41d. A process according to 30d wherein said pest is selected from Order Isoptera.

42d. A process according to 30d wherein said pest is selected from Order Lepidoptera.

43d. A process according to 30d wherein said pest is selected from Order Mallophaga.

44d. A process according to 30d wherein said pest is selected from Order Orthoptera.

45d. A process according to 30d wherein said pest is selected from Order Psocoptera.

46d. A process according to 30d wherein said pest is selected from Order Siphonaptera.

47d. A process according to 30d wherein said pest is selected from Order Thysanoptera.

48d. A process according to 30d wherein said pest is selected from Order Thysanura.

49d. A process according to 30d wherein said pest is selected from Order Acarina.

50d. A process according to 30d wherein said pest is selected from Order Araneae.

51d. A process according to 30d wherein said pest is selected from Class Symphyla.

52d. A process according to 30d wherein said pest is selected from Subclass Collembola.

53d. A process according to 30d wherein said pest is selected from Phylum Nematoda.

54d. A process according to details 30d wherein said pest is selected from Phylum Mollusca.

55d. A process according to 30d wherein said pest is a sap-feeding pest.

56d. A process according to 30d wherein said pest is selected from aphids, leafhoppers, moths, scales, thrips, psyllids, mealybugs, stinkbugs, and whiteflies.

57d. A process according to 30d wherein said pest is selected from Orders Anoplura and Hemiptera.

58d. A process according to 30d wherein said pest is selected from Aulacaspis spp., Aphrophora spp., Aphis spp., Bemisia spp., Coccus spp., Euschistus spp., Lygus spp., Macrosiphum spp., Nezara spp., and Rhopalosiphum spp.

59d. A process according to 30d wherein said pest is a chewing pest.

60d. A process according to 30d wherein said pest is selected from caterpillars, beetles, grasshoppers, and locusts.

61d. A process according to 30d wherein said pest is selected from Coleoptera and Lepidoptera.

62d. A process according to 30d wherein said pest is selected from Anthonomus spp., Cerotoma spp., Chaetocnema spp., Colaspis spp., Cyclocephala spp., Diabrotica spp., Hypera spp., Phyllophaga spp., Phyllotreta spp., Sphenophorus spp., Sitophilus spp.

63d. A process according to 30d wherein said locus is where alfalfa, almonds, apples, barley, beans, canola, corn, cotton, crucifers, lettuce, oats, oranges, pears, peppers, potatoes, rice, sorghum, soybeans, strawberries, sugarcane, sugar beets, sunflowers, tobacco, tomatoes, wheat, and other valuable crops are growing or the seeds thereof are planted.

64d. A process according to 30d wherein said locus is where plants genetically modified to express specialized traits are planted.

65d. A process according to 30d wherein said applying is done to the foliar and/or fruiting portions of plants.

66d. A process according to 30d wherein said applying is done to the soil.

67d. A process according to 30d wherein said applying is done by drip irrigation, furrow application, or pre- or post-planting soil drench.

68d. A process according to 30d wherein said applying is done to the foliar and/or fruiting portions of plants, or by treating the seeds of a plant before planting.

69d. A process comprising applying a molecule according to any of the previous detail details, 1d through 27d inclusive, to a locus that includes a non-human animal to control endoparasites, ectoparasites, or both.

### Table Section

TABLE 1
Structure and Preparation Method for F Compounds

| Cmpd. No. | Structure | Prep.* |
|---|---|---|
| F1 | 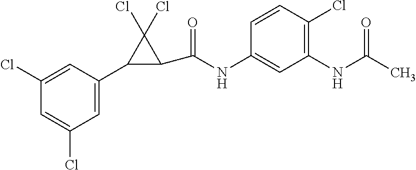 [SMILES: CC(=O)Nc1cc(NC(=O)C2C(c3cc(Cl)cc(Cl)c3)C2(Cl)Cl)ccc1Cl] | 6 |
| F2 | 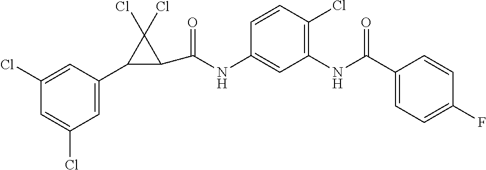 [SMILES: O=C(Nc1cc(NC(=O)C2C(c3cc(Cl)cc(Cl)c3)C2(Cl)Cl)ccc1Cl)c1ccc(F)cc1] | 6 |
| F3 | 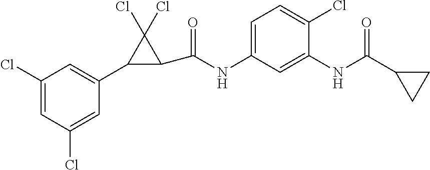 [SMILES: O=C(Nc1cc(NC(=O)C2C(c3cc(Cl)cc(Cl)c3)C2(Cl)Cl)ccc1Cl)C1CC1] | 6 |
| F4 | 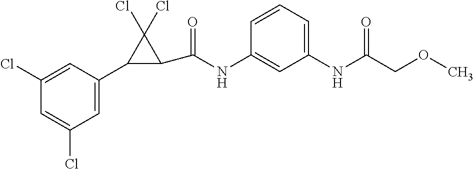 [SMILES: COCC(=O)Nc1cccc(NC(=O)C2C(c3cc(Cl)cc(Cl)c3)C2(Cl)Cl)c1] | 4 |
| F5 | 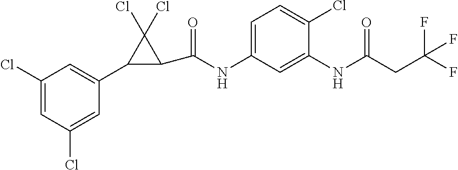 [SMILES: O=C(CC(F)(F)F)Nc1cc(NC(=O)C2C(c3cc(Cl)cc(Cl)c3)C2(Cl)Cl)ccc1Cl] | 2 |
| F6 | 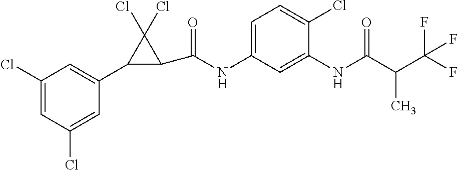 [SMILES: CC(C(=O)Nc1cc(NC(=O)C2C(c3cc(Cl)cc(Cl)c3)C2(Cl)Cl)ccc1Cl)C(F)(F)F] | 2 |
| F7 | 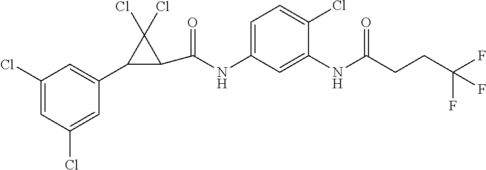 [SMILES: O=C(CCC(F)(F)F)Nc1cc(NC(=O)C2C(c3cc(Cl)cc(Cl)c3)C2(Cl)Cl)ccc1Cl] | 2 |
| F8 | 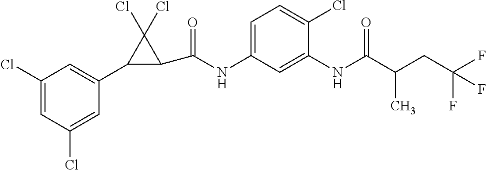 [SMILES: CC(CC(F)(F)F)C(=O)Nc1cc(NC(=O)C2C(c3cc(Cl)cc(Cl)c3)C2(Cl)Cl)ccc1Cl] | 2 |
| F9 | 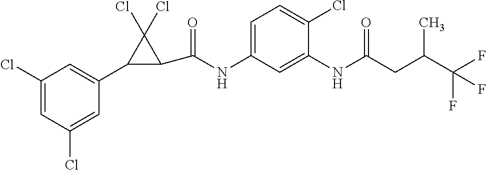 [SMILES: CC(CC(=O)Nc1cc(NC(=O)C2C(c3cc(Cl)cc(Cl)c3)C2(Cl)Cl)ccc1Cl)C(F)(F)F] | 2 |
| F10 | 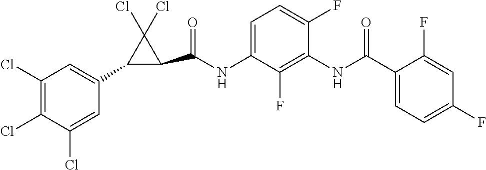 [SMILES: O=C(Nc1c(F)ccc(NC(=O)[C@H]2[C@H](c3cc(Cl)c(Cl)c(Cl)c3)C2(Cl)Cl)c1F)c1ccc(F)cc1F] | 1 |
| F11 | 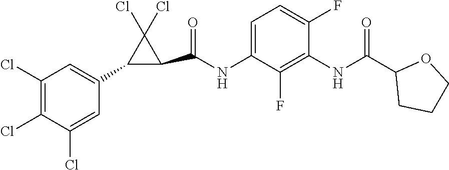 [SMILES: O=C(Nc1c(F)ccc(NC(=O)[C@H]2[C@H](c3cc(Cl)c(Cl)c(Cl)c3)C2(Cl)Cl)c1F)C1CCCO1] | 1 |
| F12 | 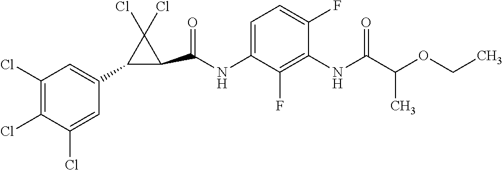 [SMILES: CCOC(C)C(=O)Nc1c(F)ccc(NC(=O)[C@H]2[C@H](c3cc(Cl)c(Cl)c(Cl)c3)C2(Cl)Cl)c1F] | 1 |
| F13 | 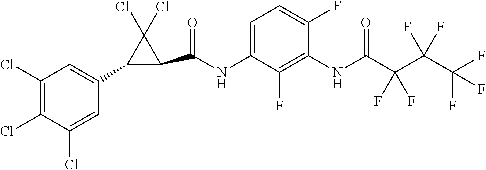 [SMILES: O=C(Nc1ccc(F)c(NC(=O)C(F)(F)C(F)(F)C(F)(F)F)c1F)[C@H]1[C@H](c2cc(Cl)c(Cl)c(Cl)c2)C1(Cl)Cl] | 1 |
| F14 | 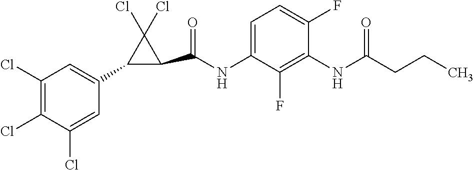 [SMILES: CCCC(=O)Nc1c(F)ccc(NC(=O)[C@H]2[C@H](c3cc(Cl)c(Cl)c(Cl)c3)C2(Cl)Cl)c1F] | 1 |
| F15 | 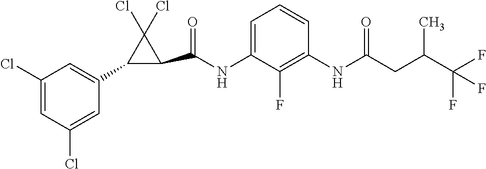 [SMILES: CC(CC(=O)Nc1cccc(NC(=O)[C@H]2[C@H](c3cc(Cl)cc(Cl)c3)C2(Cl)Cl)c1F)C(F)(F)F] | 1 |
| F16 | 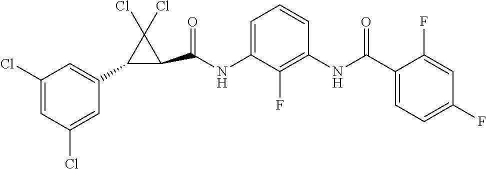 [SMILES: O=C(Nc1cccc(NC(=O)[C@H]2[C@H](c3cc(Cl)cc(Cl)c3)C2(Cl)Cl)c1F)c1ccc(F)cc1F] | 1 |
| F17 | 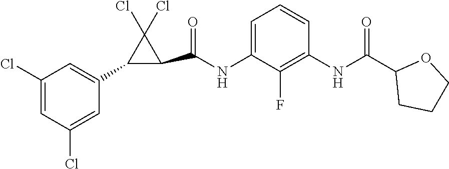 [SMILES: O=C(Nc1cccc(NC(=O)[C@H]2[C@H](c3cc(Cl)cc(Cl)c3)C2(Cl)Cl)c1F)C1CCCO1] | 1 |
| F18 | 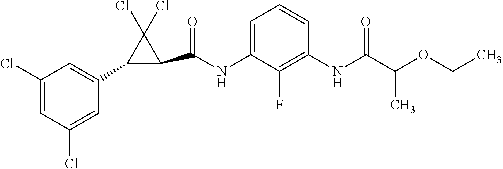 [SMILES: CCOC(C)C(=O)Nc1cccc(NC(=O)[C@H]2[C@H](c3cc(Cl)cc(Cl)c3)C2(Cl)Cl)c1F] | 1 |
| F19 | 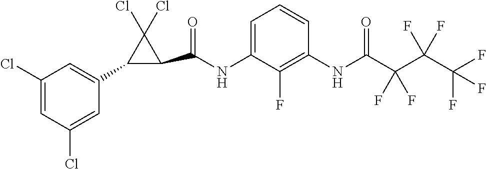 [SMILES: O=C(Nc1cccc(NC(=O)C(F)(F)C(F)(F)C(F)(F)F)c1F)[C@H]1[C@H](c2cc(Cl)cc(Cl)c2)C1(Cl)Cl] | 1 |
| F20 | 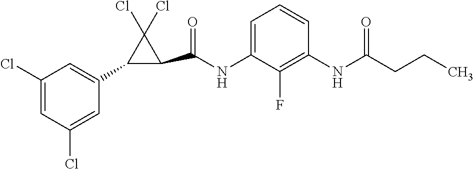 [SMILES: CCCC(=O)Nc1cccc(NC(=O)[C@H]2[C@H](c3cc(Cl)cc(Cl)c3)C2(Cl)Cl)c1F] | 1 |
| F21 | 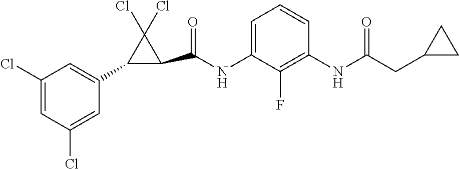 [SMILES: O=C(CC1CC1)Nc1cccc(NC(=O)[C@H]2[C@H](c3cc(Cl)cc(Cl)c3)C2(Cl)Cl)c1F] | 1 |
| F22 | 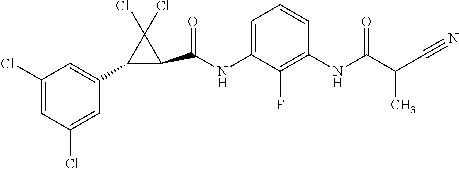 [SMILES: CC(C#N)C(=O)Nc1cccc(NC(=O)[C@H]2[C@H](c3cc(Cl)cc(Cl)c3)C2(Cl)Cl)c1F] | 1 |
| F23 | 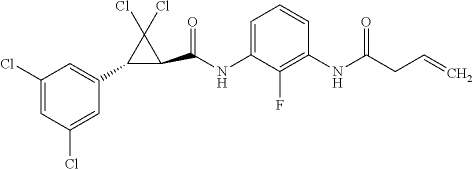 [SMILES: C=CCC(=O)Nc1cccc(NC(=O)[C@H]2[C@H](c3cc(Cl)cc(Cl)c3)C2(Cl)Cl)c1F] | 1 |
| F24 | 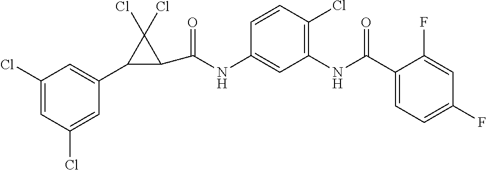 [SMILES: O=C(Nc1cc(NC(=O)C2C(c3cc(Cl)cc(Cl)c3)C2(Cl)Cl)ccc1Cl)c1ccc(F)cc1F] | 2 |
| F25 | 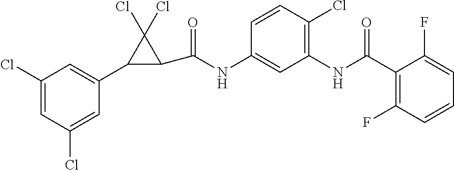 [SMILES: O=C(Nc1cc(NC(=O)C2C(c3cc(Cl)cc(Cl)c3)C2(Cl)Cl)ccc1Cl)c1c(F)cccc1F] | 2 |
| F26 | 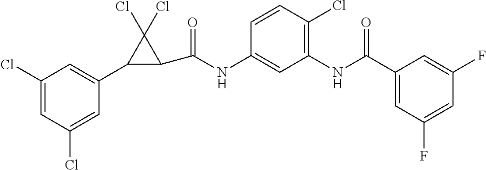 [SMILES: O=C(Nc1cc(NC(=O)C2C(c3cc(Cl)cc(Cl)c3)C2(Cl)Cl)ccc1Cl)c1cc(F)cc(F)c1] | 5 |
| F27 | 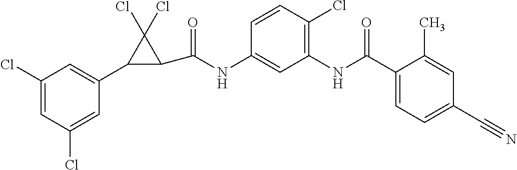 [SMILES: Cc1cc(C#N)ccc1C(=O)Nc1cc(NC(=O)C2C(c3cc(Cl)cc(Cl)c3)C2(Cl)Cl)ccc1Cl] | 2 |
| F28 | 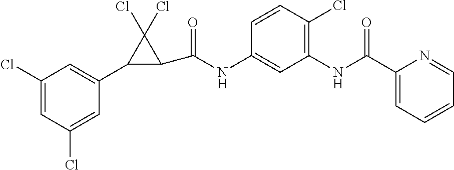 [SMILES: O=C(Nc1cc(NC(=O)C2C(c3cc(Cl)cc(Cl)c3)C2(Cl)Cl)ccc1Cl)c1ccccn1] | 3 |
| F29 | 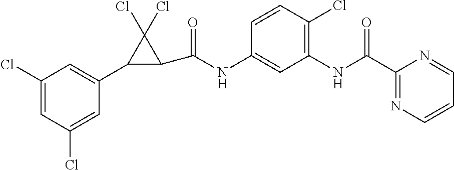 [SMILES: O=C(Nc1cc(NC(=O)C2C(c3cc(Cl)cc(Cl)c3)C2(Cl)Cl)ccc1Cl)c1ncccn1] | 3 |
| F30 | 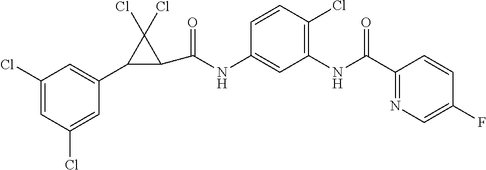 [SMILES: O=C(Nc1cc(NC(=O)C2C(c3cc(Cl)cc(Cl)c3)C2(Cl)Cl)ccc1Cl)c1ccc(F)cn1] | 3 |
| F31 | 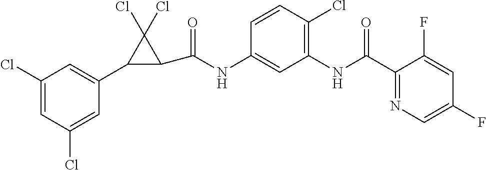 [SMILES: O=C(Nc1cc(NC(=O)C2C(c3cc(Cl)cc(Cl)c3)C2(Cl)Cl)ccc1Cl)c1ncc(F)cc1F] | 3 |
| F32 | 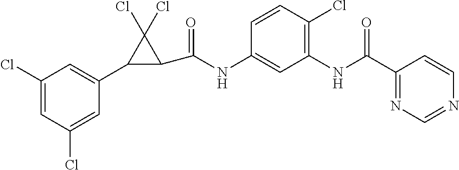 [SMILES: O=C(Nc1cc(NC(=O)C2C(c3cc(Cl)cc(Cl)c3)C2(Cl)Cl)ccc1Cl)c1ccncn1] | 3 |
| F33 | 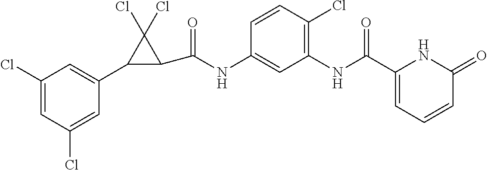 [SMILES: O=C(Nc1cc(NC(=O)C2C(c3cc(Cl)cc(Cl)c3)C2(Cl)Cl)ccc1Cl)c1cccc(=O)[nH]1] | 6 |
| F34 | 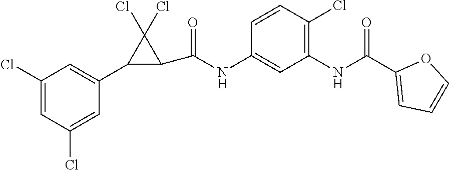 [SMILES: O=C(Nc1cc(NC(=O)C2C(c3cc(Cl)cc(Cl)c3)C2(Cl)Cl)ccc1Cl)c1ccco1] | 2 |
| F35 | 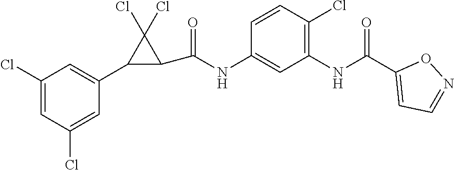 [SMILES: O=C(Nc1cc(NC(=O)C2C(c3cc(Cl)cc(Cl)c3)C2(Cl)Cl)ccc1Cl)c1ccno1] | 5 |
| F36 | 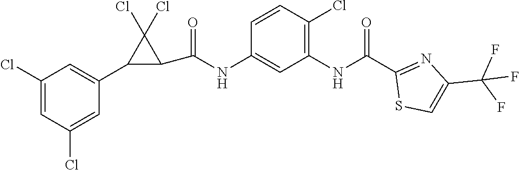 [SMILES: O=C(Nc1cc(NC(=O)C2C(c3cc(Cl)cc(Cl)c3)C2(Cl)Cl)ccc1Cl)c1nc(C(F)(F)F)cs1] | 6 |
| F37 | 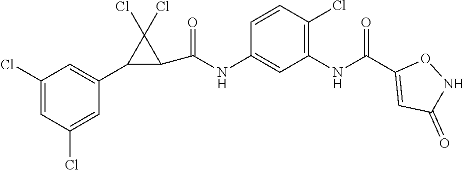 [SMILES: O=C(Nc1cc(NC(=O)C2C(c3cc(Cl)cc(Cl)c3)C2(Cl)Cl)ccc1Cl)c1cc(=O)[nH]o1] | 6 |
| F38 | 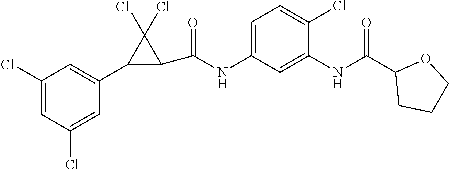 [SMILES: O=C(Nc1cc(NC(=O)C2C(c3cc(Cl)cc(Cl)c3)C2(Cl)Cl)ccc1Cl)C1CCCO1] | 2 |
| F39 | 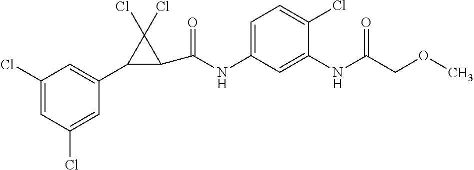 [SMILES: COCC(=O)Nc1cc(NC(=O)C2C(c3cc(Cl)cc(Cl)c3)C2(Cl)Cl)ccc1Cl] | 6 |
| F40 | 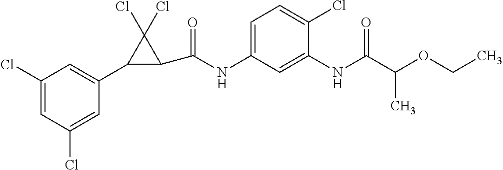 [SMILES: CCOC(C)C(=O)Nc1cc(NC(=O)C2C(c3cc(Cl)cc(Cl)c3)C2(Cl)Cl)ccc1Cl] | 4 |
| F41 | 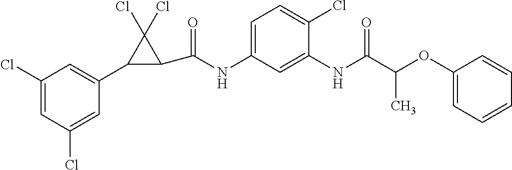 [SMILES: CC(Oc1ccccc1)C(=O)Nc1cc(NC(=O)C2C(c3cc(Cl)cc(Cl)c3)C2(Cl)Cl)ccc1Cl] | 2 |
| F42 | 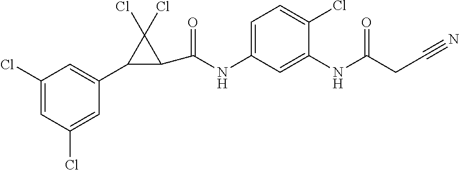 [SMILES: N#CCC(=O)Nc1cc(NC(=O)C2C(c3cc(Cl)cc(Cl)c3)C2(Cl)Cl)ccc1Cl] | 6 |
| F43 | 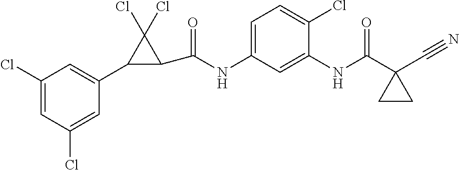 [SMILES: N#CC1(C(=O)Nc2cc(NC(=O)C3C(c4cc(Cl)cc(Cl)c4)C3(Cl)Cl)ccc2Cl)CC1] | 6 |
| F44 | 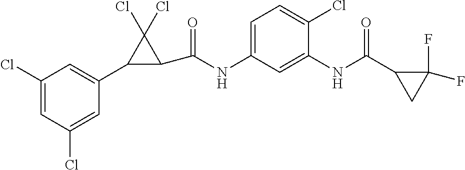 [SMILES: O=C(Nc1cc(NC(=O)C2C(c3cc(Cl)cc(Cl)c3)C2(Cl)Cl)ccc1Cl)C1CC1(F)F] | 1 |
| F45 | 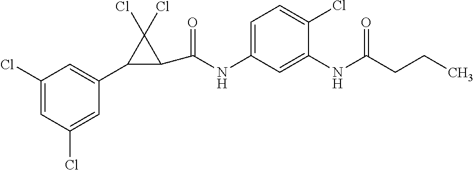 [SMILES: CCCC(=O)Nc1cc(NC(=O)C2C(c3cc(Cl)cc(Cl)c3)C2(Cl)Cl)ccc1Cl] | 2 |
| F46 | 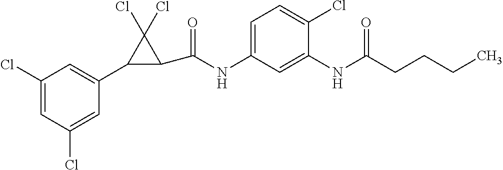 [SMILES: CCCCC(=O)Nc1cc(NC(=O)C2C(c3cc(Cl)cc(Cl)c3)C2(Cl)Cl)ccc1Cl] | 2 |
| F47 | 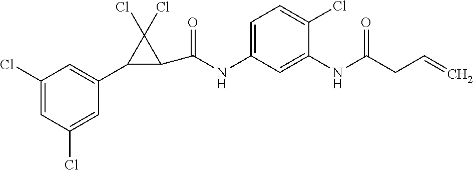 [SMILES: C=CCC(=O)Nc1cc(NC(=O)C2C(c3cc(Cl)cc(Cl)c3)C2(Cl)Cl)ccc1Cl] | 6 |
| F48 | 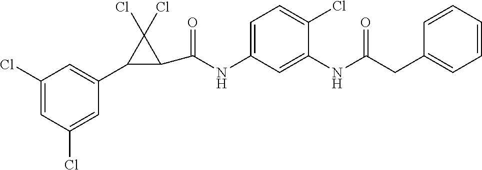 [SMILES: O=C(Cc1ccccc1)Nc1cc(NC(=O)C2C(c3cc(Cl)cc(Cl)c3)C2(Cl)Cl)ccc1Cl] | 4 |
| F49 | 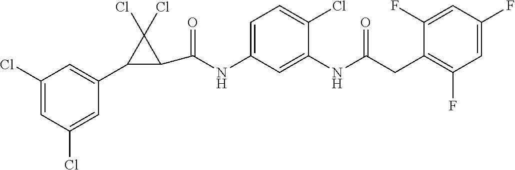 [SMILES: O=C(Cc1c(F)cc(F)cc1F)Nc1cc(NC(=O)C2C(c3cc(Cl)cc(Cl)c3)C2(Cl)Cl)ccc1Cl] | 4 |
| F50 | 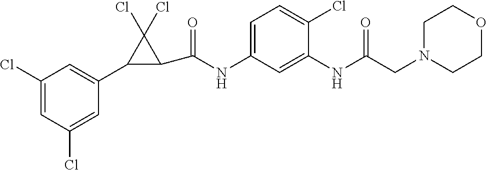 [SMILES: O=C(CN1CCOCC1)Nc1cc(NC(=O)C2C(c3cc(Cl)cc(Cl)c3)C2(Cl)Cl)ccc1Cl] | 2 |
| F51 | 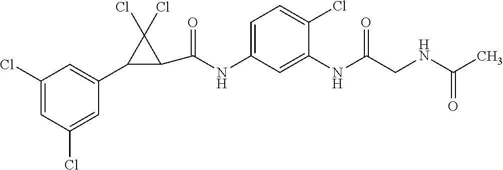 [SMILES: CC(=O)NCC(=O)Nc1cc(NC(=O)C2C(c3cc(Cl)cc(Cl)c3)C2(Cl)Cl)ccc1Cl] | 5 |
| F52 | 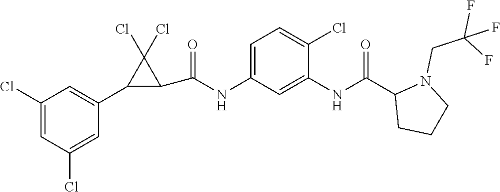 [SMILES: O=C(Nc1cc(NC(=O)C2C(c3cc(Cl)cc(Cl)c3)C2(Cl)Cl)ccc1Cl)C1CCCN1CC(F)(F)F] | 5 |
| F53 | 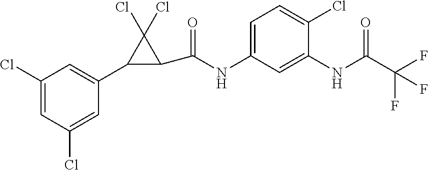 [SMILES: O=C(Nc1ccc(Cl)c(NC(=O)C(F)(F)F)c1)C1C(c2cc(Cl)cc(Cl)c2)C1(Cl)Cl] | 7 |
| F54 | 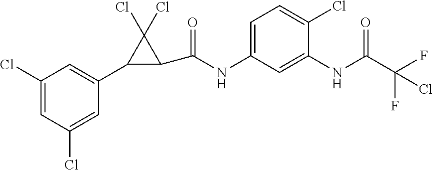 [SMILES: O=C(Nc1ccc(Cl)c(NC(=O)C(F)(F)Cl)c1)C1C(c2cc(Cl)cc(Cl)c2)C1(Cl)Cl] | 6 |
| F55 | 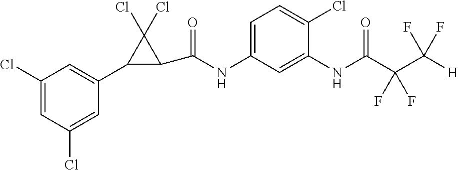 [SMILES: O=C(Nc1ccc(Cl)c(NC(=O)C(F)(F)C(F)F)c1)C1C(c2cc(Cl)cc(Cl)c2)C1(Cl)Cl] | 6 |
| F56 | 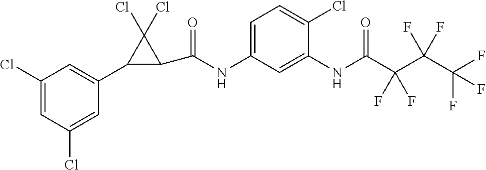 [SMILES: O=C(Nc1ccc(Cl)c(NC(=O)C(F)(F)C(F)(F)C(F)(F)F)c1)C1C(c2cc(Cl)cc(Cl)c2)C1(Cl)Cl] | 6 |
| F57 | 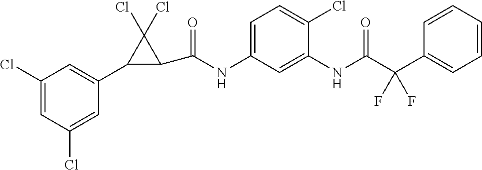 [SMILES: O=C(Nc1ccc(Cl)c(NC(=O)C(F)(F)c2ccccc2)c1)C1C(c2cc(Cl)cc(Cl)c2)C1(Cl)Cl] | 4 |
| F58 | 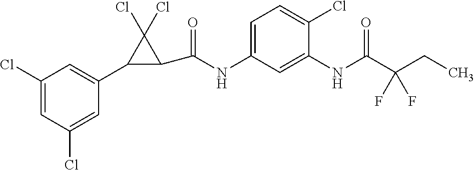 [SMILES: CCC(F)(F)C(=O)Nc1cc(NC(=O)C2C(c3cc(Cl)cc(Cl)c3)C2(Cl)Cl)ccc1Cl] | 6 |
| F60 | 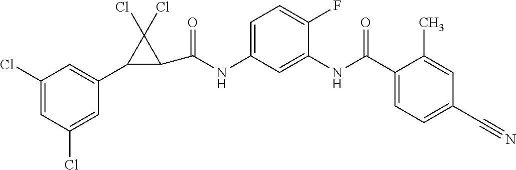 [SMILES: Cc1cc(C#N)ccc1C(=O)Nc1cc(NC(=O)C2C(c3cc(Cl)cc(Cl)c3)C2(Cl)Cl)ccc1F] | 1 |
| F61 | 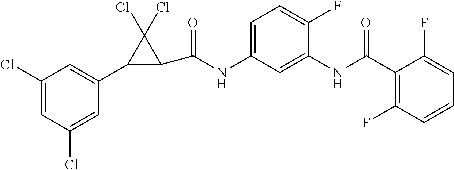 [SMILES: O=C(Nc1cc(NC(=O)C2C(c3cc(Cl)cc(Cl)c3)C2(Cl)Cl)ccc1F)c1c(F)cccc1F] | 1 |
| F62 | 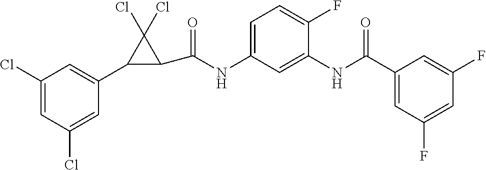 [SMILES: O=C(Nc1cc(NC(=O)C2C(c3cc(Cl)cc(Cl)c3)C2(Cl)Cl)ccc1F)c1cc(F)cc(F)c1] | 1 |
| F63 | 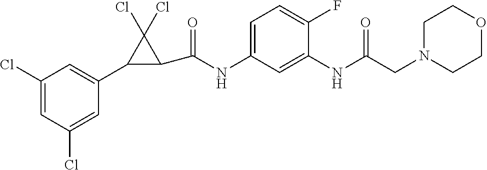 [SMILES: O=C(CN1CCOCC1)Nc1cc(NC(=O)C2C(c3cc(Cl)cc(Cl)c3)C2(Cl)Cl)ccc1F] | 1 |
| F64 | 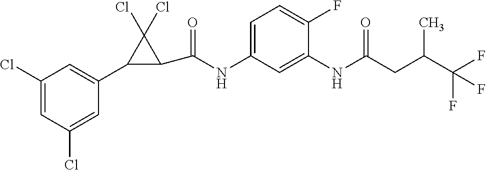 [SMILES: CC(CC(=O)Nc1cc(NC(=O)C2C(c3cc(Cl)cc(Cl)c3)C2(Cl)Cl)ccc1F)C(F)(F)F] | 1 |
| F65 | 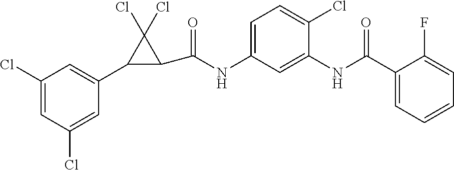 [SMILES: O=C(Nc1cc(NC(=O)C2C(c3cc(Cl)cc(Cl)c3)C2(Cl)Cl)ccc1Cl)c1ccccc1F] | 2 |
| F66 | 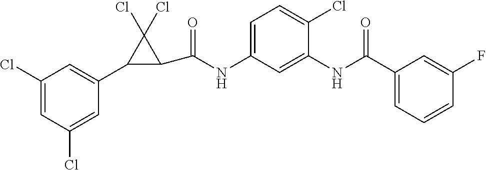 [SMILES: O=C(Nc1cc(NC(=O)C2C(c3cc(Cl)cc(Cl)c3)C2(Cl)Cl)ccc1Cl)c1cccc(F)c1] | 2 |
| F67 | 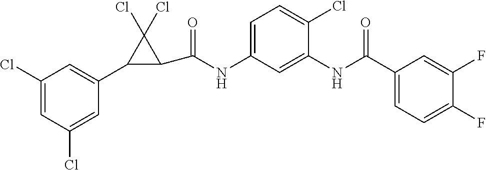 [SMILES: O=C(Nc1cc(NC(=O)C2C(c3cc(Cl)cc(Cl)c3)C2(Cl)Cl)ccc1Cl)c1ccc(F)c(F)c1] | 2 |
| F68 | 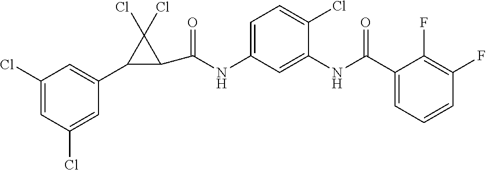 [SMILES: O=C(Nc1cc(NC(=O)C2C(c3cc(Cl)cc(Cl)c3)C2(Cl)Cl)ccc1Cl)c1cccc(F)c1F] | 2 |
| F69 | 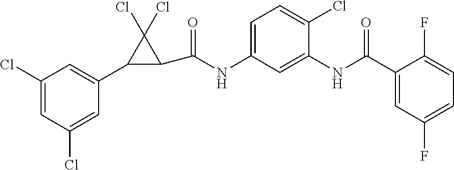 [SMILES: O=C(Nc1cc(NC(=O)C2C(c3cc(Cl)cc(Cl)c3)C2(Cl)Cl)ccc1Cl)c1cc(F)ccc1F] | 2 |
| F70 | 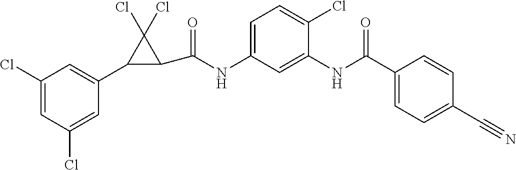 [SMILES: N#Cc1ccc(C(=O)Nc2cc(NC(=O)C3C(c4cc(Cl)cc(Cl)c4)C3(Cl)Cl)ccc2Cl)cc1] | 6 |
| F71 | 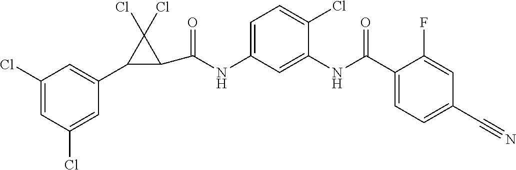 [SMILES: N#Cc1ccc(C(=O)Nc2cc(NC(=O)C3C(c4cc(Cl)cc(Cl)c4)C3(Cl)Cl)ccc2Cl)c(F)c1] | 6 |
| F72 | 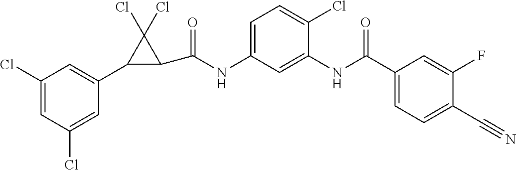 [SMILES: N#Cc1ccc(C(=O)Nc2cc(NC(=O)C3C(c4cc(Cl)cc(Cl)c4)C3(Cl)Cl)ccc2Cl)cc1F] | 6 |
| F73 | 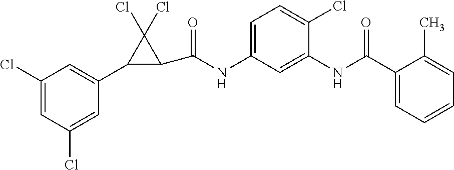 [SMILES: Cc1ccccc1C(=O)Nc1cc(NC(=O)C2C(c3cc(Cl)cc(Cl)c3)C2(Cl)Cl)ccc1Cl] | 6 |
| F74 | 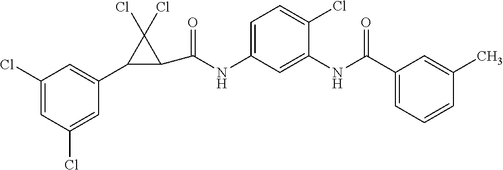 [SMILES: Cc1cccc(C(=O)Nc2cc(NC(=O)C3C(c4cc(Cl)cc(Cl)c4)C3(Cl)Cl)ccc2Cl)c1] | 6 |
| F75 | 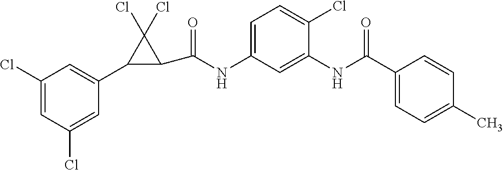 [SMILES: Cc1ccc(C(=O)Nc2cc(NC(=O)C3C(c4cc(Cl)cc(Cl)c4)C3(Cl)Cl)ccc2Cl)cc1] | 2 |
| F76 | 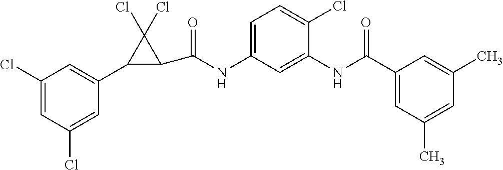 [SMILES: Cc1cc(C)cc(C(=O)Nc2cc(NC(=O)C3C(c4cc(Cl)cc(Cl)c4)C3(Cl)Cl)ccc2Cl)c1] | 2 |
| F77 | 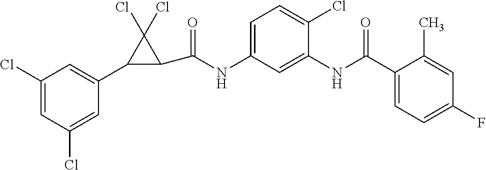 [SMILES: Cc1cc(F)ccc1C(=O)Nc1cc(NC(=O)C2C(c3cc(Cl)cc(Cl)c3)C2(Cl)Cl)ccc1Cl] | 2 |
| F78 | 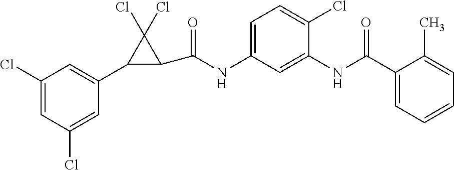 [SMILES: Cc1ccccc1C(=O)Nc1cc(NC(=O)C2C(c3cc(Cl)cc(Cl)c3)C2(Cl)Cl)ccc1Cl] | 2 |
| F79 | 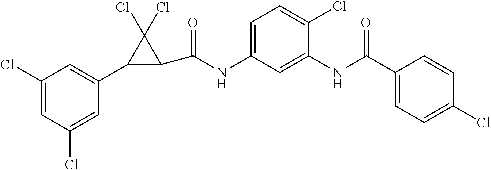 [SMILES: O=C(Nc1cc(NC(=O)C2C(c3cc(Cl)cc(Cl)c3)C2(Cl)Cl)ccc1Cl)c1ccc(Cl)cc1] | 2 |
| F80 | 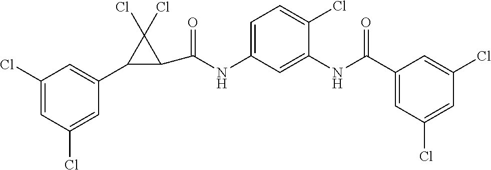 [SMILES: O=C(Nc1cc(NC(=O)C2C(c3cc(Cl)cc(Cl)c3)C2(Cl)Cl)ccc1Cl)c1cc(Cl)cc(Cl)c1] | 6 |
| F81 | 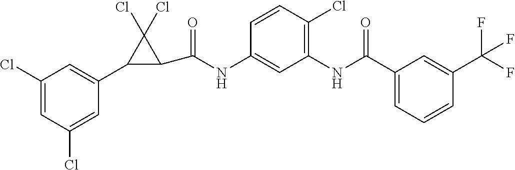 [SMILES: O=C(Nc1cc(NC(=O)C2C(c3cc(Cl)cc(Cl)c3)C2(Cl)Cl)ccc1Cl)c1cccc(C(F)(F)F)c1] | 6 |
| F82 | 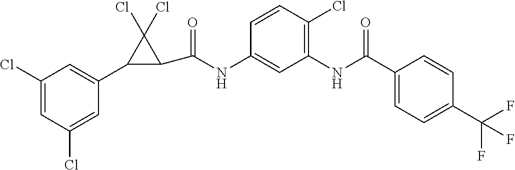 [SMILES: O=C(Nc1cc(NC(=O)C2C(c3cc(Cl)cc(Cl)c3)C2(Cl)Cl)ccc1Cl)c1ccc(C(F)(F)F)cc1] | 1 |
| F83 | 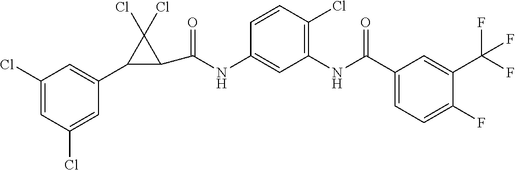 [SMILES: O=C(Nc1cc(NC(=O)C2C(c3cc(Cl)cc(Cl)c3)C2(Cl)Cl)ccc1Cl)c1ccc(F)c(C(F)(F)F)c1] | 2 |
| F84 | 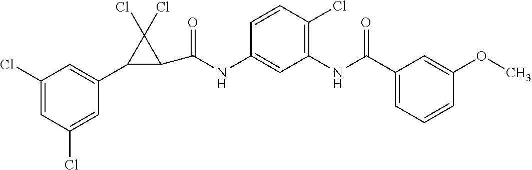 [SMILES: COc1cccc(C(=O)Nc2cc(NC(=O)C3C(c4cc(Cl)cc(Cl)c4)C3(Cl)Cl)ccc2Cl)c1] | 2 |
| F85 | 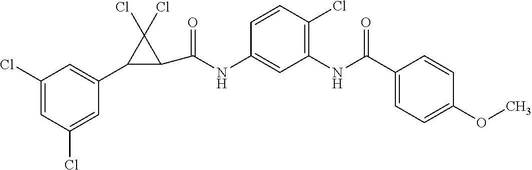 [SMILES: COc1ccc(C(=O)Nc2cc(NC(=O)C3C(c4cc(Cl)cc(Cl)c4)C3(Cl)Cl)ccc2Cl)cc1] | 2 |
| F86 | 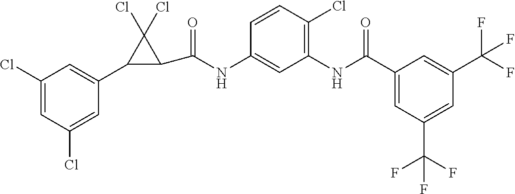 [SMILES: O=C(Nc1cc(NC(=O)C2C(c3cc(Cl)cc(Cl)c3)C2(Cl)Cl)ccc1Cl)c1cc(C(F)(F)F)cc(C(F)(F)F)c1] | 6 |
| F87 | 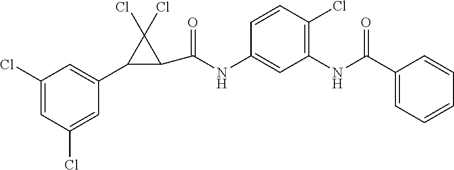 [SMILES: O=C(Nc1cc(NC(=O)C2C(c3cc(Cl)cc(Cl)c3)C2(Cl)Cl)ccc1Cl)c1ccccc1] | 2 |
| F88 | 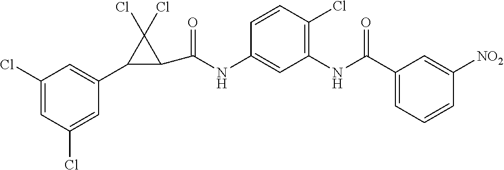 [SMILES: O=C(Nc1cc(NC(=O)C2C(c3cc(Cl)cc(Cl)c3)C2(Cl)Cl)ccc1Cl)c1cccc([N+](=O)[O-])c1] | 9 |
| F89 | 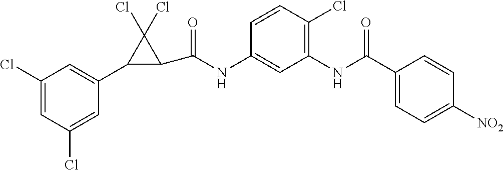 [SMILES: O=C(Nc1cc(NC(=O)C2C(c3cc(Cl)cc(Cl)c3)C2(Cl)Cl)ccc1Cl)c1ccc([N+](=O)[O-])cc1] | 9 |
| F90 | 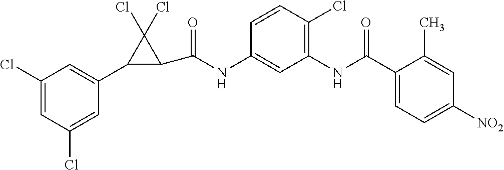 [SMILES: Cc1cc([N+](=O)[O-])ccc1C(=O)Nc1cc(NC(=O)C2C(c3cc(Cl)cc(Cl)c3)C2(Cl)Cl)ccc1Cl] | 9 |
| F91 | 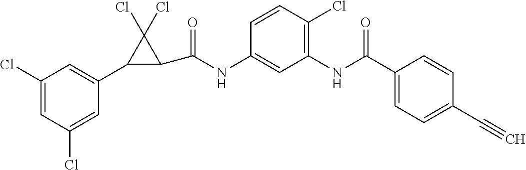 [SMILES: C#Cc1ccc(C(=O)Nc2cc(NC(=O)C3C(c4cc(Cl)cc(Cl)c4)C3(Cl)Cl)ccc2Cl)cc1] | 6 |
| F92 | 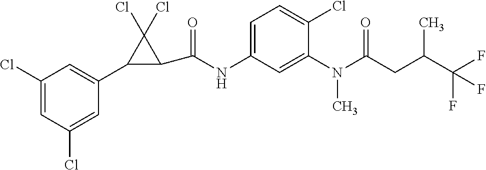 [SMILES: CC(CC(=O)N(C)c1cc(NC(=O)C2C(c3cc(Cl)cc(Cl)c3)C2(Cl)Cl)ccc1Cl)C(F)(F)F] | 18 |
| F93 | 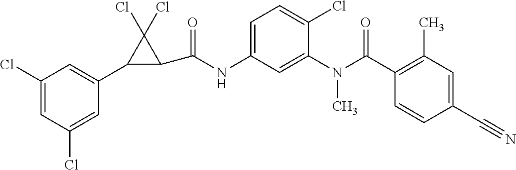 [SMILES: Cc1cc(C#N)ccc1C(=O)N(C)c1cc(NC(=O)C2C(c3cc(Cl)cc(Cl)c3)C2(Cl)Cl)ccc1Cl] | 18 |
| F94 | 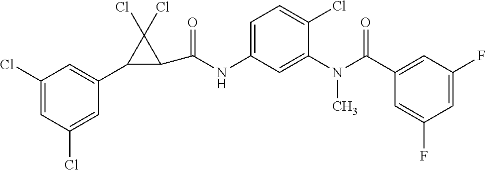 [SMILES: CN(C(=O)c1cc(F)cc(F)c1)c1cc(NC(=O)C2C(c3cc(Cl)cc(Cl)c3)C2(Cl)Cl)ccc1Cl] | 18 |
| F95 | 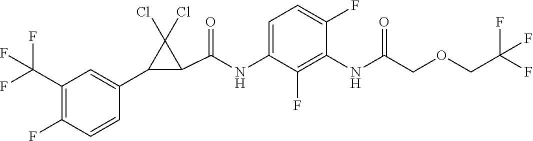 [SMILES: O=C(COCC(F)(F)F)Nc1c(F)ccc(NC(=O)C2C(c3ccc(F)c(C(F)(F)F)c3)C2(Cl)Cl)c1F] | 8 |
| F96 | 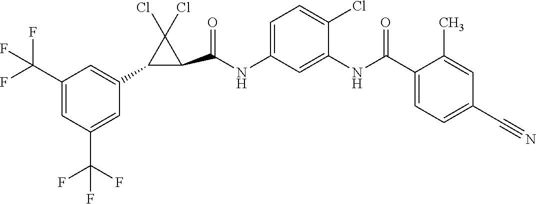 [SMILES: Cc1cc(C#N)ccc1C(=O)Nc1cc(NC(=O)[C@H]2[C@H](c3cc(C(F)(F)F)cc(C(F)(F)F)c3)C2(Cl)Cl)ccc1Cl] | 8 |
| F97 | 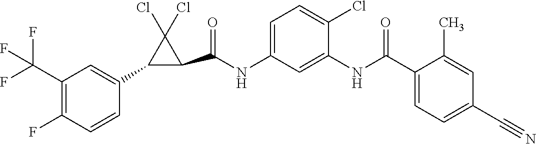 [SMILES: Cc1cc(C#N)ccc1C(=O)Nc1cc(NC(=O)[C@H]2[C@H](c3ccc(F)c(C(F)(F)F)c3)C2(Cl)Cl)ccc1Cl] | 8 |
| F98 | 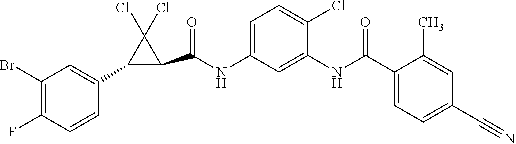 [SMILES: Cc1cc(C#N)ccc1C(=O)Nc1cc(NC(=O)[C@H]2[C@H](c3ccc(F)c(Br)c3)C2(Cl)Cl)ccc1Cl] | 8 |
| F99 | 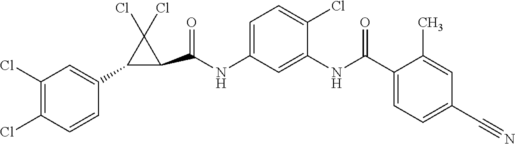 [SMILES: Cc1cc(C#N)ccc1C(=O)Nc1cc(NC(=O)[C@H]2[C@H](c3ccc(Cl)c(Cl)c3)C2(Cl)Cl)ccc1Cl] | 8 |
| F100 | 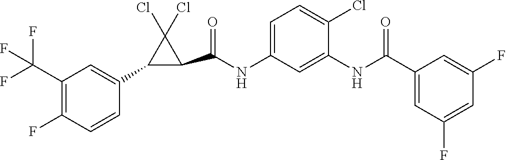 [SMILES: O=C(Nc1cc(NC(=O)[C@H]2[C@H](c3ccc(F)c(C(F)(F)F)c3)C2(Cl)Cl)ccc1Cl)c1cc(F)cc(F)c1] | 8 |
| F101 | 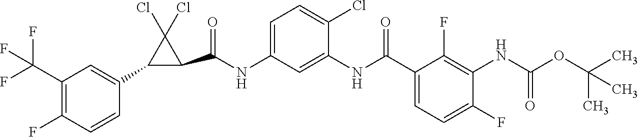 [SMILES: CC(C)(C)OC(=O)Nc1c(F)ccc(C(=O)Nc2cc(NC(=O)[C@H]3[C@H](c4ccc(F)c(C(F)(F)F)c4)C3(Cl)Cl)ccc2Cl)c1F] | 8 |
| F102 | 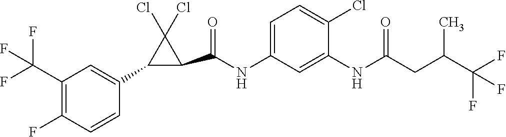 [SMILES: CC(CC(=O)Nc1cc(NC(=O)[C@H]2[C@H](c3ccc(F)c(C(F)(F)F)c3)C2(Cl)Cl)ccc1Cl)C(F)(F)F] | 8 |
| F103 | 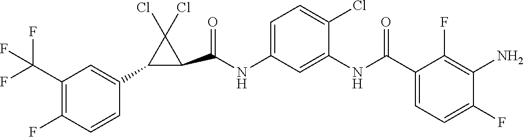 [SMILES: Nc1c(F)ccc(C(=O)Nc2cc(NC(=O)[C@H]3[C@H](c4ccc(F)c(C(F)(F)F)c4)C3(Cl)Cl)ccc2Cl)c1F] | 10 |
| F104 | 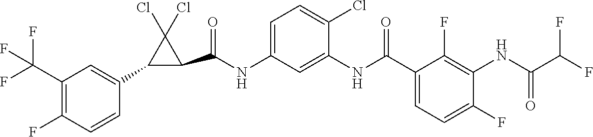 [SMILES: O=C(Nc1cc(NC(=O)[C@H]2[C@H](c3ccc(F)c(C(F)(F)F)c3)C2(Cl)Cl)ccc1Cl)c1ccc(F)c(NC(=O)C(F)F)c1F] | 11 |
| F105 | 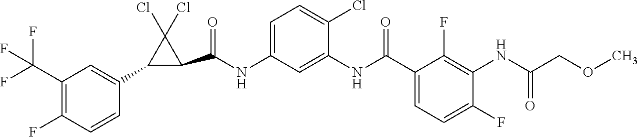 [SMILES: COCC(=O)Nc1c(F)ccc(C(=O)Nc2cc(NC(=O)[C@H]3[C@H](c4ccc(F)c(C(F)(F)F)c4)C3(Cl)Cl)ccc2Cl)c1F] | 12 |
| F106 | 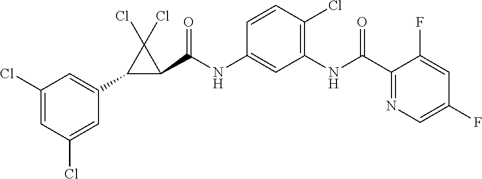 [SMILES: O=C(Nc1cc(NC(=O)[C@H]2[C@H](c3cc(Cl)cc(Cl)c3)C2(Cl)Cl)ccc1Cl)c1ncc(F)cc1F] | 8 |
| F107 | 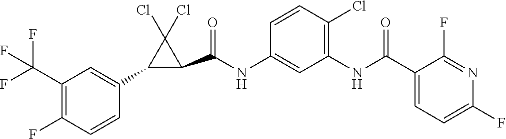 [SMILES: O=C(Nc1cc(NC(=O)[C@H]2[C@H](c3ccc(F)c(C(F)(F)F)c3)C2(Cl)Cl)ccc1Cl)c1ccc(F)nc1F] | 13 |
| F108 | 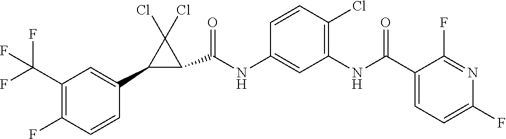 [SMILES: O=C(Nc1cc(NC(=O)[C@@H]2[C@@H](c3ccc(F)c(C(F)(F)F)c3)C2(Cl)Cl)ccc1Cl)c1ccc(F)nc1F] | 13 |
| F109 | 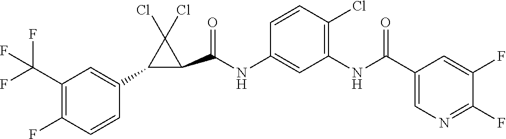 [SMILES: O=C(Nc1cc(NC(=O)[C@H]2[C@H](c3ccc(F)c(C(F)(F)F)c3)C2(Cl)Cl)ccc1Cl)c1cnc(F)c(F)c1] | 13 |
| F110 | 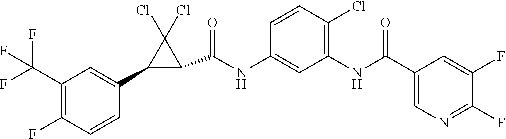 [SMILES: O=C(Nc1cc(NC(=O)[C@@H]2[C@@H](c3ccc(F)c(C(F)(F)F)c3)C2(Cl)Cl)ccc1Cl)c1cnc(F)c(F)c1] | 13 |
| F111 | 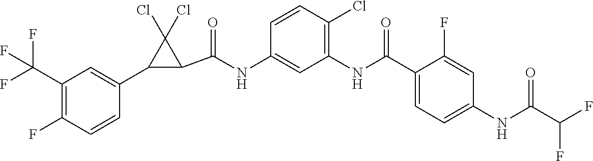 [SMILES: O=C(Nc1cc(NC(=O)C2C(c3ccc(F)c(C(F)(F)F)c3)C2(Cl)Cl)ccc1Cl)c1ccc(NC(=O)C(F)F)cc1F] | 11 |
| F112 | 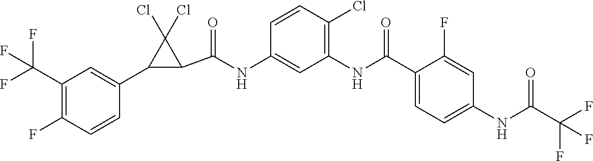 [SMILES: O=C(Nc1cc(NC(=O)C2C(c3ccc(F)c(C(F)(F)F)c3)C2(Cl)Cl)ccc1Cl)c1ccc(NC(=O)C(F)(F)F)cc1F] | 11 |
| F113 | 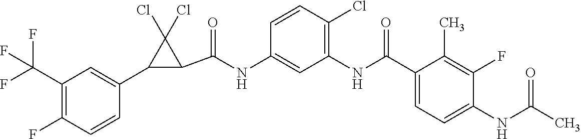 [SMILES: CC(=O)Nc1ccc(C(=O)Nc2cc(NC(=O)C3C(c4ccc(F)c(C(F)(F)F)c4)C3(Cl)Cl)ccc2Cl)c(C)c1F] | 1 |
| F114 | 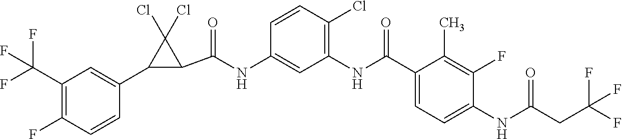 [SMILES: Cc1c(C(=O)Nc2cc(NC(=O)C3C(c4ccc(F)c(C(F)(F)F)c4)C3(Cl)Cl)ccc2Cl)ccc(NC(=O)CC(F)(F)F)c1F] | 1 |
| F115 | 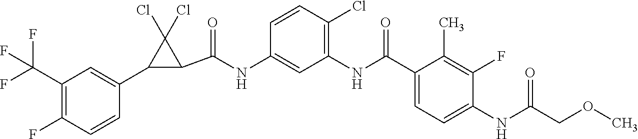 [SMILES: COCC(=O)Nc1ccc(C(=O)Nc2cc(NC(=O)C3C(c4ccc(F)c(C(F)(F)F)c4)C3(Cl)Cl)ccc2Cl)c(C)c1F] | 1 |
| F116 | 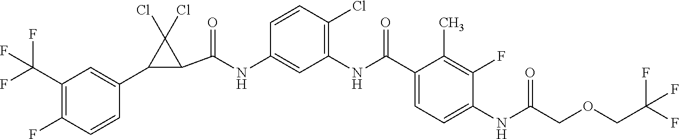 [SMILES: Cc1c(C(=O)Nc2cc(NC(=O)C3C(c4ccc(F)c(C(F)(F)F)c4)C3(Cl)Cl)ccc2Cl)ccc(NC(=O)COCC(F)(F)F)c1F] | 1 |
| F117 | 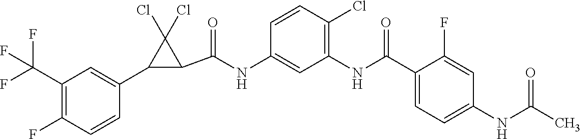 [SMILES: CC(=O)Nc1ccc(C(=O)Nc2cc(NC(=O)C3C(c4ccc(F)c(C(F)(F)F)c4)C3(Cl)Cl)ccc2Cl)c(F)c1] | 1 |
| F118 | 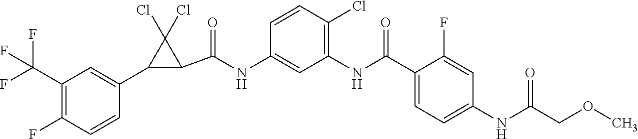 [SMILES: COCC(=O)Nc1ccc(C(=O)Nc2cc(NC(=O)C3C(c4ccc(F)c(C(F)(F)F)c4)C3(Cl)Cl)ccc2Cl)c(F)c1] | 1 |
| F119 | 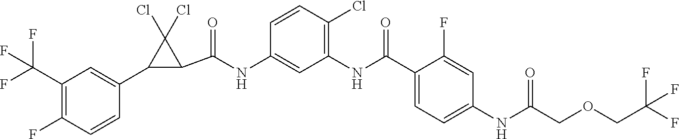 [SMILES: O=C(COCC(F)(F)F)Nc1ccc(C(=O)Nc2cc(NC(=O)C3C(c4ccc(F)c(C(F)(F)F)c4)C3(Cl)Cl)ccc2Cl)c(F)c1] | 1 |
| F120 | 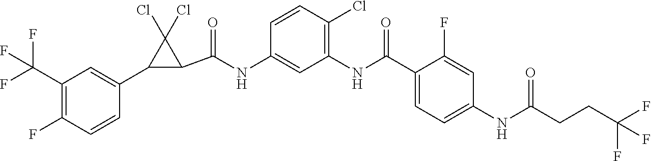 [SMILES: O=C(CCC(F)(F)F)Nc1ccc(C(=O)Nc2cc(NC(=O)C3C(c4ccc(F)c(C(F)(F)F)c4)C3(Cl)Cl)ccc2Cl)c(F)c1] | 1 |
| F121 | 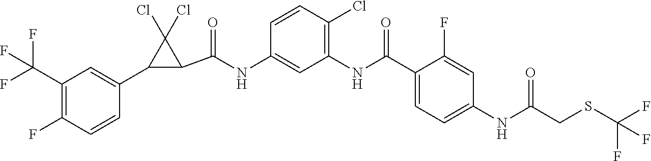 [SMILES: O=C(CSC(F)(F)F)Nc1ccc(C(=O)Nc2cc(NC(=O)C3C(c4ccc(F)c(C(F)(F)F)c4)C3(Cl)Cl)ccc2Cl)c(F)c1] | 1 |
| C2 | 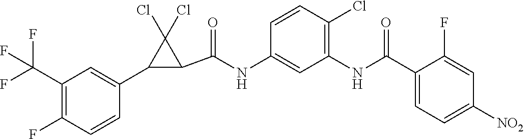 [SMILES: O=C(Nc1cc(NC(=O)C2C(c3ccc(F)c(C(F)(F)F)c3)C2(Cl)Cl)ccc1Cl)c1ccc([N+](=O)[O-])cc1F] | 1 |

*prepared according to example number

TABLE 2
Analytical Data for Molecules in Table 1

| | Melting | | | |
|---|---|---|---|---|
| Cmpd. | Point | IR | | |
| No. | (° C.) | (cm⁻¹) | MASS SPEC | NMR |
| F1 | | (thin | ESIMS m/z 468 | ¹H NMR (400 MHz, DMSO-d₆) δ 10.80 |
| | | film) | ([M + H]⁺) | (s, 1H), 9.49 (s, 1H), 8.08 (d, J = 1.9 Hz, |
| | | 3284, | | 1H), 7.62 (t, J = 1.8 Hz, 1H), 7.59-7.52 |
| | | 3073, | | (m, 3H), 7.45 (d, J = 8.8 Hz, 1H), 3.60 (d, |
| | | 1668 | | J = 8.5 Hz, 1H), 3.49 (d, J = 8.5 Hz, 1H), |
| | | | | 2.12 (s, 3H) |
| F2 | | (thin | ESIMS m/z 548 | ¹H NMR (400 MHz, DMSO-d₆) δ 10.85 |
| | | film) | ([M + H]⁺) | (s, 1H), 10.13 (s, 1H), 8.12-8.03 (m, |
| | | 3242, | | 2H), 8.00 (d, J = 2.4 Hz, 1H), 7.63 (t, J = |
| | | 3059, | | 1.8 Hz, 1H), 7.61-7.51 (m, 4H), 7.44- |
| | | 1646 | | 7.36 (m, 2H), 3.61 (d, J = 8.5 Hz, 1H), |
| | | | | 3.51 (d, J = 8.5 Hz, 1H) |
| F3 | | (thin | ESIMS m/z 494 | ¹H NMR (400 MHz, DMSO-d₆) δ 10.78 |
| | | film) | ([M + H]⁺) | (s, 1H), 9.75 (s, 1H), 8.07 (d, J = 2.5 Hz, |
| | | 3277, | | 1H), 7.62 (t, J = 1.8 Hz, 1H), 7.59-7.52 |
| | | 3071, | | (m, 3H), 7.45 (d, J = 8.8 Hz, 1H), 3.59 (d, |
| | | 1662 | | J = 8.6 Hz, 1H), 3.48 (d, J = 8.5 Hz, 1H), |
| | | | | 2.07-2.00 (m, 1H), 0.85-0.79 (m, 4H) |
| F4 | 155.3-155.6 | | ESIMS m/z 461 | ¹H NMR (400 MHz, CDCl₃) δ 8.33 (s, |
| | | | ([M − H]⁻) | 1H), 7.97 (s, 1H), 7.90 (s, 1H), 7.43 (s, |
| | | | | 1H), 7.36-7.31 (m, 3H), 7.19 (s, 2H), |
| | | | | 4.03 (s, 2H), 3.55 (d, J = 7.9 Hz, 1H), |
| | | | | 3.51 (s, 3H), 2.76 (d, J = 7.9 Hz, 1H) |
| F5 | 211-214 | | HRMS-ESI (m/z) | ¹H NMR (500 MHz, DMSO-d₆) δ 10.86 |
| | | | [M+]⁺ calcd | (s, 1H), 9.95 (s, 1H), 8.08 (d, J = 2.2 Hz, |
| | | | forC₁₉H₁₂C₁₅F₃N₂O₂, | 1H), 7.61 (dd, J = 8.7, 2.2 Hz, 2H), 7.54 |
| | | | 532.9366; found, | (d, J = 1.5 Hz, 2H), 7.49 (d, J = 8.8 Hz, |
| | | | 532.9360 | 1H), 3.66 (q, J = 11.1 Hz, 2H), 3.60 (d, J = |
| | | | | 8.5 Hz, 1H), 3.49 (d, J = 8.5 Hz, 1H); |
| | | | | 13C NMR (126 MHz, DMSO-d₆) δ |
| | | | | 162.35, 162.00, 137.84, 137.25, 134.13, |
| | | | | 133.95, 129.68, 127.80, 127.57, 125.75, |
| | | | | 123.55, 120.45, 117.30, 116.30, 62.07, |
| | | | | 38.31, 36.67; |
| | | | | ¹⁹F NMR (471 MHz, DMSO-d₆) δ −61.41, |
| | | | | −61.43, −61.46 |
| F6 | 111-116 | | HRMS-ESI (m/z) | ¹H NMR (500 MHz, DMSO-d₆) δ 10.83 |
| | | | [M + NH₄]⁺ calcd for | (s, 1H), 9.99 (s, 1H), 8.02 (t, J = 2.5 Hz, |
| | | | C₂₀H₁₈C₁₅F₃N₃O₂, | 1H), 7.61 (m, 2H), 7.54 (d, J = 1.5 Hz, |
| | | | 565.9760; found, | 2H), 7.50 (d, J = 8.8 Hz, 1H), 3.78 (m, |
| | | | 565.9763 | 1H), 3.60 (d, J = 8.5 Hz, 1H), 3.48 (d, J = |
| | | | | 8.5 Hz, 1H), 1.37 (d, J = 7.0 Hz, 3H); |
| | | | | ¹⁹F NMR (471 MHz, DMSO-d₆) δ −68.47, −68.49 |
| F7 | 113-118 | | HRMS-ESI (m/z) | ¹H NMR (500 MHz, DMSO-d₆) δ 10.80 |
| | | | [M + NH₄]⁺ calcd for | (s, 1H), 9.70 (s, 1H), 8.08 (m, 1H), 7.62 |
| | | | C₂₀H₁₈C₁₅F₃N₃O₂, | (d, J = 1.6 Hz, 1H), 7.57 (dd, J = 8.8, 2.4 |
| | | | 565.9760; found, | Hz, 1H), 7.54 (d, J = 1.5 Hz, 2H), 7.46 (d, |
| | | | 565.9769 | J = 8.8 Hz, 1H), 3.59 (d, J = 8.5 Hz, 1H), |
| | | | | 3.48 (d, J = 8.5 Hz, 1H), 2.72 (t, J = 7.3 |
| | | | | Hz, 2H), 2.60 (ddt, J = 15.5, 7.6, 4.0 Hz, 2H); |
| | | | | ¹⁹F NMR (471 MHz, DMSO-d₆) δ −64.99, |
| | | | | −65.02, −65.04 |
| F8 | 133-138 | | HRMS-ESI (m/z) | ¹H NMR (500 MHz, DMSO-d₆) δ 10.80 |
| | | | [M + H]⁺ calcd for | (s, 1H), 9.76 (s, 1H), 7.94 (s, 1H), 7.61 |
| | | | C₂₁H₁₇C₁₅F₃N₂O₂, | (m, 2H), 7.54 (d, J = 1.3 Hz, 2H), 7.47 (d, |
| | | | 560.9679; found, | J = 8.8 Hz, 1H), 3.60 (d, J = 8.5 Hz, 1H), |
| | | | 560.9678 | 3.47 (d, J = 8.5 Hz, 1H), 3.05 (m, 1H), |
| | | | | 2.70 (m, 1H), 2.37 (ddt, J = 11.7, 7.7, 4.0 |
| | | | | Hz, 1H), 1.24 (d, J = 7.0 Hz, 3H); |
| | | | | ¹³C NMR (126 MHz, DMSO-d₆) δ |
| | | | | 172.81, 162.30, 137.73, 137.27, 134.65, |
| | | | | 133.94, 129.54, 127.96, 127.79, 127.56, |
| | | | | 125.76, 121.27, 117.15, 117.03, 62.07, |
| | | | | 38.32, 36.66, 36.04, 35.83, 35.62, 35.40, |
| | | | | 34.05, 18.48; |
| | | | | ¹⁹F NMR (471 MHz, DMSO-d₆) δ −63.36, |
| | | | | −63.37, −63.39, −63.40, −63.41, −63.42 |
| F9 | 129-134 | | HRMS-ESI (m/z) | ¹H NMR (500 MHz, DMSO-d₆) δ 10.80 |
| | | | [M + H]⁺ calcd for | (s, 1H), 9.75 (s, 1H), 8.03 (d, J = 2.0 Hz, |
| | | | C₂₁H₁₇C₁₅F₃N₂O₂, | 1H), 7.62 (t, J = 1.6 Hz, 1H), 7.58 (dt, J = |
| | | | 560.9679; found, | 8.8, 2.4 Hz, 1H), 7.54 (d, J = 1.5 Hz, 2H), |
| | | | 560.9684 | 7.47 (d, J = 8.8 Hz, 1H), 3.60 (d, J = 8.5 |
| | | | | Hz, 1H), 3.48 (d, J = 8.5 Hz, 1H), 2.88 |
| | | | | (m, 1H), 2.76 (dd, J = 15.1, 4.8 Hz, 1H), |
| | | | | 2.55 (m, 1H), 1.15 (d, J = 6.9 Hz, 3H); |
| | | | | ¹³C NMR (126 MHz, DMSO-d₆) δ |
| | | | | 168.33, 162.30, 137.75, 137.25, 134.66, |
| | | | | 133.94, 129.56, 129.33, 127.79, 127.56, |
| | | | | 127.11, 120.80, 117.07, 116.63, 62.08, |
| | | | | 40.00, 38.30, 36.65, 35.57, 34.68, 34.47, |
| | | | | 34.26, 34.05, 12.56 |
| F10 | | | ESIMS m/z 599 | ¹H NMR (400 MHz, DMSO-d₆) δ 10.55 |
| | | | ([M − H]⁻) | (s, 1H), 10.24 (s, 1H), 7.85 (ddd, J = 15.7, |
| | | | | 10.7, 6.5 Hz, 2H), 7.78 (s, 2H), 7.47 (td, J = |
| | | | | 10.2, 9.4, 2.4 Hz, 1H), 7.25 (qd, J = |
| | | | | 10.0, 9.3, 5.2 Hz, 2H), 3.70 (d, J = 8.5 Hz, |
| | | | | 1H), 3.63 (d, J = 8.5 Hz, 1H); |
| | | | | ¹⁹F NMR (376 MHz, DMSO-d₆) δ |
| | | | | −105.12 (d, J = 9.7 Hz), −108.68 (d, J = |
| | | | | 10.1 Hz), −121.65, −126.11 |
| F11 | | | ESIMS m/z 557 | ¹H NMR (400 MHz, DMSO-d₆) δ 10.50 |
| | | | ([M − H]⁻) | (s, 1H), 9.62 (s, 1H), 7.81 (dt, J = 11.0, |
| | | | | 5.5 Hz, 1H), 7.78-7.74 (m, 2H), 7.17 (td, |
| | | | | J = 9.2, 1.8 Hz, 1H), 4.47 (dd, J = 8.3, 5.0 |
| | | | | Hz, 1H), 4.01 (q, J = 6.9 Hz, 1H), 3.85 |
| | | | | (dt, J = 8.0, 6.7 Hz, 1H), 3.68 (d, J = 8.6 |
| | | | | Hz, 1H), 3.61 (d, J = 8.5 Hz, 1H), 2.24 |
| | | | | (dq, J = 11.7, 7.6 Hz, 1H), 2.03-1.81 (m, 3H); |
| | | | | ¹⁹F NMR (376 MHz, DMSO-d₆) δ |
| | | | | −122.22 (dd, J = 4.9, 2.9 Hz), −126.69 (dd, |
| | | | | J = 10.1, 3.0 Hz) |
| F12 | | | ESIMS m/z 559 | ¹H NMR (400 MHz, DMSO-d₆) δ 10.50 |
| | | | ([M − H]⁻) | (s, 1H), 9.66 (s, 1H), 7.88-7.71 (m, 3H), |
| | | | | 7.17 (td, J = 9.3, 1.8 Hz, 1H), 4.07-3.96 |
| | | | | (m, 1H), 3.68 (d, J = 8.6 Hz, 1H), 3.64- |
| | | | | 3.46 (m, 3H), 1.35 (d, J = 6.7 Hz, 3H), |
| | | | | 1.21 (t, J = 7.0 Hz, 3H); |
| | | | | ¹⁹F NMR (376 MHz, DMSO-d₆) δ |
| | | | | −122.29 (t, J = 3.4 Hz), −126.57 (dd, J = |
| | | | | 8.1, 3.0 Hz) |
| F13 | | | ESIMS m/z 655 | ¹H NMR (400 MHz, DMSO-d₆) δ 11.60 |
| | | | ([M − H]⁻) | (s, 1H), 10.61 (s, 1H), 7.93 (q, J = 8.4 Hz, |
| | | | | 1H), 7.77 (d, J = 0.7 Hz, 2H), 7.38-7.22 |
| | | | | (m, 1H), 3.69 (d, J = 8.5 Hz, 1H), 3.62 (d, |
| | | | | J = 8.5 Hz, 1H); |
| | | | | ¹⁹F NMR (376 MHz, DMSO-d₆) δ −80.07 |
| | | | | (t, J = 8.2 Hz), −119.37, −122.67, −126.62, |
| | | | | −126.87 |
| F14 | | | ESIMS m/z 529 | ¹H NMR (400 MHz, DMSO-d₆) δ 10.49 |
| | | | ([M − H]⁻) | (s, 1H), 9.71 (s, 1H), 7.83-7.72 (m, 3H), |
| | | | | 7.16 (td, J = 9.3, 1.8 Hz, 1H), 3.69 (d, J = |
| | | | | 8.6 Hz, 1H), 3.61 (d, J = 8.5 Hz, 1H), |
| | | | | 2.34 (t, J = 7.1 Hz, 2H), 1.63 (h, J = 7.3 |
| | | | | Hz, 2H), 0.94 (t, J = 7.4 Hz, 3H); |
| | | | | ¹⁹F NMR (376 MHz, DMSO-d₆) δ |
| | | | | −122.02 (d, J = 3.3 Hz), −126.39 (d, J = 3.3 Hz) |
| F15 | | | ESIMS m/z 547 | ¹H NMR (400 MHz, DMSO-d₆) δ 10.48 |
| | | | ([M + H]⁺) | (s, 1H), 10.00 (s, 1H), 7.72 (t, J = 7.3 Hz, |
| | | | | 1H), 7.67-7.57 (m, 2H), 7.53 (d, J = 1.9 |
| | | | | Hz, 2H), 7.15 (td, J = 8.2, 1.4 Hz, 1H), |
| | | | | 3.72 (d, J = 8.6 Hz, 1H), 3.62 (d, J = 8.5 |
| | | | | Hz, 1H), 2.99-2.82 (m, 1H), 2.76 (dd, J = |
| | | | | 15.2, 5.0 Hz, 1H), 2.59-2.52 (m, 1H), |
| | | | | 1.15 (d, J = 6.9 Hz, 3H); |
| | | | | ¹⁹F NMR (376 MHz, DMSO-d₆) δ −72.08, |
| | | | | −134.71 (d, J = 7.4 Hz) |
| F16 | | | ESIMS m/z 549 | ¹H NMR (400 MHz, DMSO-d₆) δ 10.54 |
| | | | ([M + H]⁺) | (s, 1H), 10.26 (s, 1H), 7.86-7.79 (m, J |
| | | | | 2H), 7.64 (t, J = 1.9 Hz, 1H), 7.53 (dd, J = |
| | | | | 1.9, 0.6 Hz, 3H), 7.46 (ddd, J = 10.8, 9.5, |
| | | | | 2.5 Hz, 1H), 7.30-7.17 (m, 2H), 3.73 (d, |
| | | | | J = 8.6 Hz, 1H), 3.63 (d, J = 8.5 Hz, 1H); |
| | | | | ¹⁹F NMR (376 MHz, DMSO-d₆) δ |
| | | | | −105.65 (d, J = 9.5 Hz), −109.04 (d, J = 9.8 |
| | | | | Hz), −133.24 |
| F17 | | | ESIMS m/z 507 | ¹H NMR (400 MHz, DMSO-d₆) δ 10.48 |
| | | | ([M + H]⁺) | (s, 1H), 9.49 (s, 1H), 7.81-7.71 (m, 1H), |
| | | | | 7.63 (t, J = 1.9 Hz, 1H), 7.58-7.45 (m, |
| | | | | 3H), 7.15 (td, J = 8.2, 1.4 Hz, 1H), 4.48 |
| | | | | (dd, J = 8.3, 5.4 Hz, 1H), 4.06-3.95 (m, |
| | | | | 1H), 3.85 (dt, J = 8.3, 6.7 Hz, 1H), 3.71 |
| | | | | (d, J = 8.6 Hz, 1H), 3.61 (d, J = 8.6 Hz, |
| | | | | 1H), 2.23 (dq, J = 12.1, 7.7 Hz, 1H), 2.00 |
| | | | | (q, J = 6.5, 5.9 Hz, 1H), 1.89 (p, J = 6.9 |
| | | | | Hz, 2H); |
| | | | | ¹⁹F NMR (376 MHz, DMSO-d₆) δ |
| | | | | −134.96 (d, J = 24.8 Hz) |
| F18 | | | ESIMS m/z 509 | ¹H NMR (400 MHz, DMSO-d₆) δ 10.49 |
| | | | ([M + H]⁺) | (s, 1H), 9.54 (s, 1H), 7.76 (t, J = 7.6 Hz, |
| | | | | 1H), 7.63 (t, J = 1.9 Hz, 1H), 7.57-7.50 |
| | | | | (m, 3H), 7.16 (t, J = 8.2 Hz, 1H), 4.05 (q, |
| | | | | J = 6.7 Hz, 1H), 3.71 (d, J = 8.6 Hz, 1H), |
| | | | | 3.61 (d, J = 8.6 Hz, 1H), 3.54 (qt, J = 7.3, |
| | | | | 3.7 Hz, 2H), 1.34 (d, J = 6.7 Hz, 3H), |
| | | | | 1.20 (t, J = 7.0 Hz, 3H); |
| | | | | ¹⁹F NMR (376 MHz, DMSO-d₆) δ −135.24 |
| F19 | | | ESIMS m/z 605 | ¹H NMR (400 MHz, DMSO-d₆) δ 11.46 |
| | | | ([M + H]⁺) | (s, 1H), 10.60 (s, 1H), 7.99 (t, J = 7.3 Hz, |
| | | | | 1H), 7.63 (t, J = 1.9 Hz, 1H), 7.52 (d, J = |
| | | | | 1.9 Hz, 2H), 7.26 (t, J = 8.1 Hz, 1H), 7.22- |
| | | | | 7.15 (m, 1H), 3.71 (d, J = 8.6 Hz, 1H), |
| | | | | 3.62 (d, J = 8.5 Hz, 1H); |
| | | | | ¹⁹F NMR (376 MHz, DMSO-d₆) δ −80.03 |
| | | | | (t, J = 8.1 Hz), −119.36, −126.75, −130.97 |
| F20 | | | ESIMS m/z 479 | ¹H NMR (400 MHz, DMSO-d₆) δ 10.44 |
| | | | ([M + H]⁺) | (s, 1H), 9.72 (s, 1H), 7.68 (t, J = 7.5 Hz, |
| | | | | 1H), 7.65-7.55 (m, 2H), 7.52 (d, J = 1.9 |
| | | | | Hz, 2H), 7.16-7.07 (m, 1H), 3.71 (d, J = |
| | | | | 8.6 Hz, 1H), 3.61 (d, J = 8.6 Hz, 1H), |
| | | | | 2.37 (t, J = 7.3 Hz, 2H), 1.62 (h, J = 7.3 |
| | | | | Hz, 2H), 0.93 (t, J = 7.4 Hz, 3H); |
| | | | | ¹⁹F NMR (376 MHz, DMSO-d₆) δ −134.85 |
| F21 | | | ESIMS m/z 491 | ¹H NMR (400 MHz, DMSO-d₆) δ 10.44 |
| | | | ([M + H]⁺) | (s, 1H), 9.65 (s, 1H), 7.71-7.60 (m, 3H), |
| | | | | 7.52 (d, J = 1.9 Hz, 2H), 7.17-7.08 (m, |
| | | | | 1H), 3.71 (d, J = 8.6 Hz, 1H), 3.61 (d, J = |
| | | | | 8.6 Hz, 1H), 2.30 (d, J = 7.1 Hz, 2H), |
| | | | | 1.12-1.01 (m, 1H), 0.54-0.45 (m, 2H), |
| | | | | 0.26-0.17 (m, 2H); |
| | | | | ¹⁹F NMR (376 MHz, DMSO-d₆) δ −135.09 |
| F22 | | | ESIMS m/z 490 | ¹H NMR (400 MHz, DMSO-d₆) δ 10.50 |
| | | | ([M + H]⁺) | (s, 1H), 10.30 (s, 1H), 7.74 (t, J = 7.3 Hz, |
| | | | | 1H), 7.66-7.55 (m, 2H), 7.53 (d, J = 1.9 |
| | | | | Hz, 2H), 7.22-7.14 (m, 1H), 4.12 (q, J = |
| | | | | 7.2 Hz, 1H), 3.71 (d, J = 8.6 Hz, 1H), |
| | | | | 3.62 (d, J = 8.5 Hz, 1H), 1.53 (d, J = 7.2 |
| | | | | Hz, 3H); |
| | | | | ¹⁹F NMR (376 MHz, DMSO-d₆) δ |
| | | | | −134.43 (d, J = 18.5 Hz) |
| F23 | | | ESIMS m/z 477 | ¹H NMR (400 MHz, DMSO-d₆) δ 10.45 |
| | | | ([M + H]⁺) | (s, 1H), 9.82 (s, 1H), 7.68 (t, J = 7.6 Hz, |
| | | | | 1H), 7.65-7.57 (m, 2H), 7.53 (d, J = 1.9 |
| | | | | Hz, 2H), 7.17-7.08 (m, 1H), 5.97 (ddt, J = |
| | | | | 17.1, 10.2, 6.8 Hz, 1H), 5.20 (dq, J = |
| | | | | 17.2, 1.7 Hz, 1H), 5.14 (dt, J = 10.1, 1.6 |
| | | | | Hz, 1H), 3.71 (d, J = 8.6 Hz, 1H), 3.61 (d, |
| | | | | J = 8.6 Hz, 1H), 3.25-3.18 (m, 2H); |
| | | | | ¹⁹F NMR (376 MHz, DMSO-d₆) δ −135.00 |
| F24 | 198-202 | | ESIMS m/z 563 | ¹H NMR (400 MHz, DMSO-d₆) δ 10.95 |
| | | | ([M − H]⁻) | (s, 1H), 9.94 (d, J = 3.2 Hz, 1H), 8.21 (d, |
| | | | | J = 2.4 Hz, 1H), 7.90-7.84 (m, 1H), 7.63- |
| | | | | 7.59 (m, 2H), 7.54-7.51 (m, 3H), 7.48- |
| | | | | 7.41 (m, 1H), 7.28-7.23 (m, 1H), 3.62 |
| | | | | (d, J = 8.8 Hz, 1H), 3.51 (d, J = 8.4 Hz, 1H) |
| F25 | 232-236 | | ESIMS m/z 563 | ¹H NMR (400 MHz, CDCl₃) δ 8.50 (s, |
| | | | ([M − H]⁻) | 1H), 8.27 (s, 1H), 8.08 (s, 1H), 7.86 (d, J = |
| | | | | 6.8 Hz, 1H), 7.52-7.45 (m, 1H), 7.40 |
| | | | | (d, J = 8.2 Hz , 1H), 7.39-7.34 (m, 1H), |
| | | | | 7.19 (s, 2H), 7.05 (t, J = 8.4 Hz, 2H), 3.54 |
| | | | | (d, J = 7.6 Hz, 1H), 2.79 (d, J = 7.6 Hz, 1H); |
| | | | | ¹⁹F NMR (376 MHz, CDCl₃) δ −111.01 |
| F26 | 123-126 | | ESIMS m/z 563 | ¹H NMR (400 MHz, DMSO-d₆) δ 10.82 |
| | | | ([M − H]⁻) | (s, 1H), 10.30 (s, 1H), 7.97 (d, J = 2.0 Hz, |
| | | | | 1H), 7.70-7.68 (m, 2H), 7.62-7.53 (m, |
| | | | | 6H), 3.60 (d, J = 8.8 Hz, 1H), 3.49 (d, J = |
| | | | | 8.4 Hz, 1H); |
| | | | | ¹⁹F NMR (376 MHz, DMSO-d₆) δ −108.61 |
| F27 | 154-158 | | ESIMS m/z 566 | ¹H NMR (400 MHz, DMSO-d₆) δ 10.95 |
| | | | ([M − H]⁻) | (s, 1H), 10.25 (s, 1H), 8.02 (s, 1H), 7.84- |
| | | | | 7.79 (m, 2H), 7.69-7.61 (m, 3H), 7.58- |
| | | | | 7.51 (m, 3H), 3.62 (d, J = 8.0 Hz, 1H), |
| | | | | 3.50 (d, J = 8.4 Hz, 1H), 2.48 (s, 3H) |
| F28 | 196-200 | | ESIMS m/z 530 | ¹H NMR (400 MHz, DMSO-d₆) δ 10.91 |
| | | | ([M + H]⁺) | (s, 1H), 10.65 (s, 1H), 8.78-8.77 (m, |
| | | | | 2H), 8.23 (d, J = 8.0 Hz, 1H), 8.15-8.11 |
| | | | | (m, 1H), 7.76-7.72 (m, 1H), 7.65-7.62 |
| | | | | (m, 2H), 7.56-7.54 (m, 3H), 3.62 (d, J = |
| | | | | 8.8 Hz, 1H), 3.51 (d, J = 8.4 Hz, 1H) |
| F29 | 154-159 | | ESIMS m/z 529 | ¹H NMR (400 MHz, DMSO-d₆) δ 10.96 |
| | | | ([M − H]⁻) | (s, 1H), 10.62 (s, 1H), 9.09 (d, J = 5.2 Hz, |
| | | | | 2H), 8.66 (d, J = 2.0 Hz, 1H), 7.81-7.78 |
| | | | | (m, 1H), 7.71-7.68 (m, 1H), 7.62 (s, |
| | | | | 1H), 7.57-7.54 (m, 3H), 3.62 (d, J = 8.4 |
| | | | | Hz, 1H), 3.51 (d, J = 8.8 Hz, 1H) |
| F30 | 120-124 | | ESIMS m/z 546 | ¹H NMR (400 MHz, DMSO-d₆) δ 10.90 |
| | | | ([M − H]⁻) | (s, 1H), 10.42 (s, 1H), 8.78 (d, J = 2.4 Hz, |
| | | | | 1H), 8.73 (d, J = 2.4 Hz, 1H), 8.31-8.28 |
| | | | | (m, 1H), 8.06-8.00 (m, 1H), 7.64-7.61 |
| | | | | (m, 2H), 7.57-7.54 (m, 3H), 3.62 (d, J = |
| | | | | 8.8 Hz, 1H), 3.50 (d, J = 8.4 Hz, 1H); |
| | | | | ¹⁹F NMR (376 MHz, DMSO-d₆) δ −121.20 |
| F31 | 122-126 | | ESIMS m/z 564 | ¹H NMR (400 MHz, DMSO-d₆) δ 10.86 |
| | | | ([M − H]⁻) | (s, 1H), 10.30 (s, 1H), 8.70 (d, J = 2.4 Hz, |
| | | | | 1H), 8.58 (d, J = 1.2 Hz, 1H), 8.23-8.17 |
| | | | | (m, 1H), 7.64-7.61 (m, 2H), 7.55-7.53 |
| | | | | (m, 3H), 3.61 (d, J = 8.8 Hz, 1H), 3.50 (d, |
| | | | | J = 8.4 Hz, 1H); |
| | | | | ¹⁹F NMR (376 MHz, DMSO-d₆) δ −114.78, −116.94 |
| F32 | 215-219 | | ESIMS m/z 531 | ¹H NMR (400 MHz, DMSO-d₆) δ 10.90 |
| | | | ([M + H]⁺) | (s, 1H), 10.57 (s, 1H), 9.46 (s, 1H), 9.19 |
| | | | | (d, J = 4.8 Hz, 1H), 8.68 (s, 1H), 8.20 (d, |
| | | | | J = 4.4 Hz, 1H), 7.65-7.54 (m, 5H), 3.62 |
| | | | | (d, J = 8.0 Hz, 1H), 3.51 (d, J = 8.4 Hz, 1H) |
| F33 | 312-316 | | ESIMS m/z 544 | ¹H NMR (400 MHz, DMSO-d₆) δ 11.39 |
| | | | ([M − H]⁻) | (s, 1H), 10.91 (s, 1H), 10.45 (s, 1H), 8.80 |
| | | | | (br s, 1H), 7.92-7.87 (m, 1H), 7.69- |
| | | | | 7.61 (m, 3H), 7.58-7.53 (m, 3H), 6.99 |
| | | | | (d, J = 8.4 Hz, 1H), 3.61 (d, J = 8.0 Hz, |
| | | | | 1H), 3.50 (d, J = 8.8 Hz, 1H) |
| F34 | 157-161 | | ESIMS m/z 517 | ¹H NMR (400 MHz, DMSO-d₆) δ 10.95 |
| | | | ([M − H]⁻) | (s, 1H), 9.85 (s, 1H), 8.06 (s, 1H), 7.96 (s, |
| | | | | 1H), 7.61-7.51 (m, 5H), 7.35 (s, 1H), |
| | | | | 6.72 (s, 1H), 3.59 (d, J = 8.4 Hz, 1H), |
| | | | | 3.50 (d, J = 8.4 Hz, 1H) |
| F35 | 189-192 | | ESIMS m/z 518 | ¹H NMR (400 MHz, DMSO-d₆) δ 10.83 |
| | | | ([M − H]⁻) | (s, 1H), 10.58 (s, 1H), 8.83 (d, J = 2.0 Hz, |
| | | | | 1H), 7.97 (d, J = 2.0 Hz, 1H), 7.61-7.59 |
| | | | | (m, 2H), 7.57-7.55 (m, 3H), 7.28 (d, J = |
| | | | | 2.0 Hz, 1H), 3.61 (d, J = 8.8 Hz, 1H), |
| | | | | 3.49 (d, J = 8.4 Hz, 1H) |
| F36 | 228-232 | | ESIMS m/z 602 | ¹H NMR (400 MHz, DMSO-d₆) δ 10.90 |
| | | | ([M − H]⁻) | (s, 1H), 10.42 (s, 1H), 8.90 (s, 1H), 8.21 |
| | | | | (d, J = 2.0 Hz, 1H), 7.63-7.60 (m, 2H), |
| | | | | 7.57-7.54 (m, 3H), 3.61 (d, J = 8.4 Hz, |
| | | | | 1H), 3.50 (d, J = 8.4 Hz, 1H); |
| | | | | ¹⁹F NMR (376 MHz, DMSO-d₆) δ −62.11 |
| F37 | 159-163 | | ESIMS m/z 534 | ¹H NMR (400 MHz, DMSO-d₆) δ 11.90 |
| | | | ([M − H]⁻) | (br s, 1H), 10.92 (s, 1H), 10.41 (s, 1H), |
| | | | | 7.94 (d, J = 2.0 Hz, 1H), 7.61-7.58 (m, |
| | | | | 2H), 7.56-7.54 (m, 3H), 6.78 (s, 1H), |
| | | | | 3.60 (d, J = 8.0 Hz, 1H), 3.49 (d, J = 8.8 |
| | | | | Hz, 1H) |
| F38 | 120-124 | | ESIMS m/z 521 | ¹H NMR (300 MHz, DMSO-d₆) δ 10.92 |
| | | | ([M − H]⁻) | (s, 1H), 9.30 (s, 1H), 8.35 (dd, J = 2.1, 6.0 |
| | | | | Hz, 1H), 7.63-7.47 (m, 5H), 4.52-4.47 |
| | | | | (m, 1H), 4.05-3.98 (m, 1H), 3.93-3.85 |
| | | | | (m, 1H), 3.61 (d, J = 8.7 Hz, 1H), 3.49 (d, |
| | | | | J = 8.4 Hz, 1H), 2.29-2.20 (m, 1H), 2.05- |
| | | | | 1.98 (m, 1H), 1.93-1.85 (m, 2H) |
| F39 | 123-127 | | ESIMS m/z 495 | ¹H NMR (400 MHz, DMSO-d₆) δ 10.81 |
| | | | ([M − H]⁻) | (s, 1H), 9.21 (s, 1H), 8.33 (d, J = 2.0 Hz, |
| | | | | 1H), 7.61-7.59 (m, 2H), 7.53-7.47 (m, |
| | | | | 3H), 4.08 (s, 2H), 3.60 (d, J = 8.4 Hz, |
| | | | | 1H), 3.48 (d, J = 8.8 Hz, 1H), 3.45 (s, 3H) |
| F40 | 91-95 | | ESIMS m/z 523 | ¹H NMR (400 MHz, DMSO-d₆) δ 10.81 |
| | | | ([M − H]⁻) | (s, 1H), 9.32 (s, 1H), 8.38-8.36 (m, 1H), |
| | | | | 7.61-7.57 (m, 2H), 7.53-7.48 (m, 3H), |
| | | | | 4.09-4.02 (m, 1H), 3.64-3.58 (m, 3H), |
| | | | | 3.49-3.29 (m, 1H), 1.36 (d, J = 6.8 Hz, |
| | | | | 3H), 1.23 (t, J = 6.8 Hz, 3H) |
| F41 | 104-108 | | ESIMS m/z 571 | ¹H NMR (400 MHz, DMSO-d₆) δ 10.81 |
| | | | ([M − H]⁻) | (s, 1H), 9.62 (s, 1H), 8.08 (s, 1H), 7.62- |
| | | | | 7.59 (m, 2H), 7.52 (d, J = 1.6 Hz, 2H), |
| | | | | 7.47 (d, J = 8.8 Hz, 1H), 7.32 (t, J = 8.0 |
| | | | | Hz, 2H), 7.04-6.97 (m, 3H), 5.05 (q, J = |
| | | | | 6.8 Hz, 1H), 3.59 (d, J = 8.4 Hz, 1H), |
| | | | | 3.46 (d, J = 8.0 Hz, 1H), 1.58 (d, J = 6.8 |
| | | | | Hz, 3H) |
| F42 | 91-95 | | ESIMS m/z 490 | ¹H NMR (400 MHz, DMSO-d₆) δ 10.80 |
| | | | ([M − H]⁻) | (s, 1H), 9.92 (s, 1H), 8.10 (d, J = 2.5 Hz, |
| | | | | 1H), 7.64-7.55 (m, 2H), 7.53 (d, J = 1.9 |
| | | | | Hz, 2H), 7.48 (d, J = 8.8 Hz, 1H), 4.02 (s, |
| | | | | 2H), 3.59 (d, J = 8.6 Hz, 1H), 3.48 (d, J = |
| | | | | 8.5 Hz, 1H) |
| F43 | 133-137 | | ESIMS m/z 516 | ¹H NMR (400 MHz, DMSO-d₆) δ 8.77 (s, |
| | | | ([M − H]⁻) | 1H), 8.46 (d, J = 2.4 Hz, 1H), 7.74 (s, |
| | | | | 1H), 7.58 (dd, J = 2.0, 8.8 Hz, 1H), 7.40 |
| | | | | (d, J = 8.8 Hz, 1H), 7.34 (s, 1H), 7.18 (s, |
| | | | | 2H), 3.55 (d, J = 8.4 Hz, 1H), 2.74 (d, J = |
| | | | | 7.6 Hz, 1H), 1.84-1.80 (m, 2H), 1.68- |
| | | | | 1.54 (m, 2H) |
| F44 | 141-145 | | ESIMS m/z 529 | ¹H NMR (400 MHz, DMSO-d₆) δ 10.80 |
| | | | ([M + H]⁺) | (s, 1H), 10.00 (s, 1H), 8.04 (br s, 1H), |
| | | | | 7.62-7.54 (m, 2H), 7.53-7.48 (m, 2H), |
| | | | | 7.45 (d, J = 8.8 Hz, 1H), 3.56 (d, J = 8.8 |
| | | | | Hz, 1H), 3.43 (d, J = 8.8 Hz, 1H), 3.09- |
| | | | | 2.94 (m, 1H), 2.04-1.93 (m, 2H); |
| | | | | ¹⁹F NMR (376 MHz, DMSO-d₆) δ −124.54, −140.28 |
| F45 | 211-215 | | ESIMS m/z 493 | ¹H NMR (400 MHz, DMSO-d₆) δ 8.37 (d, |
| | | | ([M − H]⁻) | J = 2.0 Hz, 1H), 7.98 (s, 1H), 7.72 (dd, J = |
| | | | | 2.0, 8.8 Hz, 1H), 7.65 (s, 1H), 7.35- |
| | | | | 7.33 (m, 2H), 7.18 (s, 2H), 3.54 (d, J = |
| | | | | 8.0 Hz, 1H), 2.76 (d, J = 7.6 Hz, 1H), |
| | | | | 2.44 (t, J = 7.6 Hz, 2H), 1.82-1.76 (m, |
| | | | | 2H), 1.04 (t, J = 7.2 Hz, 3H) |
| F46 | 184-188 | | ESIMS m/z 507 | ¹H NMR (400 MHz, DMSO-d₆) δ 8.36 (d, |
| | | | ([M + H]⁺) | J = 2.0 Hz, 1H), 8.06 (s, 1H), 7.72 (d, J = |
| | | | | 2.0, 8.8 Hz, 1H), 7.65 (s, 1H), 7.35-7.33 |
| | | | | (m, 2H), 7.18 (s, 2H), 3.53 (d, J = 8.0 Hz, |
| | | | | 1H), 2.76 (d, J = 8.4 Hz, 1H), 2.46 (t, J = |
| | | | | 7.2 Hz, 2H), 1.78-1.70 (m, 2H), 1.47- |
| | | | | 1.41 (m, 2H), 0.97 (t, J = 7.2 Hz, 3H) |
| F47 | 195-199 | | ESIMS m/z 493 | ¹H NMR (400 MHz, DMSO-d₆) δ 10.76 |
| | | | ([M + H]⁺) | (s, 1H), 9.46 (s, 1H), 8.06 (d, J = 2.4 Hz, |
| | | | | 1H), 7.61 (s, 1H), 7.58-7.52 (m, 3H), |
| | | | | 7.45 (d, J = 8.8 Hz, 1H), 6.01-5.94 (m, |
| | | | | 1H), 5.25-5.15 (m, 2H), 3.59 (d, J = 8.8 |
| | | | | Hz, 1H), 3.47 (d, J = 8.4 Hz, 1H), 3.21 (d, |
| | | | | J = 6.8 Hz, 2H) |
| F48 | 108-120 | | ESIMS m/z 543 | ¹H NMR (400 MHz, DMSO-d₆) δ 10.79 |
| | | | ([M + H]⁺) | (s, 1H), 9.62 (s, 1H), 8.03 (d, J = 2.4 Hz, |
| | | | | 1H), 7.61-7.56 (m, 2H), 7.52 (s, 2H), |
| | | | | 7.45 (d, J = 8.8 Hz, 1H), 7.40-7.32 (m, |
| | | | | 4H), 7.29-7.22 (m, 1H), 3.75 (s, 2H), |
| | | | | 3.58 (d, J = 8.4 Hz, 1H), 3.45 (d, J = 8.4 |
| | | | | Hz, 1H) |
| F49 | 103-107 | | ESIMS m/z 595 | ¹H NMR (400 MHz, DMSO-d₆) δ 10.79 |
| | | | ([M − H]⁻) | (s, 1H), 9.82 (s, 1H), 8.04 (d, J = 2.4 Hz, |
| | | | | 1H), 7.61-7.60 (m, 1H), 7.57 (dd, J = |
| | | | | 2.4, 8.8 Hz, 1H), 7.52 (d, J = 1.2 Hz, 2H), |
| | | | | 7.47 (d, J = 8.8 Hz, 1H), 7.22-7.16 (m, |
| | | | | 2H), 3.84 (s, 2H), 3.58 (d, J = 8.4 Hz, |
| | | | | 1H), 3.45 (d, J = 8.4 Hz, 1H); |
| | | | | ¹⁹F NMR (376 MHz, DMSO-d₆) δ −109.89, −111.52 |
| F50 | 188-192 | | ESIMS m/z 552 | ¹H NMR (400 MHz, DMSO-d₆) δ 10.02 |
| | | | ([M + H]⁺) | (s, 1H), 8.40 (d, J = 2.0 Hz, 1H), 8.11 (br |
| | | | | s, 1H), 7.80 (dd, J = 2.8, 8.8 Hz, 1H), 7.38- |
| | | | | 7.34 (m, 2H), 7.19 (s, 2H), 3.81 (t, J = |
| | | | | 4.4 Hz, 4H), 3.55 (d, J = 7.6 Hz, 1H), |
| | | | | 3.22(s, 2H), 2.80 (d, J = 7.6 Hz, 1H), 2.68 |
| | | | | (t, J = 4.0 Hz, 4H) |
| F51 | 153-157 | | ESIMS m/z 522 | ¹H NMR (400 MHz, DMSO-d₆) δ 10.78 |
| | | | ([M − H]⁻) | (s, 1H), 9.41 (s, 1H), 8.33-8.30 (m, 1H), |
| | | | | 8.18 (d, J = 2.0 Hz, 1H), 7.61 (s, 1H), |
| | | | | 7.57-7.52 (m, 3H), 7.46 (d, J = 8.8 Hz, |
| | | | | 1H), 3.93 (d, J = 5.6 Hz, 2H) 3.59 (d, J = |
| | | | | 8.8 Hz, 1H), 3.47 (d, J = 8.4 Hz, 1H), |
| | | | | 1.91 (s, 3H) |
| F52 | 96-100 | | ESIMS m/z 604 | ¹H NMR (400 MHz, DMSO-d₆) δ 10.83 |
| | | | ([M − H]⁺) | (s, 1H), 9.82 (s, 1H), 8.59-8.56 (m, 1H), |
| | | | | 7.61-7.57 (m, 2H), 7.53 (s, 2H), 7.47 (d, |
| | | | | J = 8.8 Hz, 1H), 3.72-3.58 (m, 3H), 3.50- |
| | | | | 3.45 (m, 2H), 3.44-3.40 (m, 1H), 2.79- |
| | | | | 2.72 (m, 1H), 2.23-2.18 (m, 1H), 1.92- |
| | | | | 1.86 (m, 2H), 1.78-1.70 (m, 1H); |
| | | | | ¹⁹F NMR (376 MHz, DMSO-d₆) δ −69.20 |
| F53 | 150-152 | | ESIMS m/z 519 | ¹H NMR (400 MHz, DMSO-d₆) δ 11.29 |
| | | | ([M − H]⁻) | (s, 1H), 10.85 (s, 1H), 7.87 (s, 1H), 7.62- |
| | | | | 7.57 (m, 3H), 7.54 (s, 2H), 3.60 (d, J = |
| | | | | 8.4 Hz, 1H), 3.49 (d, J = 8.4 Hz, 1H); |
| | | | | ¹⁹F NMR (376 MHz, DMSO-d₆) δ −74.10 |
| F54 | 147-149 | | ESIMS m/z 535 | ¹H NMR (400 MHz, DMSO-d₆) δ 11.15 |
| | | | ([M − H]⁻) | (s, 1H), 10.85 (s, 1H), 7.84 (d, J = 2.0 Hz, |
| | | | | 1H), 7.62-7.56 (m, 3H), 7.54 (d, J = 2.0 |
| | | | | Hz, 2H), 3.61 (d, J = 8.0 Hz, 1H), 3.49 (d, |
| | | | | J = 8.8 Hz, 1H); |
| | | | | ¹⁹F NMR (376 MHz, DMSO-d₆) δ −63.02 |
| F55 | 89-93 | | ESIMS m/z 551 | ¹H NMR (400 MHz, DMSO-d₆) δ 11.10 |
| | | | ([M − H]⁻) | (s, 1H), 10.86 (s, 1H), 7.82 (s, 1H), 7.62- |
| | | | | 7.54 (m, 5H), 6.98-6.72 (m , 1H), 3.60 |
| | | | | (d, J = 8.4 Hz, 1H), 3.48 (d, J = 8.8 Hz, 1H); |
| | | | | ¹⁹F NMR (376 MHz, DMSO-d₆) δ −124.00, −138.95 |
| F56 | 81-84 | | ESIMS m/z 619 | ¹H NMR (400 MHz, DMSO-d₆) δ 11.40 |
| | | | ([M − H]⁻) | (s, 1H), 10.90 (s, 1H), 7.81 (d, J = 2.4 Hz, |
| | | | | 1H), 7.65-7.58 (m, 3H), 7.54 (d, J = 1.6 |
| | | | | Hz, 2H), 3.61 (d, J = 8.4 Hz, 1H), 3.49 (d, |
| | | | | J = 8.8 Hz, 1H); |
| | | | | ¹⁹F NMR (376 MHz, DMSO-d₆) δ −80.01, −119.27 |
| F57 | 92-96 | | ESIMS m/z 577 | ¹H NMR (400 MHz, DMSO-d₆) δ 10.80 |
| | | | ([M − H]⁻) | (s, 1H), 10.76 (s, 1H), 7.81-7.68 (m, |
| | | | | 3H), 7.68-7.58 (m, 5H), 7.54-7.46 (m, |
| | | | | 3H), 3.59 (d, J = 8.0 Hz, 1H), 3.46 (d, J = |
| | | | | 8.8 Hz, 1H); |
| | | | | ¹⁹F NMR (376 MHz, DMSO-d₆) δ −101.50 |
| F58 | 127-130 | | ESIMS m/z 529 | ¹H NMR (400 MHz, DMSO-d₆) δ 10.82 |
| | | | ([M − H]⁻) | (s, 1H), 10.40 (s, 1H), 7.83 (d, J = 1.6 Hz, |
| | | | | 1H), 7.61-7.58 (m, 2H), 7.55-7.52 (m, |
| | | | | 3H), 3.61 (d, J = 8.8 Hz, 1H), 3.48 (d, J = |
| | | | | 8.0 Hz, 1H), 2.23-2.13 (m, 2H), 1.03 (t, |
| | | | | J = 7.6 Hz, 3H); |
| | | | | ¹⁹F NMR (376 MHz, DMSO-d₆) δ −106.21 |
| F60 | | | ESIMS m/z 550 | ¹H NMR (400 MHz, DMSO-d₆) δ 10.76 |
| | | | ([M − H]⁻) | (s, 1H), 10.39 (s, 1H), 8.19-8.10 (m, |
| | | | | 1H), 7.84 (s, 1H), 7.79 (d, J = 8.1 Hz, |
| | | | | 1H), 7.66 (d, J = 7.9 Hz, 1H), 7.63 (t, J = |
| | | | | 1.9 Hz, 1H), 7.61-7.51 (m, 3H), 7.29 (t, |
| | | | | J = 9.6 Hz, 1H), 3.61 (d, J = 8.5 Hz, 1H), |
| | | | | 3.49 (d, J = 8.5 Hz, 1H), 2.44 (s, 3H); |
| | | | | ¹⁹F NMR (376 MHz, DMSO-d₆) δ −127.43 |
| F61 | | | ESIMS m/z 547 | ¹H NMR (400 MHz, DMSO-d₆) δ 10.78 |
| | | | ([M − H]⁻) | (s, 1H), 10.72 (s, 1H), 8.23 (dd, J = 7.0, |
| | | | | 2.7 Hz, 1H), 7.64-7.57 (m, 3H), 7.55 |
| | | | | (dd, J = 1.9, 0.6 Hz, 2H), 7.34-7.20 (m, |
| | | | | 3H), 3.61 (d, J = 8.4 Hz, 1H), 3.49 (d, J = |
| | | | | 8.5 Hz, 1H); |
| | | | | ¹⁹F NMR (376 MHz, DMSO-d₆) δ −114.11, −128.48 |
| F62 | | | ESIMS m/z 547 | ¹H NMR (400 MHz, DMSO-d₆) δ 10.76 |
| | | | ([M − H]⁻) | (s, 1H), 10.35 (s, 1H), 8.01 (dd, J = 6.9, |
| | | | | 2.7 Hz, 1H), 7.69 (dt, J = 6.7, 2.1 Hz, |
| | | | | 2H), 7.62 (t, J = 1.9 Hz, 1H), 7.60-7.49 |
| | | | | (m, 4H), 7.31 (dd, J = 10.2, 9.0 Hz, 1H), |
| | | | | 3.61 (d, J = 8.5 Hz, 1H), 3.49 (d, J = 8.5 |
| | | | | Hz, 1H); |
| | | | | ¹⁹F NMR (376 MHz, DMSO-d₆) δ −108.68, −125.92 |
| F63 | | | ESIMS m/z 534 | ¹H NMR (400 MHz, DMSO-d₆) δ 10.73 |
| | | | ([M − H]⁻) | (s, 1H), 9.66 (d, J = 1.8 Hz, 1H), 8.32 (dd, |
| | | | | J = 7.1, 2.7 Hz, 1H), 7.62 (t, J = 1.9 Hz, |
| | | | | 1H), 7.57-7.50 (m, 3H), 7.26 (dd, J = |
| | | | | 10.6, 9.0 Hz, 1H), 3.64 (t, J = 5.7, 3.5 Hz, |
| | | | | 4H), 3.59 (d, J = 8.5 Hz, 1H), 3.47 (d, J = |
| | | | | 8.6 Hz, 1H), 3.20 (s, 2H), 2.55 (dd, J = |
| | | | | 5.7, 3.5 Hz, 4H); |
| | | | | ¹⁹F NMR (376 MHz, DMSO-d₆) δ −133.21 |
| F64 | | | ESIMS m/z 545 | ¹H NMR (400 MHz, DMSO-d₆) δ 10.70 |
| | | | ([M − H]⁻) | (s, 1H), 9.95 (s, 1H), 8.21 (dd, J = 7.1, 2.6 |
| | | | | Hz, 1H), 7.62 (t, J = 1.9 Hz, 1H), 7.56- |
| | | | | 7.48 (m, 3H), 7.24 (dd, J = 10.6, 8.9 Hz, |
| | | | | 1H), 3.59 (d, J = 8.5 Hz, 1H), 3.47 (d, J = |
| | | | | 8.6 Hz, 1H), 2.96-2.82 (m, 1H), 2.76 |
| | | | | (dd, J = 15.2, 5.1 Hz, 1H), 2.60-2.52 (m, |
| | | | | 1H), 1.14 (d, J = 6.9 Hz, 3H); |
| | | | | ¹⁹F NMR (376 MHz, DMSO-d₆) δ −72.06, |
| | | | | −129.88 (d, J = 6.6 Hz) |
| F65 | 100-104 | | ESIMS m/z 545 | ¹H NMR (400 MHz, DMSO-d₆) δ 10.87 |
| | | | ([M − H]⁻) | (s, 1H), 9.95 (d, J = 4.4 Hz, 1H), 8.21 (d, |
| | | | | J = 2.0 Hz, 1H), 7.82-7.78 (m, 1H), 7.65- |
| | | | | 7.61 (m, 3H), 7.56-7.52 (m, 3H), 7.41- |
| | | | | 7.35 (m, 2H), 3.62 (d, J = 8.4 Hz, 1H), |
| | | | | 3.51 (d, J = 8.0 Hz, 1H); |
| | | | | ¹⁹F NMR (376 MHz, DMSO-d₆) δ −113.59 |
| F66 | 206-210 | | ESIMS m/z 545 | ¹H NMR (400 MHz, DMSO-d₆) δ 10.84 |
| | | | ([M − H]⁻) | (s, 1H), 10.21 (s, 1H), 7.98 (d, J = 2.0 Hz, |
| | | | | 1H), 7.85 (d, J = 8.0 Hz , 1H), 7.78 (d, J = |
| | | | | 10.0 Hz, 1H), 7.65-7.53 (m, 6H), 7.51- |
| | | | | 7.46 (m, 1H), 3.61 (d, J = 8.8 Hz, 1H), |
| | | | | 3.50 (d, J = 8.8 Hz, 1H); |
| | | | | ¹⁹F NMR (376 MHz, DMSO-d₆) δ −112.46 |
| F67 | 128-132 | | ESIMS m/z 565 | ¹H NMR (400 MHz, DMSO-d₆) δ 10.86 |
| | | | ([M + H]⁺) | (s, 1H), 10.24 (s, 1H), 8.06-8.01 (m, |
| | | | | 1H), 7.98-7.89 (m, 2H), 7.68-7.54 (m, |
| | | | | 6H), 3.61 (d, J = 8.4 Hz, 1H), 3.50 (d, J = |
| | | | | 8.4 Hz, 1H) |
| F68 | 195-199 | | ESIMS m/z 565 | ¹H NMR (400 MHz, DMSO-d₆) δ 10.87 |
| | | | ([M + H]⁺) | (s, 1H), 10.19 (s, 1H), 8.15 (s, 1H), 7.69- |
| | | | | 7.61 (m, 2H), 7.58-7.53 (m, 4H), 7.40- |
| | | | | 7.35 (m, 1H), 3.62 (d, J = 8.4 Hz, 1H), |
| | | | | 3.50 (d, J = 8.4 Hz, 1H); |
| | | | | ¹⁹F NMR (376 MHz, DMSO-d₆) δ −138.25, −139.80 |
| F69 | 185-190 | | ESIMS m/z 565 | ¹H NMR (400 MHz, DMSO-d₆) δ 10.88 |
| | | | ([M + H]⁺) | (s, 1H), 10.10 (s, 1H), 8.19 (s, 1H), 7.63- |
| | | | | 7.43 (m, 8H), 3.62 (d, J = 8.4 Hz, 1H), |
| | | | | 3.50 (d, J = 8.8 Hz, 1H); |
| | | | | ¹⁹F NMR (376 MHz, DMSO-d₆) δ −117.96, −119.08 |
| F70 | 238-242 | | ESIMS m/z 552 | ¹H NMR (400 MHz, DMSO-d₆) δ 10.86 |
| | | | ([M − H]⁻) | (s, 1H), 10.39 (s, 1H), 8.14 (d, J = 8.4 Hz, |
| | | | | 2H), 8.05 (d, J = 8.4 Hz, 2H), 8.00 (d, J = |
| | | | | 2.4 Hz, 1H), 7.63-7.54 (m, 5H), 3.61 (d, |
| | | | | J = 8.8 Hz, 1H), 3.50 (d, J = 8.0 Hz, 1H) |
| F71 | 227-232 | | ESIMS m/z 570 | ¹H NMR (400 MHz, DMSO-d₆) δ 10.83 |
| | | | ([M − H]⁻) | (s, 1H), 10.30 (s, 1H), 8.16 (s, 1H), 8.08 |
| | | | | (d, J = 9.6 Hz, 1H), 7.94-7.85 (m, 2H), |
| | | | | 7.63-7.61 (m, 2H), 7.56-7.53 (m, 3H), |
| | | | | 3.61 (d, J = 8.0 Hz, 1H), 3.50 (d, J = 8.8 |
| | | | | Hz, 1H); |
| | | | | ¹⁹F NMR (376 MHz, DMSO-d₆) δ −111.69 |
| F72 | 229-233 | | ESIMS m/z 570 | ¹H NMR (400 MHz, DMSO-d₆) δ 10.87 |
| | | | ([M − H]⁻) | (s, 1H), 10.47 (s, 1H), 8.18-8.13 (m, |
| | | | | 1H), 8.05-7.96 (m, 3H), 7.63-7.62 (m, |
| | | | | 1H), 7.58-7.55 (m, 4H), 3.61 (d, J = 8.4 |
| | | | | Hz, 1H), 3.50 (d, J = 8.8 Hz, 1H); |
| | | | | ¹⁹F NMR (376 MHz, DMSO-d₆) δ −107.46 |
| F73 | 177-181 | | ESIMS m/z 541 | ¹H NMR (400 MHz, DMSO-d₆) δ 10.85 |
| | | | ([M − H]⁻) | (s, 1H), 9.99 (s, 1H), 8.00 (d, J = 2.0 Hz, |
| | | | | 1H), 7.64-7.61 (m, 2H), 7.55-7.50 (m, |
| | | | | 4H), 7.41-7.38 (m, 1H), 7.33-7.29 (m, |
| | | | | 2H), 3.62 (d, J = 8.8 Hz, 1H), 3.50 (d, J = |
| | | | | 8.4 Hz, 1H), 2.46 (s, 3H) |
| F74 | 104-108 | | ESIMS m/z 541 | ¹H NMR (400 MHz, DMSO-d₆) δ 10.84 |
| | | | ([M − H]⁻) | (s, 1H), 10.01 (s, 1H), 7.99 (d, J = 2.4 Hz, |
| | | | | 1H), 7.81-7.77 (m, 2H), 7.63-7.52 (m, |
| | | | | 5H), 7.43 (d, J = 4.8 Hz, 2H), 3.61 (d, J = |
| | | | | 8.4 Hz, 1H), 3.50 (d, J = 8.0 Hz, 1H), |
| | | | | 2.41 (s, 3H) |
| F75 | 124-126 | | ESIMS m/z 543 | ¹H NMR (400 MHz, DMSO-d₆) δ 10.83 |
| | | | ([M + H]⁺) | (s, 1H), 9.96 (s, 1H), 8.01 (d, J = 2.4 Hz, |
| | | | | 1H), 7.90 (d, J = 8.0 Hz, 2H), 7.63-7.50 |
| | | | | (m, 5H), 7.35 (d, J = 8.4 Hz, 2H), 3.61 (d, |
| | | | | J = 8.4 Hz, 1H), 3.50 (d, J = 8.4 Hz, 1H), |
| | | | | 2.40 (s, 3H) |
| F76 | 204-207 | | ESIMS m/z 557 | ¹H NMR (400 MHz, DMSO-d₆) δ 10.83 |
| | | | ([M + H]⁺) | (s, 1H), 9.95 (s, 1H), 7.97 (d, J = 2.4 Hz, |
| | | | | 1H), 7.63-7.51 (m, 7H), 7.25 (s, 1H), |
| | | | | 3.61 (d, J = 8.4 Hz, 1H), 3.50 (d, J = 8.4 |
| | | | | Hz, 1H), 2.36 (s, 6H) |
| F77 | 189-193 | | ESIMS m/z 561 | ¹H NMR (400 MHz, DMSO-d₆) δ 10.85 |
| | | | ([M + H]⁺) | (s, 1H), 10.02 (s, 1H), 8.00 (d, J = 2.0 Hz, |
| | | | | 1H), 7.63-7.50 (m, 6H), 7.22-7.13 (m, |
| | | | | 2H), 3.62 (d, J = 8.4 Hz, 1H), 3.50 (d, J = |
| | | | | 8.4 Hz, 1H), 2.47 (s, 3H); |
| | | | | ¹⁹F NMR (376 MHz, DMSO-d₆) δ −111.42 |
| F78 | 196-199 | | ESIMS m/z 563 | ¹H NMR (400 MHz, DMSO-d₆) δ 10.87 |
| | | | ([M + H]⁺) | (s, 1H), 10.25 (s, 1H), 8.04 (s, 1H), 7.68- |
| | | | | 7.45 (m, 9H), 3.62 (d, J = 8.4 Hz, 1H), |
| | | | | 3.50 (d, J = 8.4 Hz, 1H) |
| F79 | 201-206 | | ESIMS m/z 561 | ¹H NMR (400 MHz, DMSO-d₆) δ 10.86 |
| | | | ([M − H]⁻) | (s, 1H), 10.18 (s, 1H), 8.02-8.00 (m, |
| | | | | 3H), 7.64-7.60 (m, 3H), 7.59-7.51 (m, |
| | | | | 4H), 3.61 (d, J = 8.8 Hz, 1H), 3.50 (d, J = |
| | | | | 8.4 Hz, 1H) |
| F80 | 215-218 | | ESIMS m/z 597 | ¹H NMR (400 MHz, DMSO-d₆) δ 10.86 |
| | | | ([M − H]⁻) | (s, 1H), 10.39 (s, 1H), 8.00 (d, J = 2.0 Hz, |
| | | | | 2H), 7.96 (d, J = 2.0 Hz, 1H), 7.93-7.90 |
| | | | | (m, 1H), 7.63-7.62 (m, 1H), 7.60-7.54 |
| | | | | (m, 4H), 3.61 (d, J = 8.4 Hz, 1H), 3.52 (d, |
| | | | | J = 8.4 Hz, 1H) |
| F81 | 106-110 | | ESIMS m/z 595 | ¹H NMR (400 MHz, DMSO-d₆) δ 10.86 |
| | | | ([M − H]⁻) | (s, 1H), 10.42 (s, 1H), 8.33-8.27 (m, |
| | | | | 2H), 8.02-7.98 (m, 2H), 7.84-7.79 (m, |
| | | | | 1H), 7.63-7.53 (m, 5H), 3.61 (d, J = 8.4 |
| | | | | Hz, 1H), 3.50 (d, J = 8.4 Hz, 1H); |
| | | | | ¹⁹F NMR (376 MHz, DMSO-d₆) δ −61.16 |
| F82 | 170-174 | | ESIMS m/z 595 | ¹H NMR (400 MHz, DMSO-d₆) δ 10.83 |
| | | | ([M − H]⁻) | (s, 1H), 10.38 (s, 1H), 8.18 (d, J = 8.0 Hz, |
| | | | | 2H), 8.01 (d, J = 2.0 Hz, 1H), 7.94 (d, J = |
| | | | | 8.4 Hz, 2H), 7.63-7.54 (m, 5H), 3.61 (d, |
| | | | | J = 8.0 Hz, 1H), 3.51 (d, J = 8.4 Hz, 1H); |
| | | | | ¹⁹F NMR (376 MHz, DMSO-d₆) δ −61.40 |
| F83 | 165-169 | | ESIMS m/z 615 | ¹H NMR (400 MHz, DMSO-d₆) δ 10.86 |
| | | | ([M + H]⁺) | (s, 1H), 10.44 (s, 1H), 8.40-8.36 (m, |
| | | | | 2H), 7.98 (d, J = 2.0 Hz, 1H), 7.76-7.71 |
| | | | | (m, 1H), 7.63-7.53 (m, 5H), 3.61 (d, J = |
| | | | | 4.0 Hz, 1H), 3.51 (d, J = 8.4 Hz, 1H); |
| | | | | ¹⁹F NMR (376 MHz, DMSO-d₆) δ −60.24, −111.24 |
| F84 | 190-195 | | ESIMS m/z 557 | ¹H NMR (400 MHz, DMSO-d₆) δ 10.81 |
| | | | ([M − H]⁻) | (s, 1H), 10.08 (s, 1H), 8.00 (s, 1H), 7.63- |
| | | | | 7.51 (m, 7H), 7.49-7.43 (m, 1H), 7.18 |
| | | | | (d, J = 8.4 Hz, 1H), 3.84 (s, 3H), 3.61 (d, |
| | | | | J = 8.8 Hz, 1H), 3.51 (d, J = 8.8 Hz, 1H) |
| F85 | 186-191 | | ESIMS m/z 559 | ¹H NMR (400 MHz, DMSO-d₆) δ 10.82 |
| | | | ([M + H]⁺) | (s, 1H), 9.85 (s, 1H), 8.01-7.97 (s, 3H), |
| | | | | 7.63-7.50 (m, 5H), 7.08 (d, J = 9.2 Hz, |
| | | | | 2H), 3.85 (s, 3H), 3.61 (d, J = 8.4 Hz, |
| | | | | 1H), 3.50 (d, J = 8.8 Hz, 1H) |
| F86 | 109-113 | | ESIMS m/z 663 | ¹H NMR (400 MHz, DMSO-d₆) δ 10.88 |
| | | | ([M − H]⁻) | (s, 1H), 10.68 (s, 1H), 8.63 (s, 2H), 8.43 |
| | | | | (s, 1H), 8.01 (s, 1H), 7.63 (s, 1H), 7.58- |
| | | | | 7.55 (m, 4H), 3.61 (d, J = 8.8 Hz, 1H), |
| | | | | 3.51 (d, J = 8.4 Hz, 1H); |
| | | | | ¹⁹F NMR (376 MHz, DMSO-d₆) δ −61.30 |
| F87 | 119-124 | | ESIMS m/z 527 | ¹H NMR (400 MHz, DMSO-d₆) δ 10.85 |
| | | | ([M − H] ) | (s, 1H), 10.07 (s, 1H), 8.01-7.98 (m, |
| | | | | 3H), 7.64-7.52 (m, 8H), 3.61 (d, J = 8.0 |
| | | | | Hz, 1H), 3.50 (d, J = 8.4 Hz, 1H) |
| F88 | 130-134 | | ESIMS m/z 574 | ¹H NMR (400 MHz, DMSO-d₆) δ 10.88 |
| | | | ([M + H]⁺) | (s, 1H), 10.56 (s, 1H), 8.82 (s, 1H), 8.49- |
| | | | | 8.41 (m, 2H), 8.00 (d, J = 2.0 Hz, 1H), |
| | | | | 7.89-7.85 (m, 1H), 7.63-7.55 (m, 5H), |
| | | | | 3.60 (d, J = 8.8 Hz, 1H), 3.51 (d, J = 8.8 |
| | | | | Hz, 1H) |
| F89 | 145-149 | | ESIMS m/z 574 | ¹H NMR (400 MHz, DMSO-d₆) δ 10.85 |
| | | | ([M + H]⁺) | (s, 1H), 10.50 (s, 1H), 8.39 (d, J = 8.8 Hz, |
| | | | | 2H), 8.22 (d, J = 8.0 Hz, 2H), 8.01 (d, J = |
| | | | | 2.4 Hz, 1H), 7.63-7.55 (m, 5H), 3.61 (d, |
| | | | | J = 8.8 Hz, 1H), 3.51 (d, J = 8.4 Hz, 1H) |
| F90 | 260-265 | | ESIMS m/z 586 | ¹H NMR (400 MHz, DMSO-d₆) δ 10.88 |
| | | | ([M − H]⁻) | (s, 1H), 10.36 (s, 1H), 8.23 (s, 1H), 8.18 |
| | | | | (d, J = 8.0 Hz, 1H), 8.04 (s, 1H), 7.76 (d, |
| | | | | J = 8.4 Hz, 1H), 7.65-7.63 (m, 2H), 7.55- |
| | | | | 7.53 (m, 3H), 3.62 (d, J = 8.8 Hz, 1H), |
| | | | | 3.51 (d, J = 8.0 Hz, 1H), 2.58 (s, 3H) |
| F91 | 223-227 | | ESIMS m/z 553 | ¹H NMR (400 MHz, DMSO-d₆) δ 10.85 |
| | | | ([M + H]⁺) | (s, 1H), 10.19 (s, 1H), 8.00-7.98 (m, |
| | | | | 3H), 7.66-7.52 (m, 7H), 4.45 (s, 1H), |
| | | | | 3.61 (d, J = 8.4 Hz, 1H), 3.50 (d, J = 8.8 |
| | | | | Hz, 1H) |
| F92 | | | ESIMS m/z 575 | ¹H NMR (400 MHz, DMSO-d₆) rotational |
| | | | ([M − H]⁻) | isomers: δ 10.93 (t, J = 4.4 Hz, 1H), 7.89- |
| | | | | 7.83 (m, 1H), 7.81-7.71 (m, 1H), 7.69- |
| | | | | 7.60 (m, 2H), 7.55 (t, J = 1.8 Hz, 2H), |
| | | | | 3.62 (d, J = 8.5 Hz, 1H), 3.53-3.46 (m, |
| | | | | 1H), 3.13 (d, J = 3.5 Hz, 3H), 2.87 (s, |
| | | | | 1H), 2.33 (dt, J = 16.0, 4.5 Hz, 0.5H), |
| | | | | 2.19 (ddd, J = 16.1, 8.1, 4.2 Hz, 0.5H), |
| | | | | 2.10-1.96 (m, 0.5H), 1.84 (ddd, J = 16.1, |
| | | | | 8.9, 7.3 Hz, 0.5H), 1.04 (td, J = 7.3, 3.0 |
| | | | | Hz, 3H); |
| | | | | ¹⁹F NMR (376 MHz, DMSO-d₆) |
| | | | | rotational isomers δ −72.14 (d, J = 13.5 |
| | | | | Hz), −72.24 (d, J = 3.7 Hz) |
| F93 | | (thin | ESIMS m/z 582 | ¹H NMR (400 MHz, DMSO-d₆) rotational |
| | | film) | ([M + H]⁺) | isomers δ 10.93-10.55 (m, 1H), 7.89 |
| | | 3278, | | (dd, J = 5.7, 2.0 Hz, 1H), 7.63 (ddd, J = |
| | | 2232, | | 7.3, 4.5, 1.6 Hz, 2H), 7.55 (dd, J = 6.1, |
| | | 1698, | | 2.0 Hz, 2H), 7.51-7.43 (m, 2H), 7.32 |
| | | 1633, | | (dd, J = 8.9, 2.4 Hz, 1H), 7.20 (dd, J = |
| | | 1591, | | 9.7, 7.9 Hz, 1H), 3.60 (d, J = 8.5 Hz, 1H), |
| | | 1418 | | 3.45 (d, J = 8.5 Hz, 1H), 3.31 (d, 3H), |
| | | | | 2.40 (d, J = 4.1 Hz, 3H) |
| F94 | | | ESIMS m/z 577 | ¹H NMR (400 MHz, CDCl₃) rotational |
| | | | ([M − H]⁻) | isomers δ 10.81 (d, J = 4.0 Hz, 1H), 7.73- |
| | | | | 7.58 (m, 2H), 7.54 (d, J = 1.9 Hz, 2H), |
| | | | | 7.51-7.42 (m, 2H), 7.29-7.19 (m, 1H), |
| | | | | 6.96 (t, J = 7.2 Hz, 2H), 3.60 (dd, J = 8.5, |
| | | | | 3.2 Hz, 1H), 3.47 (dd, J = 8.5, 4.1 Hz, |
| | | | | 1H), 3.28 (d, J = 2.7 Hz, 3H); |
| | | | | ¹⁹F NMR (376 MHz, CDCl₃) δ −104.22 (d, |
| | | | | J = 13.4 Hz) |
| F95 | | | HRMS-ESI (m/z) | ¹H NMR (500 MHz, DMSO-d₆) δ 10.54 |
| | | | [M + H]⁺ calcd for | (s, 1H), 9.84 (s, 1H), 7.82 (dddd, J = 16.1, |
| | | | C₂₁H₁₃C₁₂F₉N₂O₃, | 7.9, 5.2, 3.0 Hz, 3H), 7.59 (dd, J = 10.7, |
| | | | 583.0232; found, | 8.6 Hz, 1H), 7.19 (td, J = 9.3, 1.8 Hz, |
| | | | 583.0252 | 1H), 4.39 (s, 2H), 4.26 (q, J = 9.3 Hz, |
| | | | | 2H), 3.67 (d, J = 1.8 Hz, 2H); |
| | | | | ¹⁹F NMR (471 MHz, DMSO-d₆) δ −59.94 |
| | | | | (d, J = 12.3 Hz), −72.90 (q, J = 8.3, 6.7 |
| | | | | Hz), −73.00, −116.96 (ddp, J = 18.2, 12.1, |
| | | | | 5.8 Hz), −121.75 (d, J = 7.8 Hz), −126.32, −126.63 |
| F96 | | | ESIMS m/z 633 | ¹H NMR (400 MHz, DMSO-d₆) δ 10.91 |
| | | | ([M − H]⁻) | (s, 1H), 10.30 (s, 1H), 8.22 (d, J = 1.6 Hz, |
| | | | | 2H), 8.13 (s, 1H), 8.05 (d, J = 2.4 Hz, |
| | | | | 1H), 7.91-7.78 (m, 2H), 7.72-7.62 (m, |
| | | | | 2H), 7.55 (d, J = 8.7 Hz, 1H), 3.85 (d, J = |
| | | | | 8.6 Hz, 1H), 3.69 (d, J = 8.6 Hz, 1H), |
| | | | | 2.49 (s, 3H); |
| | | | | ¹⁹F NMR (376 MHz, DMSO-d₆) δ −61.20 |
| F97 | | | ESIMS m/z 584 | ¹H NMR (400 MHz, DMSO-d₆) δ 10.92 |
| | | | ([M − H]⁻) | (s, 1H), 10.29 (s, 1H), 8.03 (d, J = 2.4 Hz, |
| | | | | 1H), 7.91-7.78 (m, 4H), 7.72-7.61 (m, |
| | | | | 2H), 7.59 (dd, J = 10.7, 8.7 Hz, 1H), 7.54 |
| | | | | (d, J = 8.8 Hz, 1H), 3.69 (d, J = 8.5 Hz, |
| | | | | 1H), 3.50 (d, J = 8.5 Hz, 1H), 2.48 (s, 3H); |
| | | | | ¹⁹F NMR (376 MHz, DMSO-d₆) δ −59.93 |
| | | | | (d, J = 12.3 Hz), −116.96 (q, J = 12.4 Hz) |
| F98 | | | ESIMS m/z 594 | ¹H NMR (400 MHz, DMSO-d₆) δ 10.90 |
| | | | ([M − H]⁻) | (s, 1H), 10.29 (s, 1H), 8.03 (d, J = 2.4 Hz, |
| | | | | 1H), 7.88-7.78 (m, 3H), 7.76-7.61 (m, |
| | | | | 2H), 7.57-7.39 (m, 3H), 3.59 (d, J = 8.4 |
| | | | | Hz, 1H), 3.43 (d, J = 8.5 Hz, 1H), 2.48 (s, 3H); |
| | | | | ¹⁹F NMR (376 MHz, DMSO-d₆) δ −109.32 |
| F99 | | (thin | MS m/z 568 | ¹H NMR (400 MHz, DMSO-d₆) δ 10.91 |
| | | film) | ([M + H]⁺) | (s, 1H), 10.29 (s, 1H), 8.02 (d, J = 2.4 Hz, |
| | | 3288, | | 1H), 7.88-7.78 (m, 2H), 7.76 (d, J = 2.2 |
| | | 1666, | | Hz, 1H), 7.70 (d, J = 5.1 Hz, 1H), 7.71- |
| | | 1520, | | 7.60 (m, 2H), 7.54 (d, J = 8.8 Hz, 1H), |
| | | 1478, | | 7.47-7.39 (m, 1H), 3.60 (d, J = 8.5 Hz, |
| | | 1410 | | 1H), 3.46 (d, J = 8.6 Hz, 1H), 2.48 (s, 3H) |
| F100 | | | ESIMS m/z 581 | ¹H NMR (400 MHz, DMSO-d₆) δ 10.92 |
| | | | ([M − H]⁻) | (s, 1H), 10.32 (s, 1H), 7.99 (d, J = 2.4 Hz, |
| | | | | 1H), 7.91-7.79 (m, 2H), 7.71 (ddd, J = |
| | | | | 6.5, 4.6, 3.3 Hz, 2H), 7.65-7.51 (m, 4H), |
| | | | | 3.68 (d, J = 8.5 Hz, 1H), 3.51 (d, J = 8.5 |
| | | | | Hz, 1H); |
| | | | | ¹⁹F NMR (376 MHz, DMSO-d₆) δ −59.84-−59.94 |
| | | | | (m), −108.53, −116.95 (d, J = 12.5 Hz) |
| F101 | | | ESIMS m/z 695 | ¹H NMR (400 MHz, DMSO-d₆) δ 10.91 |
| | | | ([M − H]⁻) | (s, 1H), 10.08 (d, J = 2.6 Hz, 1H), 9.02 (s, |
| | | | | 1H), 8.16 (d, J = 2.5 Hz, 1H), 7.91-7.79 |
| | | | | (m, 2H), 7.75-7.50 (m, 4H), 7.30 (td, J = |
| | | | | 9.0, 1.4 Hz, 1H), 3.68 (d, J = 8.4 Hz, 1H), |
| | | | | 3.50 (d, J = 8.5 Hz, 1H), 1.45 (s, 9H); |
| | | | | ¹⁹F NMR (376 MHz, DMSO-d₆) δ −59.92 |
| | | | | (d, J = 12.5 Hz), −113.26, −116.98 (q, J = |
| | | | | 12.6 Hz), −118.62 (d, J = 8.9 Hz) |
| F102 | | | ESIMS m/z 579 | ¹H NMR (400 MHz, DMSO-d₆) δ 10.86 |
| | | | ([M − H]⁻) | (s, 1H), 9.77 (s, 1H), 8.04 (d, J = 2.5 Hz, |
| | | | | 1H), 7.89-7.78 (m, 2H), 7.64-7.54 (m, |
| | | | | 2H), 7.48 (d, J = 8.8 Hz, 1H), 3.67 (d, J = |
| | | | | 8.5 Hz, 1H), 3.48 (d, J = 8.5 Hz, 1H), |
| | | | | 2.89 (tdt, J = 11.8, 9.2, 6.0 Hz, 1H), 2.76 |
| | | | | (dd, J = 15.1, 5.0 Hz, 1H), 2.60-2.52 (m, |
| | | | | 1H), 1.16 (d, J = 6.9 Hz, 3H); |
| | | | | ¹⁹F NMR (376 MHz, DMSO-d₆) δ −59.94 |
| | | | | (d, J = 12.8 Hz), −72.06, −116.99 (q, J = |
| | | | | 12.5 Hz) |
| F103 | | | ESIMS m/z 596 | ¹H NMR (400 MHz, DMSO-d₆) δ 10.91 |
| | | | ([M − H]⁻) | (s, 1H), 9.82 (d, J = 4.2 Hz, 1H), 8.22 (d, |
| | | | | J = 2.5 Hz, 1H), 7.91-7.79 (m, 2H), 7.67- |
| | | | | 7.54 (m, 2H), 7.53 (d, J = 8.8 Hz, 1H), |
| | | | | 7.06 (ddd, J = 10.2, 8.6, 1.4 Hz, 1H), 6.94 |
| | | | | (td, J = 8.1, 6.1 Hz, 1H), 5.52 (s, 2H), |
| | | | | 3.68 (d, J = 8.5 Hz, 1H), 3.50 (d, J = 8.5 |
| | | | | Hz, 1H); |
| | | | | ¹⁹F NMR (376 MHz, DMSO-d₆) δ −59.92 |
| | | | | (d, J = 12.5 Hz), −116.99 (q, J = 12.5 Hz), |
| | | | | −126.39 (d, J = 18.8 Hz), −130.85 (d, J = 18.5 Hz) |
| F104 | | | ESIMS m/z 674 | ¹H NMR (400 MHz, DMSO-d₆) δ 10.92 |
| | | | ([M − H]⁻) | (s, 1H), 10.87 (s, 1H), 10.15 (d, J = 2.4 |
| | | | | Hz, 1H), 8.17 (d, J = 2.5 Hz, 1H), 7.87 |
| | | | | (dd, J = 7.1, 2.2 Hz, 1H), 7.82 (q, J = 8.2 |
| | | | | Hz, 2H), 7.67-7.51 (m, 3H), 7.41 (td, J = |
| | | | | 9.0, 1.5 Hz, 1H), 6.58 (t, J = 53.2 Hz, |
| | | | | 1H), 3.68 (d, J = 8.5 Hz, 1H), 3.50 (d, J = |
| | | | | 8.5 Hz, 1H); |
| | | | | ¹⁹F NMR (376 MHz, DMSO-d₆) δ −59.91 |
| | | | | (d, J = 12.5 Hz), −112.54 (d, J = 6.6 Hz), −116.98 |
| | | | | (q, J = 12.5 Hz), −117.73 (d, J = 6.6 Hz), −125.76 |
| F105 | | | ESIMS m/z 668 | ¹H NMR (400 MHz, DMSO-d₆) δ 10.92 |
| | | | ([M − H]⁻) | (s, 1H), 10.09 (d, J = 2.8 Hz, 1H), 9.79 (s, |
| | | | | 1H), 8.17 (d, J = 2.5 Hz, 1H), 7.91-7.79 |
| | | | | (m, 2H), 7.83-7.71 (m, 1H), 7.67-7.51 |
| | | | | (m, 3H), 7.34 (td, J = 8.9, 1.4 Hz, 1H), |
| | | | | 4.10 (s, 2H), 3.68 (d, J = 8.5 Hz, 1H), |
| | | | | 3.50 (d, J = 8.5 Hz, 1H), 3.42 (s, 3H); |
| | | | | ¹⁹F NMR (376 MHz, DMSO-d₆) δ −59.92 |
| | | | | (d, J = 12.7 Hz), −112.30 (d, J = 7.2 Hz), −116.98 |
| | | | | (q, J = 12.6 Hz), −117.64 (d, J = 7.4 Hz) |
| F106 | | | ESIMS m/z 566 | ¹H NMR (400 MHz, DMSO-d₆) δ 10.89 |
| | | | ([M + H]⁺) | (s, 1H), 10.32 (s, 1H), 8.71 (d, J = 2.3 Hz, |
| | | | | 1H), 8.59 (d, J = 2.4 Hz, 1H), 8.22 (ddd, J = |
| | | | | 11.1, 9.0, 2.3 Hz, 1H), 7.67-7.60 (m, |
| | | | | 2H), 7.58-7.52 (m, 3H), 3.62 (d, J = 8.5 |
| | | | | Hz, 1H), 3.51 (d, J = 8.5 Hz, 1H); |
| | | | | ¹⁹F NMR (377 MHz, DMSO-d₆) δ −114.74 |
| | | | | (t, J = 11.9 Hz), −116.87 (t, J = 11.0 Hz) |
| F107 | 112-116 | | ESIMS m/z 584 | ¹H NMR (400 MHz, DMSO-d₆) δ 10.91 |
| | | | ([M + H]⁺) | (s, 1H), 10.18 (s, 1H), 8.55-8.49 (m, |
| | | | | 1H), 8.24 (br, 1H), 7.87-7.81 (m, 2H), |
| | | | | 7.63-7.53 (m, 3H), 7.37 (dd, J = 2.4, 8.0 |
| | | | | Hz, 1H), 3.68 (d, J = 8.4 Hz, 1H), 3.51 (d, |
| | | | | J = 8.4 Hz, 1H); |
| | | | | ¹⁹F NMR (376 MHz, DMSO-d₆) δ −59.90, |
| | | | | −65.45, −66.61, −116.97 |
| F108 | 116-120 | | ESIMS m/z 584 | ¹H NMR (400 MHz, DMSO-d₆) δ 10.91 |
| | | | ([M + H]⁺) | (s, 1H), 10.18 (s, 1H), 8.55-8.49 (m, |
| | | | | 1H), 8.24 (br, 1H), 7.87-7.81 (m, 2H), |
| | | | | 7.63-7.53 (m, 3H), 7.37 (dd, J = 2.0, 8.0 |
| | | | | Hz, 1H), 3.68 (d, J = 8.8 Hz, 1H), 3.51 (d, |
| | | | | J = 8.4 Hz, 1H); |
| | | | | ¹⁹F NMR (376 MHz, DMSO-d₆) δ −59.90, |
| | | | | −65.45, −66.61, −116.97 |
| F109 | 116-120 | | ESIMS m/z 584 | ¹H NMR (400 MHz, DMSO-d₆) δ 10.90 |
| | | | ([M + H]⁺) | (s, 1H), 10.43 (s, 1H), 8.66-8.65 (m, |
| | | | | 1H), 8.54-8.49 (m, 1H), 8.02 (d, J = 2.0 |
| | | | | Hz, 1H), 7.87-7.81 (m, 2H), 7.61-7.54 |
| | | | | (m, 3H), 3.68 (d, J = 8.4 Hz, 1H), 3.51 (d, |
| | | | | J = 8.4 Hz, 1H); |
| | | | | ¹⁹F NMR (376 MHz, DMSO-d₆) δ −59.90, |
| | | | | −84.50, −116.96, −139.86 |
| F110 | 115-119 | | ESIMS m/z 584 | ¹H NMR (400 MHz, DMSO-d₆) δ 10.90 |
| | | | ([M + H]⁺) | (s, 1H), 10.43 (s, 1H), 8.67-8.66 (m, |
| | | | | 1H), 8.54-8.49 (m, 1H), 8.02 (d, J = 2.0 |
| | | | | Hz, 1H), 7.87-7.81 (m, 2H), 7.61-7.55 |
| | | | | (m, 3H), 3.68 (d, J = 8.4 Hz, 1H), 3.51 (d, |
| | | | | J = 8.8 Hz, 1H); |
| | | | | ¹⁹F NMR (376 MHz, DMSO-d₆) δ −59.90, |
| | | | | −84.54, −116.96, −139.81 |
| F111 | 168-172 | | ESIMS m/z 658 | ¹H NMR (400 MHz, DMSO-d₆) δ 11.18 |
| | | | ([M + H]⁺) | (s, 1H), 10.89 (s, 1H), 9.82 (s, 1H), 8.29 |
| | | | | (d, J = 2.4 Hz, 1H), 7.88-7.81 (m, 3H), |
| | | | | 7.78 (dd, J = 1.6, 12.8 Hz, 1H), 7.64- |
| | | | | 7.52 (m, 4H), 6.57-6.30 (m, 1H), 3.68 |
| | | | | (d, J = 8.4 Hz, 1H), 3.50 (d, J = 8.4 Hz, 1H); |
| | | | | ¹⁹F NMR (376 MHz, DMSO-d₆) δ −59.93, |
| | | | | −110.66, −116.98, −125.62 |
| F112 | 190-194 | | ESIMS m/z 676 | ¹H NMR (400 MHz, DMSO-d₆) δ 11.68 |
| | | | ([M + H]⁺) | (s, 1H), 10.89 (s, 1H), 9.88 (s, 1H), 8.26 |
| | | | | (d, J = 2.0 Hz, 1H), 7.90-7.81 (m, 3H), |
| | | | | 7.75 (dd, J = 1.6, 12.8 Hz, 1H), 7.66- |
| | | | | 7.56 (m, 3H), 7.54 (d, J = 8.8 Hz, 1H), |
| | | | | 3.68 (d, J = 8.8 Hz, 1H), 3.50 (d, J = 8.4 |
| | | | | Hz, 1H); |
| | | | | ¹⁹F NMR (376 MHz, DMSO-d₆) δ −59.93, |
| | | | | −110.66, −73.95, −110.73, −116.98 |
| F113 | 232-236 | | ESIMS m/z 636 | ¹H NMR (400 MHz, DMSO-d₆) δ 10.87 |
| | | | ([M + H]⁺) | (s, 1H), 10.03 (s, 1H), 9.84 (s, 1H), 8.02 |
| | | | | (d, J = 2.4 Hz, 1H), 7.93-7.81 (m, 3H), |
| | | | | 7.63-7.56 (m, 2H), 7.53 (d, J = 8.8 Hz, |
| | | | | 1H), 7.3 (d, J = 8.4 Hz, 1H), 3.69 (d, J = |
| | | | | 8.4 Hz, 1H), 3.51 (d, J = 8.4 Hz, 1H), |
| | | | | 2.38-2.32 (m, 3H), 2.13 (s, 3H); |
| | | | | ¹⁹F NMR (376 MHz, DMSO-d₆) δ −59.90, |
| | | | | −116.96, −128.03 |
| F114 | 138-142 | | ESIMS m/z 702 | ¹H NMR (400 MHz, DMSO-d₆) δ 10.88 |
| | | | ([M − H]⁻) | (s, 1H), 10.23 (s, 1H), 10.08 (s, 1H), 8.02 |
| | | | | (d, J = 2.4, Hz, 1H), 7.91-7.81 (m, 3H), |
| | | | | 7.64-7.56 (m, 2H), 7.53 (d, J = 8.8, Hz, |
| | | | | 1H), 7.4 (d, J = 8.4 Hz, 1H), 3.71-3.63 |
| | | | | (m, 3H), 3.51 (d, J = 8.4 Hz, 1H), 2.38- |
| | | | | 2.32 (m, 3H); |
| | | | | ¹⁹F NMR (376 MHz, DMSO-d₆) δ −59.90, |
| | | | | −61.44, −116.96, −127.64 |
| F115 | 275-279 | | ESIMS m/z 664 | ¹H NMR (400 MHz, DMSO-d₆) δ 10.88 |
| | | | ([M − H]⁻) | (s, 1H), 10.08 (s, 1H), 9.54 (s, 1H), 8.02 |
| | | | | (d, J = 2.4 Hz, 1H), 7.87-7.81 (m, 3H), |
| | | | | 7.64-7.56 (m, 2H), 7.53 (d, J = 10.8 Hz, |
| | | | | 1H), 7.4 (d, J = 8.0 Hz, 1H), 4.08 (br s, |
| | | | | 2H), 3.69 (d, J = 8.4 Hz, 1H), 3.50 (d, J = |
| | | | | 8.4 Hz, 1H), 3.40 (s, 3H), 2.37 (d, J = 2.4 |
| | | | | Hz, 3H); |
| | | | | ¹⁹F NMR (376 MHz, DMSO-d₆) δ −59.89, |
| | | | | −59.92, −116.98-−117.03, −127.92 |
| F116 | 197-201 | | ESIMS m/z 732 | ¹H NMR (400 MHz, DMSO-d₆) δ 10.88 |
| | | | ([M − H]⁻) | (s, 1H), 10.08 (s, 1H), 9.75 (s, 1H), 8.02 |
| | | | | (d, J = 2.4 Hz, 1H), 7.92-7.81 (m, 3H), |
| | | | | 7.63-7.51 (m, 3H), 7.54 (d, J = 8.4 Hz, |
| | | | | 1H), 4.39 (br s, 2H), 4.30-4.23 (m, 2H), |
| | | | | 3.69 (d, J = 8.4 Hz, 1H), 3.50 (d, J = 8.4 |
| | | | | Hz, 1H), 2.38 (d, J = 2.4 Hz, 3H); |
| | | | | ¹⁹F NMR (376 MHz, DMSO-d₆) δ −59.93, |
| | | | | −73.03, −116.98, −128.1 |
| F117 | 165-169 | | ESIMS m/z 622 | ¹H NMR (400 MHz, DMSO-d₆) δ 10.89 |
| | | | ([M + H]⁺) | (s, 1H), 10.43 (s, 1H), 9.67 (s, 1H), 8.32 |
| | | | | (d, J = 2.4 Hz, 1H), 7.87-7.79 (m, 3H), |
| | | | | 7.77 (dd, J = 1.6, 14.0 Hz, 1H), 7.63- |
| | | | | 7.53 (m, 2H), 7.53 (d, J = 8.8 Hz, 1H), |
| | | | | 7.41 (dd, J = 1.6, 8.4 Hz, 1H), 3.68 (d, J = |
| | | | | 8.4 Hz, 1H), 3.50 (d, J = 8.4 Hz, 1H), |
| | | | | 2.10 (s, 3H); |
| | | | | ¹⁹F NMR (376 MHz, DMSO-d₆) δ −59.93, |
| | | | | −110.77, −116.99 |
| F118 | 103-107 | | ESIMS m/z 652 | ¹H NMR (400 MHz, DMSO-d₆) δ 10.89 |
| | | | ([M + H]⁺) | (s, 1H), 10.26 (s, 1H), 9.71 (s, 1H), 8.32 |
| | | | | (d, J = 2.4 Hz, 1H), 7.87-7.79 (m, 4H), |
| | | | | 7.64-7.56 (m, 3H), 7.54 (d, J = 8.8 Hz, |
| | | | | 1H), 4.06 (s, 2H), 3.68 (d, J = 8.4 Hz, |
| | | | | 1H), 3.51 (d, J = 8.8 Hz, 1H), 3.39 (s, 3H); |
| | | | | ¹⁹F NMR (376 MHz, DMSO-d₆) δ −59.93, |
| | | | | −110.98, −73.95, −116.99 |
| F119 | 140-144 | | ESIMS m/z 720 | ¹H NMR (400 MHz, DMSO-d₆) δ 10.89 |
| | | | ([M + H]⁺) | (s, 1H), 10.40 (s, 1H), 9.72 (s, 1H), 8.31 |
| | | | | (d, J = 2.4 Hz, 1H), 7.87-7.75 (m, 4H), |
| | | | | 7.63-7.48 (m, 4H), 4.35 (s, 2H), 4.29- |
| | | | | 4.22 (m, 2H), 3.68 (d, J = 8.4 Hz, 1H), |
| | | | | 3.51 (d, J = 8.4 Hz, 1H); |
| | | | | ¹⁹F NMR (376 MHz, DMSO-d₆) δ −59.92, |
| | | | | −72.96, −110.77, −116.97 |
| F120 | 138-142 | | ESIMS m/z 704 | ¹H NMR (400 MHz, DMSO-d₆) δ 10.89 |
| | | | ([M + H]⁺) | (s, 1H), 10.57 (s, 1H), 9.70 (s, 1H), 8.32 |
| | | | | (d, J = 2.4 Hz, 1H), 7.87-7.81 (m, 3H), |
| | | | | 7.77 (dd, J = 2.0, 14.0 Hz, 1H), 7.63- |
| | | | | 7.58 (m, 2H), 7.53 (d, J = 8.8 Hz, 1H), |
| | | | | 7.42 (d, J = 1.6, 8.2 Hz, 1H), 3.68 (d, J = |
| | | | | 8.4 Hz, 1H), 3.51 (d, J = 8.4 Hz, 1H), |
| | | | | 2.70-2.51 (m, 4H); |
| | | | | ¹⁹F NMR (376 MHz, DMSO-d₆) δ −59.93, |
| | | | | −65.08, −110.63, −116.99 |
| F121 | 199-203 | | ESIMS m/z 722 | ¹H NMR (400 MHz, DMSO-d₆) δ 10.89 |
| | | | ([M + H]⁺) | (s, 1H), 10.82 (s, 1H), 9.74 (s, 1H), 8.31 |
| | | | | (d, J = 2.0 Hz, 1H), 7.87-7.81 (m, 3H), |
| | | | | 7.73 (dd, J = 2.0, 13.2 Hz, 1H), 7.64- |
| | | | | 7.51 (m, 3H), 7.42 (d, J = 2.0, 8.4 Hz, |
| | | | | 1H), 4.07 (s, 2H), 3.68 (d, J = 8.8 Hz, |
| | | | | 1H), 3.51 (d, J = 8.8 Hz, 1H); |
| | | | | ¹⁹F NMR (376 MHz, DMSO-d₆) δ −40.82, |
| | | | | −59.93, −110.52, −116.99 |
| C2 | | | ESIMS m/z 608 | |
| | | | ([M − H]⁻) | |

1. A molecule having the following formula 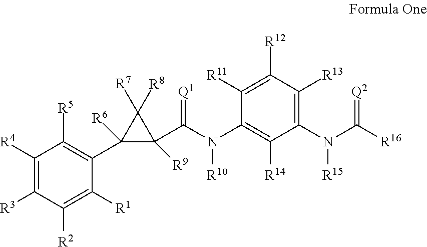
wherein:
(A) R¹ is selected from the group consisting of H, F, Cl, Br, I, and (C₁-C₃)haloalkyl;
(B) R² is selected from the group consisting of H, F, Cl, Br, I, and (C₁-C₃)haloalkyl;
(C) R³ is selected from the group consisting of H, F, Cl, Br, I, and (C₁-C₃)haloalkyl;
(D) R⁴ is selected from the group consisting of H, F, Cl, Br, I, and (C₁-C₃)haloalkyl;
(E) R⁵ is selected from the group consisting of H, F, Cl, Br, I, and (C₁-C₃)haloalkyl;
(F) R⁶ is H;
(G) R⁷ is selected from the group consisting of F, Cl, and Br;
(H) R⁸ is selected from the group consisting of F, Cl, and Br;
(I) R⁹ is H;
(J) Q¹ is selected from the group consisting of O and S;
(K) Q² is selected from the group consisting of O and S;
(L) R¹⁰ is selected from the group consisting of H, (C₁-C₃)alkyl, (C₂-C₃)alkenyl, and (C₂-C₃)alkynyl;
(M) R¹¹ is selected from the group consisting of H, F, Cl, Br, I, (C₁-C₃)alkyl, (C₁-C₃)haloalkyl, and (C₁-C₃)alkoxy;
(N) R¹² is selected from the group consisting of H, F, Cl, Br, I, (C₁-C₃)alkyl, (C₁-C₃)haloalkyl, and (C₁-C₃)alkoxy;
(O) R¹³ is selected from the group consisting of H, F, Cl, Br, I, (C₁-C₃)alkyl, (C₁-C₃)haloalkyl, and (C₁-C₃)alkoxy;
(P) R¹⁴ is selected from the group consisting of H, F, Cl, Br, I, (C₁-C₃)alkyl, (C₁-C₃)haloalkyl, and (C₁-C₃)alkoxy;
(Q) R¹⁵ is selected from the group consisting of H, (C₁-C₃)alkyl, (C₂-C₃)alkenyl, and (C₂-C₃)alkynyl;
(R) R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, NHC(═O)(C₁-C₆)alkyl-S—(C₁-C₆)haloalkyl; and
resolved stereoisomers of the molecules of Formula One.

2. The molecule according to claim 1 wherein said molecule has the following formula

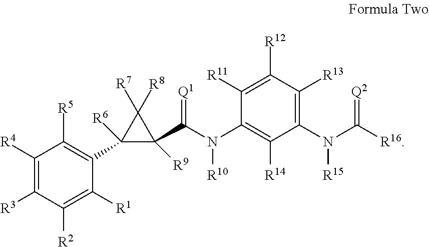

3. The molecule according to claim 1
wherein:
(A) R¹ is selected from the group consisting of H, F, Cl, Br, I, and CF₃;
(B) R² is selected from the group consisting of H, F, Cl, Br, I, and CF₃;
(C) R³ is selected from the group consisting of H, F, Cl, Br, I, and CF₃;
(D) R⁴ is selected from the group consisting of H, F, Cl, Br, I, and CF₃;
(E) R⁵ is selected from the group consisting of H, F, Cl, Br, I, and CF₃;
(F) R⁶ is H;
(G) R⁷ is selected from the group consisting of F, Cl, and Br;
(H) R⁸ is selected from the group consisting of F, Cl, and Br;
(I) R⁹ is H;
(J) Q¹ is selected from the group consisting of O and S;
(K) Q² is selected from the group consisting of O and S;
(L) R¹⁰ is selected from the group consisting of H, (C₁-C₃)alkyl, (C₂-C₃)alkenyl, and (C₂-C₃)alkynyl;
(M) R¹¹ is selected from the group consisting of H, F, Cl, Br, I, (C₁-C₃)alkyl, (C₁-C₃)haloalkyl, and (C₁-C₃)alkoxy;
(N) R¹² is selected from the group consisting of H, F, Cl, Br, I, (C₁-C₃)alkyl, (C₁-C₃)haloalkyl, and (C₁-C₃)alkoxy;
(O) R¹³ is selected from the group consisting of H, F, Cl, Br, I, (C₁-C₃)alkyl, (C₁-C₃)haloalkyl, and (C₁-C₃)alkoxy;
(P) R¹⁴ is selected from the group consisting of H, F, Cl, Br, I, (C₁-C₃)alkyl, (C₁-C₃)haloalkyl, and (C₁-C₃)alkoxy;
(Q) R¹⁵ is selected from the group consisting of H, (C₁-C₃)alkyl, (C₂-C₃)alkenyl, and (C₂-C₃)alkynyl;
(R) R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl, has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, and NHC(═O)(C₁-C₆)alkyl-S—(C₁-C₆)haloalkyl.

4. The molecule according to claim 1
wherein:
(A) R¹ is selected from the group consisting of H, F, and Cl;
(B) R² is selected from the group consisting of H, F, Cl, Br, and CF₃;
(C) R³ is selected from the group consisting of H, F, Cl, Br, and CF₃;
(D) R⁴ is selected from the group consisting of H, F, Cl, Br, and CF₃;
(E) R⁵ is selected from the group consisting of H, F, and Cl;
(F) R⁶ is H;
(G) R⁷ is selected from the group consisting of F and Cl;
(H) R⁸ is selected from the group consisting of F and Cl;
(I) R⁹ is H;
(J) Q¹ is selected from the group consisting of O and S;
(K) Q² is selected from the group consisting of O and S;
(L) R¹⁰ is selected from the group consisting of H and (C₁-C₃)alkyl;
(M) R¹¹ is selected from the group consisting of H, F, and Cl;
(N) R¹² is selected from the group consisting of H, F, and Cl;
(O) R¹³ is selected from the group consisting of H, F, Cl, (C₁-C₃)alkyl, and (C₁-C₃)haloalkyl;
(P) R¹⁴ is selected from the group consisting of H, F, and Cl;
(Q) R¹⁵ is selected from the group consisting of H and (C₁-C₃)alkyl;
(R) R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl, has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, and NHC(═O)(C₁-C₆)alkyl-S—(C₁-C₆)haloalkyl.

5. The molecule according to claim 1 wherein said heterocyclyl is selected from the group consisting of furanyl, isoxazolonyl, isoxazolyl, morpholinyl, substituted pyridinyl, pyrimidinyl, substituted pyrrolidinyl, tetrahydrofuranyl, and substituted thiazolyl, wherein said substituted pyridinyl, substituted pyrrolidinyl, and substituted thiazolyl have one or more substituents selected from the group consisting of H, F, Cl, Br, CN, (C₁-C₆)alkyl, and (C₁-C₆)haloalkyl.

6. The molecule according to claim 1 wherein said substituted phenyl has one or more substituents selected from the group consisting of H, F, Cl, CN, NH₂, NO₂, (C₁-C₆)alkyl, (C₂-C₆)alkynyl, (C₁-C₆)haloalkyl, (C₁-C₆)alkoxy, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, and NHC(═O)(C₁-C₆)alkyl-S—(C₁-C₆)haloalkyl.

7. The molecule according to claim 1
wherein:
(A) R¹ is H;
(B) R² is selected from the group consisting of H, Cl, Br, and CF₃;
(C) R³ is selected from the group consisting of H, F, and Cl;
(D) R⁴ is selected from the group consisting of H, Cl, Br, and CF₃;
(E) R⁵ is H;
(F) R⁶ is H;
(G) R⁷ is Cl;
(H) R⁸ is Cl;
(I) R⁹ is H;
(J) Q¹ is O;
(K) Q² is O;
(L) R¹⁰ is H;
(M) R¹¹ is H;
(N) R¹² is H;
(O) R¹³ is selected from the group consisting of H, F, and Cl;
(P) R¹⁴ is selected from the group consisting of H and F;
(Q) R¹⁵ is selected from the group consisting of H and CH₃;
(R) R¹⁶ is selected from the group consisting of CF₂CF₂CF₃, CF₂CH₂CH₃, CF₂CHF₂, CF₂Cl, CF₂phenyl, CF₃, CH(CH₃)Ophenyl, CH(CH₃)CF₃, CH(CH₃)OCH₂CH₃, CH₂morpholinyl, CH₂CN, CH₂CF₃, CH₂CH(CF₃)CH₃, CH₂CH═CH₂, CH₂CH₂CF₃, CH₂CH₂CH₂CF₃, CH₂CH₂CH₃, CH₂NHC(═O)CH₃, CH₂OCH₂CF₃, CH₂OCH₃, CH₂phenyl, substituted CH₂phenyl, CH₃, cyclopropyl, substituted cyclopropyl, furanyl, isoxazolonyl, phenyl, substituted phenyl, substituted pyridinyl, pyrimidinyl, substituted pyrrolidinyl, tetrahydrofuranyl, and substituted thiazolyl,
wherein said substituted CH₂phenyl, substituted cyclopropyl, substituted phenyl, substituted pyridinyl, substituted pyrrolidinyl, and substituted thiazolyl has one or more substituents selected from the group consisting of C≡CH, CF₃, CH₂CF₃, CH₃, Cl, CN, F, H, NH₂, NHC(═O)CF₃, NHC(═O)CH₃, NHC(═O)CHF₂, NHC(═O)CH₂CF₃, NHC(═O)CH₂CH₂CF₃, NHC(═O)CH₂OCH₃, NHC(═O)CH₂SCF₃, NHC(═O)CH₂OCH₂CF₃, NO₂, and OCH₃.

8. The molecule according to claim 1 wherein the molecule is selected from the following molecules:

| Cmpd. No. | Structure |
|---|---|
| F1 | 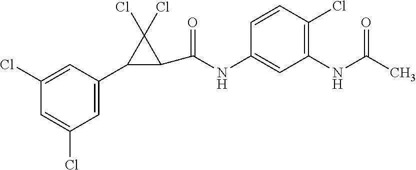 [SMILES: CC(=O)Nc1cc(NC(=O)C2C(c3cc(Cl)cc(Cl)c3)C2(Cl)Cl)ccc1Cl] |
| F2 | 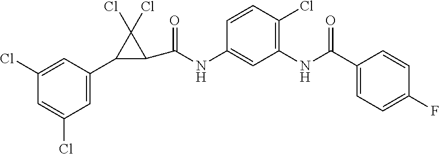 [SMILES: O=C(Nc1cc(NC(=O)C2C(c3cc(Cl)cc(Cl)c3)C2(Cl)Cl)ccc1Cl)c1ccc(F)cc1] |
| F3 | 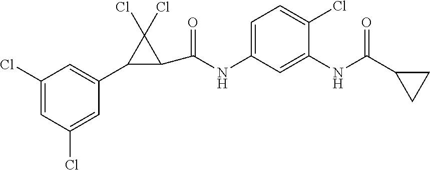 [SMILES: O=C(Nc1cc(NC(=O)C2C(c3cc(Cl)cc(Cl)c3)C2(Cl)Cl)ccc1Cl)C1CC1] |
| F4 | 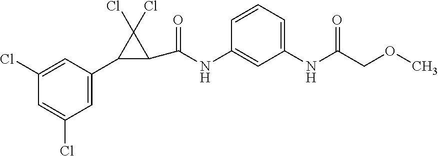 [SMILES: COCC(=O)Nc1cccc(NC(=O)C2C(c3cc(Cl)cc(Cl)c3)C2(Cl)Cl)c1] |
| F5 | 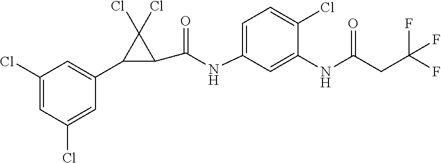 [SMILES: O=C(CC(F)(F)F)Nc1cc(NC(=O)C2C(c3cc(Cl)cc(Cl)c3)C2(Cl)Cl)ccc1Cl] |
| F6 | 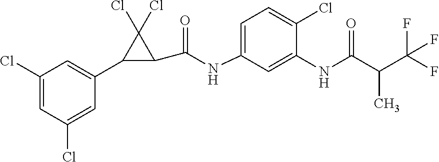 [SMILES: CC(C(=O)Nc1cc(NC(=O)C2C(c3cc(Cl)cc(Cl)c3)C2(Cl)Cl)ccc1Cl)C(F)(F)F] |
| F7 | 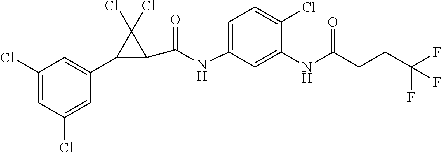 [SMILES: O=C(CCC(F)(F)F)Nc1cc(NC(=O)C2C(c3cc(Cl)cc(Cl)c3)C2(Cl)Cl)ccc1Cl] |
| F8 | 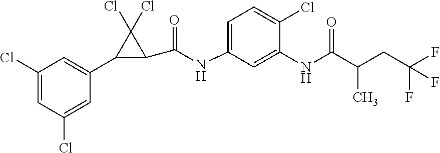 [SMILES: CC(CC(F)(F)F)C(=O)Nc1cc(NC(=O)C2C(c3cc(Cl)cc(Cl)c3)C2(Cl)Cl)ccc1Cl] |
| F9 | 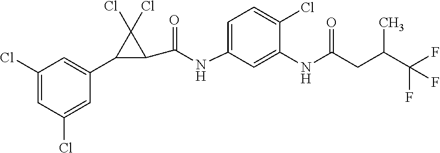 [SMILES: CC(CC(=O)Nc1cc(NC(=O)C2C(c3cc(Cl)cc(Cl)c3)C2(Cl)Cl)ccc1Cl)C(F)(F)F] |
| F10 | 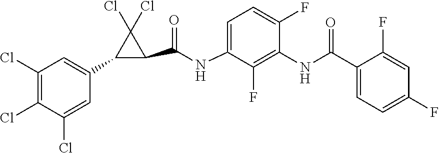 [SMILES: O=C(Nc1c(F)ccc(NC(=O)[C@H]2[C@H](c3cc(Cl)c(Cl)c(Cl)c3)C2(Cl)Cl)c1F)c1ccc(F)cc1F] |
| F11 | 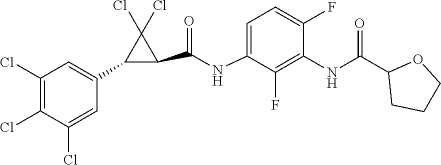 [SMILES: O=C(Nc1c(F)ccc(NC(=O)[C@H]2[C@H](c3cc(Cl)c(Cl)c(Cl)c3)C2(Cl)Cl)c1F)C1CCCO1] |
| F12 | 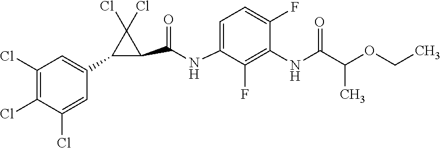 [SMILES: CCOC(C)C(=O)Nc1c(F)ccc(NC(=O)[C@H]2[C@H](c3cc(Cl)c(Cl)c(Cl)c3)C2(Cl)Cl)c1F] |
| F13 | 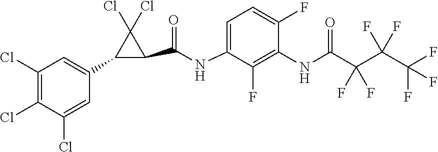 [SMILES: O=C(Nc1ccc(F)c(NC(=O)C(F)(F)C(F)(F)C(F)(F)F)c1F)[C@H]1[C@H](c2cc(Cl)c(Cl)c(Cl)c2)C1(Cl)Cl] |
| F14 | 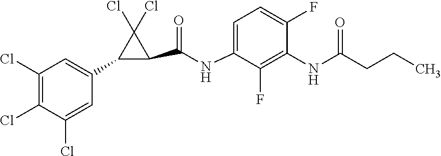 [SMILES: CCCC(=O)Nc1c(F)ccc(NC(=O)[C@H]2[C@H](c3cc(Cl)c(Cl)c(Cl)c3)C2(Cl)Cl)c1F] |
| F15 | 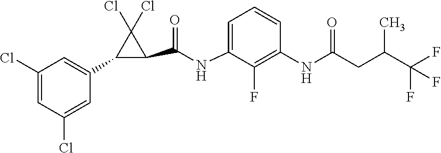 [SMILES: CC(CC(=O)Nc1cccc(NC(=O)[C@H]2[C@H](c3cc(Cl)cc(Cl)c3)C2(Cl)Cl)c1F)C(F)(F)F] |
| F18 | 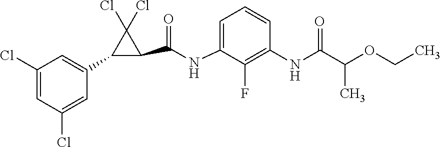 [SMILES: CCOC(C)C(=O)Nc1cccc(NC(=O)[C@H]2[C@H](c3cc(Cl)cc(Cl)c3)C2(Cl)Cl)c1F] |
| F20 | 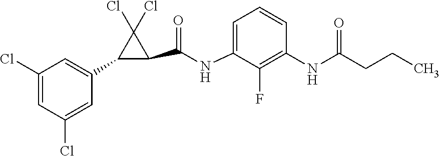 [SMILES: CCCC(=O)Nc1cccc(NC(=O)[C@H]2[C@H](c3cc(Cl)cc(Cl)c3)C2(Cl)Cl)c1F] |
| F24 | 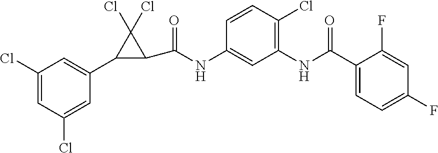 [SMILES: O=C(Nc1cc(NC(=O)C2C(c3cc(Cl)cc(Cl)c3)C2(Cl)Cl)ccc1Cl)c1ccc(F)cc1F] |
| F25 | 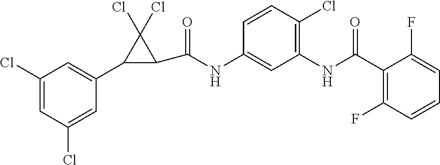 [SMILES: O=C(Nc1cc(NC(=O)C2C(c3cc(Cl)cc(Cl)c3)C2(Cl)Cl)ccc1Cl)c1c(F)cccc1F] |
| F26 | 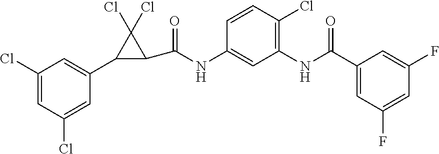 [SMILES: O=C(Nc1cc(NC(=O)C2C(c3cc(Cl)cc(Cl)c3)C2(Cl)Cl)ccc1Cl)c1cc(F)cc(F)c1] |
| F27 | 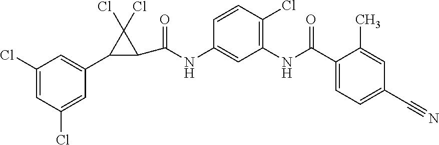 [SMILES: Cc1cc(C#N)ccc1C(=O)Nc1cc(NC(=O)C2C(c3cc(Cl)cc(Cl)c3)C2(Cl)Cl)ccc1Cl] |
| F30 | 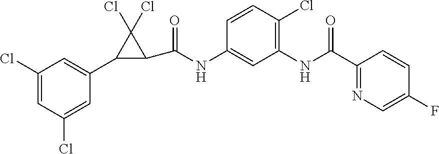 [SMILES: O=C(Nc1cc(NC(=O)C2C(c3cc(Cl)cc(Cl)c3)C2(Cl)Cl)ccc1Cl)c1ccc(F)cn1] |
| F32 | 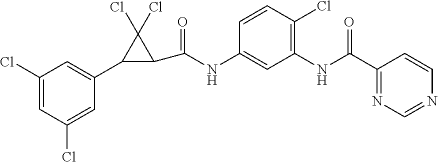 [SMILES: O=C(Nc1cc(NC(=O)C2C(c3cc(Cl)cc(Cl)c3)C2(Cl)Cl)ccc1Cl)c1ccncn1] |
| F34 | 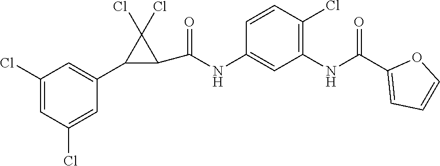 [SMILES: O=C(Nc1cc(NC(=O)C2C(c3cc(Cl)cc(Cl)c3)C2(Cl)Cl)ccc1Cl)c1ccco1] |
| F36 | 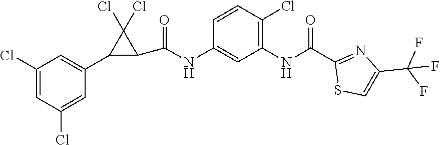 [SMILES: O=C(Nc1cc(NC(=O)C2C(c3cc(Cl)cc(Cl)c3)C2(Cl)Cl)ccc1Cl)c1nc(C(F)(F)F)cs1] |
| F37 | 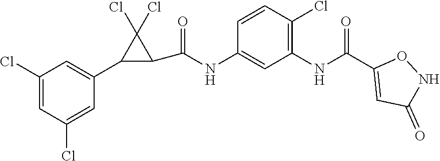 [SMILES: O=C(Nc1cc(NC(=O)C2C(c3cc(Cl)cc(Cl)c3)C2(Cl)Cl)ccc1Cl)c1cc(=O)[nH]o1] |
| F38 | 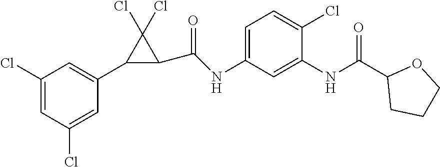 [SMILES: O=C(Nc1cc(NC(=O)C2C(c3cc(Cl)cc(Cl)c3)C2(Cl)Cl)ccc1Cl)C1CCCO1] |
| F39 | 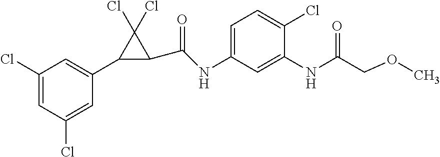 [SMILES: COCC(=O)Nc1cc(NC(=O)C2C(c3cc(Cl)cc(Cl)c3)C2(Cl)Cl)ccc1Cl] |
| F40 | 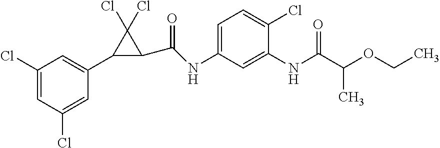 [SMILES: CCOC(C)C(=O)Nc1cc(NC(=O)C2C(c3cc(Cl)cc(Cl)c3)C2(Cl)Cl)ccc1Cl] |
| F41 | 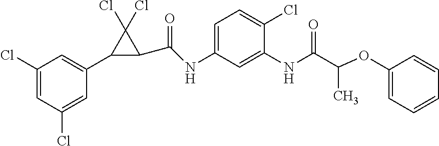 [SMILES: CC(Oc1ccccc1)C(=O)Nc1cc(NC(=O)C2C(c3cc(Cl)cc(Cl)c3)C2(Cl)Cl)ccc1Cl] |
| F42 | 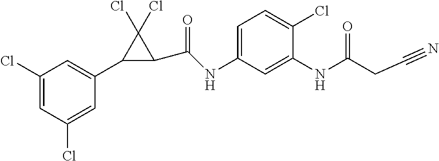 [SMILES: N#CCC(=O)Nc1cc(NC(=O)C2C(c3cc(Cl)cc(Cl)c3)C2(Cl)Cl)ccc1Cl] |
| F43 | 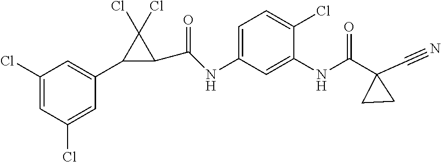 [SMILES: N#CC1(C(=O)Nc2cc(NC(=O)C3C(c4cc(Cl)cc(Cl)c4)C3(Cl)Cl)ccc2Cl)CC1] |
| F44 | 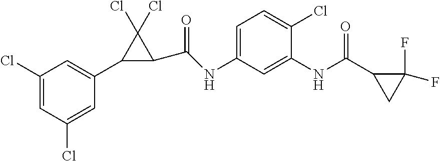 [SMILES: O=C(Nc1cc(NC(=O)C2C(c3cc(Cl)cc(Cl)c3)C2(Cl)Cl)ccc1Cl)C1CC1(F)F] |
| F47 | 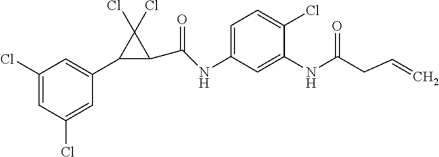 [SMILES: C=CCC(=O)Nc1cc(NC(=O)C2C(c3cc(Cl)cc(Cl)c3)C2(Cl)Cl)ccc1Cl] |
| F48 | 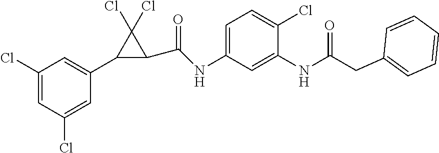 [SMILES: O=C(Cc1ccccc1)Nc1cc(NC(=O)C2C(c3cc(Cl)cc(Cl)c3)C2(Cl)Cl)ccc1Cl] |
| F49 | 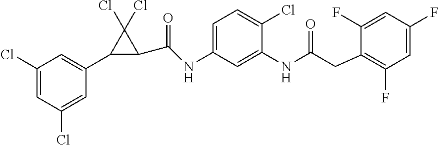 [SMILES: O=C(Cc1c(F)cc(F)cc1F)Nc1cc(NC(=O)C2C(c3cc(Cl)cc(Cl)c3)C2(Cl)Cl)ccc1Cl] |
| F50 | 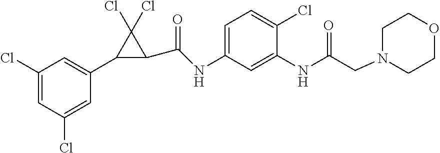 [SMILES: O=C(CN1CCOCC1)Nc1cc(NC(=O)C2C(c3cc(Cl)cc(Cl)c3)C2(Cl)Cl)ccc1Cl] |
| F51 | 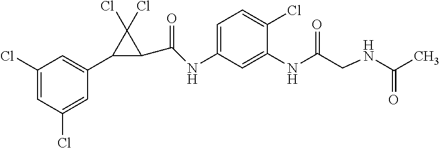 [SMILES: CC(=O)NCC(=O)Nc1cc(NC(=O)C2C(c3cc(Cl)cc(Cl)c3)C2(Cl)Cl)ccc1Cl] |
| F52 | 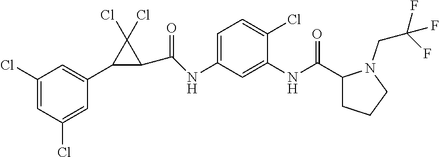 [SMILES: O=C(Nc1cc(NC(=O)C2C(c3cc(Cl)cc(Cl)c3)C2(Cl)Cl)ccc1Cl)C1CCCN1CC(F)(F)F] |
| F53 | 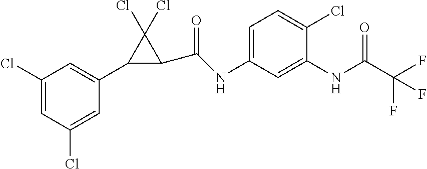 [SMILES: O=C(Nc1ccc(Cl)c(NC(=O)C(F)(F)F)c1)C1C(c2cc(Cl)cc(Cl)c2)C1(Cl)Cl] |
| F54 | 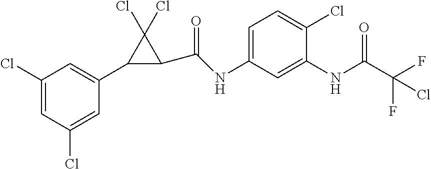 [SMILES: O=C(Nc1ccc(Cl)c(NC(=O)C(F)(F)Cl)c1)C1C(c2cc(Cl)cc(Cl)c2)C1(Cl)Cl] |
| F55 | 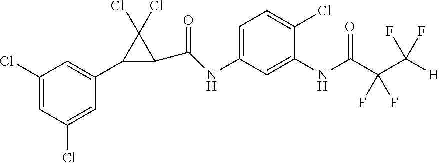 [SMILES: O=C(Nc1ccc(Cl)c(NC(=O)C(F)(F)C(F)F)c1)C1C(c2cc(Cl)cc(Cl)c2)C1(Cl)Cl] |
| F56 | 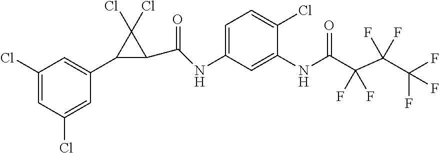 [SMILES: O=C(Nc1ccc(Cl)c(NC(=O)C(F)(F)C(F)(F)C(F)(F)F)c1)C1C(c2cc(Cl)cc(Cl)c2)C1(Cl)Cl] |
| F57 | 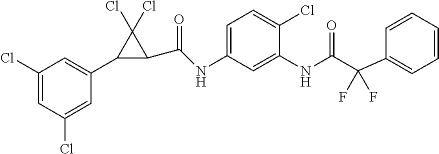 [SMILES: O=C(Nc1ccc(Cl)c(NC(=O)C(F)(F)c2ccccc2)c1)C1C(c2cc(Cl)cc(Cl)c2)C1(Cl)Cl] |
| F58 | 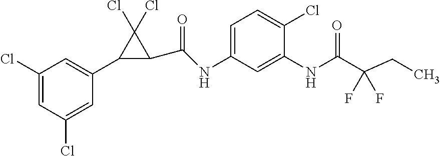 [SMILES: CCC(F)(F)C(=O)Nc1cc(NC(=O)C2C(c3cc(Cl)cc(Cl)c3)C2(Cl)Cl)ccc1Cl] |
| F60 | 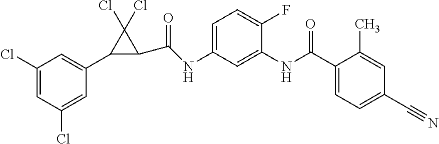 [SMILES: Cc1cc(C#N)ccc1C(=O)Nc1cc(NC(=O)C2C(c3cc(Cl)cc(Cl)c3)C2(Cl)Cl)ccc1F] |
| F61 | 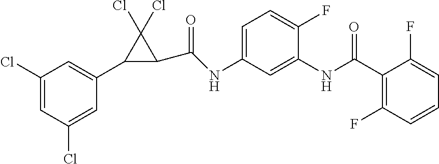 [SMILES: O=C(Nc1cc(NC(=O)C2C(c3cc(Cl)cc(Cl)c3)C2(Cl)Cl)ccc1F)c1c(F)cccc1F] |
| F62 | 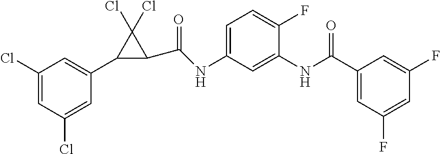 [SMILES: O=C(Nc1cc(NC(=O)C2C(c3cc(Cl)cc(Cl)c3)C2(Cl)Cl)ccc1F)c1cc(F)cc(F)c1] |
| F63 | 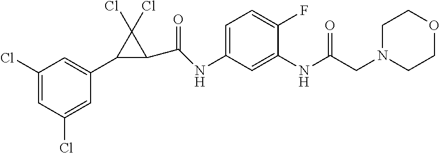 [SMILES: O=C(CN1CCOCC1)Nc1cc(NC(=O)C2C(c3cc(Cl)cc(Cl)c3)C2(Cl)Cl)ccc1F] |
| F64 | 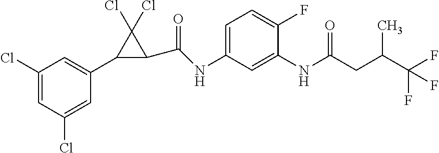 [SMILES: CC(CC(=O)Nc1cc(NC(=O)C2C(c3cc(Cl)cc(Cl)c3)C2(Cl)Cl)ccc1F)C(F)(F)F] |
| F65 | 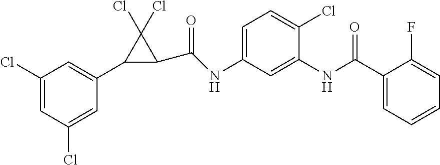 [SMILES: O=C(Nc1cc(NC(=O)C2C(c3cc(Cl)cc(Cl)c3)C2(Cl)Cl)ccc1Cl)c1ccccc1F] |
| F66 | 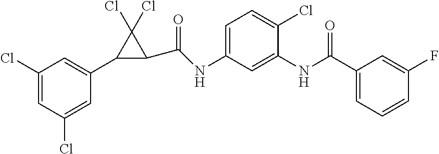 [SMILES: O=C(Nc1cc(NC(=O)C2C(c3cc(Cl)cc(Cl)c3)C2(Cl)Cl)ccc1Cl)c1cccc(F)c1] |
| F67 | 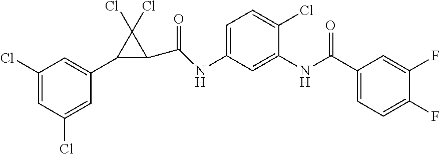 [SMILES: O=C(Nc1cc(NC(=O)C2C(c3cc(Cl)cc(Cl)c3)C2(Cl)Cl)ccc1Cl)c1ccc(F)c(F)c1] |
| F68 | 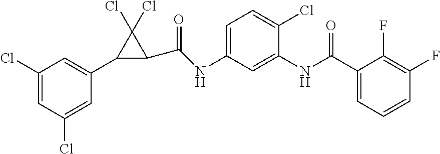 [SMILES: O=C(Nc1cc(NC(=O)C2C(c3cc(Cl)cc(Cl)c3)C2(Cl)Cl)ccc1Cl)c1cccc(F)c1F] |
| F69 | 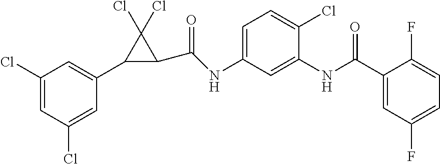 [SMILES: O=C(Nc1cc(NC(=O)C2C(c3cc(Cl)cc(Cl)c3)C2(Cl)Cl)ccc1Cl)c1cc(F)ccc1F] |
| F70 | 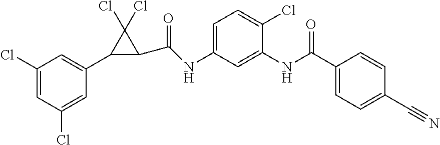 [SMILES: N#Cc1ccc(C(=O)Nc2cc(NC(=O)C3C(c4cc(Cl)cc(Cl)c4)C3(Cl)Cl)ccc2Cl)cc1] |
| F71 | 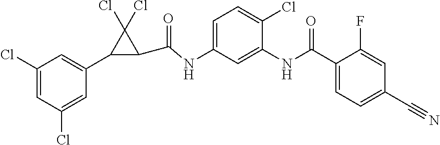 [SMILES: N#Cc1ccc(C(=O)Nc2cc(NC(=O)C3C(c4cc(Cl)cc(Cl)c4)C3(Cl)Cl)ccc2Cl)c(F)c1] |
| F72 | 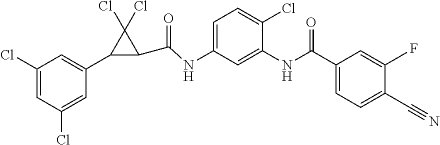 [SMILES: N#Cc1ccc(C(=O)Nc2cc(NC(=O)C3C(c4cc(Cl)cc(Cl)c4)C3(Cl)Cl)ccc2Cl)cc1F] |
| F73 | 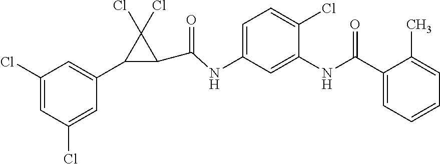 [SMILES: Cc1ccccc1C(=O)Nc1cc(NC(=O)C2C(c3cc(Cl)cc(Cl)c3)C2(Cl)Cl)ccc1Cl] |
| F74 | 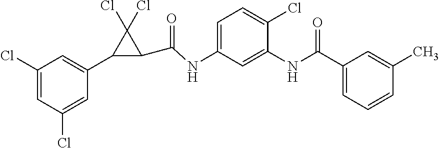 [SMILES: Cc1cccc(C(=O)Nc2cc(NC(=O)C3C(c4cc(Cl)cc(Cl)c4)C3(Cl)Cl)ccc2Cl)c1] |
| F75 | 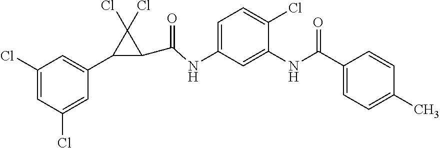 [SMILES: Cc1ccc(C(=O)Nc2cc(NC(=O)C3C(c4cc(Cl)cc(Cl)c4)C3(Cl)Cl)ccc2Cl)cc1] |
| F77 | 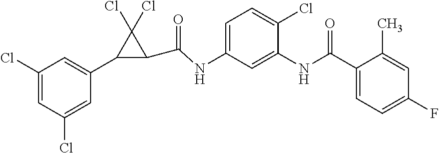 [SMILES: Cc1cc(F)ccc1C(=O)Nc1cc(NC(=O)C2C(c3cc(Cl)cc(Cl)c3)C2(Cl)Cl)ccc1Cl] |
| F78 | 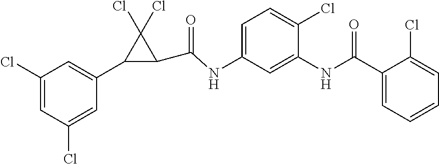 [SMILES: O=C(Nc1cc(NC(=O)C2C(c3cc(Cl)cc(Cl)c3)C2(Cl)Cl)ccc1Cl)c1ccccc1Cl] |
| F79 | 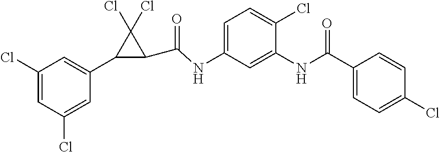 [SMILES: O=C(Nc1cc(NC(=O)C2C(c3cc(Cl)cc(Cl)c3)C2(Cl)Cl)ccc1Cl)c1ccc(Cl)cc1] |
| F81 | 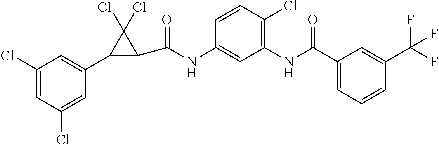 [SMILES: O=C(Nc1cc(NC(=O)C2C(c3cc(Cl)cc(Cl)c3)C2(Cl)Cl)ccc1Cl)c1cccc(C(F)(F)F)c1] |
| F82 | 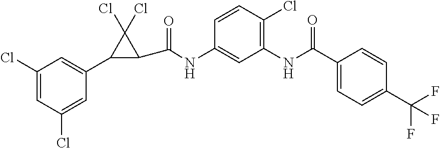 [SMILES: O=C(Nc1cc(NC(=O)C2C(c3cc(Cl)cc(Cl)c3)C2(Cl)Cl)ccc1Cl)c1ccc(C(F)(F)F)cc1] |
| F83 | 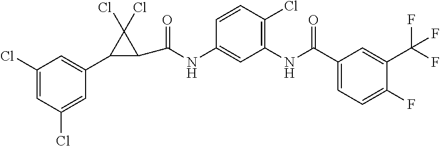 [SMILES: O=C(Nc1cc(NC(=O)C2C(c3cc(Cl)cc(Cl)c3)C2(Cl)Cl)ccc1Cl)c1ccc(F)c(C(F)(F)F)c1] |
| F84 | 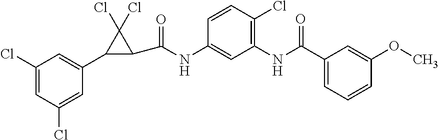 [SMILES: COc1cccc(C(=O)Nc2cc(NC(=O)C3C(c4cc(Cl)cc(Cl)c4)C3(Cl)Cl)ccc2Cl)c1] |
| F85 | 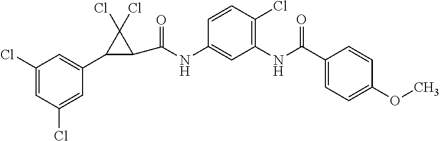 [SMILES: COc1ccc(C(=O)Nc2cc(NC(=O)C3C(c4cc(Cl)cc(Cl)c4)C3(Cl)Cl)ccc2Cl)cc1] |
| F87 | 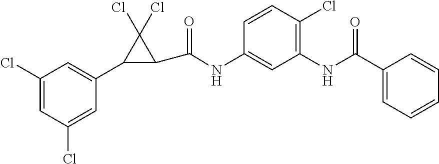 [SMILES: O=C(Nc1cc(NC(=O)C2C(c3cc(Cl)cc(Cl)c3)C2(Cl)Cl)ccc1Cl)c1ccccc1] |
| F88 | 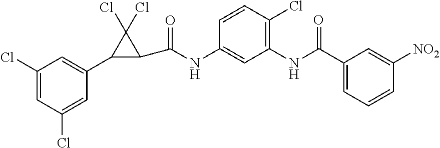 [SMILES: O=C(Nc1cc(NC(=O)C2C(c3cc(Cl)cc(Cl)c3)C2(Cl)Cl)ccc1Cl)c1cccc([N+](=O)[O-])c1] |
| F89 | 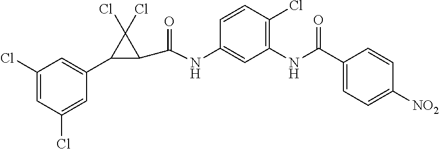 [SMILES: O=C(Nc1cc(NC(=O)C2C(c3cc(Cl)cc(Cl)c3)C2(Cl)Cl)ccc1Cl)c1ccc([N+](=O)[O-])cc1] |
| F90 | 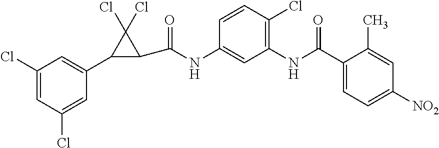 [SMILES: Cc1cc([N+](=O)[O-])ccc1C(=O)Nc1cc(NC(=O)C2C(c3cc(Cl)cc(Cl)c3)C2(Cl)Cl)ccc1Cl] |
| F91 | 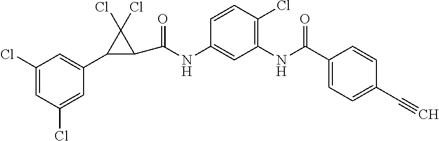 [SMILES: C#Cc1ccc(C(=O)Nc2cc(NC(=O)C3C(c4cc(Cl)cc(Cl)c4)C3(Cl)Cl)ccc2Cl)cc1] |
| F92 | 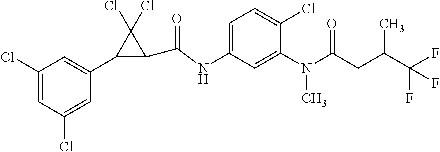 [SMILES: CC(CC(=O)N(C)c1cc(NC(=O)C2C(c3cc(Cl)cc(Cl)c3)C2(Cl)Cl)ccc1Cl)C(F)(F)F] |
| F93 | 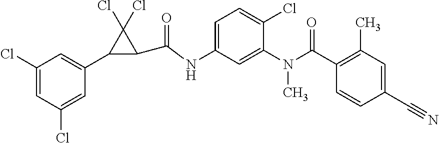 [SMILES: Cc1cc(C#N)ccc1C(=O)N(C)c1cc(NC(=O)C2C(c3cc(Cl)cc(Cl)c3)C2(Cl)Cl)ccc1Cl] |
| F94 | 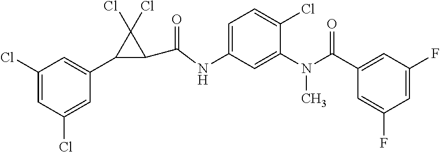 [SMILES: CN(C(=O)c1cc(F)cc(F)c1)c1cc(NC(=O)C2C(c3cc(Cl)cc(Cl)c3)C2(Cl)Cl)ccc1Cl] |
| F95 | 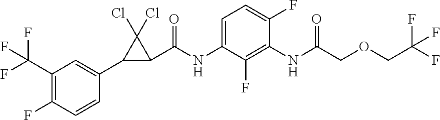 [SMILES: O=C(COCC(F)(F)F)Nc1c(F)ccc(NC(=O)C2C(c3ccc(F)c(C(F)(F)F)c3)C2(Cl)Cl)c1F] |
| F96 | 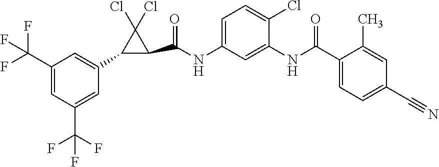 [SMILES: Cc1cc(C#N)ccc1C(=O)Nc1cc(NC(=O)[C@H]2[C@H](c3cc(C(F)(F)F)cc(C(F)(F)F)c3)C2(Cl)Cl)ccc1Cl] |
| F97 | 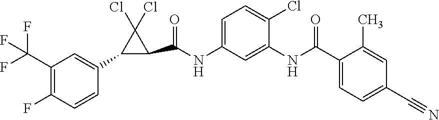 [SMILES: Cc1cc(C#N)ccc1C(=O)Nc1cc(NC(=O)[C@H]2[C@H](c3ccc(F)c(C(F)(F)F)c3)C2(Cl)Cl)ccc1Cl] |
| F98 | 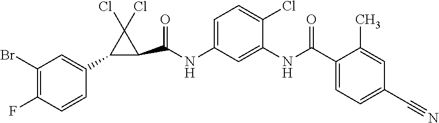 [SMILES: Cc1cc(C#N)ccc1C(=O)Nc1cc(NC(=O)[C@H]2[C@H](c3ccc(F)c(Br)c3)C2(Cl)Cl)ccc1Cl] |
| F99 | 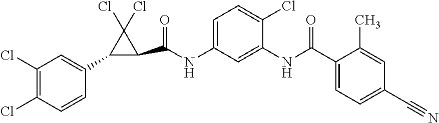 [SMILES: Cc1cc(C#N)ccc1C(=O)Nc1cc(NC(=O)[C@H]2[C@H](c3ccc(Cl)c(Cl)c3)C2(Cl)Cl)ccc1Cl] |
| F100 | 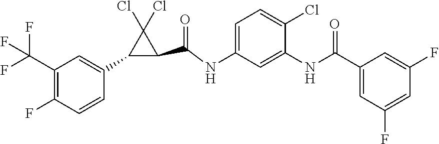 [SMILES: O=C(Nc1cc(NC(=O)[C@H]2[C@H](c3ccc(F)c(C(F)(F)F)c3)C2(Cl)Cl)ccc1Cl)c1cc(F)cc(F)c1] |
| F102 | 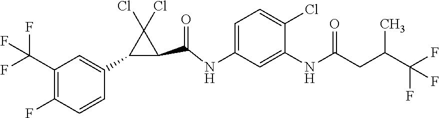 [SMILES: CC(CC(=O)Nc1cc(NC(=O)[C@H]2[C@H](c3ccc(F)c(C(F)(F)F)c3)C2(Cl)Cl)ccc1Cl)C(F)(F)F] |
| F103 | 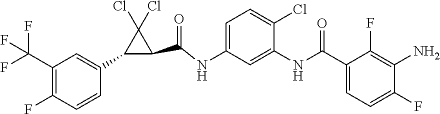 [SMILES: Nc1c(F)ccc(C(=O)Nc2cc(NC(=O)[C@H]3[C@H](c4ccc(F)c(C(F)(F)F)c4)C3(Cl)Cl)ccc2Cl)c1F] |
| F104 | 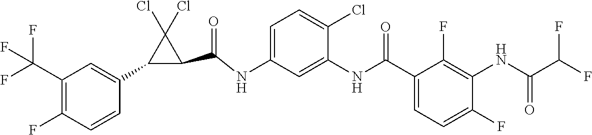 [SMILES: O=C(Nc1cc(NC(=O)[C@H]2[C@H](c3ccc(F)c(C(F)(F)F)c3)C2(Cl)Cl)ccc1Cl)c1ccc(F)c(NC(=O)C(F)F)c1F] |
| F105 | 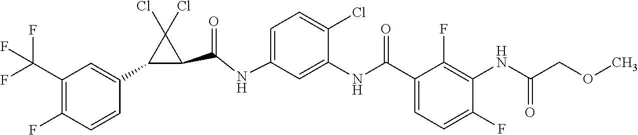 [SMILES: COCC(=O)Nc1c(F)ccc(C(=O)Nc2cc(NC(=O)[C@H]3[C@H](c4ccc(F)c(C(F)(F)F)c4)C3(Cl)Cl)ccc2Cl)c1F] |
| F106 | 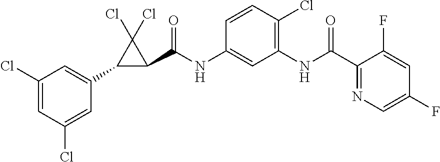 [SMILES: O=C(Nc1cc(NC(=O)[C@H]2[C@H](c3cc(Cl)cc(Cl)c3)C2(Cl)Cl)ccc1Cl)c1ncc(F)cc1F] |
| F109 | 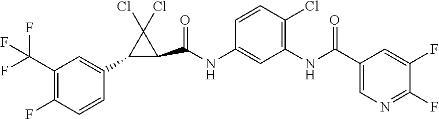 [SMILES: O=C(Nc1cc(NC(=O)[C@H]2[C@H](c3ccc(F)c(C(F)(F)F)c3)C2(Cl)Cl)ccc1Cl)c1cnc(F)c(F)c1] |
| F110 | 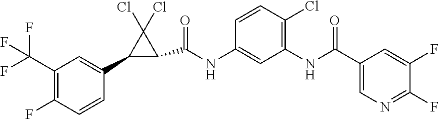 [SMILES: O=C(Nc1cc(NC(=O)[C@@H]2[C@@H](c3ccc(F)c(C(F)(F)F)c3)C2(Cl)Cl)ccc1Cl)c1cnc(F)c(F)c1] |
| F111 | 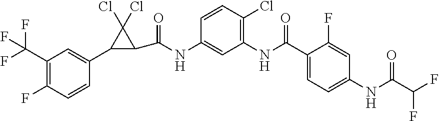 [SMILES: O=C(Nc1cc(NC(=O)C2C(c3ccc(F)c(C(F)(F)F)c3)C2(Cl)Cl)ccc1Cl)c1ccc(NC(=O)C(F)F)cc1F] |
| F112 | 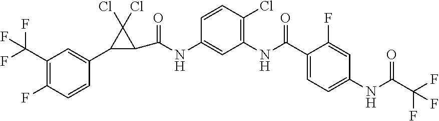 [SMILES: O=C(Nc1cc(NC(=O)C2C(c3ccc(F)c(C(F)(F)F)c3)C2(Cl)Cl)ccc1Cl)c1ccc(NC(=O)C(F)(F)F)cc1F] |
| F113 | 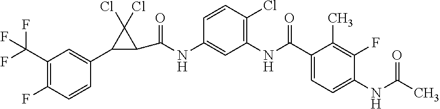 [SMILES: CC(=O)Nc1ccc(C(=O)Nc2cc(NC(=O)C3C(c4ccc(F)c(C(F)(F)F)c4)C3(Cl)Cl)ccc2Cl)c(C)c1F] |
| F114 | 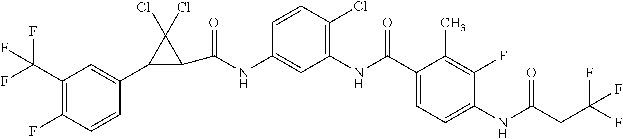 [SMILES: Cc1c(C(=O)Nc2cc(NC(=O)C3C(c4ccc(F)c(C(F)(F)F)c4)C3(Cl)Cl)ccc2Cl)ccc(NC(=O)CC(F)(F)F)c1F] |
| F115 | 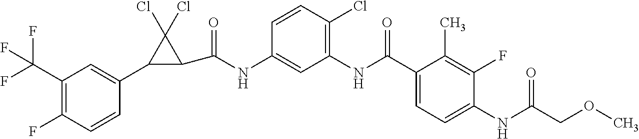 [SMILES: COCC(=O)Nc1ccc(C(=O)Nc2cc(NC(=O)C3C(c4ccc(F)c(C(F)(F)F)c4)C3(Cl)Cl)ccc2Cl)c(C)c1F] |
| F116 | 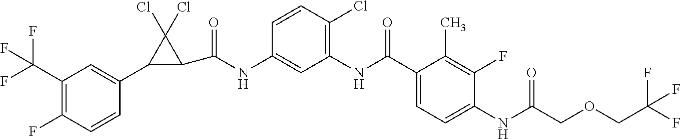 [SMILES: Cc1c(C(=O)Nc2cc(NC(=O)C3C(c4ccc(F)c(C(F)(F)F)c4)C3(Cl)Cl)ccc2Cl)ccc(NC(=O)COCC(F)(F)F)c1F] |
| F117 | 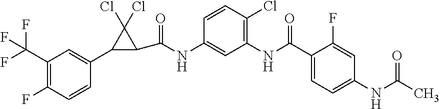 [SMILES: CC(=O)Nc1ccc(C(=O)Nc2cc(NC(=O)C3C(c4ccc(F)c(C(F)(F)F)c4)C3(Cl)Cl)ccc2Cl)c(F)c1] |
| F118 | 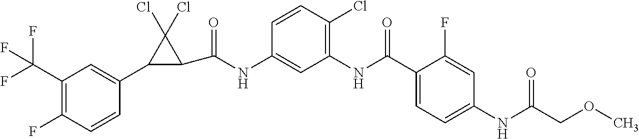 [SMILES: COCC(=O)Nc1ccc(C(=O)Nc2cc(NC(=O)C3C(c4ccc(F)c(C(F)(F)F)c4)C3(Cl)Cl)ccc2Cl)c(F)c1] |
| F119 | 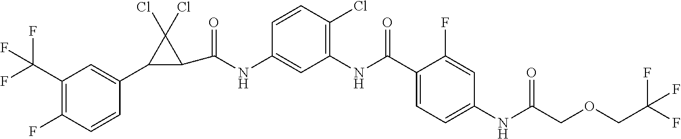 [SMILES: O=C(COCC(F)(F)F)Nc1ccc(C(=O)Nc2cc(NC(=O)C3C(c4ccc(F)c(C(F)(F)F)c4)C3(Cl)Cl)ccc2Cl)c(F)c1] |
| F120 | 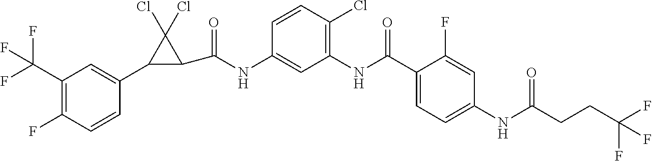 [SMILES: O=C(CCC(F)(F)F)Nc1ccc(C(=O)Nc2cc(NC(=O)C3C(c4ccc(F)c(C(F)(F)F)c4)C3(Cl)Cl)ccc2Cl)c(F)c1] |
| F121 | 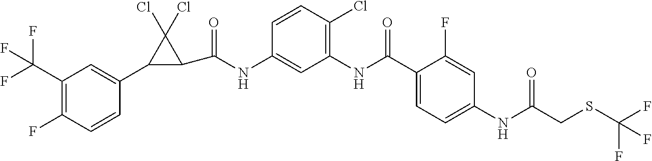 [SMILES: O=C(CSC(F)(F)F)Nc1ccc(C(=O)Nc2cc(NC(=O)C3C(c4ccc(F)c(C(F)(F)F)c4)C3(Cl)Cl)ccc2Cl)c(F)c1] |

9. A pesticidal composition comprising a molecule according to claim 1, further comprising a carrier.

10. A process to control a pest said process comprising applying to a locus, a pesticidally effective amount of a molecule according to claim 1.

11. The process according to claim 10 wherein said locus is a plant, seed, soil, material, or environment, in which a pest is growing, may grow, or may traverse.

12. A process to control a pest said process comprising applying to a locus, a pesticidally effective amount of a composition according to claim 9.

13. The process according to claim 12, wherein said locus is a plant, seed, soil, material, or environment, in which a pest is growing, may grow, or may traverse.

14. The molecule according to claim 2
wherein:
(A) R¹ is selected from the group consisting of H, F, Cl, Br, I, and CF₃;
(B) R² is selected from the group consisting of H, F, Cl, Br, I, and CF₃;
(C) R³ is selected from the group consisting of H, F, Cl, Br, I, and CF₃;
(D) R⁴ is selected from the group consisting of H, F, Cl, Br, I, and CF₃;
(E) R⁵ is selected from the group consisting of H, F, Cl, Br, I, and CF₃;
(F) R⁶ is H;
(G) R⁷ is selected from the group consisting of F, Cl, and Br;
(H) R⁸ is selected from the group consisting of F, Cl, and Br;
(I) R⁹ is H;
(J) Q¹ is selected from the group consisting of O and S;
(K) Q² is selected from the group consisting of O and S;
(L) R¹⁰ is selected from the group consisting of H, (C₁-C₃)alkyl, (C₂-C₃)alkenyl, and (C₂-C₃)alkynyl;
(M) R¹¹ is selected from the group consisting of H, F, Cl, Br, I, (C₁-C₃)alkyl, (C₁-C₃)haloalkyl, and (C₁-C₃)alkoxy;
(N) R¹² is selected from the group consisting of H, F, Cl, Br, I, (C₁-C₃)alkyl, (C₁-C₃)haloalkyl, and (C₁-C₃)alkoxy;
(O) R¹³ is selected from the group consisting of H, F, Cl, Br, I, (C₁-C₃)alkyl, (C₁-C₃)haloalkyl, and (C₁-C₃)alkoxy;
(P) R¹⁴ is selected from the group consisting of H, F, Cl, Br, I, (C₁-C₃)alkyl, (C₁-C₃)haloalkyl, and (C₁-C₃)alkoxy;
(Q) R¹⁵ is selected from the group consisting of H, (C₁-C₃)alkyl, (C₂-C₃)alkenyl, and (C₂-C₃)alkynyl;
(R) R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl, has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, and NHC(═O)(C₁-C₆)alkyl-S—(C₁-C₆)haloalkyl.

15. The molecule according to claim 2
wherein:
(A) R¹ is selected from the group consisting of H, F, and Cl;
(B) R² is selected from the group consisting of H, F, Cl, Br, and CF₃;
(C) R³ is selected from the group consisting of H, F, Cl, Br, and CF₃;
(D) R⁴ is selected from the group consisting of H, F, Cl, Br, and CF₃;
(E) R⁵ is selected from the group consisting of H, F, and Cl;
(F) R⁶ is H;
(G) R⁷ is selected from the group consisting of F and Cl;
(H) R⁸ is selected from the group consisting of F and Cl;
(I) R⁹ is H;
(J) Q¹ is selected from the group consisting of O and S;
(K) Q² is selected from the group consisting of O and S;
(L) R¹⁰ is selected from the group consisting of H and (C₁-C₃)alkyl;
(M) R¹¹ is selected from the group consisting of H, F, and Cl;
(N) R¹² is selected from the group consisting of H, F, and Cl;
(O) R¹³ is selected from the group consisting of H, F, Cl, (C₁-C₃)alkyl, and (C₁-C₃)haloalkyl;
(P) R¹⁴ is selected from the group consisting of H, F, and Cl;
(Q) R¹⁵ is selected from the group consisting of H and (C₁-C₃)alkyl;
(R) R¹⁶ is selected from the group consisting of (C₁-C₆)alkyl, (C₂-C₆)alkenyl, (C₂-C₆)alkynyl, (C₁-C₆)haloalkyl, (C₃-C₆)cycloalkyl, (C₁-C₆)alkyl(C₃-C₆)cycloalkyl, (C₁-C₆)alkylphenyl, (C₁-C₆)haloalkylphenyl, (C₁-C₆)alkylheterocyclyl, (C₁-C₆)haloalkylheterocyclyl, (C₁-C₆)alkyl-O—(C₁-C₆)alkyl, (C₁-C₆)alkyl-O—(C₁-C₆)haloalkyl, (C₁-C₆)alkyl-S—(C₁-C₆)alkyl, (C₁-C₆)alkyl-S—(C₁-C₆)haloalkyl, (C₁-C₆)alkyl-C(═O)NH—(C₁-C₆)haloalkyl, (C₁-C₆)alkyl-NHC(═O)—(C₁-C₆)alkyl, phenyl, substituted phenyl, heterocyclyl, substituted heterocyclyl, and (C₁-C₆)alkyl-O-phenyl,
wherein said alkyl, alkenyl, alkynyl, cycloalkyl, haloalkyl, phenyl, substituted phenyl, heterocyclyl, and substituted heterocyclyl, has one or more substituents selected from the group consisting of H, F, Cl, Br, I, CN, NH₂, NO₂, (C₁-C₆)alkyl, (C₂-C₆)alkenyl, (C₂-C₆)alkynyl, (C₁-C₆)haloalkyl, (C₁-C₆)alkoxy, NHC(═O)O(C₁-C₆)alkyl, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, NHC(═O)(C₁-C₆)alkyl-S—(C₁-C₆)alkyl, and NHC(═O)(C₁-C₆)alkyl-S—(C₁-C₆)haloalkyl.

16. The molecule according to claim 2 wherein said heterocyclyl is selected from the group consisting of furanyl, isoxazolonyl, isoxazolyl, morpholinyl, substituted pyridinyl, pyrimidinyl, substituted pyrrolidinyl, tetrahydrofuranyl, and substituted thiazolyl, wherein said substituted pyridinyl, substituted pyrrolidinyl, and substituted thiazolyl have one or more substituents selected from the group consisting of H, F, Cl, Br, CN, (C₁-C₆)alkyl, and (C₁-C₆)haloalkyl.

17. The molecule according to claim 2 wherein said substituted phenyl has one or more substituents selected from the group consisting of H, F, Cl, CN, NH₂, NO₂, (C₁-C₆)alkyl, (C₂-C₆)alkynyl, (C₁-C₆)haloalkyl, (C₁-C₆)alkoxy, NHC(═O)(C₁-C₆)alkyl, NHC(═O)(C₁-C₆)haloalkyl, NHC(═O)(C₁-C₆)alkyl-O—(C₁-C₆)alkyl, NHC(═O)(C₁-C₆)alkyl-O—(C₁-C₆)haloalkyl, and NHC(═O)(C₁-C₆)alkyl-S—(C₁-C₆)haloalkyl.

18. The molecule according to claim 2
wherein:
(A) R¹ is H;
(B) R² is selected from the group consisting of H, Cl, Br, and CF₃;
(C) R³ is selected from the group consisting of H, F, and Cl;
(D) R⁴ is selected from the group consisting of H, Cl, Br, and CF₃;
(E) R⁵ is H;
(F) R⁶ is H;
(G) R⁷ is Cl;
(H) R⁸ is Cl;
(I) R⁹ is H;
(J) Q¹ is O;
(K) Q² is O;
(L) R¹⁰ is H;
(M) R¹¹ is H;
(N) R¹² is H;
(O) R¹³ is selected from the group consisting of H, F, and Cl;
(P) R¹⁴ is selected from the group consisting of H and F;
(Q) R¹⁵ is selected from the group consisting of H and CH₃;
(R) R¹⁶ is selected from the group consisting of CF₂CF₂CF₃, CF₂CH₂CH₃, CF₂CHF₂, CF₂Cl, CF₂phenyl, CF₃, CH(CH₃)Ophenyl, CH(CH₃)CF₃, CH(CH₃)OCH₂CH₃, CH₂morpholinyl, CH₂CN, CH₂CF₃, CH₂CH(CF₃)CH₃, CH₂CH═CH₂, CH₂CH₂CF₃, CH₂CH₂CH₂CF₃, CH₂CH₂CH₃, CH₂NHC(═O)CH₃, CH₂OCH₂CF₃, CH₂OCH₃, CH₂phenyl, substituted CH₂phenyl, CH₃, cyclopropyl, substituted cyclopropyl, furanyl, isoxazolonyl, phenyl, substituted phenyl, substituted pyridinyl, pyrimidinyl, substituted pyrrolidinyl, tetrahydrofuranyl, and substituted thiazolyl,
wherein said substituted CH₂phenyl, substituted cyclopropyl, substituted phenyl, substituted pyridinyl, substituted pyrrolidinyl, and substituted thiazolyl has one or more substituents selected from the group consisting of C≡CH, CF₃, CH₂CF₃, CH₃, Cl, CN, F, H, NH₂, NHC(═O)CF₃, NHC(═O)CH₃, NHC(═O)CHF₂, NHC(═O)CH₂CF₃, NHC(═O)CH₂CH₂CF₃, NHC(═O)CH₂OCH₃, NHC(═O)CH₂SCF₃, NHC(═O)CH₂OCH₂CF₃, NO₂, and OCH₃.
